SEQUENCE LISTING (68 sequences)

SEQ ID NO: 1 - DNA, 1541 bp, Homo sapiens
  source 1..1541: mol_type = genomic DNA
ccgcagaatcctatcagtttccccctttcgtgctgtgtgcatcgagcaggaaggggcttggcaggttttacctgccctctttcctttctgaaaagtctgggcctcctcaccccgaaaggagtcacctccttgcagttccccagttgcgaaaagaggaggaagttggctgggccgggggccgcggggggcaccctccgcagatggcgggacccccctgccggccatggcaaaaacgaggcttgtctctcccaccgcccccaaccttagtccttggcacattgttgaaagtaattgaataaaatcggaaattcgagaaggcgttcgttcggattggtgagattttgaggggagaaagaagcggggacttcgccggcaccagcggcgccccctcctcggccaccgttaacccccattccagagggcactgccccgccacccagcctaggtccccctgcgagagcctcgcgggcccgcgcagcctccgcgactcgaacagatcttcagtccttggaggaatgcctgtttctctaacaataaaaaattaaagaagcgctcataaatgccaagtcctctcgcactatgcggagtacagaggacaacgaccacagccatccctgaaccccgcccacggcacagcgccggagccggggtctggggcgccgcttcctggggggtcccgactctcagccgcccccgcttcacccgggccgccaaggggctgggggaggcggcgctcggggtaaccgggggagactcagggcgctgggggcacttggggaactcatgggggctcaaaggaactaggagatcgggacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcaggtgagcccgccggccccggactgcccggccaggaacctggcgcggggagggaccgcgagacccagagcggttgcccggccgcgtgggtctcggggaaccggggggctggaccaacacacgtccttgggccggggggcgggggccgccttctggagcgggcgtttctgcggccgagctccggagctggaatggggcggccggggaagtggacgcgatggcaccgcccggggtgcgagtggggccgggcgcgcgcgggaggggaaaaaggcgcgggcgagccgccagcgcgaggtttgtggtgtcgccgatgtcccttcggggtactctagcgcagccgcctggctacttgacccactgccaccaaacgttttaaattcaccg

SEQ ID NO: 2 - DNA, 1418 bp, Homo sapiens
  source 1..1418: mol_type = genomic DNA
ttcgtggccacctctctcctgttagcctctgaccaaggtggcactggatggtttctgcctgaccttggtgccccgtggcagcgactgtgggtcatgaaagacattcactacgagcctgcttctggagtccatcagaaaacgggatgcaacttgcctaaaatgaggagaggaggatgcttttaagaaaaagaagaaggaggattcactaccagctctgaagggtggaaaagagatgattcatccggattgtggagagggtggaatcttgtttaggagagcgttggttgtggcaggcagggtgtaactatgaatcagtgaagacaattcacatcctgggatgaaaagaaggccatgggctcacaggagattatccactggcctctccacatccgcttgcagtaaggagtgtgggactctcccaagcttcagcgctgaactgcaatgcagtgacgtcgcttagctgggccagtaaccgagggagttgaattttctgtcattttaaaataatgtgtcttttaagaaacactttgaaattaaaaccacagcccacaattataatgcactgttgcagcacttatcaaaacagatatgctaactgagccatcagtgccagcctgacagtgaggccaccaagccatccacaaagcctacacgaaagtctgtgctcacagtggcttttctccatgaagagggcattcctaacctcttcctttcacgtaggaggaagcaaggtcctttgtaaaattttaactcggggtgcctcaaatgtaaacttaaccactggtaacaacagtttcactgctacatgccacgtctgtgaaaattcattcaagacattaaggaaagtggctcagcagagagactagacatcttatcctcacggttctcctgtacttggcctctcagcctttgagcaaggttggcccaagctagtatcggccccagtggtacagccaaaacttgagactgcaaatggatgcagctgttgaacgctgagtaacttctgcagagtcaggaagacccaaggaagctctgcagaggatgcaggggtacggtcagaacccctgagtgcctttcagctaacgaggactttatgacactccccagcacagcaaatttttatgatgtgtttaaagattgggtgaattactcaggtgaacaagctactttttatcagagaacacctaaaaacacgttcaagagggtttgggaactatacatttaatcctatgacaaactaagttggttctgtcttcacctgttttggtgaggttgtgtaagagttggtgtttgctcaggaagagatttaagcatgcttgcttacccagactcagagaagtctccctgttctgtcctagctagtgattcctgtgttgtgtgcattcgtcttttccagagcaaaccgcccagagtagaag

SEQ ID NO: 3 - DNA, 739 bp, Homo sapiens
  source 1..739: mol_type = genomic DNA
acagaggacaacgaccacagccatccctgaaccccgcccacggcacagcgccggagccggggtctggggcgccgcttcctggggggtcccgactctcagccgcccccgcttcacccgggccgccaaggggctgggggaggcggcgctcggggtaaccgggggagactcagggcgctgggggcacttggggaactcatgggggctcaaaggaactaggagatcgggacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcaggtgagcccgccggccccggactgcccggccaggaacctggcgcggggagggaccgcgagacccagagcggttgcccggccgcgtgggtctcggggaaccggggggctggaccaacacacgtccttgggccggggggcgggggccgccttctggagcgggcgtttctg

SEQ ID NO: 4 - DNA, 569 bp, Homo sapiens
  source 1..569: mol_type = genomic DNA
acagaggacaacgaccacagccatccctgaaccccgcccacggcacagcgccggagccggggtctggggcgccgcttcctggggggtcccgactctcagccgcccccgcttcacccgggccgccaaggggctgggggaggcggcgctcggggtaaccgggggagactcagggcgctgggggcacttggggaactcatgggggctcaaaggaactaggagatcgggacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacg

SEQ ID NO: 5 - DNA, 366 bp, Homo sapiens
  source 1..366: mol_type = genomic DNA
acagaggacaacgaccacagccatccctgaaccccgcccacggcacagcgccggagccggggtctggggcgccgcttcctggggggtcccgactctcagccgcccccgcttcacccgggccgccaaggggctgggggaggcggcgctcggggtaaccgggggagactcagggcgctgggggcacttggggaactcatgggggctcaaaggaactaggagatcgggacctcgaaggggacttggggggttcggggctttcgggggcggtcgggggttcgcggacccgggaagctctgaggacccagaggccgggcgcgctccgcccgcggcgccgccccctccgtaactttcccagtctccgaggga

SEQ ID NO: 6 - DNA, 100 bp, Homo sapiens
  source 1..100: mol_type = genomic DNA
agaggcggggtgtggggtgcggttaaaaggcgccacggcgggagacaggtgttgcggccccgcagcgcccgcgcgctcctctccccgactcggagcccct

SEQ ID NO: 7 - DNA, 225 bp, Homo sapiens
  source 1..225: mol_type = genomic DNA
gctcaaaggaactaggagatcgggacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccc

SEQ ID NO: 8 - DNA, 539 bp, Homo sapiens
  source 1..539: mol_type = genomic DNA
ggctcaaaggaactaggagatcgggacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcaggtgagcccgccggccccggactgcccggccaggaacctggcgcggggagggaccgcgagacccagagcggttgcccggccgcgtgggtctcggggaaccggggggctggaccaacacacgtccttgggccggggggcgggggccgccttctggagcgggcgtttctg

SEQ ID NO: 9 - DNA, 426 bp, Homo sapiens
  source 1..426: mol_type = genomic DNA
acagaggacaacgaccacagccatccctgaaccccgcccacggcacagcgccggagccggggtctggggcgccgcttcctggggggtcccgactctcagccgcccccgcttcacccgggccgccaaggggctgggggaggcggcgctcggggtaaccgggggagactcagggcgctgggggcacttggggaactcatgggggctcaaaggaactaggagatcgggacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccc

SEQ ID NO: 10 - DNA, 128 bp, Homo sapiens
  source 1..128: mol_type = genomic DNA
cgggaagctctgaggacccagaggccgggcgcgctccgcccgcggcgccgccccctccgtaactttcccagtctccgagggaagaggcggggtgtggggtgcggttaaaaggcgccacggcgggagac

SEQ ID NO: 11 - DNA, 128 bp, Homo sapiens
  source 1..128: mol_type = genomic DNA
aagctctgaggacccagaggccgggcgcgctccgcccgcggcgccgccccctccgtaactttcccagtctccgagggaagaggcggggtgtggggtgcggttaaaaggcgccacggcgggagacaggt

SEQ ID NO: 12 - DNA, 164 bp, Homo sapiens
  source 1..164: mol_type = genomic DNA
agcccgccggccccggactgcccggccaggaacctggcgcggggagggaccgcgagacccagagcggttgcccggccgcgtgggtctcggggaaccggggggctggaccaacacacgtccttgggccggggggcgggggccgccttctggagcgggcgtttctg

SEQ ID NO: 13 - DNA, 733 bp, synthetic construct
  source 1..733: mol_type = other DNA
acagaggacaacgaccacagccatccctgaaccccgcccacggcacagcgccggagccggggtctggggcgccgcttcctggggggtcccgactctcagccgcccccgcttcacccgggccgccaaggggctgggggaggcggcgctcggggtaaccgggggagactcagggcgctgggggcacttggggaactcatgggggctcaaaggaactaggagatcgggacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgagcccgccggccccggactgcccggccaggaacctggcgcggggagggaccgcgagacccagagcggttgcccggccgcgtgggtctcggggaaccggggggctggaccaacacacgtccttgggccggggggcgggggccgccttctggagcgggcgtttctg

SEQ ID NO: 14 - DNA, 530 bp, synthetic construct
  source 1..530: mol_type = other DNA
acagaggacaacgaccacagccatccctgaaccccgcccacggcacagcgccggagccggggtctggggcgccgcttcctggggggtcccgactctcagccgcccccgcttcacccgggccgccaaggggctgggggaggcggcgctcggggtaaccgggggagactcagggcgctgggggcacttggggaactcatgggggctcaaaggaactaggagatcgggacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagcccgccggccccggactgcccggccaggaacctggcgcggggagggaccgcgagacccagagcggttgcccggccgcgtgggtctcggggaaccggggggctggaccaacacacgtccttgggccggggggcgggggccgccttctggagcgggcgtttctg

SEQ ID NO: 15 - DNA, 264 bp, synthetic construct
  source 1..264: mol_type = other DNA
agaggcggggtgtggggtgcggttaaaaggcgccacggcgggagacaggtgttgcggccccgcagcgcccgcgcgctcctctccccgactcggagcccctagcccgccggccccggactgcccggccaggaacctggcgcggggagggaccgcgagacccagagcggttgcccggccgcgtgggtctcggggaaccggggggctggaccaacacacgtccttgggccggggggcgggggccgccttctggagcgggcgtttctg

SEQ ID NO: 16 - DNA, 389 bp, synthetic construct
  source 1..389: mol_type = other DNA
gctcaaaggaactaggagatcgggacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggcccagcccgccggccccggactgcccggccaggaacctggcgcggggagggaccgcgagacccagagcggttgcccggccgcgtgggtctcggggaaccggggggctggaccaacacacgtccttgggccggggggcgggggccgccttctggagcgggcgtttctg

SEQ ID NO: 17 - DNA, 590 bp, synthetic construct
  source 1..590: mol_type = other DNA
acagaggacaacgaccacagccatccctgaaccccgcccacggcacagcgccggagccggggtctggggcgccgcttcctggggggtcccgactctcagccgcccccgcttcacccgggccgccaaggggctgggggaggcggcgctcggggtaaccgggggagactcagggcgctgggggcacttggggaactcatgggggctcaaaggaactaggagatcgggacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggcccagcccgccggccccggactgcccggccaggaacctggcgcggggagggaccgcgagacccagagcggttgcccggccgcgtgggtctcggggaaccggggggctggaccaacacacgtccttgggccggggggcgggggccgccttctggagcgggcgtttctg

SEQ ID NO: 18 - DNA, 292 bp, synthetic construct
  source 1..292: mol_type = other DNA
cgggaagctctgaggacccagaggccgggcgcgctccgcccgcggcgccgccccctccgtaactttcccagtctccgagggaagaggcggggtgtggggtgcggttaaaaggcgccacggcgggagacagcccgccggccccggactgcccggccaggaacctggcgcggggagggaccgcgagacccagagcggttgcccggccgcgtgggtctcggggaaccggggggctggaccaacacacgtccttgggccggggggcgggggccgccttctggagcgggcgtttctg

SEQ ID NO: 19 - DNA, 292 bp, synthetic construct
  source 1..292: mol_type = other DNA
aagctctgaggacccagaggccgggcgcgctccgcccgcggcgccgccccctccgtaactttcccagtctccgagggaagaggcggggtgtggggtgcggttaaaaggcgccacggcgggagacaggtagcccgccggccccggactgcccggccaggaacctggcgcggggagggaccgcgagacccagagcggttgcccggccgcgtgggtctcggggaaccggggggctggaccaacacacgtccttgggccggggggcgggggccgccttctggagcgggcgtttctg

SEQ ID NO: 20 - DNA, 300 bp, Homo sapiens
  source 1..300: mol_type = genomic DNA
tttatgatgtgtttaaagattgggtgaattactcaggtgaacaagctactttttatcagagaacacctaaaaacacgttcaagagggtttgggaactatacatttaatcctatgacaaactaagttggttctgtcttcacctgttttggtgaggttgtgtaagagttggtgtttgctcaggaagagatttaagcatgcttgcttacccagactcagagaagtctccctgttctgtcctagctagtgattcctgtgttgtgtgcattcgtcttttccagagcaaaccgcccagagtagaag

SEQ ID NO: 21 - DNA, 883 bp, Homo sapiens
  source 1..883: mol_type = genomic DNA
aaaaccacagcccacaattataatgcactgttgcagcacttatcaaaacagatatgctaactgagccatcagtgccagcctgacagtgaggccaccaagccatccacaaagcctacacgaaagtctgtgctcacagtggcttttctccatgaagagggcattcctaacctcttcctttcacgtaggaggaagcaaggtcctttgtaaaattttaactcggggtgcctcaaatgtaaacttaaccactggtaacaacagtttcactgctacatgccacgtctgtgaaaattcattcaagacattaaggaaagtggctcagcagagagactagacatcttatcctcacggttctcctgtacttggcctctcagcctttgagcaaggttggcccaagctagtatcggccccagtggtacagccaaaacttgagactgcaaatggatgcagctgttgaacgctgagtaacttctgcagagtcaggaagacccaaggaagctctgcagaggatgcaggggtacggtcagaacccctgagtgcctttcagctaacgaggactttatgacactccccagcacagcaaatttttatgatgtgtttaaagattgggtgaattactcaggtgaacaagctactttttatcagagaacacctaaaaacacgttcaagagggtttgggaactatacatttaatcctatgacaaactaagttggttctgtcttcacctgttttggtgaggttgtgtaagagttggtgtttgctcaggaagagatttaagcatgcttgcttacccagactcagagaagtctccctgttctgtcctagctagtgattcctgtgttgtgtgcattcgtcttttccagagcaaaccgcccagagtagaag

SEQ ID NO: 22 - DNA, 100 bp, Homo sapiens
  source 1..100: mol_type = genomic DNA
gcttacccagactcagagaagtctccctgttctgtcctagctagtgattcctgtgttgtgtgcattcgtcttttccagagcaaaccgcccagagtagaag

SEQ ID NO: 23 - DNA, 535 bp, Homo sapiens
  source 1..535: mol_type = genomic DNA
ttcgtggccacctctctcctgttagcctctgaccaaggtggcactggatggtttctgcctgaccttggtgccccgtggcagcgactgtgggtcatgaaagacattcactacgagcctgcttctggagtccatcagaaaacgggatgcaacttgcctaaaatgaggagaggaggatgcttttaagaaaaagaagaaggaggattcactaccagctctgaagggtggaaaagagatgattcatccggattgtggagagggtggaatcttgtttaggagagcgttggttgtggcaggcagggtgtaactatgaatcagtgaagacaattcacatcctgggatgaaaagaaggccatgggctcacaggagattatccactggcctctccacatccgcttgcagtaaggagtgtgggactctcccaagcttcagcgctgaactgcaatgcagtgacgtcgcttagctgggccagtaaccgagggagttgaattttctgtcattttaaaataatgtgtcttttaagaaacactttgaaatt

SEQ ID NO: 24 - DNA, 299 bp, Homo sapiens
  source 1..299: mol_type = genomic DNA
catgaaagacattcactacgagcctgcttctggagtccatcagaaaacgggatgcaacttgcctaaaatgaggagaggaggatgcttttaagaaaaagaagaaggaggattcactaccagctctgaagggtggaaaagagatgattcatccggattgtggagagggtggaatcttgtttaggagagcgttggttgtggcaggcagggtgtaactatgaatcagtgaagacaattcacatcctgggatgaaaagaaggccatgggctcacaggagattatccactggcctctccacatcc

SEQ ID NO: 25 - DNA, 835 bp, synthetic construct
  source 1..835: mol_type = other DNA
ttcgtggccacctctctcctgttagcctctgaccaaggtggcactggatggtttctgcctgaccttggtgccccgtggcagcgactgtgggtcatgaaagacattcactacgagcctgcttctggagtccatcagaaaacgggatgcaacttgcctaaaatgaggagaggaggatgcttttaagaaaaagaagaaggaggattcactaccagctctgaagggtggaaaagagatgattcatccggattgtggagagggtggaatcttgtttaggagagcgttggttgtggcaggcagggtgtaactatgaatcagtgaagacaattcacatcctgggatgaaaagaaggccatgggctcacaggagattatccactggcctctccacatccgcttgcagtaaggagtgtgggactctcccaagcttcagcgctgaactgcaatgcagtgacgtcgcttagctgggccagtaaccgagggagttgaattttctgtcattttaaaataatgtgtcttttaagaaacactttgaaatttttatgatgtgtttaaagattgggtgaattactcaggtgaacaagctactttttatcagagaacacctaaaaacacgttcaagagggtttgggaactatacatttaatcctatgacaaactaagttggttctgtcttcacctgttttggtgaggttgtgtaagagttggtgtttgctcaggaagagatttaagcatgcttgcttacccagactcagagaagtctccctgttctgtcctagctagtgattcctgtgttgtgtgcattcgtcttttccagagcaaaccgcccagagtagaag

SEQ ID NO: 26 - DNA, 599 bp, synthetic construct
  source 1..599: mol_type = other DNA
catgaaagacattcactacgagcctgcttctggagtccatcagaaaacgggatgcaacttgcctaaaatgaggagaggaggatgcttttaagaaaaagaagaaggaggattcactaccagctctgaagggtggaaaagagatgattcatccggattgtggagagggtggaatcttgtttaggagagcgttggttgtggcaggcagggtgtaactatgaatcagtgaagacaattcacatcctgggatgaaaagaaggccatgggctcacaggagattatccactggcctctccacatcctttatgatgtgtttaaagattgggtgaattactcaggtgaacaagctactttttatcagagaacacctaaaaacacgttcaagagggtttgggaactatacatttaatcctatgacaaactaagttggttctgtcttcacctgttttggtgaggttgtgtaagagttggtgtttgctcaggaagagatttaagcatgcttgcttacccagactcagagaagtctccctgttctgtcctagctagtgattcctgtgttgtgtgcattcgtcttttccagagcaaaccgcccagagtagaag

SEQ ID NO: 27 - DNA, 635 bp, synthetic construct
  source 1..635: mol_type = other DNA
ttcgtggccacctctctcctgttagcctctgaccaaggtggcactggatggtttctgcctgaccttggtgccccgtggcagcgactgtgggtcatgaaagacattcactacgagcctgcttctggagtccatcagaaaacgggatgcaacttgcctaaaatgaggagaggaggatgcttttaagaaaaagaagaaggaggattcactaccagctctgaagggtggaaaagagatgattcatccggattgtggagagggtggaatcttgtttaggagagcgttggttgtggcaggcagggtgtaactatgaatcagtgaagacaattcacatcctgggatgaaaagaaggccatgggctcacaggagattatccactggcctctccacatccgcttgcagtaaggagtgtgggactctcccaagcttcagcgctgaactgcaatgcagtgacgtcgcttagctgggccagtaaccgagggagttgaattttctgtcattttaaaataatgtgtcttttaagaaacactttgaaattgcttacccagactcagagaagtctccctgttctgtcctagctagtgattcctgtgttgtgtgcattcgtcttttccagagcaaaccgcccagagtagaag

SEQ ID NO: 28 - DNA, 399 bp, synthetic construct
  source 1..399: mol_type = other DNA
catgaaagacattcactacgagcctgcttctggagtccatcagaaaacgggatgcaacttgcctaaaatgaggagaggaggatgcttttaagaaaaagaagaaggaggattcactaccagctctgaagggtggaaaagagatgattcatccggattgtggagagggtggaatcttgtttaggagagcgttggttgtggcaggcagggtgtaactatgaatcagtgaagacaattcacatcctgggatgaaaagaaggccatgggctcacaggagattatccactggcctctccacatccgcttacccagactcagagaagtctccctgttctgtcctagctagtgattcctgtgttgtgtgcattcgtcttttccagagcaaaccgcccagagtagaag

SEQ ID NO: 29 - DNA, 2959 bp, synthetic construct
  source 1..2959: mol_type = other DNA
ccgcagaatcctatcagtttccccctttcgtgctgtgtgcatcgagcaggaaggggcttggcaggttttacctgccctctttcctttctgaaaagtctgggcctcctcaccccgaaaggagtcacctccttgcagttccccagttgcgaaaagaggaggaagttggctgggccgggggccgcggggggcaccctccgcagatggcgggacccccctgccggccatggcaaaaacgaggcttgtctctcccaccgcccccaaccttagtccttggcacattgttgaaagtaattgaataaaatcggaaattcgagaaggcgttcgttcggattggtgagattttgaggggagaaagaagcggggacttcgccggcaccagcggcgccccctcctcggccaccgttaacccccattccagagggcactgccccgccacccagcctaggtccccctgcgagagcctcgcgggcccgcgcagcctccgcgactcgaacagatcttcagtccttggaggaatgcctgtttctctaacaataaaaaattaaagaagcgctcataaatgccaagtcctctcgcactatgcggagtacagaggacaacgaccacagccatccctgaaccccgcccacggcacagcgccggagccggggtctggggcgccgcttcctggggggtcccgactctcagccgcccccgcttcacccgggccgccaaggggctgggggaggcggcgctcggggtaaccgggggagactcagggcgctgggggcacttggggaactcatgggggctcaaaggaactaggagatcgggacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcaggtgagcccgccggccccggactgcccggccaggaacctggcgcggggagggaccgcgagacccagagcggttgcccggccgcgtgggtctcggggaaccggggggctggaccaacacacgtccttgggccggggggcgggggccgccttctggagcgggcgtttctgcggccgagctccggagctggaatggggcggccggggaagtggacgcgatggcaccgcccggggtgcgagtggggccgggcgcgcgcgggaggggaaaaaggcgcgggcgagccgccagcgcgaggtttgtggtgtcgccgatgtcccttcggggtactctagcgcagccgcctggctacttgacccactgccaccaaacgttttaaattcaccgttcgtggccacctctctcctgttagcctctgaccaaggtggcactggatggtttctgcctgaccttggtgccccgtggcagcgactgtgggtcatgaaagacattcactacgagcctgcttctggagtccatcagaaaacgggatgcaacttgcctaaaatgaggagaggaggatgcttttaagaaaaagaagaaggaggattcactaccagctctgaagggtggaaaagagatgattcatccggattgtggagagggtggaatcttgtttaggagagcgttggttgtggcaggcagggtgtaactatgaatcagtgaagacaattcacatcctgggatgaaaagaaggccatgggctcacaggagattatccactggcctctccacatccgcttgcagtaaggagtgtgggactctcccaagcttcagcgctgaactgcaatgcagtgacgtcgcttagctgggccagtaaccgagggagttgaattttctgtcattttaaaataatgtgtcttttaagaaacactttgaaattaaaaccacagcccacaattataatgcactgttgcagcacttatcaaaacagatatgctaactgagccatcagtgccagcctgacagtgaggccaccaagccatccacaaagcctacacgaaagtctgtgctcacagtggcttttctccatgaagagggcattcctaacctcttcctttcacgtaggaggaagcaaggtcctttgtaaaattttaactcggggtgcctcaaatgtaaacttaaccactggtaacaacagtttcactgctacatgccacgtctgtgaaaattcattcaagacattaaggaaagtggctcagcagagagactagacatcttatcctcacggttctcctgtacttggcctctcagcctttgagcaaggttggcccaagctagtatcggccccagtggtacagccaaaacttgagactgcaaatggatgcagctgttgaacgctgagtaacttctgcagagtcaggaagacccaaggaagctctgcagaggatgcaggggtacggtcagaacccctgagtgcctttcagctaacgaggactttatgacactccccagcacagcaaatttttatgatgtgtttaaagattgggtgaattactcaggtgaacaagctactttttatcagagaacacctaaaaacacgttcaagagggtttgggaactatacatttaatcctatgacaaactaagttggttctgtcttcacctgttttggtgaggttgtgtaagagttggtgtttgctcaggaagagatttaagcatgcttgcttacccagactcagagaagtctccctgttctgtcctagctagtgattcctgtgttgtgtgcattcgtcttttccagagcaaaccgcccagagtagaag

SEQ ID NO: 30 - DNA, 1039 bp, synthetic construct
  source 1..1039: mol_type = other DNA
acagaggacaacgaccacagccatccctgaaccccgcccacggcacagcgccggagccggggtctggggcgccgcttcctggggggtcccgactctcagccgcccccgcttcacccgggccgccaaggggctgggggaggcggcgctcggggtaaccgggggagactcagggcgctgggggcacttggggaactcatgggggctcaaaggaactaggagatcgggacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcaggtgagcccgccggccccggactgcccggccaggaacctggcgcggggagggaccgcgagacccagagcggttgcccggccgcgtgggtctcggggaaccggggggctggaccaacacacgtccttgggccggggggcgggggccgccttctggagcgggcgtttctgtttatgatgtgtttaaagattgggtgaattactcaggtgaacaagctactttttatcagagaacacctaaaaacacgttcaagagggtttgggaactatacatttaatcctatgacaaactaagttggttctgtcttcacctgttttggtgaggttgtgtaagagttggtgtttgctcaggaagagatttaagcatgcttgcttacccagactcagagaagtctccctgttctgtcctagctagtgattcctgtgttgtgtgcattcgtcttttccagagcaaaccgcccagagtagaag

SEQ ID NO: 31 - DNA, 2157 bp, synthetic construct
  source 1..2157: mol_type = other DNA
acagaggacaacgaccacagccatccctgaaccccgcccacggcacagcgccggagccggggtctggggcgccgcttcctggggggtcccgactctcagccgcccccgcttcacccgggccgccaaggggctgggggaggcggcgctcggggtaaccgggggagactcagggcgctgggggcacttggggaactcatgggggctcaaaggaactaggagatcgggacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcaggtgagcccgccggccccggactgcccggccaggaacctggcgcggggagggaccgcgagacccagagcggttgcccggccgcgtgggtctcggggaaccggggggctggaccaacacacgtccttgggccggggggcgggggccgccttctggagcgggcgtttctgttcgtggccacctctctcctgttagcctctgaccaaggtggcactggatggtttctgcctgaccttggtgccccgtggcagcgactgtgggtcatgaaagacattcactacgagcctgcttctggagtccatcagaaaacgggatgcaacttgcctaaaatgaggagaggaggatgcttttaagaaaaagaagaaggaggattcactaccagctctgaagggtggaaaagagatgattcatccggattgtggagagggtggaatcttgtttaggagagcgttggttgtggcaggcagggtgtaactatgaatcagtgaagacaattcacatcctgggatgaaaagaaggccatgggctcacaggagattatccactggcctctccacatccgcttgcagtaaggagtgtgggactctcccaagcttcagcgctgaactgcaatgcagtgacgtcgcttagctgggccagtaaccgagggagttgaattttctgtcattttaaaataatgtgtcttttaagaaacactttgaaattaaaaccacagcccacaattataatgcactgttgcagcacttatcaaaacagatatgctaactgagccatcagtgccagcctgacagtgaggccaccaagccatccacaaagcctacacgaaagtctgtgctcacagtggcttttctccatgaagagggcattcctaacctcttcctttcacgtaggaggaagcaaggtcctttgtaaaattttaactcggggtgcctcaaatgtaaacttaaccactggtaacaacagtttcactgctacatgccacgtctgtgaaaattcattcaagacattaaggaaagtggctcagcagagagactagacatcttatcctcacggttctcctgtacttggcctctcagcctttgagcaaggttggcccaagctagtatcggccccagtggtacagccaaaacttgagactgcaaatggatgcagctgttgaacgctgagtaacttctgcagagtcaggaagacccaaggaagctctgcagaggatgcaggggtacggtcagaacccctgagtgcctttcagctaacgaggactttatgacactccccagcacagcaaatttttatgatgtgtttaaagattgggtgaattactcaggtgaacaagctactttttatcagagaacacctaaaaacacgttcaagagggtttgggaactatacatttaatcctatgacaaactaagttggttctgtcttcacctgttttggtgaggttgtgtaagagttggtgtttgctcaggaagagatttaagcatgcttgcttacccagactcagagaagtctccctgttctgtcctagctagtgattcctgtgttgtgtgcattcgtcttttccagagcaaaccgcccagagtagaag

SEQ ID NO: 32 - DNA, 1622 bp, synthetic construct
  source 1..1622: mol_type = other DNA
acagaggacaacgaccacagccatccctgaaccccgcccacggcacagcgccggagccggggtctggggcgccgcttcctggggggtcccgactctcagccgcccccgcttcacccgggccgccaaggggctgggggaggcggcgctcggggtaaccgggggagactcagggcgctgggggcacttggggaactcatgggggctcaaaggaactaggagatcgggacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcaggtgagcccgccggccccggactgcccggccaggaacctggcgcggggagggaccgcgagacccagagcggttgcccggccgcgtgggtctcggggaaccggggggctggaccaacacacgtccttgggccggggggcgggggccgccttctggagcgggcgtttctgaaaaccacagcccacaattataatgcactgttgcagcacttatcaaaacagatatgctaactgagccatcagtgccagcctgacagtgaggccaccaagccatccacaaagcctacacgaaagtctgtgctcacagtggcttttctccatgaagagggcattcctaacctcttcctttcacgtaggaggaagcaaggtcctttgtaaaattttaactcggggtgcctcaaatgtaaacttaaccactggtaacaacagtttcactgctacatgccacgtctgtgaaaattcattcaagacattaaggaaagtggctcagcagagagactagacatcttatcctcacggttctcctgtacttggcctctcagcctttgagcaaggttggcccaagctagtatcggccccagtggtacagccaaaacttgagactgcaaatggatgcagctgttgaacgctgagtaacttctgcagagtcaggaagacccaaggaagctctgcagaggatgcaggggtacggtcagaacccctgagtgcctttcagctaacgaggactttatgacactccccagcacagcaaatttttatgatgtgtttaaagattgggtgaattactcaggtgaacaagctactttttatcagagaacacctaaaaacacgttcaagagggtttgggaactatacatttaatcctatgacaaactaagttggttctgtcttcacctgttttggtgaggttgtgtaagagttggtgtttgctcaggaagagatttaagcatgcttgcttacccagactcagagaagtctccctgttctgtcctagctagtgattcctgtgttgtgtgcattcgtcttttccagagcaaaccgcccagagtagaag

SEQ ID NO: 33 - DNA, 1574 bp, synthetic construct
  source 1..1574: mol_type = other DNA
acagaggacaacgaccacagccatccctgaaccccgcccacggcacagcgccggagccggggtctggggcgccgcttcctggggggtcccgactctcagccgcccccgcttcacccgggccgccaaggggctgggggaggcggcgctcggggtaaccgggggagactcagggcgctgggggcacttggggaactcatgggggctcaaaggaactaggagatcgggacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcaggtgagcccgccggccccggactgcccggccaggaacctggcgcggggagggaccgcgagacccagagcggttgcccggccgcgtgggtctcggggaaccggggggctggaccaacacacgtccttgggccggggggcgggggccgccttctggagcgggcgtttctgttcgtggccacctctctcctgttagcctctgaccaaggtggcactggatggtttctgcctgaccttggtgccccgtggcagcgactgtgggtcatgaaagacattcactacgagcctgcttctggagtccatcagaaaacgggatgcaacttgcctaaaatgaggagaggaggatgcttttaagaaaaagaagaaggaggattcactaccagctctgaagggtggaaaagagatgattcatccggattgtggagagggtggaatcttgtttaggagagcgttggttgtggcaggcagggtgtaactatgaatcagtgaagacaattcacatcctgggatgaaaagaaggccatgggctcacaggagattatccactggcctctccacatccgcttgcagtaaggagtgtgggactctcccaagcttcagcgctgaactgcaatgcagtgacgtcgcttagctgggccagtaaccgagggagttgaattttctgtcattttaaaataatgtgtcttttaagaaacactttgaaatttttatgatgtgtttaaagattgggtgaattactcaggtgaacaagctactttttatcagagaacacctaaaaacacgttcaagagggtttgggaactatacatttaatcctatgacaaactaagttggttctgtcttcacctgttttggtgaggttgtgtaagagttggtgtttgctcaggaagagatttaagcatgcttgcttacccagactcagagaagtctccctgttctgtcctagctagtgattcctgtgttgtgtgcattcgtcttttccagagcaaaccgcccagagtagaag

SEQ ID NO: 34 - DNA, 1338 bp, synthetic construct
  source 1..1338: mol_type = other DNA
acagaggacaacgaccacagccatccctgaaccccgcccacggcacagcgccggagccggggtctggggcgccgcttcctggggggtcccgactctcagccgcccccgcttcacccgggccgccaaggggctgggggaggcggcgctcggggtaaccgggggagactcagggcgctgggggcacttggggaactcatgggggctcaaaggaactaggagatcgggacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcaggtgagcccgccggccccggactgcccggccaggaacctggcgcggggagggaccgcgagacccagagcggttgcccggccgcgtgggtctcggggaaccggggggctggaccaacacacgtccttgggccggggggcgggggccgccttctggagcgggcgtttctgcatgaaagacattcactacgagcctgcttctggagtccatcagaaaacgggatgcaacttgcctaaaatgaggagaggaggatgcttttaagaaaaagaagaaggaggattcactaccagctctgaagggtggaaaagagatgattcatccggattgtggagagggtggaatcttgtttaggagagcgttggttgtggcaggcagggtgtaactatgaatcagtgaagacaattcacatcctgggatgaaaagaaggccatgggctcacaggagattatccactggcctctccacatcctttatgatgtgtttaaagattgggtgaattactcaggtgaacaagctactttttatcagagaacacctaaaaacacgttcaagagggtttgggaactatacatttaatcctatgacaaactaagttggttctgtcttcacctgttttggtgaggttgtgtaagagttggtgtttgctcaggaagagatttaagcatgcttgcttacccagactcagagaagtctccctgttctgtcctagctagtgattcctgtgttgtgtgcattcgtcttttccagagcaaaccgcccagagtagaag

SEQ ID NO: 35 - DNA, 1641 bp, synthetic construct
  source 1..1641: mol_type = other DNA
ccgcagaatcctatcagtttccccctttcgtgctgtgtgcatcgagcaggaaggggcttggcaggttttacctgccctctttcctttctgaaaagtctgggcctcctcaccccgaaaggagtcacctccttgcagttccccagttgcgaaaagaggaggaagttggctgggccgggggccgcggggggcaccctccgcagatggcgggacccccctgccggccatggcaaaaacgaggcttgtctctcccaccgcccccaaccttagtccttggcacattgttgaaagtaattgaataaaatcggaaattcgagaaggcgttcgttcggattggtgagattttgaggggagaaagaagcggggacttcgccggcaccagcggcgccccctcctcggccaccgttaacccccattccagagggcactgccccgccacccagcctaggtccccctgcgagagcctcgcgggcccgcgcagcctccgcgactcgaacagatcttcagtccttggaggaatgcctgtttctctaacaataaaaaattaaagaagcgctcataaatgccaagtcctctcgcactatgcggagtacagaggacaacgaccacagccatccctgaaccccgcccacggcacagcgccggagccggggtctggggcgccgcttcctggggggtcccgactctcagccgcccccgcttcacccgggccgccaaggggctgggggaggcggcgctcggggtaaccgggggagactcagggcgctgggggcacttggggaactcatgggggctcaaaggaactaggagatcgggacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcaggtgagcccgccggccccggactgcccggccaggaacctggcgcggggagggaccgcgagacccagagcggttgcccggccgcgtgggtctcggggaaccggggggctggaccaacacacgtccttgggccggggggcgggggccgccttctggagcgggcgtttctgcggccgagctccggagctggaatggggcggccggggaagtggacgcgatggcaccgcccggggtgcgagtggggccgggcgcgcgcgggaggggaaaaaggcgcgggcgagccgccagcgcgaggtttgtggtgtcgccgatgtcccttcggggtactctagcgcagccgcctggctacttgacccactgccaccaaacgttttaaattcaccggcttacccagactcagagaagtctccctgttctgtcctagctagtgattcctgtgttgtgtgcattcgtcttttccagagcaaaccgcccagagtagaag

SEQ ID NO: 36 - DNA, 3773 bp, Homo sapiens
  source 1..3773: mol_type = genomic DNA
acagaggacaacgaccacagccatccctgaaccccgcccacggcacagcgccggagccggggtctggggcgccgcttcctggggggtcccgactctcagccgcccccgcttcacccgggccgccaaggggctgggggaggcggcgctcggggtaaccgggggagactcagggcgctgggggcacttggggaactcatgggggctcaaaggaactaggagatcgggacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcaggtgagcccgccggccccggactgcccggccaggaacctggcgcggggagggaccgcgagacccagagcggttgcccggccgcgtgggtctcggggaaccggggggctggaccaacacacgtccttgggccggggggcgggggccgccttctggagcgggcgtttctgcggccgagctccggagctggaatggggcggccggggaagtggacgcgatggcaccgcccggggtgcgagtggggccgggcgcgcgcgggaggggaaaaaggcgcgggcgagccgccagcgcgaggtttgtggtgtcgccgatgtcccttcggggtactctagcgcagccgcctggctacttgacccactgccaccaaacgttttaaattcaccgaaagcttagcttcgaagcaaagctccgtttcgccggtgaagcaggaagccttcgctgcaggaactgacctttacctcttggagcggcttctgcagaaaaatccccgggcagagatttgggcggagtttgcctagaactaacgcggagccagccgatcccggcctaccccggggccaagatttcagtggcttccctttttcctaaacacttcacgagggtctgtttccgggctgtgctccccgcctagaaggaaaatttttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtggtacggccgtgtccccatgtgaccttagagtccctttcgaaactgctgtgcacagtcggtcacaatttcagacactggtgagaagggtggaggaaccctctggggacagccaggcaaggtcgaccacccatcacctaagggtggagaaatttaaggggtgaagagtcccttttgccttttctggatcctggtgattcacctagtgtcttccctaaggaactgaaccaactcctccgctggcctctggcagccctccaggcggtgcaggatggcgtgggcccggtaggaagctgcatgtaaccgcccagggtcgggaggccaggagggcagctcctcctctgacttgaatattgaaaacaacttcgtcctgcttctgagcccctcttaacccatgaccccctagcccattggggagtaaatcttaatttactcctcttcctgaaaaaggatctttaaaacaggtagcttcaactcaagctttataaaataacaatatagggtttctcggaactgtatttttctcagctgatggtaactggacaggtctgtagaagggtgtatgacctgggtttggcaggtggaagagggcaaaggataaacccctcctcctgcagccccatattcttggccaggtgtattgttgtaaaccaggagagagtttacttcggggagtatcctgttttccactcagtgagggccaatgaagaatgtctaattccataagatgcttttgttaaaatcggaatgttgctgtcctcggtggttctgctgttgggacgggactggcctgagctgtgggtgctgtagcaggacaaccagctcacctaagggcctcccagtctggattatcaatgggtcagtgctgaacctgggctaaaatattgttttttccaatgatgttgtctttcccaagctcagtgaagctaaatgtttcacaggcctatgtcaatctgatgtaactttcgtggccacctctctcctgttagcctctgaccaaggtggcactggatggtttctgcctgaccttggtgccccgtggcagcgactgtgggtcatgaaagacattcactacgagcctgcttctggagtccatcagaaaacgggatgcaacttgcctaaaatgaggagaggaggatgcttttaagaaaaagaagaaggaggattcactaccagctctgaagggtggaaaagagatgattcatccggattgtggagagggtggaatcttgtttaggagagcgttggttgtggcaggcagggtgtaactatgaatcagtgaagacaattcacatcctgggatgaaaagaaggccatgggctcacaggagattatccactggcctctccacatccgcttgcagtaaggagtgtgggactctcccaagcttcagcgctgaactgcaatgcagtgacgtcgcttagctgggccagtaaccgagggagttgaattttctgtcattttaaaataatgtgtcttttaagaaacactttgaaattaaaaccacagcccacaattataatgcactgttgcagcacttatcaaaacagatatgctaactgagccatcagtgccagcctgacagtgaggccaccaagccatccacaaagcctacacgaaagtctgtgctcacagtggcttttctccatgaagagggcattcctaacctcttcctttcacgtaggaggaagcaaggtcctttgtaaaattttaactcggggtgcctcaaatgtaaacttaaccactggtaacaacagtttcactgctacatgccacgtctgtgaaaattcattcaagacattaaggaaagtggctcagcagagagactagacatcttatcctcacggttctcctgtacttggcctctcagcctttgagcaaggttggcccaagctagtatcggccccagtggtacagccaaaacttgagactgcaaatggatgcagctgttgaacgctgagtaacttctgcagagtcaggaagacccaaggaagctctgcagaggatgcaggggtacggtcagaacccctgagtgcctttcagctaacgaggactttatgacactccccagcacagcaaatttttatgatgtgtttaaagattgggtgaattactcaggtgaacaagctactttttatcagagaacacctaaaaacacgttcaagagggtttgggaactatacatttaatcctatgacaaactaagttggttctgtcttcacctgttttggtgaggttgtgtaagagttggtgtttgctcaggaagagatttaagcatgcttgcttacccagactcagagaagtctccctgttctgtcctagctagtgattcctgtgttgtgtgcattcgtcttttccagagcaaaccgcccagagtagaag

SEQ ID NO: 37 - DNA, 3236 bp, synthetic construct
  source 1..3236: mol_type = other DNA
acagaggacaacgaccacagccatccctgaaccccgcccacggcacagcgccggagccggggtctggggcgccgcttcctggggggtcccgactctcagccgcccccgcttcacccgggccgccaaggggctgggggaggcggcgctcggggtaaccgggggagactcagggcgctgggggcacttggggaactcatgggggctcaaaggaactaggagatcgggacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcaggtgagcccgccggccccggactgcccggccaggaacctggcgcggggagggaccgcgagacccagagcggttgcccggccgcgtgggtctcggggaaccggggggctggaccaacacacgtccttgggccggggggcgggggccgccttctggagcgggcgtttctgcggccgagctccggagctggaatggggcggccggggaagtggacgcgatggcaccgcccggggtgcgagtggggccgggcgcgcgcgggaggggaaaaaggcgcgggcgagccgccagcgcgaggtttgtggtgtcgccgatgtcccttcggggtactctagcgcagccgcctggctacttgacccactgccaccaaacgttttaaattcaccgaaagcttagcttcgaagcaaagctccgtttcgccggtgaagcaggaagccttcgctgcaggaactgacctttacctcttggagcggcttctgcagaaaaatccccgggcagagatttgggcggagtttgcctagaactaacgcggagccagccgatcccggcctaccccggggccaagatttcagtggcttccctttttcctaaacacttcacgagggtctgtttccgggctgtgctccccgcctagaaggaaaatttttaggacccttgttcgcgaagaggtggtgtgcggctgagacccgcgtcctcaggacggttccatcagtgcctcgatcctgccccactggaggaggaaggcagcccgaacagcgctcacctaactaacagctgctgagagctgggttccgtggccatgcacctgggactgccttgagaagcgtggtacggccgtgtccccatgtgaccttagagtccctttcgaaactgctgtgcacagtcggtcacaatttcagacactggtgagaagggtggaggaaccctctggggacagccaggcaaggtcgaccacccatcacctaagggtggagaaatttaaggggtgaagagtcccttttgccttttctggatcctggtgattcacctagtgtcttccctaaggaactgaaccaactcctccgctggcctctggcagccctccaggcggtgcaggatggcgtgggcccggtaggaagctgcatgtaaccgcccagggtcgggaggccaggagggcagctcctcctctgacttgaatattgaaaacaacttcgtcctgcttctgagcccctcttaacccatgaccccctagcccattggggagtaaatcttaatttactcctcttcctgaaaaaggatctttaaaacaggtagcttcaactcaagctttataaaataacaatatagggtttctcggaactgtatttttctcagctgatggtaactggacaggtctgtagaagggtgtatgacctgggtttggcaggtggaagagggcaaaggataaacccctcctcctgcagccccatattcttggccaggtgtattgttgtaaaccaggagagagtttacttcggggagtatcctgttttccactcagtgagggccaatgaagaatgtctaattccataagatgcttttgttaaaatcggaatgttgctgtcctcggtggttctgctgttgggacgggactggcctgagctgtgggtgctgtagcaggacaaccagctcacctaagggcctcccagtctggattatcaatgggtcagtgctgaacctgggctaaaatattgttttttccaatgatgttgtctttcccaagctcagtgaagctaaatgtttcacaggcctatgtcaatctgatgtaaaaaaccacagcccacaattataatgcactgttgcagcacttatcaaaacagatatgctaactgagccatcagtgccagcctgacagtgaggccaccaagccatccacaaagcctacacgaaagtctgtgctcacagtggcttttctccatgaagagggcattcctaacctcttcctttcacgtaggaggaagcaaggtcctttgtaaaattttaactcggggtgcctcaaatgtaaacttaaccactggtaacaacagtttcactgctacatgccacgtctgtgaaaattcattcaagacattaaggaaagtggctcagcagagagactagacatcttatcctcacggttctcctgtacttggcctctcagcctttgagcaaggttggcccaagctagtatcggccccagtggtacagccaaaacttgagactgcaaatggatgcagctgttgaacgctgagtaacttctgcagagtcaggaagacccaaggaagctctgcagaggatgcaggggtacggtcagaacccctgagtgcctttcagctaacgaggactttatgacactccccagcacagcaaatttttatgatgtgtttaaagattgggtgaattactcaggtgaacaagctactttttatcagagaacacctaaaaacacgttcaagagggtttgggaactatacatttaatcctatgacaaactaagttggttctgtcttcacctgttttggtgaggttgtgtaagagttggtgtttgctcaggaagagatttaagcatgcttgcttacccagactcagagaagtctccctgttctgtcctagctagtgattcctgtgttgtgtgcattcgtcttttccagagcaaaccgcccagagtagaag

SEQ ID NO: 38 - protein, 226 aa, Homo sapiens
MDWGTLQTILGGVNKHSTSIGKIWLTVLFIFRIMILVVAAKEVWGDEQADFVCNTLQPGCKNVCYDHYFPISHIRLWALQLIFVSTPALLVAMHVAYRRHEKKRKFIKGEIKSEFKDIEEIKTQKVRIEGSLWWTYTSSIFFRVIFEAAFMYVFYVMYDGFSMQRLVKCNAWPCPNTVDCFVSRPTEKTVFTVFMIAVSGICILLNVTELCYLLIRYCSGKSKKPV

SEQ ID NO: 39 - DNA, 678 bp, Homo sapiens
  source 1..678: mol_type = other DNA
atggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtt

SEQ ID NO: 40 - DNA, 681 bp, Homo sapiens
  source 1..681: mol_type = other DNA
atggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtttaa

SEQ ID NO: 41 - DNA, 678 bp, synthetic construct
  source 1..678: mol_type = other DNA
atggactggggcaccctgcagactatcctggggggcgtcaataagcattcaactagcatcggaaagatttggctgactgtcctgtttatctttcggatcatgatcctggtggtggcagcaaaggaagtgtggggcgacgagcaggccgatttcgtgtgcaacacactgcagccaggctgcaagaacgtgtgctacgaccactattttcccatctctcacatcaggctgtgggccctgcagctgatcttcgtgagcacccctgccctgctggtggcaatgcacgtggcctatcggagacacgagaagaagcgcaagtttatcaagggcgagatcaagagcgagttcaaggatatcgaggagatcaagacacagaaggtgaggatcgagggctccctgtggtggacctacacaagctccatcttctttcgcgtgatcttcgaggccgcctttatgtacgtgttctatgtgatgtacgacggcttttctatgcagcggctggtgaagtgcaacgcctggccctgtcctaatacagtggattgtttcgtgtccagacccaccgagaagacagtgttcaccgtgtttatgatcgccgtgtctggcatctgcatcctgctgaacgtgaccgagctgtgctatctgctgatccggtactgtagtggaaagagcaaaaaacccgtg

SEQ ID NO: 42 - DNA, 678 bp, synthetic construct
  source 1..678: mol_type = other DNA
atggactggggaacattgcaaactattttgggaggagtcaacaagcattcaactagcatcgggaagatctggctgaccgtgctgttcatctttcgcatcatgattctcgtggtggccgctaaggaagtctggggcgatgaacaggccgacttcgtgtgtaacacgctgcagcccggttgcaaaaacgtctgctacgatcactacttccccatctcacacattagactgtgggcgctgcagctgattttcgtgtccaccccggcacttcttgtggcgatgcacgtggcctaccggcggcacgagaagaaaaggaagttcattaagggcgaaatcaagtccgagttcaaggacatcgaagaaatcaagacccagaaggtccgcattgagggctccctctggtggacctacacctcgtccatcttcttccgggtcatattcgaggccgcctttatgtacgtgttttacgtgatgtacgacggtttcagcatgcaaagactcgtcaagtgcaacgcttggccttgccccaataccgtggattgcttcgtgtcccgcccgaccgagaaaactgtgttcactgtgttcatgatcgccgtgtccggcatctgcatcctgctgaacgtgaccgagctgtgctatctcctgatccggtactgtagcggaaagtcgaagaagcctgtg

SEQ ID NO: 43 - DNA, 678 bp, synthetic construct
  source 1..678: mol_type = other DNA
atggattgggggacgctccagactatacttggcggggtaaacaaacattccacctcaattggcaaaatctggctcacagtcctcttcatcttcagaataatgatactcgtggttgccgctaaagaagtttggggtgacgagcaagccgatttcgtctgtaacaccctccaaccaggttgcaaaaatgtctgttacgatcactactttcctattagccatattagactctgggccctgcaacttatcttcgtttccactcctgctctgctcgtcgctatgcacgttgcctatcgccgccatgaaaaaaaacggaaattcattaagggagagattaagagtgaattcaaggatattgaagagattaaaacgcaaaaagttagaattgagggatcactgtggtggacttataccagtagcatcttttttagggtcattttcgaagctgctttcatgtatgttttctatgtaatgtacgacggtttctccatgcaacgcttggttaaatgtaacgcctggccatgccctaatacggttgattgctttgtctcccgccctactgaaaagacagtgtttaccgttttcatgatcgccgtaagtggaatttgtatccttcttaacgtgaccgagttgtgctatctccttattcgctactgttcaggaaaaagtaaaaaaccagta

SEQ ID NO: 44 - DNA, 681 bp, synthetic construct
  source 1..681: mol_type = other DNA
atggactggggcacgctgcagactatcctggggggtgtcaacaagcattcaactagcatcggaaagatctggctgaccgtcctgttcatctttcgcatcatgatcctcgtggtggccgctaaggaagtgtggggcgacgagcaggccgatttcgtgtgtaacaccctgcagccaggttgcaaaaacgtctgctacgatcactactttcccatctcccacattagactgtgggccctgcagctgatcttcgtgtccacccctgcgctgctagtggccatgcacgtggcctatcggcgacacgagaagaaacggaagttcattaagggcgagatcaagagcgagttcaaggatatcgaagagatcaagacccagaaggtccgcattgagggctccctgtggtggacctacaccagctccatcttctttcgggtcatcttcgaggccgcctttatgtacgtgttctatgtgatgtacgacggtttctccatgcaacggctggtgaagtgcaacgcctggccttgccctaatactgtggattgcttcgtgtcccgccccaccgagaagacagtgttcaccgtgttcatgatcgccgtgtctggcatctgcatcctgctgaacgtgaccgagctgtgctatctcctgatccggtactgtagtggaaagtcaaaaaaaccagtgtaa

SEQ ID NO: 45 - protein, 226 aa, Mus musculus
MDWGTLQSILGGVNKHSTSIGKIWLTVLFIFRIMILVVAAKEVWGDEQADFVCNTLQPGCKNVCYDHHFPISHIRLWALQLIMVSTPALLVAMHVAYRRHEKKRKFMKGEIKNEFKDIEEIKTQKVRIEGSLWWTYTTSIFFRVIFEAVFMYVFYIMYNGFFMQRLVKCNAWPCPNTVDCFISRPTEKTVFTVFMISVSGICILLNITELCYLFVRYCSGKSKRPV

SEQ ID NO: 46 - DNA, 678 bp, Mus musculus
  source 1..678: mol_type = other DNA
atggattggggcacactccagagcatcctcgggggtgtcaacaaacactccaccagcattggaaagatctggctcacggtcctcttcatcttccgcatcatgatcctcgtggtggctgcaaaggaggtgtggggagatgagcaagccgattttgtctgcaacacgctccagcctggctgcaagaatgtatgctacgaccaccacttccccatctctcacatccggctctgggctctgcagctgatcatggtgtccacgccagccctcctggtagctatgcatgtggcctaccggagacatgaaaagaaacggaagttcatgaagggagagataaagaacgagtttaaggacatcgaagagatcaaaacccagaaggtccgtatcgaagggtccctgtggtggacctacaccaccagcatcttcttccgggtcatctttgaagccgtcttcatgtacgtcttttacatcatgtacaatggcttcttcatgcaacgtctggtgaaatgcaacgcttggccctgccccaatacagtggactgcttcatttccaggcccacagaaaagactgtcttcaccgtgtttatgatttctgtgtctggaatttgcattctgctaaatatcacagagctgtgctatttgttcgttaggtattgctcaggaaagtccaaaagaccagtc

SEQ ID NO: 47 - protein, 261 aa, Homo sapiens
MDWGTLHTFIGGVNKHSTSIGKVWITVIFIFRVMILVVAAQEVWGDEQEDFVCNTLQPGCKNVCYDHFFPVSHIRLWALQLIFVSTPALLVAMHVAYYRHETTRKFRRGEKRNDFKDIEDIKKQKVRIEGSLWWTYTSSIFFRIIFEAAFMYVFYFLYNGYHLPWVLKCGIDPCPNLVDCFISRPTEKTVFTIFMISASVICMLLNVAELCYLLLKVCFRRSKRAQTQKNHPNHALKESKQNEMNELISDSGQNAITGFPS

SEQ ID NO: 48 - DNA, 783 bp, Homo sapiens
  source 1..783: mol_type = other DNA
atggattgggggacgctgcacactttcatcgggggtgtcaacaaacactccaccagcatcgggaaggtgtggatcacagtcatctttattttccgagtcatgatcctcgtggtggctgcccaggaagtgtggggtgacgagcaagaggacttcgtctgcaacacactgcaaccgggatgcaaaaatgtgtgctatgaccactttttcccggtgtcccacatccggctgtgggccctccagctgatcttcgtctccaccccagcgctgctggtggccatgcatgtggcctactacaggcacgaaaccactcgcaagttcaggcgaggagagaagaggaatgatttcaaagacatagaggacattaaaaagcagaaggttcggatagaggggtcgctgtggtggacgtacaccagcagcatctttttccgaatcatctttgaagcagcctttatgtatgtgttttacttcctttacaatgggtaccacctgccctgggtgttgaaatgtgggattgacccctgccccaaccttgttgactgctttatttctaggccaacagagaagaccgtgtttaccatttttatgatttctgcgtctgtgatttgcatgctgcttaacgtggcagagttgtgctacctgctgctgaaagtgtgttttaggagatcaaagagagcacagacgcaaaaaaatcaccccaatcatgccctaaaggagagtaagcagaatgaaatgaatgagctgatttcagatagtggtcaaaatgcaatcacaggtttcccaagc

SEQ ID NO: 49 - DNA, 786 bp, Homo sapiens
  source 1..786: mol_type = other DNA
atggattgggggacgctgcacactttcatcgggggtgtcaacaaacactccaccagcatcgggaaggtgtggatcacagtcatctttattttccgagtcatgatcctcgtggtggctgcccaggaagtgtggggtgacgagcaagaggacttcgtctgcaacacactgcaaccgggatgcaaaaatgtgtgctatgaccactttttcccggtgtcccacatccggctgtgggccctccagctgatcttcgtctccaccccagcgctgctggtggccatgcatgtggcctactacaggcacgaaaccactcgcaagttcaggcgaggagagaagaggaatgatttcaaagacatagaggacattaaaaagcagaaggttcggatagaggggtcgctgtggtggacgtacaccagcagcatctttttccgaatcatctttgaagcagcctttatgtatgtgttttacttcctttacaatgggtaccacctgccctgggtgttgaaatgtgggattgacccctgccccaaccttgttgactgctttatttctaggccaacagagaagaccgtgtttaccatttttatgatttctgcgtctgtgatttgcatgctgcttaacgtggcagagttgtgctacctgctgctgaaagtgtgttttaggagatcaaagagagcacagacgcaaaaaaatcaccccaatcatgccctaaaggagagtaagcagaatgaaatgaatgagctgatttcagatagtggtcaaaatgcaatcacaggtttcccaagctaa

SEQ ID NO: 50 - protein, 261 aa, Mus musculus
MDWGTLHTVIGGVNKHSTSIGKVWITVIFIFRVMILVVAAQEVWGDEQEDFVCNTLQPGCKNVCYDHFFPVSHIRLWALQLIFVSTPALLVAMHVAYYRHETARKFIRGEKRNEFKDLEDIKRQKVRIEGSLWWTYTSSIFFRIIFEAAFMYVFYFLYNGYHLPWVLKCGIDPCPNLVDCFISRPTEKTVFTVFMISASVICMLLNVAELCYLLLKLCFRRSKRTQAQRNHPNHALKESKQNEMNELISDSGQNAITSFPS

SEQ ID NO: 51 - DNA, 783 bp, Mus musculus
  source 1..783: mol_type = other DNA
atggactgggggaccctgcacaccgtcatcggtggcgtgaacaagcactctaccagcatagggaaggtgtggatcacggtcatctttattttccgagtcatgatcctagtggtggctgcccaggaagtgtggggtgatgagcaggaggactttgtctgcaacactctgcagccagggtgcaagaacgtctgctatgaccatttcttcccggtgtctcacatccggctctgggccctgcagctgatctttgtgtctaccccagccctgttggtggccatgcacgtggcctactacagacatgaaactgcccgaaagtttatacgtggggagaagagaaacgagtttaaagacctggaggacatcaaacggcagaaggtgcgcattgagggctccctgtggtggacgtacaccagcagcattttcttccgcatcatcttcgaagccgccttcatgtatgtgttctacttcctctacaatgggtaccacctaccctgggtactgaaatgtggcattgacccctgccccaatctcgtggactgcttcatttcgaggccaactgagaaaacggtgttcactgtttttatgatttccgcatccgtgatttgcatgctgctcaatgtggccgagttgtgttacctgctgcttaaattgtgctttaggagatccaaaagaacacaggcgcagagaaaccaccccaaccatgccctgaaagagagcaagcagaatgaaatgaatgagctgatctcagatagtggccagaatgcaatcacaagtttcccaagt

SEQ ID NO: 52 - DNA, 340 bp, Homo sapiens
  source 1..340: mol_type = genomic DNA
aaaaactcttacataattgtaacagatttgagtttcctctggttcaggttttcttgcctctttgataatcaaatgatctgaagaaaggcatagaatttcaagggagaatctgcatgacaggattacaataaggctattcatggagactcttttattagcttacacaggatctgcatcattcttcgtctctggctttgctacaaggctccatttaaacttaacccaacttgcaggcttaactgatccaggaatgactcaatacaatgagccagctactgtagctctttttccccttttgataaggggagttaacacaatgggctttacagttcttaaatga

SEQ ID NO: 53 - DNA, 56 bp, Homo sapiens
  source 1..56: mol_type = genomic DNA
tttccgacaagtctcagtgccatctactgtgcgctctgggtattgcaattgctttt

SEQ ID NO: 54 - DNA, 402 bp, Homo sapiens
  source 1..402: mol_type = genomic DNA
atataagggaacgctgctgttctgttgtagagattacagagagcaatttaggagagccaggcactggggccaagagggaaatgaaacaaaaaccgaagggatttgtttaggaagaaaaatgaaaacagataaaaggtgttcatttcaaagcttccctctttcccagcatttttctgaagtagagtttgaaaagaaagcaaaataactgcaaaccaatacagcggcacggagttcactgacgcagagctagaaatgacgttcagagatccgccagcccagcctcctgttctgggtcacctggctccttgacaaccctgaaagctgcctgtgcaaatccccaggcatgttatacccatgagcagggacgcgtggcactgacaaagggactcgtacacctttgaa

SEQ ID NO: 55 - DNA, 562 bp, Homo sapiens
  source 1..562: mol_type = genomic DNA
acacagcagtcaagttctcaaacaatcttcaggagccttccaaaactggacataaaaaaaagtaccttcattggggaaaagaaaagaaaaaaaaaagaatcacaattgccaccaaggctctacatttttgcaaaagtccagcatttaaaagaaacttccctggatggcctacatctgctgattggtaatttgtcattcaggttaaaaacaaaacagaagtgggcattgttgtgatatcatccttgataacatcccaagaaaactctagagctggcaggagaggaaagcagataatggtcaaagctgtcatctgagttttaaaaacactgtgatttttcttttaaaggaacatcttcagtttccaaggccatacacacagttcctaactgcagcttcaaattttccaccgggctcccttctgagaacaaacgctattcagtggcgagccccggacaccactgcgctttcaaaggcagctgccagaggacactcaggacttcacagccgccggtgagccagactggggtcagtcactcccccatcagaattattttgtttctcc

SEQ ID NO: 56 - DNA, 528 bp, Homo sapiens
  source 1..528: mol_type = genomic DNA
ccagtggttccctactgtgcctaagtcataggagtactggctaaaaatgcagactccagacatctcccccagaattacaagcatcccaaggaattccgctaaaggtgatatgcaaaccacattctgataagcagataactggggaccaggggctgcacaagtcccacacccactaggagtggcatttattccatctcttcacctgacaggtacaaagtctttcatctttggaaagaatggtttttccttggggcatctcaagcactactttcatggaagaggaggtagtatgggactgttattgctttctatgggaaaaaaaaattgaaaactaatattgttttgctttggggtgaaatgaagagtagtaattggaccttttgttttctttgtcttgattgtccatttttgtaagaaagtgaatgccaagcaaaatacaaatggatatagaaaattctgcctaactcagcaatgtctttaaatgtaaatactgcactagaaacagctaatctccagatcacacaccat

SEQ ID NO: 57 - DNA, 510 bp, Homo sapiens
  source 1..510: mol_type = genomic DNA
ccgttcctgcaggtattggaaaacctgcatcgtatgcctgctgctttaggagacaacaaactcagcagcccctcccccgaagctgaagtgccaaacttaaggctccaggtaaactgcccttccgatgagagctttacatttaggcagtagccacaggcctaaacaaagtcaaatggatgaaacttgtgatctctccttggaaatatggccaagagtgaaacaactacttagttattattttaaacgcaggctgctaaatggcttgtgttggtatgtaatggcatcctagataatgggggctgccaagaaaatattcacactggcaagtcaattaaaataccgttctcttcctaagaggcctggctggattccagagcttcctttagggtaacgctcaaacctgtcatagtttattttgtttctgttttcaggcaacagatttggttattgcttttattgtatgagtgttgaaatggtgtcctaacagccatctgtactgccatcccctgg

SEQ ID NO: 58 - DNA, 259 bp, Homo sapiens
  source 1..259: mol_type = genomic DNA
tctctctgcctttccagaaaggagatgaagttgctggagcaggcaggttctctgaaaggctccctgagtgtggaagagcagctgtcactcatcagtggttgtcccaatatccaagaagcagtagagggtgccatgcacattcaggtgagtgaaggacactggcgccgcatctgtggggcctgagacattgtctcccagcctctccggaggaacagggcaggatacagagcagtggcaggtgtgggcattttgcatcttc

SEQ ID NO: 59 - DNA, 307 bp, Homo sapiens
  source 1..307: mol_type = genomic DNA
tgtaatctccaaacaaagtctgcgctaggccctttcaggctataaaggacagggtactctgatttctgctcttgatgagggttgattgtgataccaggcgagggagtaagagatacaggacaccatctctgcggctgcatctttgtcgcagaaacagacattgttcaggggacacctgagcctctgatagtcagccagtggctctggcagctcacagcagttatagggtggctgtgtccttttctaaagtcctgactttttatggcctgtggtcatggtgactccactacgggctgtgtttctgttg

SEQ ID NO: 60 - DNA, 1477 bp, Homo sapiens
  source 1..1477: mol_type = genomic DNA
tgaacttaagtcatttgcccaaggttatacagccagtaggctggaaagccagggtaaggagccggccggctggctccacaggccagaaccaccattctgagggcccgtctttcaggggtgttgtttgtaaaaggagccaaaaggcaatggcacgctggcctcctgtttctcagaaactatttcattagattaaaaataaatcctaaaatatttgcactagttctagataaatggtagcttttcaggctaataactacccttaacctgagatgctggctttatgaaaacagtatcagaaaagaagaaaaattctcccaactccaatcttgatatattctgggtgacacagggaagaatgataaacatttctatgggttcccagccagatcttataactctccaactctggtctttggaatgctctgaagttgaaaactacatccgtccaccattatcatatgaagccaattaggttttcttatcgtatctgctttgtttgaaaaattgttatgggcctccttcctgtctgccactctatttgattcttgagtttctggtttcattggcttctgtttctatttgtttgaaagtcaaagctgttctcagttctgtccctatctatttcttatgattcctcaattatcttatttttctcaagaaaaaaaagggagaaagaaaaacacagtggttatcaggcagcgtctgtatacatttcaaatgtataaatttctatacatttcaaatgcttaagtatatatacaaatctatatacatttcaaatgcttaaatatgctcacaagttttgaggttctggttgaaaacaccatttgaaatgatctaagactttgtgttaataatacaagaaaactcttgtttcactgcatgaggccatctttctctacattatcttcttctattcagtggttatcttcccataaaactaacacacacgtgctggagcagattcccctcttcccttcactcctcttagctcttcttagcttgattctgataaggactgaagcagagaacacacggcccatccacttcagaaatcatctgtgcacctgctgtatgcaggaacatgtctagtaaagcttccttgaattattcattggacagcatatttcattaactactgtgaaaattgactgaactattaccaaaatgtaatactgaaattatagaactgaagtcaaacagactgagagaggtcaccaactctcaacctcccctctcgcagagggagtttcctctcaacatctaaacctcccacaggcaggcaaccacatccccttttcagcttacccacttaaagcatgggcctttcctggacatcagctcctgttactaggaagttcttcctgccatgtactgagcccaaacctgtctgtctgcttgccatttccagtctttcactccacttccgcccccacaaaacatacagaccgagagacatacgagagactctggtcac

SEQ ID NO: 61 - DNA, 761 bp, Homo sapiens
  source 1..761: mol_type = genomic DNA
aaggagccaaaaggcaatggcacgctggcctcctgtttctcagaaactatttcattagattaaaaataaatcctaaaatatttgcactagttctagataaatggtagcttttcaggctaataactacccttaacctgagatgctggctttatgaaaacagtatcagaaaagaagaaaaattctcccaactccaatcttgatatattctgggtgacacagggaagaatgataaacatttctatgggttcccagccagatcttataactctccaactctggtctttggaatgctctgaagttgaaaactacatccgtccaccattatcatatgaagccaattaggttttcttatcgtatctgctttgtttgaaaaattgttatgggcctccttcctgtctgccactctatttgattcttgagtttctggtttcattggcttctgtttctatttgtttgaaagtcaaagctgttctcagttctgtccctatctatttcttatgattcctcaattatcttatttttctcaagaaaaaaaagggagaaagaaaaacacagtggttatcaggcagcgtctgtatacatttcaaatgtataaatttctatacatttcaaatgcttaagtatatatacaaatctatatacatttcaaatgcttaaatatgctcacaagttttgaggttctggttgaaaacaccatttgaaatgatctaagactttgtgttaataatacaagaaaactcttgtttcactgcatgaggcca

SEQ ID NO: 62 - DNA, 1043 bp, Homo sapiens
  source 1..1043: mol_type = genomic DNA
ctgcttcggcctccaaatagctgggattacaggcatgcgtcaccacgcccagctaattttgtatttttagtagagacacagtttctccatgttggtcaggctggtctcaaactcctgacctcaagtgatccacccgcctcagcctcccaaagtgctgggattacaggtgtgagccaccacatctggataattttgtatttttagtagcgatggggtttctccatgttggtcagactggtctcaaactcctgacctcaagtgatccacccagagtgctgagattacaggtgtgagccaccacatccggcctagattaagtttatattgagaactaccacaatgattcatgagtctctcatgagaaactagttggaattatgagtttgttccctccacttatacatactgtacccacatgttttcgttttctatactcttcaccacatgttgcttcccccacatgcaacaggaaactggctccacggcaagggtgaaaaagatctgacccagtggttcccacccacgccaccttccagggagaaggtgaggccaagatgagactttaataagtcgaaaataaaagactgctaagtgctgccccacaattactcacaggcttgtggtcaggtttgtgagctgcttggcctgttgccggcacttacaagctttcttggaagcaagacacagacattcctgcggaatgacttgagcaaattttgctaacgctacaagggttgattgaacaccaccacattgaactgatgcaagaggacactgaatcacacccataaaacacccgtcctgtcaagagtcttccaagaggacgcgcaggagtcagcgctccggggaaagaagatgagaaacaccaggtggaccaccctggccaggctgctgggaacaagcctacgtgcacagctgccggaggagaggcgggtctcccctctgggaggcatctcagctacatctatcaagttataaaaatgttactctcccgcaggctgagcccactcccagaattattccagaaaagactccagaagaag

SEQ ID NO: 63 - DNA, 588 bp, Homo sapiens
  source 1..588: mol_type = genomic DNA
caccacatccggcctagattaagtttatattgagaactaccacaatgattcatgagtctctcatgagaaactagttggaattatgagtttgttccctccacttatacatactgtacccacatgttttcgttttctatactcttcaccacatgttgcttcccccacatgcaacaggaaactggctccacggcaagggtgaaaaagatctgacccagtggttcccacccacgccaccttccagggagaaggtgaggccaagatgagactttaataagtcgaaaataaaagactgctaagtgctgccccacaattactcacaggcttgtggtcaggtttgtgagctgcttggcctgttgccggcacttacaagctttcttggaagcaagacacagacattcctgcggaatgacttgagcaaattttgctaacgctacaagggttgattgaacaccaccacattgaactgatgcaagaggacactgaatcacacccataaaacacccgtcctgtcaagagtcttccaagaggacgcgcaggagtcagcgctccggggaaagaagatgagaaacaccaggtggaccaccctgg

SEQ ID NO: 64 - DNA, 330 bp, Homo sapiens
  source 1..330: mol_type = genomic DNA
ttgactgaactattaccaaaatgtaatactgaaattatagaactgaagtcaaacagactgagagaggtcaccaactctcaacctcccctctcgcagagggagtttcctctcaacatctaaacctcccacaggcaggcaaccacatccccttttcagcttacccacttaaagcatgggcctttcctggacatcagctcctgttactaggaagttcttcctgccatgtactgagcccaaacctgtctgtctgcttgccatttccagtctttcactccacttccgcccccacaaaacatacagaccgagagacatacgagagactctggtcac

SEQ ID NO: 65 - DNA, 728 bp, Homo sapiens
  source 1..728: mol_type = genomic DNA
agtatatatacaaatctatatacatttcaaatgcttaaatatgctcacaagttttgaggttctggttgaaaacaccatttgaaatgatctaagactttgtgttaataatacaagaaaactcttgtttcactgcatgaggccatctttctctacattatcttcttctattcagtggttatcttcccataaaactaacacacacgtgctggagcagattcccctcttcccttcactcctcttagctcttcttagcttgattctgataaggactgaagcagagaacacacggcccatccacttcagaaatcatctgtgcacctgctgtatgcaggaacatgtctagtaaagcttccttgaattattcattggacagcatatttcattaactactgtgaaaattgactgaactattaccaaaatgtaatactgaaattatagaactgaagtcaaacagactgagagaggtcaccaactctcaacctcccctctcgcagagggagtttcctctcaacatctaaacctcccacaggcaggcaaccacatccccttttcagcttacccacttaaagcatgggcctttcctggacatcagctcctgttactaggaagttcttcctgccatgtactgagcccaaacctgtctgtctgcttgccatttccagtctttcactccacttccgcccccacaaaacatacagaccgagagacatacgagagactctggtcac

SEQ ID NO: 66 - DNA, 1000 bp, Homo sapiens
  source 1..1000: mol_type = genomic DNA
ctcataaatgccaagtcctctcgcactatgcggagtacagaggacaacgaccacagccatccctgaaccccgcccacggcacagcgccggagccggggtctggggcgccgcttcctggggggtcccgactctcagccgcccccgcttcacccgggccgccaaggggctgggggaggcggcgctcggggtaaccgggggagactcagggcgctgggggcacttggggaactcatgggggctcaaaggaactaggagatcgggacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcaggtgagcccgccggccccggactgcccggccaggaacctggcgcggggagggaccgcgagacccagagcggttgcccggccgcgtgggtctcggggaaccggggggctggaccaacacacgtccttgggccggggggcgggggccgccttctggagcgggcgtttctgcggccgagctccggagctggaatggggcggccggggaagtggacgcgatggcaccgcccggggtgcgagtggggccgggcgcgcgcgggaggggaaaaaggcgcgggcgagccgccagcgcgaggtttgtggtgtcgccgatgtcccttcggggtactctagcgcagccgcctggctacttgacccactgccaccaaacgttttaaattcaccgaaagcttagct

SEQ ID NO: 67 - DNA, 1553 bp, synthetic construct
  source 1..1553: mol_type = other DNA
aagcttcggtgaatttaaaacgtttggtggcagtgggtcaagtagccaggcggctgcgctagagtaccccgaagggacatcggcgacaccacaaacctcgcgctggcggctcgcccgcgcctttttcccctcccgcgcgcgcccggccccactcgcaccccgggcggtgccatcgcgtccacttccccggccgccccattccagctccggagctcggccgcagaaacgcccgctccagaaggcggcccccgccccccggcccaaggacgtgtgttggtccagccccccggttccccgagacccacgcggccgggcaaccgctctgggtctcgcggtccctccccgcgccaggttcctggccgggcagtccggggccggcgggctcacctgcgtcgggaggaagcgcggcggggccggggcgggggtctcggcgttggggtctctgcgctggggctcctgcgctcctaggcgggtcctgggccgggcgccgccgaggggctccgagtcggggagaggagcgcgcgggcgctgcggggccgcaacacctgtctcccgccgtggcgccttttaaccgcaccccacaccccgcctcttccctcggagactgggaaagttacggagggggcggcgccgcgggcggagcgcgcccggcctctgggtcctcagagcttcccgggtccgcgaacccccgaccgcccccgaaagccccgaaccccccaagtccccttcgaggtcccgatctcctagttcctttgagcccccatgagttccccaagtgcccccagcgccctgagtctcccccggttaccccgagcgccgcctcccccagccccttggcggcccgggtgaagcgggggcggctgagagtcgggaccccccaggaagcggcgccccagaccccggctccggcgctgtgccgtgggcggggttcagggatggctgtggtcgttgtcctctgtactccgcatagtgcgagaggacttggcatttatgagcgcttctttaattttttattgttagagaaacaggcattcctccaaggactgaagatctgttcgagtcgcggaggctgcgcgggcccgcgaggctctcgcagggggacctaggctgggtggcggggcagtgccctctggaatgggggttaacggtggccgaggagggggcgccgctggtgccggcgaagtccccgcttctttctcccctcaaaatctcaccaatccgaacgaacgccttctcgaatttccgattttattcaattactttcaacaatgtgccaaggactaaggttgggggcggtgggagagacaagcctcgtttttgccatggccggcaggggggtcccgccatctgcggagggtgccccccgcggcccccggcccagccaacttcctcctcttttcgcaactggggaactgcaaggaggtgactcctttcggggtgaggaggcccagacttttcagaaaggaaagagggcaggtaaaacctgccaagccccttcctgctcgatgcacacagcacgaaagggggaaactgataggattctgcggaagctt

SEQ ID NO: 68 - DNA, 1010 bp, synthetic construct
  source 1..1010: mol_type = other DNA
aagcttccgcagaatcctatcagtttccccctttcgtgctgtgtgcatcgagcaggaaggggcttggcaggttttacctgccctctttcctttctgaaaagtctgggcctcctcaccccgaaaggagtcacctccttgcagttccccagttgcgaaaagaggaggaagttggctgggccgggggccgcggggggcaccctccgcagatggcgggacccccctgccggccatggcaaaaacgaggcttgtctctcccaccgcccccaaccttagtccttggcacattgttgaaagtaattgaataaaatcggaaattcgagaaggcgttcgttcggattggtgagattttgaggggagaaagaagcggggacttcgccggcaccagcggcgccccctcctcggccaccgttaacccccattccagagggcactgccccgccacccagcctaggtccccctgcgagagcctcgcgggcccgcgcagcctccgcgactcgaacagatcttcagtccttggaggaatgcctgtttctctaacaataaaaaattaaagaagcgctcataaatgccaagtcctctcgcactatgcggagtacagaggacaacgaccacagccatccctgaaccccgcccacggcacagcgccggagccggggtctggggcgccgcttcctggggggtcccgactctcagccgcccccgcttcacccgggccgccaaggggctgggggaggcggcgctcggggtaaccgggggagactcagggcgctgggggcacttggggaactcatgggggctcaaaggaactaggagatcgggacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggt

(19) United States
(12) Patent Application Publication
(10) Pub. No.: US 2025/0127928 A1
(43) Pub. Date: Apr. 24, 2025

(54) GJB2 REGULATORY ELEMENTS AND USES THEREOF

(71) Applicants: Decibel Therapeutics, Inc., Boston, MA (US); Regeneron Pharmaceuticals, Inc., Tarrytown, NY (US)

(72) Inventors: Joseph BURNS, Newton, MA (US); Kathryn ELLIS, Arlington, MA (US); Tyler GIBSON, Boston, MA (US); Kevin LEBO, Weymouth, MA (US); Gabriela PREGERNIG, Cambridge, MA (US); Meghan DRUMMOND SAMUELSON, Katonah, NY (US); Sarah CANCELARICH, Bloomingdale, NJ (US); Leah SABIN, Goldens Bridge, NY (US); Daniela DI BATTISTA MIANI, Yorktown Heights, NY (US)

(21) Appl. No.: 18/835,452

(22) PCT Filed: Feb. 3, 2023
(86) PCT No.: PCT/US2023/061953

§ 371 Date: Aug. 2, 2024

Related U.S. Application Data
(60) Provisional application No. 63/306,941, filed on Feb. 4, 2022.
(60) Provisional application No. 63/306,928, filed on Feb. 4, 2022.

(51) Int. Cl. A61K 48/00; A61K 9/00; A61K 38/17; A61P 27/16; C12N 15/86

(52) U.S. Cl. CPC A61K 48/0058; A61K 9/0046; A61K 38/1709; A61K 48/0075; A61P 27/16; C12N 15/86; C12N 2750/14143

(57) ABSTRACT

The disclosure provides gap junction protein beta 2 (GJB2) promoters and enhancers, as well as vectors containing the same, that can be used to express an expression product in GJB2-expressing cells, including cochlear supporting cells. The GJB2 promoters and enhancers described herein may be operably linked to a polynucleotide, such as a transgene encoding a wild-type form of Gjb2, for treatment of subjects having or at risk of developing hearing loss (e.g., GJB2-related hearing loss).

### BACKGROUND

Hearing loss is a major public health issue that is estimated to affect nearly 15% of school-age children and one out of three people by age sixty-five. The most common type of hearing loss is sensorineural hearing loss, a type of hearing loss caused by defects in the cells of the inner ear, such as cochlear hair cells and cochlear supporting cells, or the neural pathways that project from the inner ear to the brain. Sensorineural hearing loss is often acquired, and has a variety of causes, including acoustic trauma, disease or infection, head trauma, ototoxic drugs, and aging. There are also genetic causes of sensorineural hearing loss, such as mutations in genes involved in the development and function of the inner ear. Mutations in over 90 such genes have been identified, including mutations inherited in an autosomal recessive, autosomal dominant, and X-linked pattern.

In recent years, efforts to treat hearing loss have increasingly focused on gene therapy as a possible solution. There remain, however, few approaches to target specific cell populations in the cochlea. Gene therapy approaches for hearing loss that induce expression of an exogenous gene in all cells of the inner ear may have off-target effects or result in toxicity. Accordingly, there is a need for new approaches to target specific cell populations in the cochlea for the treatment of hearing loss.

### SUMMARY OF THE INVENTION

The invention provides compositions and methods for promoting the expression of a gene of interest, such as a gene that is endogenously expressed in GJB2-expressing cells, a gene that can induce the differentiation of cochlear supporting cells into cochlear hair cells, or a gene expressed in cochlear supporting cells that is mutated in subjects with hearing loss, in specific cell types. The compositions and methods described herein relate to polynucleotides that can induce expression of a transgene in GJB2-expressing cells (e.g., GJB2-expressing inner ear cells). The polynucleotides described herein may be operably linked, e.g., to a polynucleotide encoding an expression product such as a protein or an inhibitory RNA, and may be administered to a subject, such as a human subject, to treat or prevent hearing loss (e.g., sensorineural hearing loss, such as GJB2-related hearing loss).

In a first aspect, the invention provides a polynucleotide including a GJB2 promoter containing a first region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1 or a functional portion or derivative thereof including at least one of (e.g., one or more of) SEQ ID NOs: 3-12 operably linked to a second region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or a functional portion or derivative thereof including at least one of (e.g., one of more of) SEQ ID NOs: 20-24, in which the distance between the first region and the second region in the polynucleotide (e.g., the distance between the 3′ end of the first region and the 5′ end of the second region) is no more than 1 kb, optionally including a linker containing one to one hundred nucleotides between the first region and the second region. In some embodiments, the distance between the first region and the second region in the polynucleotide is no more than 0.5 kb. In some embodiments, the distance between the first region and the second region in the polynucleotide is no more than 0.25 kb.

In another aspect, the invention provides a polynucleotide including a GJB2 promoter containing a first region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1 or a functional portion or derivative thereof including at least one of (e.g., one or more of) SEQ ID NOs: 3-12 operably linked to a second region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or a functional portion or derivative thereof including at least one of (e.g., one of more of) SEQ ID NOs: 20-24, in which when the first region and the second region not joined directly (e.g., fused) the first region is operably linked to the second region by a nucleic acid sequence that differs from the intervening genomic sequence.

In another aspect, the invention provides a polynucleotide including a GJB2 promoter containing a first region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1 or a functional portion or derivative thereof including at least one of (e.g., one or more of) SEQ ID NOs: 3-12 operably linked to a second region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or a functional portion or derivative thereof including at least one of (e.g., one of more of) SEQ ID NOs: 20-24, in which the GJB2 promoter is operably linked to a heterologous polynucleotide, optionally including a linker containing one to one hundred nucleotides between the first region and the second region. In some embodiments, the heterologous polynucleotide is a codon-optimized nucleic acid sequence encoding wild-type GJB2 (e.g., the heterologous polynucleotide has a sequence set forth in SEQ ID NOs: 41-44). In some embodiments, the heterologous polynucleotide encodes a Gjb2 protein including one or more conservative amino acid substitutions relative to SEQ ID NO: 38 that retains the therapeutic function of wild-type Gjb2. In some embodiments, the heterologous polynucleotide encodes Gjb6 (e.g., encodes SEQ ID NO: 47). In some embodiments, the heterologous polynucleotide encodes brain derived neurotrophic factor (BDNF) or neurotrophin 3 (NTF3). In some embodiments, the heterologous polynucleotide is a polynucleotide encoding a protein or inhibitory RNA that can induce differentiation of a cochlear supporting cell into a cochlear hair cell or induce or increase cochlear supporting cell proliferation (e.g., a polynucleotide listed in Table 5), or a transgene corresponding to a wild-type form of a gene that is expressed in cochlear supporting cells and mutated in a subject with hearing loss (e.g., a transgene corresponding to a wild-type form of a gene listed in Table 6). In some embodiments, the heterologous polynucleotide encodes (e.g., can be transcribed to produce) a short hairpin RNA (shRNA), an antisense oligonucleotide (ASO), a component of a gene editing system (e.g., a nuclease, such as a CRISPR Associated Protein 9 (Cas9), Transcription Activator-Like Effector Nuclease (TALEN), or Zinc Finger Nuclease (ZFN), or a guide RNA (gRNA)), or a microRNA.

In another aspect, the invention provides a polynucleotide including a GJB2 promoter having at least 85% sequence identity (e.g., 85%, 86%, 87%, 88%, 89%, 90%, 91%, 92%, 93%, 94%, 95%, 96%, 97%, 98%, 99%, or more, sequence identity) to any one of SEQ ID NOs: 13-19. In some embodiments, the GJB2 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 13. In some embodiments, the GJB2 promoter has the sequence of SEQ ID NO: 13. In some embodiments, the GJB2 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 14. In some embodiments, the GJB2 promoter has the sequence of SEQ ID NO: 14. In some embodiments, the GJB2 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 15. In some embodiments, the GJB2 promoter has the sequence of SEQ ID NO: 15. In some embodiments, the GJB2 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 16. In some embodiments, the GJB2 promoter has the sequence of SEQ ID NO: 16. In some embodiments, the GJB2 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 17. In some embodiments, the GJB2 promoter has the sequence of SEQ ID NO: 17. In some embodiments, the GJB2 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 18. In some embodiments, the GJB2 promoter has the sequence of SEQ ID NO: 18. In some embodiments, the GJB2 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 19. In some embodiments, the GJB2 promoter has the sequence of SEQ ID NO: 19.

In another aspect, the invention provides a polynucleotide including a GJB2 promoter having at least 85% sequence identity (e.g., 85%, 86%, 87%, 88%, 89%, 90%, 91%, 92%, 93%, 94%, 95%, 96%, 97%, 98%, 99%, or more, sequence identity) to any one of SEQ ID NOs: 25-28. In some embodiments, the GJB2 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 25. In some embodiments, the GJB2 promoter has the sequence of SEQ ID NO: 25. In some embodiments, the GJB2 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 26. In some embodiments, the GJB2 promoter has the sequence of SEQ ID NO: 26. In some embodiments, the GJB2 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 27. In some embodiments, the GJB2 promoter has the sequence of SEQ ID NO: 27. In some embodiments, the GJB2 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 28. In some embodiments, the GJB2 promoter has the sequence of SEQ ID NO: 28.

In another aspect, the invention provides a polynucleotide containing a GJB2 promoter having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 37. In some embodiments, GJB2 promoter has the sequence of SEQ ID NO: 37.

In another aspect, the invention provides a polynucleotide containing a GJB2 promoter including a first region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or a functional portion or derivative thereof including at least one of (e.g., one of more of) SEQ ID Nos: 20-24 operably linked to a second region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1 or a functional portion or derivative thereof including at least one of (e.g., one or more of) SEQ ID NOs: 3-12, optionally including a linker containing one to one hundred nucleotides between the first region and the second region.

In another aspect, the invention provides a nucleic acid vector containing the polynucleotide of any of the foregoing aspects and embodiments.

In another aspect, the invention provides a nucleic acid vector containing a polynucleotide including a GJB2 promoter having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 36. In some embodiments, the GJB2 promoter has the sequence of SEQ ID NO: 36.

In another aspect, the invention provides a nucleic acid vector containing a polynucleotide including a GJB2 promoter having at least 85% sequence identity (e.g., 85%, 86%, 87%, 88%, 89%, 90%, 91%, 92%, 93%, 94%, 95%, 96%, 97%, 98%, 99%, or more, sequence identity) to any one of SEQ ID NOs: 3-7. In some embodiments, the GJB2 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 3. In some embodiments, the GJB2 promoter has the sequence of SEQ ID NO: 3. In some embodiments, GJB2 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 4. In some embodiments, the GJB2 promoter has the sequence of SEQ ID NO: 4. In some embodiments, the GJB2 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 5. In some embodiments, the GJB2 promoter has the sequence of SEQ ID NO: 5. In some embodiments, the GJB2 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 6. In some embodiments, the GJB2 promoter has the sequence of SEQ ID NO: 6. In some embodiments, the GJB2 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 7. In some embodiments, GJB2 promoter has the sequence of SEQ ID NO: 7.

In another aspect, the invention provides a nucleic acid vector containing a polynucleotide including a GJB2 promoter having at least 85% sequence identity (e.g., 85%, 86%, 87%, 88%, 89%, 90%, 91%, 92%, 93%, 94%, 95%, 96%, 97%, 98%, 99%, or more, sequence identity) to any one of SEQ ID NOs: 2 and 20-24. In some embodiments, the GJB2 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2. In some embodiments, the GJB2 promoter has the sequence of SEQ ID NO: 2. In some embodiments, the GJB2 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 20. In some embodiments, the GJB2 promoter has the sequence of SEQ ID NO: 20. In some embodiments, the GJB2 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 21. In some embodiments, the GJB2 promoter has the sequence of SEQ ID NO: 21. In some embodiments, the GJB2 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 22. In some embodiments, the GJB2 promoter has the sequence of SEQ ID NO: 22. In some embodiments, the GJB2 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 23. In some embodiments, the GJB2 promoter has the sequence of SEQ ID NO: 23. In some embodiments, the GJB2 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 24. In some embodiments, the GJB2 promoter has the sequence of SEQ ID NO: 24.

In another aspect, the invention provides a nucleic acid vector containing a polynucleotide including a GJB2 promoter containing a first region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1 or a functional portion or derivative thereof including at least one of (e.g., one or more of) SEQ ID NOs: 3-12 operably linked to a second region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or a functional portion or derivative thereof including at least one of (e.g., one or more of) SEQ ID NOs: 20-24, optionally including a linker containing one to one hundred nucleotides between the first region and the second region.

In some embodiments of any of the foregoing aspects, the first region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1. In some embodiments, the first region has the sequence of SEQ ID NO: 1.

In some embodiments of any of the foregoing aspects, the first region has at least 85% sequence identity (e.g., 85%, 86%, 87%, 88%, 89%, 90%, 91%, 92%, 93%, 94%, 95%, 96%, 97%, 98%, 99%, or more, sequence identity) to any one of SEQ ID NOs: 3-12. In some embodiments, the first region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 3. In some embodiments, the first region has the sequence of SEQ ID NO: 3. In some embodiments, the first region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 4. In some embodiments, the first region has the sequence of SEQ ID NO: 4. In some embodiments, the first region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 5. In some embodiments, the first region has the sequence of SEQ ID NO: 5. In some embodiments, the first region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 6. In some embodiments, the first region has the sequence of SEQ ID NO: 6. In some embodiments, the first region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 7. In some embodiments, the first region has the sequence of SEQ ID NO: 7. In some embodiments, the first region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 8. In some embodiments, the first region has the sequence of SEQ ID NO: 8. In some embodiments, the first region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 9. In some embodiments, the first region has the sequence of SEQ ID NO: 9. In some embodiments, the first region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 10. In some embodiments, the first region has the sequence of SEQ ID NO: 10. In some embodiments, the first region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 11. In some embodiments, the first region has the sequence of SEQ ID NO: 11. In some embodiments, the first region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 12. In some embodiments, the first region has the sequence of SEQ ID NO: 12.

In some embodiments of any of the foregoing aspects, the functional portion of SEQ ID NO: 1 comprises the sequence of SEQ ID NO: 4 and the sequence of SEQ ID NO: 12. In some embodiments, the first region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 13. In some embodiments, the first region has the sequence of SEQ ID NO: 13.

In some embodiments of any of the foregoing aspects, the functional portion of SEQ ID NO: 1 comprises the sequence of SEQ ID NO: 5 and the sequence of SEQ ID NO: 12. In some embodiments, the first region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 14. In some embodiments, the first region has the sequence of SEQ ID NO: 14.

In some embodiments of any of the foregoing aspects, the functional portion of SEQ ID NO: 1 comprises the sequence of SEQ ID NO: 6 and the sequence of SEQ ID NO: 12. In some embodiments, the first region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 15. In some embodiments, the first region has the sequence of SEQ ID NO: 15.

In some embodiments of any of the foregoing aspects, the functional portion of SEQ ID NO: 1 comprises the sequence of SEQ ID NO: 7 and the sequence of SEQ ID NO: 12. In some embodiments, the first region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 16. In some embodiments, the first region has the sequence of SEQ ID NO: 16.

In some embodiments of any of the foregoing aspects, the functional portion of SEQ ID NO: 1 comprises the sequence of SEQ ID NO: 9 and the sequence of SEQ ID NO: 12. In some embodiments, the first region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 17. In some embodiments, the first region has the sequence of SEQ ID NO: 17.

In some embodiments of any of the foregoing aspects, the functional portion of SEQ ID NO: 1 comprises the sequence of SEQ ID NO: 10 and the sequence of SEQ ID NO: 12. In some embodiments, the first region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 18. In some embodiments, the first region has the sequence of SEQ ID NO: 18.

In some embodiments of any of the foregoing aspects, the functional portion of SEQ ID NO: 1 comprises the sequence of SEQ ID NO: 11 and the sequence of SEQ ID NO: 12. In some embodiments, the first region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 19. In some embodiments, the first region has the sequence of SEQ ID NO: 19.

In some embodiments of any of the foregoing aspects, the sequence of SEQ ID NO: 12 precedes the sequence of SEQ ID NO: 4, SEQ ID NO: 5, SEQ ID NO: 6, SEQ ID NO: 7, SEQ ID NO: 9, SEQ ID NO: 10, or SEQ ID NO: 11.

In some embodiments of any of the foregoing aspects, the second region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2. In some embodiments, the second region has the sequence of SEQ ID NO: 2.

In some embodiments of any of the foregoing aspects, the second region has at least 85% sequence identity (e.g., 85%, 86%, 87%, 88%, 89%, 90%, 91%, 92%, 93%, 94%, 95%, 96%, 97%, 98%, 99%, or more, sequence identity) to any one of SEQ ID NOs: 20-24. In some embodiments, the second region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 20. In some embodiments, the second region has the sequence of SEQ ID NO: 20. In some embodiments, the second region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 21. In some embodiments, the second region has the sequence of SEQ ID NO: 21. In some embodiments, the second region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 22. In some embodiments, the second region has the sequence of SEQ ID NO: 22. In some embodiments, the second region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 23. In some embodiments, the second region has the sequence of SEQ ID NO: 23. In some embodiments, the second region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 24. In some embodiments, the second region has the sequence of SEQ ID NO: 24.

In some embodiments of any of the foregoing aspects, the functional portion of SEQ ID NO: 2 comprises the sequence of SEQ ID NO: 23 and the sequence of SEQ ID NO: 20. In some embodiments, the second region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 25. In some embodiments, the second region has the sequence of SEQ ID NO: 25.

In some embodiments of any of the foregoing aspects, the functional portion of SEQ ID NO: 2 comprises the sequence of SEQ ID NO: 24 and the sequence of SEQ ID NO: 20. In some embodiments, the second region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 26. In some embodiments, the second region has the sequence of SEQ ID NO: 26.

In some embodiments of any of the foregoing aspects, the functional portion of SEQ ID NO: 2 comprises the sequence of SEQ ID NO: 23 and the sequence of SEQ ID NO: 22. In some embodiments, the second region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 27. In some embodiments, the second region has the sequence of SEQ ID NO: 27.

In some embodiments of any of the foregoing aspects, the functional portion of SEQ ID NO: 2 comprises the sequence of SEQ ID NO: 24 and the sequence of SEQ ID NO: 22. In some embodiments, the second region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 28. In some embodiments, the second region has the sequence of SEQ ID NO: 28.

In some embodiments of any of the foregoing aspects, the sequence of SEQ ID NO: 20 precedes the sequence of SEQ ID NO: 23 or SEQ ID NO: 24. In some embodiments of any of the foregoing aspects, the sequence of SEQ ID NO: 22 precedes the sequence of SEQ ID NO: 23 or SEQ ID NO: 24.

In some embodiments of any of the foregoing aspects, the first region is joined directly to the second region without a linker (e.g., the 3′ end of the first region directly precedes the 5′ end of the second region). In some embodiments of any of the foregoing aspects, the GJB2 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 29. In some embodiments, the GJB2 promoter has the sequence of SEQ ID NO: 29. In some embodiments of any of the foregoing aspects, the GJB2 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 30. In some embodiments, the GJB2 promoter has the sequence of SEQ ID NO: 30. In some embodiments of any of the foregoing aspects, the GJB2 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 31. In some embodiments, the GJB2 promoter has the sequence of SEQ ID NO: 31. In some embodiments of any of the foregoing aspects, the GJB2 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 32. In some embodiments, the GJB2 promoter has the sequence of SEQ ID NO: 32. In some embodiments of any of the foregoing aspects, the GJB2 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 33. In some embodiments, the GJB2 promoter has the sequence of SEQ ID NO: 33. In some embodiments of any of the foregoing aspects, the GJB2 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 34. In some embodiments, the GJB2 promoter has the sequence of SEQ ID NO: 34. In some embodiments of any of the foregoing aspects, the GJB2 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 35. In some embodiments, the GJB2 promoter has the sequence of SEQ ID NO: 35.

In another aspect, the invention provides a polynucleotide including a GJB2 enhancer having at least 85% sequence identity (e.g., 85%, 86%, 87%, 88%, 89%, 90%, 91%, 92%, 93%, 94%, 95%, 96%, 97%, 98%, 99%, or more, sequence identity) to any one of SEQ ID NOs: 52-63 (e.g., any one of SEQ ID NOs: 52-59) operably linked to a promoter, in which the distance between the enhancer and the promoter in the polynucleotide is less than 3 kilobases (3 kb). In some embodiments, the distance between the enhancer and the promoter in the polynucleotide is less than 2 kb. In some embodiments, the distance between the enhancer and the promoter in the polynucleotide is less than 1 kb. In some embodiments, the distance between the enhancer and the promoter in the polynucleotide is less than 0.5 kb. In some embodiments, the polynucleotide including the GJB2 enhancer is contained in a nucleic acid vector. In some embodiments, the promoter is an inner ear cell type-specific promoter (e.g., a cochlear supporting cell-specific promoter). In some embodiments, the inner ear cell type-specific promoter is a promoter listed in Table 9 (e.g., a promoter that can induce expression in a GJB2-expressing inner ear cell). In some embodiments, the promoter is a GJB2 promoter. In some embodiments, the GJB2 promoter is a promoter described herein above (e.g., a polynucleotide that contains a first region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1 or a functional portion or derivative thereof and/or a second region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or a functional portion or derivative thereof, optionally containing a linker joining the first region and the second region, such as a GJB2 promoter of any one of SEQ ID NOs: 29-35). In some embodiments, the GJB2 promoter has at least 85% sequence (e.g., 85%, 86%, 87%, 88%, 89%, 90%, 91%, 92%, 93%, 94%, 95%, 96%, 97%, 98%, 99%, or more, sequence identity) identity to any one of SEQ ID NOS: 1 and 8-11. In some embodiments, the GJB2 promoter has the sequence of any one of SEQ ID NOs: 1 and 8-11. In some embodiments, the GJB2 promoter has at least 85% sequence (e.g., 85%, 86%, 87%, 88%, 89%, 90%, 91%, 92%, 93%, 94%, 95%, 96%, 97%, 98%, 99%, or more, sequence identity) identity to a promoter listed in Table 8 (SEQ ID NOs: 66-68). In some embodiments, the GJB2 promoter has the sequence of a promoter listed in Table 8.

In another aspect, the invention provides a nucleic acid vector containing a polynucleotide including a GJB2 enhancer having at least 85% sequence identity (e.g., 85%, 86%, 87%, 88%, 89%, 90%, 91%, 92%, 93%, 94%, 95%, 96%, 97%, 98%, 99%, or more, sequence identity) to any one of SEQ ID NOs: 52-63 (e.g., any one of SEQ ID NOs: 52-59). In some embodiments, the enhancer is operably linked to a promoter. In some embodiments, the promoter is an inner ear cell type-specific promoter (e.g., a cochlear supporting cell-specific promoter). In some embodiments, the inner ear cell type-specific promoter is a promoter listed in Table 9 (e.g., a promoter that can induce expression in a GJB2-expressing inner ear cell). In some embodiments, the promoter is a GJB2 promoter. In some embodiments, the GJB2 promoter is a promoter described herein above (e.g., a polynucleotide that contains a first region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1 or a functional portion or derivative thereof and/or a second region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or a functional portion or derivative thereof, optionally containing a linker joining the first region and the second region, such as a GJB2 promoter of any one of SEQ ID NOs: 30-32). In some embodiments, the GJB2 promoter has at least 85% sequence (e.g., 85%, 86%, 87%, 88%, 89%, 90%, 91%, 92%, 93%, 94%, 95%, 96%, 97%, 98%, 99%, or more, sequence identity) identity to any one of SEQ ID NOS: 1 and 8-11. In some embodiments, the GJB2 promoter has the sequence of any one of SEQ ID NOs: 1 and 8-11. In some embodiments, the GJB2 promoter has at least 85% sequence (e.g., 85%, 86%, 87%, 88%, 89%, 90%, 91%, 92%, 93%, 94%, 95%, 96%, 97%, 98%, 99%, or more, sequence identity) identity to a promoter listed in Table 8 (SEQ ID NOs: 66-68). In some embodiments, the GJB2 promoter has the sequence of a promoter listed in Table 8.

In some embodiments of any of the foregoing aspects, the promoter (e.g., the GJB2 promoter) is operably linked to a polynucleotide encoding an expression product. In some embodiments, the expression product is a heterologous expression product. In some embodiments, the heterologous expression product is BDNF or NTF3. In some embodiments, the expression product is a protein, a short hairpin RNA (shRNA), an antisense oligonucleotide (ASO), a component of a gene editing system (e.g., a nuclease, such as a CRISPR Associated Protein 9 (Cas9), Transcription Activator-Like Effector Nuclease (TALEN), or Zinc Finger Nuclease (ZFN), or a guide RNA (gRNA)), or a microRNA. In some embodiments, the expression product is an expression product that is endogenously expressed in a GJB2-expressing cell. In some embodiments, the expression product is an expression product that is endogenously expressed in a GJB2-expressing inner ear cell. In some embodiments, the expression product is Gjb2 or Gjb6. In some embodiments, the polynucleotide encoding an expression product is a polynucleotide encoding a protein or inhibitory RNA that can induce differentiation of a cochlear supporting cell into a cochlear hair cell, a polynucleotide encoding a protein or inhibitory RNA that can induce or increase cochlear supporting cell proliferation, or a transgene corresponding to a wild-type form of a gene that is expressed in cochlear supporting cells and mutated in a subject with hearing loss. In some embodiments, the polynucleotide encoding an expression product is a polynucleotide listed in Table 5 or a transgene corresponding to a wild-type form of a gene listed in Table 6.

In some embodiments of any of the foregoing aspects, the polynucleotide further comprises a GJB2 enhancer operably linked to the GJB2 promoter.

In some embodiments of any of the foregoing aspects, the GJB2 enhancer has at least 85% sequence identity (e.g., 85%, 86%, 87%, 88%, 89%, 90%, 91%, 92%, 93%, 94%, 95%, 96%, 97%, 98%, 99%, or more, sequence identity) to any one of SEQ ID NOs: 52-63. In some embodiments, the GJB2 enhancer has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 52. In some embodiments, the GJB2 enhancer has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 53. In some embodiments, the GJB2 enhancer has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 54. In some embodiments, the GJB2 enhancer has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 55. In some embodiments, the GJB2 enhancer has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 56. In some embodiments, the GJB2 enhancer has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 57. In some embodiments, the GJB2 enhancer has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 58. In some embodiments, the GJB2 enhancer has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 59. In some embodiments, the GJB2 enhancer has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 60. In some embodiments, the GJB2 enhancer has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 61. In some embodiments, the GJB2 enhancer has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 62. In some embodiments, the GJB2 enhancer has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 63. In some embodiments, the GJB2 enhancer has the sequence of any one of SEQ ID NOs: 52-63. In some embodiments, the GJB2 enhancer has the sequence of SEQ ID NO: 52. In some embodiments, the GJB2 enhancer has the sequence of SEQ ID NO: 53. In some embodiments, the GJB2 enhancer has the sequence of SEQ ID NO: 54. In some embodiments, the GJB2 enhancer has the sequence of SEQ ID NO: 55. In some embodiments, the GJB2 enhancer has the sequence of SEQ ID NO: 56. In some embodiments, the GJB2 enhancer has the sequence of SEQ ID NO: 57. In some embodiments, the GJB2 enhancer has the sequence of SEQ ID NO: 58. In some embodiments, the GJB2 enhancer has the sequence of SEQ ID NO: 59. In some embodiments, the GJB2 enhancer has the sequence of SEQ ID NO: 60. In some embodiments, the GJB2 enhancer has the sequence of SEQ ID NO: 61. In some embodiments, the GJB2 enhancer has the sequence of SEQ ID NO: 62. In some embodiments, the GJB2 enhancer has the sequence of SEQ ID NO: 63.

In some embodiments of any of the foregoing aspects, the GJB2 enhancer is located 5′ of the promoter.

In some embodiments of any of the foregoing aspects, the GJB2 enhancer is located 3′ of the promoter.

In some embodiments of any of the foregoing aspects, the polynucleotide comprises two or more different GJB2 enhancers, in which each enhancer is independently selected from an enhancer having at least 85% sequence identity (e.g., 85%, 86%, 87%, 88%, 89%, 90%, 91%, 92%, 93%, 94%, 95%, 96%, 97%, 98%, 99%, or more, sequence identity) to any one of SEQ ID NOs: 52-63 (e.g., any one of SEQ ID NOS: 52-59). In some embodiments, each different GJB2 enhancer is independently selected from an enhancer having the sequence of one of SEQ ID NOs: 52-63 (e.g., any one of SEQ ID NOS: 52-59). In some embodiments, the polynucleotide comprises four different GJB2 enhancers. In some embodiments, the four enhancers are SEQ ID NO: 52, SEQ ID NO: 57, SEQ ID NO: 58, and SEQ ID NO: 59. In some embodiments, the four enhancers are SEQ ID NO: 53, SEQ ID NO: 54, SEQ ID NO: 55, and SEQ ID NO: 56.

In some embodiments of any of the foregoing aspects, the polynucleotide comprises two or more copies of a GJB2 enhancer having at least 85% sequence identity (e.g., 85%, 86%, 87%, 88%, 89%, 90%, 91%, 92%, 93%, 94%, 95%, 96%, 97%, 98%, 99%, or more, sequence identity) to any one of SEQ ID NOs: 52-63 (e.g., any one of SEQ ID NOS: 52-59). In some embodiments, each copy of the two or more copies of the enhancer has the sequence of one of SEQ ID NOs: 52-63 (e.g., any one of SEQ ID NOS: 52-59).

In some embodiments of any of the foregoing aspects, the nucleic acid vector is a viral vector, plasmid, cosmid, or artificial chromosome. In some embodiments, the nucleic acid vector is a viral vector. In some embodiments, the viral vector is an adeno-associated virus (AAV) vector, an adenovirus vector, or a lentivirus vector. In some embodiments, the viral vector is an AAV vector. In some embodiments, the AAV vector has an AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ, DJ/8, DJ/9, 7m8, PHP.B, PHP.eB, or PHP.S capsid. In some embodiments, the AAV vector has an AAV1 capsid. In some embodiments, the AAV vector has an AAV9 capsid. In some embodiments, the AAV vector has a 7m8 capsid. In some embodiments, the AAV vector has a PHP.S capsid. In some embodiments, the AAV vector has a DJ capsid. In some embodiments, the AAV vector has an Anc80 capsid. In some embodiments, the AAV vector has an Anc80L65 capsid. In some embodiments, the AAV vector has an AAV2 capsid. In some embodiments, the AAV vector has an AAV2quad(Y-F) capsid. In some embodiments, the AAV vector has a PHP.eB capsid. In some embodiments, the AAV vector has an AAV3 capsid. In some embodiments, the AAV vector has an AAV4 capsid. In some embodiments, the AAV vector has an AAV5 capsid. In some embodiments, the AAV vector has an AAV6 capsid. In some embodiments, the AAV vector has an AAV7 capsid. In some embodiments, the AAV vector has an AAV8 capsid. In some embodiments, the AAV vector has a PHP.B capsid.

In another aspect, the invention provides a composition containing the nucleic acid vector of any of the foregoing aspects and embodiments. In some embodiments, the composition further includes a pharmaceutically acceptable carrier, diluent, or excipient.

In another aspect, the invention provides a cell containing the polynucleotide or vector of any of the foregoing aspects and embodiments. In some embodiments, the cell is a GJB2-expressing cell. In some embodiments, the cell is a GJB2-expressing inner ear cell. In some embodiments, the cell is a mammalian cell. In some embodiments, the mammalian cell is a human cell. In some embodiments, the cell is a cochlear supporting cell.

In another aspect, the invention provides a method of expressing an expression product in a GJB2-expressing cell by contacting the GJB2-expressing cell with the nucleic acid vector of or composition of any of the foregoing aspects and embodiments. In some embodiments, the GJB2-expressing cell is a GJB2-expressing inner ear cell (e.g., a cochlear supporting cell). In some embodiments, the contacting is in a subject (e.g., in vivo).

In another aspect, the invention provides a method of treating a subject having or at risk of developing GJB2-related hearing loss by administering to an inner ear of the subject a therapeutically effective amount of the nucleic acid vector composition of any of the foregoing aspects and embodiments, in which the expression product is Gjb2 or Gjb6. In some embodiments, the GJB2-related hearing loss is DFNB1, DFNA3, or hearing loss associated with Bart-Pumphrey syndrome, hystrix-like ichthyosis with deafness, keratitis-ichthyosis-deafness syndrome, palmoplantar keratoderma with deafness, or Vohwinkel syndrome. In some embodiments, the GJB2-related hearing loss is DFNB1 or DFNA3. In some embodiments, the subject has a mutation in GJB2, a mutation in GJB6, or a mutation in both GJB2 and GJB6.

In another aspect, the invention provides a method of treating a subject having or at risk of developing hearing loss (e.g., sensorineural hearing loss or deafness) by administering to an inner ear of the subject an effective amount of the nucleic acid vector or composition of any of the foregoing aspects and embodiments.

In another aspect, the invention provides a method of treating a subject having or at risk of developing tinnitus by administering to an inner ear of the subject an effective amount of the nucleic acid vector or composition of any of the foregoing aspects and embodiments.

In another aspect, the invention provides a method of inducing or increasing cochlear hair cell regeneration in a subject in need thereof by administering to an inner ear of the subject a therapeutically effective amount of the nucleic acid vector or composition of any of the foregoing aspects and embodiments, in which the enhancer has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 59 and the expression product is an expression product that can promote or increase cochlear supporting cell proliferation or differentiation of cochlear supporting cells into cochlear hair cells. In some embodiments, the polynucleotide encoding the expression product is a polynucleotide listed in Table 5. In some embodiments, the expression product is an inhibitory RNA directed to LATS1 and/or LATS2.

In another aspect, the invention provides a method of treating a subject having or at risk of developing hearing loss associated with damage to or loss of cochlear hair cells by administering to an inner ear of the subject a therapeutically effective amount of the nucleic acid vector or composition of any of the foregoing aspects and embodiments, in which the enhancer has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 59 and the expression product is an expression product that can promote or increase cochlear supporting cell proliferation or differentiation of cochlear supporting cells into cochlear hair cells. In some embodiments, the polynucleotide encoding the expression product is a polynucleotide listed in Table 5. In some embodiments, the expression product is an inhibitory RNA directed to LATS1 and/or LATS2.

In another aspect, the invention provides a method of inducing or increasing differentiation of a cochlear supporting cell into a cochlear hair cell by contacting the cochlear supporting cell with the nucleic acid vector or composition of any of the foregoing aspects and embodiments, in which the enhancer has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 59 and the expression product is Atoh1 or an Atoh1 variant, Pou4F3, Gfi1, or Ikzf2. In some embodiments, the contacting is in vivo (e.g., in a subject).

In another aspect, the invention provides a method of inducing or increasing cochlear supporting cell proliferation by contacting the cochlear supporting cell with the nucleic acid vector or composition of any of the foregoing aspects and embodiments, in which the enhancer has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 59 and the expression product is Lgr5, Yap1, Tead2, or an inhibitory RNA directed to LATS1 and/or LATS2. In some embodiments, the contacting is in vivo (e.g., in a subject).

In another aspect, the invention provides a method of treating a subject having or at risk of developing genetic hearing loss associated with a mutation in a gene that is endogenously expressed in cochlear supporting cells by administering to an inner ear of the subject a therapeutically effective amount of the nucleic acid vector or composition of any of the foregoing aspects and embodiments, in which the enhancer has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 59 and the polynucleotide encoding the expression product is a wild-type form of the gene that is mutated in cochlear supporting cells. In some embodiments, the genetic hearing loss is associated with a disease listed in Table 6 and the polynucleotide encoding the expression product is a transgene corresponding to a wild-type form of a gene that is mutated in said disease (e.g., a gene listed in the same row as the disease in Table 6).

In another aspect, the invention provides a method of improving cochlear supporting cell function or cochlear supporting cell survival by contacting the cochlear supporting cell with the nucleic acid vector or composition of any of the foregoing aspects and embodiments. In some embodiments, the contacting is in vivo (e.g., in a subject).

In another aspect, the invention provides a method of improving cochlear supporting cell function or cochlear supporting cell survival in a subject in need thereof by administering to an inner ear of the subject a therapeutically effective amount of the nucleic acid vector or composition of any of the foregoing aspects and embodiments.

In some embodiments of any of the foregoing aspects, the subject has or is at risk of developing hearing loss (e.g., sensorineural hearing loss or deafness).

In some embodiments of any of the foregoing aspects, the hearing loss is acquired hearing loss.

In some embodiments, the acquired hearing loss is noise-induced hearing loss, age-related hearing loss, disease or infection-related hearing loss, head trauma-related hearing loss, or ototoxic drug-induced hearing loss. In some embodiments, the ototoxic drug is an aminoglycoside, an antineoplastic drug, ethacrynic acid, furosemide, a salicylate, or quinine.

In some embodiments of any of the foregoing aspects, the hearing loss is genetic hearing loss. In some embodiments, the genetic hearing loss is autosomal dominant hearing loss, autosomal recessive hearing loss, or X-linked hearing loss.

In some embodiments of any of the foregoing aspects, the cochlear supporting cell is a mammalian cochlear supporting cell. In some embodiments, the mammalian cochlear supporting cell is a human cochlear supporting cell.

In some embodiments of any of the foregoing aspects, the method further comprises evaluating the hearing of the subject prior to administering the nucleic acid vector or composition.

In some embodiments of any of the foregoing aspects, the method further comprises evaluating the hearing of the subject after administering the nucleic acid vector or composition.

In some embodiments of any of the foregoing aspects, the nucleic acid vector or composition is locally administered. In some embodiments, the nucleic acid vector or composition is administered to the inner ear. In some embodiments, the nucleic acid vector or composition is administered to the middle ear. In some embodiments, the nucleic acid vector or composition is administered transtympanically or intratympanically. In some embodiments, the nucleic acid vector or composition is administered into the perilymph. In some embodiments, the nucleic acid vector or composition is administered into the endolymph. In some embodiments, the nucleic acid vector or composition is administered to or through the oval window. In some embodiments, the nucleic acid vector or composition is administered to or through the round window.

In some embodiments of any of the foregoing aspects, the nucleic acid vector or composition is administered in an amount sufficient to prevent or reduce hearing loss, prevent or reduce tinnitus, delay the development of hearing loss, slow the progression of hearing loss, improve hearing, increase or induce expression of an expression product in GJB2-expressing cells, increase cochlear hair cell numbers, increase cochlear hair cell regeneration, increase cochlear supporting cell proliferation, promote or increase cochlear supporting cell survival, induce or increase the differentiation of cochlear supporting cells into cochlear hair cells, or improve cochlear supporting cell function.

In some embodiments of any of the foregoing aspects, the subject is a human subject.

In another aspect, the invention provides a kit including the polynucleotide, nucleic acid vector, or composition of any of the foregoing aspects and embodiments.

### Definitions

As used herein, the term “about” refers to a value that is within 10% above or below the value being described.

As used herein, “administration” refers to providing or giving a subject a therapeutic agent (e.g., a nucleic acid vector containing a GJB2 promoter and/or a GJB2 enhancer operably linked to a polynucleotide encoding an expression product), by any effective route. Exemplary routes of administration are described herein below.

As used herein, the phrase “administering to the inner ear” refers to providing or giving a therapeutic agent described herein to a subject by any route that allows for transduction of inner ear cells. Exemplary routes of administration to the inner ear include administration into the perilymph or endolymph, such as to or through the oval window, round window, or semicircular canal (e.g., horizontal canal), or by transtympanic or intratympanic injection, e.g., administration to a GJB2-expressing inner ear cell.

As used herein, the term “cell type” refers to a group of cells sharing a phenotype that is statistically separable based on gene expression data. For instance, cells of a common cell type may share similar structural and/or functional characteristics, such as similar gene activation patterns and antigen presentation profiles. Cells of a common cell type may include those that are isolated from a common tissue (e.g., epithelial tissue, neural tissue, connective tissue, or muscle tissue) and/or those that are isolated from a common organ, tissue system, blood vessel, or other structure and/or region in an organism.

As used herein, the terms “conservative mutation,” “conservative substitution,” and “conservative amino acid substitution” refer to a substitution of one or more amino acids for one or more different amino acids that exhibit similar physicochemical properties, such as polarity, electrostatic charge, and steric volume. These properties are summarized for each of the twenty naturally occurring amino acids in table 1, below.

TABLE 1
Representative physicochemical properties of naturally occurring amino acids

| | | | | Electrostatic | |
|---|---|---|---|---|---|
| | | | Side- | character at | |
| | 3 Letter | 1 Letter | chain | physiological pH | Steric |
| Amino Acid | Code | Code | Polarity | (7.4) | Volume† |
| Alanine | Ala | A | nonpolar | neutral | small |
| Arginine | Arg | R | polar | cationic | large |
| Asparagine | Asn | N | polar | neutral | intermediate |
| Aspartic acid | Asp | D | polar | anionic | intermediate |
| Cysteine | Cys | C | nonpolar | neutral | intermediate |
| Glutamic acid | Glu | E | polar | anionic | intermediate |
| Glutamine | Gln | Q | polar | neutral | intermediate |
| Glycine | Gly | G | nonpolar | neutral | small |
| Histidine | His | H | polar | Both neutral and | large |
| | | | | cationic forms in | |
| | | | | equilibrium at pH 7.4 | |
| Isoleucine | Ile | I | nonpolar | neutral | large |
| Leucine | Leu | L | nonpolar | neutral | large |
| Lysine | Lys | K | polar | cationic | large |
| Methionine | Met | M | nonpolar | neutral | large |
| Phenylalanine | Phe | F | nonpolar | neutral | large |
| Proline | Pro | P | non- | neutral | intermediate |
| | | | polar | | |
| Serine | Ser | S | polar | neutral | small |
| Threonine | Thr | T | polar | neutral | intermediate |
| Tryptophan | Trp | W | nonpolar | neutral | bulky |
| Tyrosine | Tyr | Y | polar | neutral | large |
| Valine | Val | V | nonpolar | neutral | intermediate |

†based on volume in A³: 50-100 is small, 100-150 is intermediate, 150-200 is large, and >200 is bulky

From this table it is appreciated that the conservative amino acid families include (i) G, A, V, L, and I; (ii) D and E; (iii) C, S and T; (iv) H, K and R; (v) N and Q; and (vi) F, Y and W. A conservative mutation or substitution is therefore one that substitutes one amino acid for a member of the same amino acid family (e.g., a substitution of Ser for Thr or Lys for Arg).

The terms “derived” and “derivative” as used herein refer to a nucleic acid, peptide, or protein or a variant or analog thereof comprising one or more mutations and/or chemical modifications as compared to a corresponding full-length wild-type nucleic acid, peptide, or protein. Non-limiting examples of chemical modifications involving nucleic acids include, for example, modifications to the base moiety, sugar moiety, phosphate moiety, phosphate-sugar backbone, or a combination thereof.

As used herein, the terms “effective amount,” “therapeutically effective amount,” and a “sufficient amount” of a composition, vector construct, or viral vector described herein refer to a quantity sufficient to, when administered to the subject, including a mammal, for example a human, effect beneficial or desired results, including clinical results, and, as such, an “effective amount” or synonym thereto depends upon the context in which it is being applied. For example, in the context of treating sensorineural hearing loss, it is an amount of the composition, vector construct, or viral vector sufficient to achieve a treatment response as compared to the response obtained without administration of the composition, vector construct, or viral vector. The amount of a given composition described herein that will correspond to such an amount will vary depending upon various factors, such as the given agent, the pharmaceutical formulation, the route of administration, the type of disease or disorder, the identity of the subject (e.g., age, sex, weight) or host being treated, and the like, but can nevertheless be routinely determined by one skilled in the art. Also, as used herein, a “therapeutically effective amount” of a composition, vector construct, or viral vector of the present disclosure is an amount which results in a beneficial or desired result in a subject as compared to a control. As defined herein, a therapeutically effective amount of a composition, vector construct, or viral vector of the present disclosure may be readily determined by one of ordinary skill by routine methods known in the art. Dosage regimen may be adjusted to provide the optimum therapeutic response.

As used herein, the term “endogenous” refers to a molecule (e.g., a polypeptide, nucleic acid, or cofactor) that is found naturally in a particular organism (e.g., a human) or in a particular location within an organism (e.g., an organ, a tissue, or a cell, such as a human cell, e.g., a human cochlear supporting cell).

As used herein, the term “express” refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein. The term “expression product” refers to a protein or RNA molecule produced by any of these events.

As used herein, the term “exogenous” describes a molecule (e.g., a polypeptide, nucleic acid, or cofactor) that is not found naturally in a particular organism (e.g., a human) or in a particular location within an organism (e.g., an organ, a tissue, or a cell, such as a human cell, e.g., a human cochlear supporting cell). Exogenous materials include those that are provided from an external source to an organism or to cultured matter extracted there from.

As used herein, the term “functional portion,” when referring to a promoter sequence described herein (e.g., a GJB2 promoter sequence), refers to a nucleotide sequence that is shorter than SEQ ID NO: 1 or SEQ ID NO: 2 and is capable of recruiting RNA polymerase and driving transcription of a gene to which it is operably linked. For example, in the context of the present disclosure, a functional portion of SEQ ID NO: 1 may be any one of SEQ ID NOs: 3-19 and a functional portion of SEQ ID NO: 2 may be any one of SEQ ID NOs: 20-28.

As used herein, the terms “Gjb2” and “GJB2” (also known as connexin 26 and CX26) refer to a protein encoded by the GJB2 gene and to the gene encoding this protein, respectively. GJB2 is a member of the connexin gene family. Nearly half of all hearing loss is attributed to mutations in one of four members of the connexin gene family, and GJB2 mutations are the most common. More than 100 different mutations in GJB2 have been identified that cause non-syndromic hearing loss, which is loss of hearing that is not associated with other signs and symptoms. The terms “Gjb2” and “GJB2” also refer to variants of wild-type Gjb2 and nucleic acids encoding the same, respectively, such as variant proteins having at least 85% sequence identity (e.g., 85%, 86%, 87%, 88%, 89%, 90%, 91%, 92%, 93%, 94%, 95%, 96%, 97%, 98%, 99%, or 99.9% identity, or more sequence identity) to the amino acid sequence of a wild-type Gjb2 protein (e.g., SEQ ID NO: 38 or SEQ ID NO: 45) or polynucleotides having at least 85% sequence identity (e.g., 85%, 86%, 87%, 88%, 89%, 90%, 91%, 92%, 93%, 94%, 95%, 96%, 97%, 98%, 99%, or 99.9% identity, or more sequence identity) to the nucleic acid sequence of a wild-type GJB2 gene (e.g., SEQ ID NO: 39, SEQ ID NO: 40, or SEQ ID NO: 46) or a codon-optimized sequence thereof (e.g., any one of SEQ ID NOs: 41-44), provided that the Gjb2 analog encoded retains the therapeutic function of wild-type (WT) Gjb2 (e.g., the ability form hemichannels in supporting cells).

As used herein, the term “GJB2-expressing cell” refers to a cell type in the body that is known to endogenously express GJB2. GJB2-expressing cells include epithelial cells of the esophagus, cervical cells (ectocervix), cells of the minor salivary gland, epithelial cells of the skin, epithelial cells of the vagina, respiratory epithelial cells, liver hepatocytes, epithelial cells of the kidney, cells of the testes, luminal epithelial cells of the mammary gland, pancreatic acinar cells, bladder urothelial cells, epithelial cells of the intestine, and GJB2-expressing inner ear cells.

As used herein, the term “GJB2 enhancer” refers to a polynucleotide that can be operably linked to a promoter (e.g., a GJB2 promoter or an inner ear cell-type specific promoter, such as a cochlear supporting cell-specific promoter) to regulate gene expression in GJB2-expressing cells. GJB2 enhancers for use in the compositions and methods described herein have at least 85% sequence identity to (e.g., 85%, 86%, 87%, 88%, 89%, 90%, 91%, 92%, 93%, 94%, 95%, 96%, 97%, 98%, 99%, or 99.9% identity, or more sequence identity) to any one of SEQ ID NOs: 52-63. The GJB2 enhancers described herein can be operably linked to a promoter that is operably linked to a polynucleotide encoding an expression product to increase the expression level of the expression product in GJB2-expressing cells and increase the number of GJB2-expressing cells in which the expression product is expressed.

As used herein, the term “GJB2-expressing inner ear cell” refers to a cell within the inner ear that endogenously expresses GJB2. GJB2-expressing cells within the ear are found in both the cochlea and the vestibule. Cochlear GJB2-expressing cells include inner phalangeal cells, inner border cells, inner pillar cells, outer pillar cells, Deiter cells, Hensen's cells, Claudius cells, interdental cells, inner sulcus cells, outer sulcus cells, cells of the spiral limbus, spiral prominence cells, root cells, basal cells of the stria vascularis, intermediate cells of the stria vascularis, fibrocytes of the spiral limbus and spiral ligament, and mesenchymal cells lining the scala vestibuli. Vestibular GJB2-expressing cells include supporting cells, dark cells, fibrocytes, and mesenchymal cells.

As used herein, the term “GJB2 promoter” refers to a polynucleotide that is capable of expressing a transgene specifically in GJB2-expressing cells, or a variant thereof, such as a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to a GJB2 promoter described herein. The GJB2 promoters of the disclosure contain one or more regulatory elements from the GJB2 locus and have a sequence including a first region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1 or a functional portion or derivative thereof and/or a second region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or a functional portion or derivative thereof. The first region may be joined directly to the second region (e.g., fused) or the first region may be joined to the second region by a nucleic acid linker.

As used herein, the term GJB2-related hearing loss refers to diseases and conditions that feature hearing loss associated with a mutation in GJB2, such as DFNB1, which is characterized by moderate to profound prelingual hearing loss and is inherited in an autosomal recessive pattern, and DFNA3, which is characterized by moderate to severe prelingual or postlingual hearing loss that becomes more severe over time and is inherited in an autosomal dominant pattern. GJB2-related hearing loss also occurs in Bart-Pumphrey syndrome, hystrix-like ichthyosis with deafness, keratitis-ichthyosis-deafness syndrome, palmoplantar keratoderma with deafness, and Vohwinkel syndrome, all of which are characterized by hearing loss and skin abnormalities and associated with mutations in GJB2. Two types of GJB2-related hearing loss, DFNB1 and DFNA3, can also be associated with mutations in GJB6, either alone or in combination with mutations in GJB2. For example, subjects with DFNB1 may have a mutation in GJB2, a mutation in GJB6, or a mutation in both genes.

As used herein, the term “heterologous” refers to a combination of elements that is not naturally occurring. For example, a heterologous transgene refers to a transgene that is not naturally expressed by the promoter to which it is operably linked.

As used herein, the terms “increasing” and “decreasing” refer to modulating resulting in, respectively, greater or lesser amounts, of function, expression, or activity of a metric relative to a reference. For example, subsequent to administration of a composition in a method described herein, the amount of a marker of a metric (e.g., transgene expression, ABR, or DPOAE) as described herein may be increased or decreased in a subject by at least 5%, 10%, 15%, 20%, 25%, 30%, 35%, 40%, 45%, 50%, 55%, 60%, 65%, 70%, 75%, 80%, 85%, 90%, 95% or 98% or more relative to the amount of the marker prior to administration. Generally, the metric is measured subsequent to administration at a time that the administration has had the recited effect, e.g., at least one week, one month, 3 months, or 6 months, after a treatment regimen has begun.

As used herein, “locally” or “local administration” means administration at a particular site of the body intended for a local effect and not a systemic effect. Examples of local administration are epicutaneous, inhalational, intra-articular, intrathecal, intravaginal, intravitreal, intrauterine, intra-lesional administration, lymph node administration, intratumoral administration, administration to the inner ear, and administration to a mucous membrane of the subject, wherein the administration is intended to have a local and not a systemic effect.

As used herein, the term “operably linked” refers to a first molecule joined to a second molecule, wherein the molecules are so arranged that the first molecule affects the function of the second molecule. The two molecules may or may not be part of a single contiguous molecule and may or may not be adjacent. For example, a promoter is operably linked to a transcribable polynucleotide molecule if the promoter modulates transcription of the transcribable polynucleotide molecule of interest in a cell. Additionally, two portions of a transcription regulatory element are operably linked to one another if they are joined such that the transcription-activating functionality of one portion is not adversely affected by the presence of the other portion. Two transcription regulatory elements may be operably linked to one another by way of a linker polynucleotide (e.g., an intervening non-coding polynucleotide) or may be operably linked to one another with no intervening nucleotides present.

As used herein, the term “plasmid” refers to a to an extrachromosomal circular double stranded DNA molecule into which additional DNA segments may be ligated. A plasmid is a type of vector, a nucleic acid molecule capable of transporting another nucleic acid to which it has been linked. Certain plasmids are capable of autonomous replication in a host cell into which they are introduced (e.g., bacterial plasmids having a bacterial origin of replication and episomal mammalian plasmids). Other vectors (e.g., non-episomal mammalian vectors) can be integrated into the genome of a host cell upon introduction into the host cell, and thereby are replicated along with the host genome. Certain plasmids are capable of directing the expression of genes to which they are operably linked.

As used herein, the term “polynucleotide” refers to a polymer of nucleosides. Typically, a polynucleotide is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. The term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (i.e., the succession of letters used as abbreviations for bases) that biochemically characterizes a specific nucleic acid. A polynucleotide sequence presented herein is presented in a 5′ to 3′ direction unless otherwise indicated.

As used herein, the term “promoter” refers to a recognition site on DNA that is bound by an RNA polymerase. The polymerase drives transcription of the transgene.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

As used herein, the term “pharmaceutical composition” refers to a mixture containing a therapeutic agent, optionally in combination with one or more pharmaceutically acceptable excipients, diluents, and/or carriers, to be administered to a subject, such as a mammal, e.g., a human, in order to prevent, treat or control a particular disease or condition affecting or that may affect the subject.

As used herein, the term “pharmaceutically acceptable” refers to those compounds, materials, compositions and/or dosage forms, which are suitable for contact with the tissues of a subject, such as a mammal (e.g., a human) without excessive toxicity, irritation, allergic response, and other problem complications commensurate with a reasonable benefit/risk ratio.

As used herein, the term “sample” refers to a specimen (e.g., blood, blood component (e.g., serum or plasma), urine, saliva, amniotic fluid, cerebrospinal fluid, tissue (e.g., placental or dermal), pancreatic fluid, chorionic villus sample, and cells) isolated from a subject.

As used herein, the terms “subject” and “patient” refer to an animal (e.g., a mammal, such as a human). A subject to be treated according to the methods described herein may be one who has been diagnosed with hearing loss (e.g., sensorineural hearing loss) or one at risk of developing this condition (e.g., due to a genetic mutation or a risk factor for hearing loss, such as an ototoxic drug, loud noise, head trauma, a disease or infection, or aging). Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition.

As used herein, the terms “transcription regulatory element” and “regulatory sequence” refer to a polynucleotide that controls, at least in part, the transcription of a gene of interest. Transcription regulatory elements may include promoters, enhancers, and other polynucleotides (e.g., polyadenylation signals) that control or help to control gene transcription. Examples of transcription regulatory elements are described, for example, in Lorence, Recombinant Gene Expression: Reviews and Protocols (Humana Press, New York, NY, 2012).

As used herein, the term “transfection” refers to any of a wide variety of techniques commonly used for the introduction of exogenous DNA into a prokaryotic or eukaryotic host cell, e.g., electroporation, lipofection, calcium phosphate precipitation, DEAE-dextran transfection, Nucleofection, squeeze-poration, sonoporation, optical transfection, magnetofection, impalefection and the like.

As used herein, the terms “transduction” and “transduce” refer to a method of introducing a vector construct or a part thereof into a cell. Wherein the vector construct is contained in a viral vector such as for example an AAV vector, transduction refers to viral infection of the cell and subsequent transfer and integration of the vector construct or part thereof into the cell genome.

As used herein, “treatment” and “treating” in reference to a disease or condition, refer to an approach for obtaining beneficial or desired results, e.g., clinical results. Beneficial or desired results can include, but are not limited to, alleviation or amelioration of one or more symptoms or conditions; diminishment of extent of disease or condition; stabilized (i.e., not worsening) state of disease, disorder, or condition; preventing spread of disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to have the condition or disorder or those in which the condition or disorder is to be prevented.

As used herein, the term “vector” refers to a nucleic acid vector, e.g., a DNA vector, such as a plasmid, cosmid, or artificial chromosome, an RNA vector, a virus, or any other suitable replicon (e.g., viral vector). A variety of vectors have been developed for the delivery of polynucleotides encoding exogenous proteins into a prokaryotic or eukaryotic cell. Examples of such expression vectors are described in, e.g., Gellissen, Production of Recombinant Proteins: Novel Microbial and Eukaryotic Expression Systems (John Wiley & Sons, Marblehead, MA, 2006). Expression vectors suitable for use with the compositions and methods described herein contain a polynucleotide sequence as well as, e.g., additional sequence elements used for the expression of proteins and/or the integration of these polynucleotide sequences into the genome of a mammalian cell. Certain vectors that can be used for the expression of transgene as described herein include vectors that contain regulatory sequences, such as promoter and enhancer regions, which direct gene transcription. Other useful vectors for expression of a transgene contain polynucleotide sequences that enhance the rate of translation of the transgene or improve the stability or nuclear export of the mRNA that results from gene transcription. These sequence elements include, e.g., 5′ and 3′ untranslated regions and a polyadenylation signal site in order to direct efficient transcription of the gene carried on the expression vector. The expression vectors suitable for use with the compositions and methods described herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

As used herein, the term “wild-type” refers to a genotype with the highest frequency for a particular gene in a given organism.

### BRIEF DESCRIPTION OF THE DRAWINGS

FIGS. 1A-1B are a graph and a series of images showing that Gjb2 delivery with a ubiquitous promoter contributed to elevated ABR thresholds and IHC loss in wild-type animals. ABR thresholds of injected (triangles), uninjected contralateral (circles, 3 frequencies measured), and naïve ears (black diamonds) are shown (FIG. 1A). Seven out of nine wild-type ears treated with CMV-Gjb2 showed elevated ABR thresholds (bracket). Inner hair cell loss in wild-type ears that had elevated vs. normal ABR responses (Pou4f3: inner hair cells, bottom bracket; Prestin: outer hair cells, top bracket) is shown in FIG. 1B. Animals with elevated ABR responses had no remaining inner hair cells.

FIGS. 2A-2B are a series of graphs showing luciferase activity (detected using the Nano-Glo Luciferase Assay) resulting from transfection of HeLa cells with plasmids containing a promoter sequence and a NanoLuc reporter. GJB2 promoters having the sequences of SEQ ID NOs: 4-7, 9, 13, 30-32, 36, and 37 were cloned upstream of a NanoLuc reporter. HeLa cells were transfected with plasmids containing the promoter and reporter. Twenty-four hours later, the Nano-Glo Luciferase Assay (Promega Catalog #N1110) was used to detect and quantify NanoLuc expression. Results are shown in FIGS. 2A-2B. MLP=control minimal promoter (unrelated to GJB2). pGL3 Basic+NanoLuc=control vector without a promoter.

FIGS. 3A-3E are a series of images showing GFP expression induced by different combinations of GJB2 promoters and enhancers. Organ of Corti explant cultures were established from P4-P6 mice and infected with AAVs expressing nuclear (H2B)-GFP under the control of various GJB2 promoter and enhancer combinations. Samples were infected with 2E10 vg/culture. The combinations tested included: AAV1-p.hGJB2(SEQ ID NO: 30)-H2B-GFP (FIG. 3A), AAV1-p.hGJB2(SEQ ID NO: 30)-H2B-GFP-Enhancer 9 (SEQ ID NO: 62) (FIG. 3B), AAV1-p.hGJB2(SEQ ID NO: 30)-H2B-GFP-Enhancer GH (SEQ ID NO: 60) (FIG. 3C), AAV1-p.hGJB2(SEQ ID NO: 32)-H2B-GFP (FIG. 3D), and AAV1-p.hGJB2(SEQ ID NO: 32)-H2B-GFP-Enhancer 9 (SEQ ID NO: 62) (FIG. 3E). Samples infected with a GJB2 promoter alone (FIGS. 3A and 3D) showed lower expression of the reporter protein compared to samples infected with a vector containing a GJB2 promoter and an enhancer (FIGS. 3B, 3C, and 3E).

FIGS. 4A-4F are a series of images showing GFP expression induced by different combinations of GJB2 promoters and enhancers. Organ of Corti explant cultures were established from P4-P6 mice and infected with AAVs expressing nuclear (H2B)-GFP under the control of various GJB2 promoter and enhancer combinations. Samples were infected with 2E10 vg/culture. The combinations tested included: AAV1-p.hGJB2(SEQ ID NO: 1)-H2B-GFP (FIG. 4A), AAV1-p.hGJB2(SEQ ID NO: 1)-H2B-GFP-Enhancer 9 (SEQ ID NO: 62) (FIG. 4B), AAV1-p.hGJB2(SEQ ID NO: 1)-H2B-GFP-Enhancer GH (SEQ ID NO: 60) (FIG. 4C), AAV1-p.hGJB2(SEQ ID NO: 35)-H2B-GFP (FIG. 4D), AAV1-p.hGJB2(SEQ ID NO: 35)-H2B-GFP-Enhancer 9 (SEQ ID NO: 62) (FIG. 4E), and AAV1-p.hGJB2(SEQ ID NO: 35)-H2B-GFP-Enhancer GH (SEQ ID NO: 60) (FIG. 4F). Samples infected with the GJB2 promoter of SEQ ID NO: 1 alone showed the lowest expression of the reporter protein (FIG. 4A). Adding a Splice Acceptor (SEQ ID NO: 22) resulted in increased expression of the promoter alone (FIG. 4D). For both the promoter and promoter+spice acceptor (SEQ ID NO: 35), the addition of enhancer elements showed the highest levels of reporter protein expression (FIGS. 4B, 4C, 4E, and 4F).

FIGS. 5A-5D are a series of images showing GFP expression induced by a GJB2 promoter in combination with different enhancer elements. To identify DNA elements within the GH enhancer responsible for enhancer activity, three truncations of Enhancer GH were tested in combination with the GJB2 promoter of SEQ ID NO: 30: GHA, GHB, and GHC. Organ of Corti explant cultures were established from P4-P6 mice and infected with AAVs expressing nuclear (H2B)-GFP under the control of various GJB2 promoter and enhancer combinations. Samples were infected with 2E10 vg/culture. Explant cultures infected with AAV1-p.hGJB2(SEQ ID NO: 30)-H2B-GFP-Enhancer GH (SEQ ID NO: 60) (FIG. 5A) or AAV1-p.hGJB2(SEQ ID NO: 30)-H2B-GFP-Enhancer GHA (SEQ ID NO: 61) (FIG. 5B) retained strong reporter expression, whereas truncations of Enhancer GH (AAV1-p.hGJB2(SEQ ID NO: 30)-H2B-GFP-Enhancer GHB (SEQ ID NO: 64) (FIG. 5C), and AAV1-p.hGJB2(SEQ ID NO: 30)-H2B-GFP-Enhancer GHC (SEQ ID NO: 65) (FIG. 5D)) lost enhancer activity.

FIGS. 6A-6C are a series of images showing GFP expression induced by a GJB2 promoter with and without a predicted histone mark found within intron 1 of the GJB2 locus. To determine whether methylated DNA elements within the first intron of the GJB2 locus affect promoter activity, we tested the addition of these DNA elements within the GJB2 promoter of SEQ ID NO: 30. Organ of Corti explant cultures were established from P4-P6 mice and infected with AAVs expressing nuclear (H2B)-GFP under the control of various GJB2 promoters. Samples were infected with 2E10 vg/culture. Similar levels of reporter protein were detected in all samples (AAV1-p.hGJB2(SEQ ID NO: 30)-H2B-GFP-Enhancer GH (SEQ ID NO: 60) (FIG. 6A), AAV1-p.hGJB2(SEQ ID NO: 33)-H2B-GFP-Enhancer GH (SEQ ID NO: 60), AAV1-p.hGJB2(SEQ ID NO: 34)-H2B-GFP-Enhancer GH (SEQ ID NO: 60) (FIG. 6C)) indicating the methylated DNA (histone mark) does not influence expression.

FIGS. 7A-7F are a series of images showing GFP expression induced by a GJB2 promoter combined with different enhancers. Organ of Corti explant cultures were established from P4-P6 mice and infected with AAVs expressing nuclear (H2B)-GFP under the control of various GJB2 promoter and enhancer combinations. Samples were infected with 2E10 vg/culture. The combinations tested included: AAV1-p.hGJB2(SEQ ID NO: 30)-H2B-GFP (FIG. 7A), AAV1-p.hGJB2(SEQ ID NO: 30)-H2B-GFP-Enhancer GH (SEQ ID NO: 60) (FIG. 7B), AAV1-p.hGJB2(SEQ ID NO: 30)-H2B-GFP-Enhancer GHA (SEQ ID NO: 61) (FIG. 7C), AAV1-p.hGJB2(SEQ ID NO: 30)-H2B-GFP-Enhancer 9 (SEQ ID NO: 62) (FIG. 7D), AAV1-p.hGJB2(SEQ ID NO: 30)-H2B-GFP-Enhancer 9C (SEQ ID NO: 63) (FIG. 7E), and AAV1-CMV-H2B-GFP (FIG. 7F). Samples infected with the combination of promoter and the full-length enhancers (Enhancer GH and Enhancer 9) showed higher expression of the reporter protein (FIGS. 7B and 7D) when compared to the truncated enhancers (FIGS. 7C and 7E). Expression of the reporter protein was observed in all cells in the control sample (AAV1-CMV-H2B-GFP, FIG. 7F).

FIGS. 8A-8D are a series of images showing that in an AAV-DJ vector Enhancer GH induced more GFP expression in adult mouse cochlea compared to Enhancer 9. Representative images of whole mount cochlea show GFP expression (GFP, white) in supporting cells of the cochlea (FIGS. 8A-8B). GFP was excluded from hair cells (Myo7a, grey) in the sensory epithelium (white brackets). Cross-section of adult mouse cochlea confirmed that GFP expression was restricted to supporting cells (GFP+ nuclei, dark grey; black arrows) and excluded from hair cells in the sensory epithelium (bracket) (FIGS. 8C-8D).

FIGS. 9A-9B are a series of images showing that Enhancer GH induced more GFP expression in neonatal mouse cochlea compared to Enhancer 9 in an AAV-DJ vector. Representative images of whole mount cochlea show GFP expression (GFP, white) in supporting cells of the cochlea (FIGS. 9A-9B). GFP was excluded from hair cells (Myo7a, grey) in the sensory epithelium (white brackets).

FIGS. 10A-10B are a series of images and a graph showing that Enhancer GH in AAV1 induced GFP expression in supporting cells of the neonatal mouse ear. Representative images of whole mount cochlea show GFP expression at specific frequency regions (FIG. 10A, GFP, white; Pou4f3 hair cells, light grey). The top panel is a merged image showing GFP and Pou4f3 and the bottom panel shows GFP alone. GFP expression was excluded from hair cells in the sensory epithelium (bracket). Quantification of GFP+ supporting cells shows more GFP expression in medial supporting cells compared to lateral supporting cells (FIG. 10B).

FIGS. 11A-11B are a series of images showing that Enhancer GH in AAV1 induced GFP expression in the lateral wall and stria vascularis of the neonatal mouse ear. Two representative images of whole mount lateral wall show GFP expression in the lateral wall and stria vascularis (GFP, white; DAPI, dark grey) (A and B of FIGS. 11A-11B). Whole mount preparation looking at marginal cell layer of stria vascularis. GFP positive nuclei highlighted with arrows. z, y orthogonal projection through lateral wall (A′ and B′ of FIGS. 11A-11B). Basal cell layer of stria vascularis is on the left, marginal cell layer is on the right.

FIGS. 12A-12B are a series of images and a graph showing that Enhancer GH in Php.B drives GFP expression in supporting cells of the neonatal mouse ear. Representative images of whole mount cochlea show GFP expression at specific frequency regions (FIG. 12A, GFP, white; Pou4f3 hair cells, light grey). The top panel is a merged image showing GFP and Pou4f3 and the bottom panel shows GFP alone. GFP expression was excluded from hair cells in the sensory epithelium (bracket). FIG. 12B shows the quantification of GFP+ supporting cells.

FIGS. 13A-13B are a series of images showing GFP expression from Enhancer GH in the Php.B serotype delivered at neonatal ages. Cross-sections of mouse cochlea show GFP expression was restricted to supporting cells and the lateral wall (FIGS. 13A-13B; GFP+ nuclei, dark grey; black arrows) and excluded from hair cells in the sensory epithelium (bracket).

FIG. 14 is a series of graphs and images showing that the GJB2 promoter of SEQ ID NO: 30 in combination with Enhancer GH restored hearing and prevented outer hair cell loss in a gene replacement gene therapy in a GJB2-deficient mouse model. Using Php.B serotype and delivering GJB2 to neonatal mice resulted in an average of 40 dB hearing improvement. The left panels show ABR and DPOAE results comparing injected to contralateral ear four weeks post injection. The middle panel shows ABR waveforms of a representative responder. The images on the right show that outer hair cells (OHCs) were preserved in the treated ear compared to the contralateral control (Prestin, white).

FIG. 15 is a series of graphs and images showing that the GJB2 promoter of SEQ ID NO: 30 in combination with Enhancer GH restored hearing and prevented outer hair cell loss in a gene replacement gene therapy in a GJB2-deficient mouse model. Using AAV1 serotype and delivering GJB2 to neonatal mice resulted in an average of 20 dB hearing improvement. The left panels show ABR and DPOAE results comparing injected to contralateral ear four weeks post injection. The middle panel shows ABR waveforms of a representative responder. The images on the right show that outer hair cells (OHCs) were preserved in the treated ear compared to the contralateral control (Prestin, white). Similar results were observed using the AAV-DJ serotype.

FIG. 16 is a series of images showing GFP expression induced by different enhancers in neonatal cochlear explants. Enhancers driving histone-tagged GFP were screened in explants using AAV-DJ. Expression pattern, GFP intensity, and exclusion from hair cells were assessed. The sensory epithelium is marked with a bracket (GFP, white; Myo7a, gray). High GFP expression was observed with Enhancer 1, Enhancer, 8, and the two enhancer combinations (Enhancers 1+6+7+8 and Enhancers 2+3+4+5). Complete exclusion from hair cells was observed with Enhancers 1, 4, 5, and 6 and with the two enhancer combinations. GFP was observed in the sensory epithelium region for all enhancers and enhancer combinations tested with the exception of Enhancer 7. Enhancer 1: SEQ ID NO: 52; Enhancer 2: SEQ ID NO: 53; Enhancer 3: SEQ ID NO: 54; Enhancer 4: SEQ ID NO: 55; Enhancer 5: SEQ ID NO: 56; Enhancer 6: SEQ ID NO: 57; Enhancer 7: SEQ ID NO: 58; Enhancer 8: SEQ ID NO: 59.

FIG. 17 is a series of images showing GFP expression induced by different enhancers in vivo. Neonatal mice were injected with various enhancers paired with the GJB2 promoter of SEQ ID NO: 30 using AAV-DJ serotype. Inner ear whole mount (top row) and sections (bottom row) were collected. In Enhancer 1, GFP expression was present in all supporting cells of the sensory epithelium and many non-sensory cell types (top row). GFP was excluded from hair cells for all enhancers tested (GFP, white; Myo7a hair cells, grey). GFP was detected in the lateral wall with Enhancer 4. Section histology confirms whole mount expression (bottom row; GFP, dark grey; black arrows indicate examples of GFP+ cells).

FIG. 18 is a series of images showing GFP expression induced by different enhancers in vivo. Neonatal mice were injected with various enhancers paired with the GJB2 promoter of SEQ ID NO: 30 using AAV-DJ serotype. Inner ear whole mount (top row) and sections (bottom row) were collected. In Enhancer 8, GFP expression was restricted to supporting cells of the sensory epithelium (top row). GFP was excluded from hair cells for all enhancers tested. GFP expression was observed in Reissner's membrane for Enhancer 6 and the combination of Enhancers 2+3+4+5. In the combination of Enhancers 1+6+7+8, GFP expression was present in all supporting cells of the sensory epithelium, most non-sensory cells of the cochlear duct, cells of the lateral wall and stria vascularis (GFP, white; Myo7a hair cells, grey). Section histology confirms whole mount expression (bottom row; GFP, dark grey; black arrows indicate examples of GFP+ cells).

FIGS. 19A-19C are a series of schematics depicting the order of elements (e.g., promoter, coding sequence (GFP or mGJB2), WPRE (if present), polyadenylation sequence (polyA), and enhancer (if present)) included in the vectors used in the experiments described herein. FIG. 19A is a schematic depicting the order of the elements in the vectors used to generate the data shown in FIGS. 3A-3E, FIGS. 4A-4F, FIGS. 5A-5D, FIGS. 6A-6C, FIGS. 7A-7F, FIGS. 8A-8F, FIGS. 9A-9B, FIGS. 10A-10B, FIGS. 11A-11B, FIGS. 12A-12B, and FIGS. 13A-13B. FIG. 19B is a schematic depicting the order of the elements in the vectors used to generate the data shown in FIGS. 14 and 15. FIG. 19C is a schematic depicting the order of the elements in the vectors used to generate the data shown in FIGS. 16-18.

FIGS. 20A-20B are a series of graphs showing that overexpression of GJB2 using a GJB2 promoter/enhancer pair is safe as compared to overexpression of GJB2 using a ubiquitous promoter. Wild-type mice were injected with a vehicle control, or a human GJB2 transgene driven by a ubiquitous promoter (CMV) or a GJB2 promoter/enhancer pair (the GJB2 promoter of SEQ ID NO: 30 and the GJB2 enhancer of SEQ ID NO: 52). FIG. 20A shows ABR thresholds at baseline and two weeks after injection. Elevated ABR thresholds were seen in animals in the ubiquitous promoter group, while animals in the GJB2 promoter group had ABR thresholds on par with the baseline measurements, as seen in the vehicle group as well. This shows that non-specific hGJB2 expression leads to hearing loss. FIG. 20B shows inner and outer hair cell counts for animals across treatment groups. Inner hair cell loss was seen in the ubiquitous promoter group, which shows that off-target GJB2 expression leads to inner hair cell toxicity, which can be mitigated through use of a GJB2 promoter.

FIGS. 21A-21C are a series of graphs showing that AAV1 encoding human GJB2 driven by p.hGJB2(SEQ ID NO: 30)+Enhancer 1(SEQ ID NO: 52) restored hearing and prevented outer hair cell loss in a gene replacement gene therapy in a GJB2-deficient mouse model. FIGS. 21A-21B show ABR (FIG. 21A) and DPOAE (FIG. 21B) results comparing injected to contralateral ear 4, 8, and 12 weeks post injection. Hearing recovery was observed in the majority of animals. The variability in responses seen is thought to be due to variability in the amount of virus effectively delivered to the inner ear due to the technicality of the surgery. In some responder animals, the contralateral ear appears to also have some hearing recovery. This is due to the fact that virus crossover can occur after neonatal injection. FIG. 21C shows that outer hair cells were preserved in injected ears compared to uninjected contralateral ears.

FIGS. 22A-22C are a series of graphs showing that AAV1 encoding human GJB2 driven by AAV1-p.hGJB2(SEQ ID NO: 30)+Enhancers 1+6+7+8(SEQ ID NOs: 52, 57, 58, 59) restored hearing and prevented outer hair cell loss in a gene replacement gene therapy in a GJB2-deficient mouse model. FIGS. 22A-22B show ABR (FIG. 22A) and DPOAE (FIG. 22B) results comparing injected to contralateral ear 4, 12, and 14 weeks post injection. Hearing recovery was observed in a subset of animals. The variability in responses seen is thought to be due to variability in the amount of virus effectively delivered to the inner ear due to the technicality of the surgery. FIG. 22C shows that outer hair cells were preserved in injected ears compared to uninjected contralateral ears.

FIGS. 23A-23C are a series of graphs showing that AAV1 encoding human GJB2 driven by AAV1-p.hGJB2(SEQ ID NO: 30)+Enhancer 8(SEQ ID NO: 59) restored hearing and prevented outer hair cell loss in a gene replacement gene therapy in a GJB2-deficient mouse model. FIGS. 23A-23B show ABR (FIG. 23A) and DPOAE (FIG. 23B) results comparing injected to contralateral ear 4 and 15 weeks post injection. Hearing recovery was observed in a subset of animals. The variability in responses seen is thought to be due to variability in the amount of virus effectively delivered to the inner ear due to the technicality of the surgery. In some responder animals, the contralateral ear appears to also have some hearing recovery. This is due to the fact that virus crossover can occur after neonatal injection. FIG. 23C shows that outer hair cells were preserved in injected ears of responder animals compared to contralateral ears and non-responders.

FIG. 24 is a series of graphs showing quantification of GFP expression in supporting cells from non-human primate (NHP) whole mount histology. NHPs were injected with AAV1 virus, with nuclear GFP driven by promoter/enhancer combinations AAV1-p.hGJB2(SEQ ID NO: 30)+GJB2enh-GFP, with GJB2enh corresponding to one of: Enhancer 1 (SEQ ID NO: 52), Enhancer GH (SEQ ID NO: 60), or Enhancers 1+6+7+8(SEQ ID NOs: 52, 57, 58, 59). Whole mount histology was used to quantify the percentage of GFP expressing medial (left) and lateral (right) supporting cells. All constructs were able to drive expression in these cell types across the tonotopic axis.

FIGS. 25A-25C are a series of images showing that the GJB2 promoter of SEQ ID NO: 30 in combination with GJB2 Enhancer 1 (SEQ ID NO: 52), Enhancer GH (SEQ ID NO: 60), or Enhancers 1+6+7+8(SEQ ID NOs: 52, 57, 58, 59) induced GFP expression in NHP cochlea. FIGS. 25A-25C show representative images of NHPs injected with AAV1 and various promoter/enhancer combinations driving H2B-GFP expression. FIG. 25A: AAV1-p.hGJB2(SEQ ID NO: 30)+Enhancer 1-GFP; FIG. 25B: AAV1-p.hGJB2(SEQ ID NO: 30)+Enhancer GH-GFP; AAV1-p.hGJB2(SEQ ID NO: 30)+Enhancers 1+6+7+8-GFP. Images are shown for various frequencies and are centered around the sensory epithelium. Minimal expression was seen in hair cells. Different promoter/enhancer combinations drove expression in supporting cells to varying degrees. In each figure, left: Myo7a (hair cells), middle: native GFP, right: anti-GFP.

FIG. 26 is a table showing quantification of GFP expression across cochlear cell types from NHP sections. NHPs were injected with AAV1 virus, with nuclear GFP driven by promoter/enhancer combinations AAV1-p.hGJB2(SEQ ID NO: 30)+GJB2enh-GFP, with GJB2enh corresponding to one of: Enhancer 1 (SEQ ID NO: 52), Enhancer GH (SEQ ID NO: 60), or Enhancers 1+6+7+8(SEQ ID NOs: 52, 57, 58, 59). Cell types in sections were scored from 0 to 3, for lowest to highest expression, based on the amount of staining seen for GFP immunohistochemistry (IHC) or in situ hybridization (ISH). Note that the ISH probe labels both vector genomes and mRNA transcript. GFP expression was detected in a variety of GJB2-expressing cell types, including notably in the supporting cells (IBC through Claudius/Hensen). Hair cells appeared to primarily have ISH, but not IHC labeling. G1: Enhancer GH, G2: Enhancer 1, G3: Enhancers 1+6+7+8. IHCs: inner hair cells, OHCs: outer hair cells, SL: spiral limbus, IBC: inner border cell, IPhC: inner phalangeal cell, IPC: inner pillar cell, OPC: outer pillar cell, SGN: spiral ganglion neurons, SG Glia: Spiral ganglion glia.

FIGS. 27A-27B are a series of images and a graph showing that a GJB2 promoter/enhancer combination induced hGJB2-FLAG expression in medial and lateral supporting cells in the NHP. NHPs were injected with 60 μL of AAV1 virus (at a dose of 1× or 2×), with human FLAG-tagged GJB2 driven by the GJB2 promoter of SEQ ID NO: 30 (AAV1-p.hGJB2(SEQ ID NO: 30)+Enhancer 1(SEQ ID NO: 52)-hGJB2-FLAG). Representative images from the sensory epithelium region, taken from 2 different animals (from the 2× dose group) at the indicated frequencies are shown in FIG. 27A. The medial and lateral supporting cell regions are indicated in brackets, as defined using the DAPI labeling. FLAG staining closely mirrored the endogenous GJB2 pattern. Quantification of the percentage of FLAG-positive Deiter cells in individual animals is shown in FIG. 27B. The transgene could be detected throughout the length of the cochlea, with higher expression levels seen in the higher virus dose group.

FIG. 28 is a series of images of neonatal cochlear explants treated with AAVs containing GFP driven by two different promoters: a minimal beta-globin promoter (B-glob) that is not specific to any cells in the inner ear, and a proximal GJB2 promoter of SEQ ID NO: 1 (proxGJB2). The top row shows staining for Myosin7a, a hair cell marker. The second row shows staining for Sox2, a supporting cell marker. The bottom row shows GFP expression. The B-glob promoter induced very little GFP expression on its own, and in combination with either Enhancer 1 (SEQ ID NO: 52) or Enhancer 8 (SEQ ID NO: 59), induced expression in many cells of the sensory epithelium, including both hair cells and supporting cells. The proxGJB2 promoter alone induced no GFP expression in the sensory epithelium of the cochlea. In combination with Enhancer 1, proxGJB2 induced strong expression in lateral supporting cells and outer hair cells, and minimal expression in medial supporting cells and inner hair cells. ProxGJB2 in combination with Enhancer 8 induced strong expression in all supporting cells and was completely excluded from hair cells.

### DETAILED DESCRIPTION

Described herein are compositions and methods for inducing transgene expression specifically in GJB2-expressing cells (e.g., GJB2-expressing inner ear cells, such as cochlear supporting cells). The invention features GJB2 promoters that can induce expression of an expression product (e.g., a protein encoded by a transgene or an RNA molecule, such as an inhibitory RNA molecule) in GJB2-expressing cells (e.g., cochlear supporting cells) with minimal to no expression in cochlear hair cells. In addition, the invention features GJB2 enhancers that can be operably linked to a promoter to induce transgene expression in GJB2-expressing cells (e.g., GJB2-expressing inner ear cells) and minimize off-target expression in non-GJB2-expressing cells (e.g., cochlear hair cells). The GJB2 enhancers can also increase gene expression level and the number of GJB2-expressing cells in which gene expression can be detected. The invention also features nucleic acid vectors containing the GJB2 promoters described herein operably linked to a polynucleotide encoding an expression product (e.g., a polynucleotide encoding a protein or an inhibitory RNA) and nucleic acid vectors containing the GJB2 enhancers described herein operably linked to a promoter that is, in turn, operably linked to a polynucleotide encoding an expression product (e.g., a polynucleotide encoding a protein or an inhibitory RNA molecule). The compositions and methods described herein can be used to express an expression product (e.g., a protein, inhibitory RNA, microRNA, or a component of a gene editing system) specifically in GJB2-expressing cells, and, therefore, the compositions described herein can be administered to a subject (such as a mammalian subject, for instance, a human) to treat disorders caused by dysfunction of GJB2-expressing cells, such as hearing loss (e.g., sensorineural hearing loss, such as GJB2-related hearing loss, other genetic forms of hearing loss associated with mutations in cochlear supporting cell genes, or hearing associated with a loss of cochlear hair cells, such as age-related hearing loss, ototoxic drug-induced hearing loss, noise-induced hearing loss, head trauma-related hearing loss, or disease or infection-related hearing loss).

### Supporting Cells

Sensory epithelia of the inner ear contain two major cell types: hair cells and supporting cells. Hair cells are sensory cells of the auditory and vestibular systems that reside in the inner ear. Cochlear hair cells are the sensory cells of the auditory system and are made up of two main cell types: inner hair cells, which are responsible for sensing sound, and outer hair cells, which are thought to amplify low-level sound. Vestibular hair cells are located in the semicircular canal end organs and otolith organs of the inner ear and are involved in the sensation of movement that contributes to the sense of balance and spatial orientation. The development, function, and maintenance of inner ear sensory epithelia is highly dependent upon supporting cells, which are non-sensory cells that reside between hair cells. Supporting cells in the cochlea include Hensen's cells, Deiter cells, inner and outer pillar cells, Claudius cells, inner phalangeal cells, and border cells. Supporting cells are linked to each other and to hair cells by tight and adherens junctions and they communicate directly with other supporting cells by gap junctions. Gap junctions are made up of connexins that are encoded by connexin genes, such as CX26 (also known as GJB2) and CX30 (also known as GJB6). These connexin channels play an important role in recycling and regulating intracellular K+as well as pH homeostatic mechanisms and may also provide a pathway for rapid removal of ions from the region of the sensory cells during sound conduction in order to maintain sensitivity. Supporting cells have rigid cytoskeletons that maintain the structural integrity of the sensory organs during sound stimulation and head movements and, following trauma or toxicity, can eject injured hair cells from the epithelium, phagocytose hair cell debris, and, in some cases, generate new hair cells.

Gene therapy has recently emerged as an attractive therapeutic approach for treating hearing loss, particularly hearing loss caused by a mutation in a gene expressed in the inner ear. Mutations in many different genes have been found to cause hearing loss, including mutations in genes expressed in cochlear supporting cells. For example, mutations in GJB2 are the most common cause of recessive hearing loss, and mutations in other cochlear supporting cell genes, such as GJB6, SLC26A4, and GAS2, have also been linked to hearing loss. Another potential application for gene therapy is to induce regeneration of cochlear hair cells, which are often lost or damaged in age-related hearing loss, ototoxic drug-induced hearing loss, noise-induced hearing loss, head trauma-related hearing loss, and disease or infection-related hearing loss, by inducing differentiation of cochlear supporting cells into cochlear hair cells. However, using gene therapy to treat hearing loss associated with mutations in cochlear supporting cell genes (e.g., genes expressed in cochlear supporting cells) or to promote differentiation of cochlear supporting cells into cochlear hair cells calls for methods for inducing gene expression in cochlear supporting cells and not in cochlear hair cells, which are currently quite limited.

### GJB2

Gap junction protein beta 2 (Gjb2, also known as Connexin 26) is a protein encoded by the GJB2 gene and is a member of the connexin gene family. Connexins oligomerize into hexameric arrangements called connexons or hemichannels, which often dock with hemichannels from a contacting cell to form gap junctions. Nearly half of all hearing loss is attributed to mutations in one of four members of the connexin gene family, and GJB2 mutations are the most common. More than 100 different mutations in GJB2 have been identified that cause non-syndromic hearing loss, which is loss of hearing that is not associated with other signs and symptoms. One form of non-syndromic hearing loss that is associated with mutations in GJB2 is DFNB1, which is characterized by moderate to profound prelingual hearing loss and is inherited in an autosomal recessive pattern. DFNA3 is the other form of non-syndromic hearing loss that is associated with mutations in GJB2 and is moderate to severe prelingual or postlingual hearing loss that becomes more severe over time and is inherited in an autosomal dominant pattern. Other health conditions associated with mutations in GJB2 include Bart-Pumphrey syndrome, hystrix-like ichthyosis with deafness, keratitis-ichthyosis-deafness syndrome, palmoplantar keratoderma with deafness, and Vohwinkel syndrome, all of which are characterized by hearing loss and skin abnormalities.

The present invention is based, in part, on the discovery of regions upstream of the GJB2 coding sequence that can be used to promote expression of a transgene specifically in GJB2-expressing cells (e.g., cochlear supporting cells cells). The present inventors determined that it was desirable to identify promoters that could induce transgene expression specifically in GJB2-expressing cells after observing loss of inner hair cells and elevated ABR thresholds when GJB2 was expressed in wild-type mice using a ubiquitous promoter. The compositions and methods described herein can, thus, be used to express an expression product (e.g., a polynucleotide encoding a protein or a polynucleotide that can be transcribed to produce an inhibitory RNA molecule) in GJB2-expressing cells (e.g., GJB2-expressing inner ear cells, such as cochlear supporting cells), such as a gene that is endogenously expressed in GJB2-expressing cells, a cochlear supporting cell gene (e.g., a gene expressed in cochlear supporting cells) known to be mutated in in subjects with hearing loss, or a gene that can induce the differentiation of cochlear supporting cells into cochlear hair cells, to treat subjects having or at risk of developing hearing loss (e.g., sensorineural hearing loss), deafness, and/or tinnitus. The discovery of GJB2 promoters that induce expression in GJB2-expressing cells while minimizing or eliminating off-target expression in cells that do not express GJB2 (e.g., cochlear hair cells) can improve the safety and efficacy of gene therapy by reducing toxicity associated with off-target expression.

The polynucleotides of the compositions and methods described herein include nucleic acid sequences from regions of the GJB2 locus that are capable of expressing a transgene specifically in GJB2-expressing cells, or variants thereof, such as a nucleic acid sequences that have at least 85% sequence identity (e.g., 85%, 86%, 87%, 88%, 89%, 90%, 91%, 92%, 93%, 94%, 95%, 96%, 97%, 98%, 99%, or more, sequence identity) to regions of the GJB2 locus that are capable of expressing a transgene specifically in GJB2-expressing cells. The polynucleotides of the compositions and methods described herein can optionally include a linker operably linking the regions of the GJB2 locus that are capable of expressing a transgene specifically in GJB2-expressing cells, or the regions of the GJB2 locus can be joined directly without an intervening linker.

In some embodiments, the polynucleotides described herein contain a first region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1 or a functional portion or derivative thereof joined (e.g., operably linked) to a second region having at least 85% sequence identity (85%, 86%, 87%, 88%, 89%, 90%, 91%, 92%, 93%, 94%, 95%, 96%, 97%, 98%, 99%, or more, sequence identity) to SEQ ID NO: 2 or a functional portion or derivative thereof. The functional portion of SEQ ID NO: 1 may have the sequence of SEQ ID NO: 3, the sequence of SEQ ID NO: 4, the sequence of SEQ ID NO: 5, the sequence of SEQ ID NO: 6, the sequence SEQ ID NO: 7, the sequence of SEQ ID NO: 8, the sequence of SEQ ID NO: 9, the sequence of SEQ ID NO: 10, the sequence of SEQ ID NO: 11, the sequence of SEQ ID NO: 12, the sequence of SEQ ID NO: 4 fused to the sequence of SEQ ID NO: 12 with no intervening nucleic acids, as set forth in SEQ ID NO: 13, the sequence of SEQ ID NO: 5 fused to the sequence of SEQ ID NO: 12 with no intervening nucleic acids, as set forth in SEQ ID NO: 14, the sequence of SEQ ID NO: 6 fused to the sequence of SEQ ID NO: 12 with no intervening nucleic acids, as set forth in SEQ ID NO: 15, the sequence of SEQ ID NO: 7 fused to the sequence of SEQ ID NO: 12 with no intervening nucleic acids, as set forth in SEQ ID NO: 16, the sequence of SEQ ID NO: 9 fused to the sequence of SEQ ID NO: 12 with no intervening nucleic acids, as set forth in SEQ ID NO: 17, the sequence of SEQ ID NO: 10 fused to the sequence of SEQ ID NO: 12 with no intervening nucleic acids, as set forth in SEQ ID NO: 18, or the sequence of SEQ ID NO: 11 fused to the sequence of SEQ ID NO: 12 with no intervening nucleic acids, as set forth in SEQ ID NO: 19. The functional portion of SEQ ID NO: 1 may also have the sequence of SEQ ID NO: 7 fused to the sequence of SEQ ID NO: 12 by the endogenous intervening nucleic acid sequence, the sequence of SEQ ID NO: 10 fused to the sequence of SEQ ID NO: 12 by the endogenous intervening nucleic acid sequence, or the sequence of SEQ ID NO: 11 fused to the sequence of SEQ ID NO: 12 by the endogenous intervening nucleic acid sequence. In polynucleotides in which the first region contains the sequence of SEQ ID NO: 4, SEQ ID NO: 5, SEQ ID NO: 6, SEQ ID NO: 7, SEQ ID NO: 9, SEQ ID NO: 10, or SEQ ID NO: 11 and the sequence of SEQ ID NO: 12, the two sequences (one of SEQ ID NOs: 4-7 and 9-11 in combination with SEQ ID NO: 12) can be included in any order (e.g., one of SEQ ID NOs: 4-7 and 9-11 may be joined to (e.g., precede) SEQ ID NO: 12, as in SEQ ID NOs: 13-19, or SEQ ID NO: 12 may be joined to (e.g., precede) one of SEQ ID NOs: 4-7 and 9-11). The functional portion of SEQ ID NO: 2 may have the sequence of SEQ ID NO: 20, the sequence of SEQ ID NO: 21, the sequence of SEQ ID NO: 22, the sequence of SEQ ID NO: 23, the sequence of SEQ ID NO: 24, or the sequence of SEQ ID NO: 23 or SEQ ID NO: 24 fused to the sequence of SEQ ID NO: 20 or SEQ ID NO: 22 with no intervening nucleic acids, as set forth in SEQ ID NOs: 25-28. In polynucleotides in which the first region contains the sequence of SEQ ID NO: 23 or SEQ ID NO: 24 and the sequence of SEQ ID NO: 20 or SEQ ID NO: 22, the two sequences (one of SEQ ID NO: 23 and SEQ ID NO: 24 and one of SEQ ID NO: 20 and SEQ ID NO: 22) can be included in any order (e.g., one of SEQ ID NO: 23 and SEQ ID NO: 24 may be joined to (e.g., precede) one of SEQ ID NO: 20 and SEQ ID NO: 22, as in SEQ ID NOs: 25-28, or one of SEQ ID NO: 20 and SEQ ID NO: 22 may be joined to (e.g., precede) one of SEQ ID NO: 23 and SEQ ID NO: 24).

The first region and the second region of the polynucleotide can be joined directly or can be joined by a nucleic acid linker. For example, the polynucleotide can contain the sequence of SEQ ID NO: 1 or a functional portion or derivative thereof (e.g., any one or more of SEQ ID NOs: 3-19) fused to the sequence of SEQ ID NO: 2 or a functional portion or derivative thereof (e.g., any one or more of SEQ ID NOs: 20-28) with no intervening nucleic acids. For example, the nucleic acid sequence of the polynucleotide that results from direct fusion of SEQ ID NO: 1 to SEQ ID NO: 2 is set forth in SEQ ID NO: 29, the nucleic acid sequence of the polynucleotide that results from direct fusion of SEQ ID NO: 3 to SEQ ID NO: 20 is set forth in SEQ ID NO: 30, the nucleic acid sequence of the polynucleotide that results from direct fusion of SEQ ID NO: 3 to SEQ ID NO: 2 is set forth in SEQ ID NO: 31, the nucleic acid sequence of the polynucleotide that results from direct fusion of SEQ ID NO: 3 to SEQ ID NO: 21 is set forth in SEQ ID NO: 32, the nucleic acid sequence of the polynucleotide that results from direct fusion of SEQ ID NO: 3 to SEQ ID NO: 25 is set forth in SEQ ID NO: 33, the nucleic acid sequence of the polynucleotide that results from direct fusion of SEQ ID NO: 3 to SEQ ID NO: 26 is set forth in SEQ ID NO: 34, and the nucleic acid sequence of the polynucleotide that results from direct fusion of SEQ ID NO: 1 to SEQ ID NO: 22 is set forth in SEQ ID NO: 35. Alternatively, a linker can be used to join the sequence of SEQ ID NO: 1 or a functional portion or derivative thereof (e.g., any one of SEQ ID NOs: 3-19) to the sequence of SEQ ID NO: 2 or a functional portion or derivative thereof (e.g., any one of SEQ ID NOs: 20-28).

The length of a nucleic acid linker for use in the polynucleotides described herein can be about 5 kb or less (e.g., about 5 kb, 4.5, kb, 4, kb, 3.5 kb, 3 kb, 2.5 kb, 2 kb, 1.5 kb, 1 kb, 900 bp, 800 bp, 700 bp, 600 bp, 500 bp, 450 bp, 400 bp, 350 bp, 300 bp, 250 bp, 200 bp, 150 bp, 100 bp, 90 bp, 80 bp, 70 bp, 60 bp, 50 bp, 40 bp, 30 bp, 25 bp, 20 bp, 15, bp, 10 bp, 5 bp, 4 bp, 3 bp, 2 bp, or less). Nucleic acid linkers that can be used in the polynucleotides described herein do not disrupt the ability of the polynucleotides of the invention to induce transgene expression in GJB2-expressing cells.

In embodiments in which the first region and the second region of the polynucleotide are not joined directly, SEQ ID NO: 1 or a functional portion or derivative thereof (e.g., any one or more of SEQ ID NOs: 3-12) can operably linked to SEQ ID NO: 2 or a functional portion or derivative thereof (e.g., any one or more of SEQ ID NOs: 20-24) by a nucleic acid sequence that differs from the intervening genomic sequence. In some embodiments, the sequence that joins SEQ ID NO: 1 or a functional portion or derivative thereof and SEQ ID NO: 2 or a functional portion or derivative thereof is a shorter (e.g., truncated) version of the endogenous genomic sequence.

In some embodiments, the sequence having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1 or a functional portion or derivative thereof (e.g., any one or more of SEQ ID NOs: 3-19) is joined (e.g., operably linked) to the sequence having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or a functional portion or derivative thereof (e.g., any one or more of SEQ ID NOs: 20-28), and, in some embodiments, the order of the regions is reversed (e.g., the sequence having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or a functional portion or derivative thereof (e.g., any one or more of SEQ ID NOs: 20-28) is joined (e.g., operably linked) to the sequence having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1 or a functional portion or derivative thereof (e.g., any one or more of SEQ ID NOs: 3-19)). Regardless of order, the sequence having at least 85% sequence identity to SEQ ID NO: 1 or a functional portion or derivative thereof and the sequence having at least 85% sequence identity to SEQ ID NO: 2 or a functional portion or derivative thereof can be joined by direct fusion or a nucleic acid linker, as described above.

In some embodiments, the distance between the first region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1 or a functional portion or derivative thereof (e.g., any one or more of SEQ ID NOs: 3-19) and the second region having at least 85% sequence identity (85%, 86%, 87%, 88%, 89%, 90%, 91%, 92%, 93%, 94%, 95%, 96%, 97%, 98%, 99%, or more, sequence identity) to SEQ ID NO: 2 or a functional portion or derivative thereof (e.g., any one or more of SEQ ID NOs: 20-28) in the polynucleotide is no more than 1 kilobase (kb) (e.g., the distance between the 3′ end of the first region and the 5′ end of the second region is 1 kb or less, e.g., about 1 kb, 900 bp, 800 bp, 700 bp, 600 bp, 500 bp, 450 bp, 400 bp, 350 bp, 300 bp, 250 bp, 200 bp, 150 bp, 100 bp, 90 bp, 80 bp, 70 bp, 60 bp, 50 bp, 40 bp, 30 bp, 25 bp, 20 bp, 15, bp, 10 bp, 5 bp, 4 bp, 3 bp, 2 bp, or less). In some embodiments, there is no distance between the first region and the second region in the polynucleotide (e.g., the 3′ end of the first region is joined directly to the 5′ end of the second region).

In some embodiments, the first region and the second region of the polynucleotide can be joined by the endogenous intervening nucleic acid sequence. For example, the sequence that results from joining SEQ ID NO: 3 with SEQ ID NO: 2 using the endogenous intervening nucleic acid sequence is set forth in SEQ ID NO: 36.

In some embodiments, the polynucleotide described herein has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 37.

In some embodiments, the polynucleotides described herein have at least 85% sequence identity (e.g., 85%, 86%, 87%, 88%, 89%, 90%, 91%, 92%, 93%, 94%, 95%, 96%, 97%, 98%, 99%, or more, sequence identity) to a functional portion or derivative of SEQ ID NO: 1. For example, a polynucleotide described herein can have at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 3, SEQ ID NO: 4, SEQ ID NO: 5, SEQ ID NO: 6, SEQ ID NO: 7, SEQ ID NO: 12, SEQ ID NO: 13, SEQ ID NO: 14, SEQ ID NO: 15, SEQ ID NO: 16, SEQ ID NO: 17, SEQ ID NO: 18, or SEQ ID NO: 19.

In some embodiments, the polynucleotides described herein have at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or a functional portion or derivative thereof. The functional portion of SEQ ID NO: 2 may have the sequence of SEQ ID NO: 20, the sequence of SEQ ID NO: 21, the sequence of SEQ ID NO: 22, the sequence of SEQ ID NO: 23, or the sequence of SEQ ID NO: 24. The second region may contain the nucleic acid sequence of SEQ ID NO: 23 or SEQ ID NO: 24 fused to the nucleic acid sequence of SEQ ID NO: 20 or SEQ ID NO: 22 with no intervening nucleic acids, as set forth in SEQ ID NOs: 25-28.

The foregoing nucleic acid sequences are summarized in Table 2, below.

TABLE 2
GJB2 promoter sequences

| SEQ | Description of promoter | |
|---|---|---|
| ID NO: | sequence | Promoter sequence |
| 1 | Proximal promoter | CCGCAGAATCCTATCAGTTTCCCCCTTTCGTGCTGTGTGCA |
| | | TCGAGCAGGAAGGGGCTTGGCAGGTTTTACCTGCCCTCTT |
| | | TCCTTTCTGAAAAGTCTGGGCCTCCTCACCCCGAAAGGAG |
| | | TCACCTCCTTGCAGTTCCCCAGTTGCGAAAAGAGGAGGAA |
| | | GTTGGCTGGGCCGGGGGCCGCGGGGGGCACCCTCCGCA |
| | | GATGGCGGGACCCCCCTGCCGGCCATGGCAAAAACGAGG |
| | | CTTGTCTCTCCCACCGCCCCCAACCTTAGTCCTTGGCACAT |
| | | TGTTGAAAGTAATTGAATAAAATCGGAAATTCGAGAAGGCG |
| | | TTCGTTCGGATTGGTGAGATTTTGAGGGGAGAAAGAAGCG |
| | | GGGACTTCGCCGGCACCAGCGGCGCCCCCTCCTCGGCCA |
| | | CCGTTAACCCCCATTCCAGAGGGCACTGCCCCGCCACCCA |
| | | GCCTAGGTCCCCCTGCGAGAGCCTCGCGGGCCCGCGCAG |
| | | CCTCCGCGACTCGAACAGATCTTCAGTCCTTGGAGGAATG |
| | | CCTGTTTCTCTAACAATAAAAAATTAAAGAAGCGCTCATAAA |
| | | TGCCAAGTCCTCTCGCACTATGCGGAGTACAGAGGACAAC |
| | | GACCACAGCCATCCCTGAACCCCGCCCACGGCACAGCGC |
| | | CGGAGCCGGGGTCTGGGGCGCCGCTTCCTGGGGGGTCC |
| | | CGACTCTCAGCCGCCCCCGCTTCACCCGGGCCGCCAAGG |
| | | GGCTGGGGGAGGCGGCGCTCGGGGTAACCGGGGGAGAC |
| | | TCAGGGCGCTGGGGGCACTTGGGGAACTCATGGGGGCTC |
| | | AAAGGAACTAGGAGATCGGGACCTCGAAGGGGACTTGGG |
| | | GGGTTCGGGGCTTTCGGGGGCGGTCGGGGGTTCGCGGA |
| | | CCCGGGAAGCTCTGAGGACCCAGAGGCCGGGCGCGCTCC |
| | | GCCCGCGGCGCCGCCCCCTCCGTAACTTTCCCAGTCTCC |
| | | GAGGGAAGAGGCGGGGTGTGGGGTGCGGTTAAAAGGCGC |
| | | CACGGCGGGAGACAGGTGTTGCGGCCCCGCAGCGCCCG |
| | | CGCGCTCCTCTCCCCGACTCGGAGCCCCTCGGCGGCGCC |
| | | CGGCCCAGGACCCGCCTAGGAGCGCAGGAGCCCCAGCG |
| | | CAGAGACCCCAACGCCGAGACCCCCGCCCCGGCCCCGCC |
| | | GCGCTTCCTCCCGACGCAGGTGAGCCCGCCGGCCCCGGA |
| | | CTGCCCGGCCAGGAACCTGGCGCGGGGAGGGACCGCGA |
| | | GACCCAGAGCGGTTGCCCGGCCGCGTGGGTCTCGGGGAA |
| | | CCGGGGGGCTGGACCAACACACGTCCTTGGGCCGGGGG |
| | | GCGGGGGCCGCCTTCTGGAGCGGGCGTTTCTGCGGCCGA |
| | | GCTCCGGAGCTGGAATGGGGCGGCCGGGGAAGTGGACG |
| | | CGATGGCACCGCCCGGGGTGCGAGTGGGGCCGGGCGCG |
| | | CGCGGGAGGGGAAAAAGGCGCGGGCGAGCCGCCAGCGC |
| | | GAGGTTTGTGGTGTCGCCGATGTCCCTTCGGGGTACTCTA |
| | | GCGCAGCCGCCTGGCTACTTGACCCACTGCCACCAAACGT |
| | | TTTAAATTCACCG |
| 2 | Regulatory region | TTCGTGGCCACCTCTCTCCTGTTAGCCTCTGACCAAGGTG |
| | containing splice acceptor | GCACTGGATGGTTTCTGCCTGACCTTGGTGCCCCGTGGCA |
| | | GCGACTGTGGGTCATGAAAGACATTCACTACGAGCCTGCT |
| | | TCTGGAGTCCATCAGAAAACGGGATGCAACTTGCCTAAAAT |
| | | GAGGAGAGGAGGATGCTTTTAAGAAAAAGAAGAAGGAGGA |
| | | TTCACTACCAGCTCTGAAGGGTGGAAAAGAGATGATTCATC |
| | | CGGATTGTGGAGAGGGTGGAATCTTGTTTAGGAGAGCGTT |
| | | GGTTGTGGCAGGCAGGGTGTAACTATGAATCAGTGAAGAC |
| | | AATTCACATCCTGGGATGAAAAGAAGGCCATGGGCTCACA |
| | | GGAGATTATCCACTGGCCTCTCCACATCCGCTTGCAGTAA |
| | | GGAGTGTGGGACTCTCCCAAGCTTCAGCGCTGAACTGCAA |
| | | TGCAGTGACGTCGCTTAGCTGGGCCAGTAACCGAGGGAGT |
| | | TGAATTTTCTGTCATTTTAAAATAATGTGTCTTTTAAGAAACA |
| | | CTTTGAAATTAAAACCACAGCCCACAATTATAATGCACTGTT |
| | | GCAGCACTTATCAAAACAGATATGCTAACTGAGCCATCAGT |
| | | GCCAGCCTGACAGTGAGGCCACCAAGCCATCCACAAAGCC |
| | | TACACGAAAGTCTGTGCTCACAGTGGCTTTTCTCCATGAAG |
| | | AGGGCATTCCTAACCTCTTCCTTTCACGTAGGAGGAAGCAA |
| | | GGTCCTTTGTAAAATTTTAACTCGGGGTGCCTCAAATGTAA |
| | | ACTTAACCACTGGTAACAACAGTTTCACTGCTACATGCCAC |
| | | GTCTGTGAAAATTCATTCAAGACATTAAGGAAAGTGGCTCA |
| | | GCAGAGAGACTAGACATCTTATCCTCACGGTTCTCCTGTAC |
| | | TTGGCCTCTCAGCCTTTGAGCAAGGTTGGCCCAAGCTAGT |
| | | ATCGGCCCCAGTGGTACAGCCAAAACTTGAGACTGCAAAT |
| | | GGATGCAGCTGTTGAACGCTGAGTAACTTCTGCAGAGTCA |
| | | GGAAGACCCAAGGAAGCTCTGCAGAGGATGCAGGGGTAC |
| | | GGTCAGAACCCCTGAGTGCCTTTCAGCTAACGAGGACTTT |
| | | ATGACACTCCCCAGCACAGCAAATTTTTATGATGTGTTTAA |
| | | AGATTGGGTGAATTACTCAGGTGAACAAGCTACTTTTTATC |
| | | AGAGAACACCTAAAAACACGTTCAAGAGGGTTTGGGAACT |
| | | ATACATTTAATCCTATGACAAACTAAGTTGGTTCTGTCTTCA |
| | | CCTGTTTTGGTGAGGTTGTGTAAGAGTTGGTGTTTGCTCAG |
| | | GAAGAGATTTAAGCATGCTTGCTTACCCAGACTCAGAGAAG |
| | | TCTCCCTGTTCTGTCCTAGCTAGTGATTCCTGTGTTGTGTG |
| | | CATTCGTCTTTTCCAGAGCAAACCGCCCAGAGTAGAAG |
| 3 | Portion of SEQ ID NO: 1 | ACAGAGGACAACGACCACAGCCATCCCTGAACCCCGCCCA |
| | | CGGCACAGCGCCGGAGCCGGGGTCTGGGGCGCCGCTTC |
| | | CTGGGGGGTCCCGACTCTCAGCCGCCCCCGCTTCACCCG |
| | | GGCCGCCAAGGGGCTGGGGGAGGCGGCGCTCGGGGTAA |
| | | CCGGGGGAGACTCAGGGCGCTGGGGGCACTTGGGGAACT |
| | | CATGGGGGCTCAAAGGAACTAGGAGATCGGGACCTCGAA |
| | | GGGGACTTGGGGGGTTCGGGGCTTTCGGGGGCGGTCGG |
| | | GGGTTCGCGGACCCGGGAAGCTCTGAGGACCCAGAGGCC |
| | | GGGCGCGCTCCGCCCGCGGCGCCGCCCCCTCCGTAACTT |
| | | TCCCAGTCTCCGAGGGAAGAGGGGGGGTGTGGGGTGCGG |
| | | TTAAAAGGCGCCACGGGGGGAGACAGGTGTTGCGGCCCC |
| | | GCAGCGCCCGCGCGCTCCTCTCCCCGACTCGGAGCCCCT |
| | | CGGCGGCGCCCGGCCCAGGACCCGCCTAGGAGCGCAGG |
| | | AGCCCCAGCGCAGAGACCCCAACGCCGAGACCCCCGCCC |
| | | CGGCCCCGCCGCGCTTCCTCCCGACGCAGGTGAGCCCGC |
| | | CGGCCCCGGACTGCCCGGCCAGGAACCTGGCGCGGGGA |
| | | GGGACCGCGAGACCCAGAGCGGTTGCCCGGCCGCGTGG |
| | | GTCTCGGGGAACCGGGGGGCTGGACCAACACACGTCCTT |
| | | GGGCCGGGGGGGGGGGGCCGCCTTCTGGAGCGGGCGTT |
| | | TCTG |
| 4 | Portion of SEQ ID NO: 1 | ACAGAGGACAACGACCACAGCCATCCCTGAACCCCGCCCA |
| | | CGGCACAGCGCCGGAGCCGGGGTCTGGGGCGCCGCTTC |
| | | CTGGGGGGTCCCGACTCTCAGCCGCCCCCGCTTCACCCG |
| | | GGCCGCCAAGGGGCTGGGGGAGGCGGCGCTCGGGGTAA |
| | | CCGGGGGAGACTCAGGGCGCTGGGGGCACTTGGGGAACT |
| | | CATGGGGGCTCAAAGGAACTAGGAGATCGGGACCTCGAA |
| | | GGGGACTTGGGGGGTTCGGGGCTTTCGGGGGGGGTCGG |
| | | GGGTTCGCGGACCCGGGAAGCTCTGAGGACCCAGAGGCC |
| | | GGGCGCGCTCCGCCCGCGGCGCCGCCCCCTCCGTAACTT |
| | | TCCCAGTCTCCGAGGGAAGAGGCGGGGTGTGGGGTGCGG |
| | | TTAAAAGGCGCCACGGCGGGAGACAGGTGTTGCGGCCCC |
| | | GCAGCGCCCGCGCGCTCCTCTCCCCGACTCGGAGCCCCT |
| | | CGGCGGCGCCCGGCCCAGGACCCGCCTAGGAGCGCAGG |
| | | AGCCCCAGCGCAGAGACCCCAACGCCGAGACCCCCGCCC |
| | | CGGCCCCGCCGCGCTTCCTCCCGACG |
| 5 | Portion of SEQ ID NO: 1 | ACAGAGGACAACGACCACAGCCATCCCTGAACCCCGCCCA |
| | | CGGCACAGCGCCGGAGCCGGGGTCTGGGGCGCCGCTTC |
| | | CTGGGGGGTCCCGACTCTCAGCCGCCCCCGCTTCACCCG |
| | | GGCCGCCAAGGGGCTGGGGGAGGCGGCGCTCGGGGTAA |
| | | CCGGGGGAGACTCAGGGCGCTGGGGGCACTTGGGGAACT |
| | | CATGGGGGCTCAAAGGAACTAGGAGATCGGGACCTCGAA |
| | | GGGGACTTGGGGGGTTCGGGGCTTTCGGGGGGGGTCGG |
| | | GGGTTCGCGGACCCGGGAAGCTCTGAGGACCCAGAGGCC |
| | | GGGCGCGCTCCGCCCGCGGCGCCGCCCCCTCCGTAACTT |
| | | TCCCAGTCTCCGAGGGA |
| 6 | Portion of SEQ ID NO: 1 | AGAGGCGGGGTGTGGGGTGCGGTTAAAAGGCGCCACGGC |
| | | GGGAGACAGGTGTTGCGGCCCCGCAGCGCCCGCGCGCTC |
| | | CTCTCCCCGACTCGGAGCCCCT |
| 7 | Portion of SEQ ID NO: 1 | GCTCAAAGGAACTAGGAGATCGGGACCTCGAAGGGGACTT |
| | | GGGGGGTTCGGGGCTTTCGGGGGGGGTCGGGGGTTCGC |
| | | GGACCCGGGAAGCTCTGAGGACCCAGAGGCCGGGCGCG |
| | | CTCCGCCCGCGGCGCCGCCCCCTCCGTAACTTTCCCAGTC |
| | | TCCGAGGGAAGAGGCGGGGTGTGGGGTGCGGTTAAAAGG |
| | | CGCCACGGCGGGAGACAGGTGTTGCGGCCC |
| 8 | Portion of SEQ ID NO: 1 | GGCTCAAAGGAACTAGGAGATCGGGACCTCGAAGGGGAC |
| | | TTGGGGGGTTCGGGGCTTTCGGGGGGGGTCGGGGGTTCG |
| | | CGGACCCGGGAAGCTCTGAGGACCCAGAGGCCGGGCGC |
| | | GCTCCGCCCGCGGCGCCGCCCCCTCCGTAACTTTCCCAG |
| | | TCTCCGAGGGAAGAGGGGGGGTGTGGGGTGCGGTTAAAA |
| | | GGCGCCACGGCGGGAGACAGGTGTTGCGGCCCCGCAGC |
| | | GCCCGCGCGCTCCTCTCCCCGACTCGGAGCCCCTCGGCG |
| | | GCGCCCGGCCCAGGACCCGCCTAGGAGCGCAGGAGCCC |
| | | CAGCGCAGAGACCCCAACGCCGAGACCCCCGCCCCGGCC |
| | | CCGCCGCGCTTCCTCCCGACGCAGGTGAGCCCGCCGGCC |
| | | CCGGACTGCCCGGCCAGGAACCTGGCGCGGGGAGGGAC |
| | | CGCGAGACCCAGAGCGGTTGCCCGGCCGCGTGGGTCTCG |
| | | GGGAACCGGGGGGCTGGACCAACACACGTCCTTGGGCCG |
| | | GGGGGCGGGGGCCGCCTTCTGGAGCGGGCGTTTCTG |
| 9 | Portion of SEQ ID NO: 1 | ACAGAGGACAACGACCACAGCCATCCCTGAACCCCGCCCA |
| | | CGGCACAGCGCCGGAGCCGGGGTCTGGGGCGCCGCTTC |
| | | CTGGGGGGTCCCGACTCTCAGCCGCCCCCGCTTCACCCG |
| | | GGCCGCCAAGGGGCTGGGGGAGGCGGCGCTCGGGGTAA |
| | | CCGGGGGAGACTCAGGGCGCTGGGGGCACTTGGGGAACT |
| | | CATGGGGGCTCAAAGGAACTAGGAGATCGGGACCTCGAA |
| | | GGGGACTTGGGGGGTTCGGGGCTTTCGGGGGCGGTCGG |
| | | GGGTTCGCGGACCCGGGAAGCTCTGAGGACCCAGAGGCC |
| | | GGGCGCGCTCCGCCCGCGGCGCCGCCCCCTCCGTAACTT |
| | | TCCCAGTCTCCGAGGGAAGAGGGGGGGTGTGGGGTGCGG |
| | | TTAAAAGGCGCCACGGGGGGAGACAGGTGTTGCGGCCC |
| 10 | Portion of SEQ ID NO: 1 | CGGGAAGCTCTGAGGACCCAGAGGCCGGGCGCGCTCCGC |
| | | CCGCGGCGCCGCCCCCTCCGTAACTTTCCCAGTCTCCGAG |
| | | GGAAGAGGCGGGGTGTGGGGTGCGGTTAAAAGGCGCCAC |
| | | GGCGGGAGAC |
| 11 | Portion of SEQ ID NO: 1 | AAGCTCTGAGGACCCAGAGGCCGGGCGCGCTCCGCCCGC |
| | | GGCGCCGCCCCCTCCGTAACTTTCCCAGTCTCCGAGGGAA |
| | | GAGGCGGGGTGTGGGGTGCGGTTAAAAGGCGCCACGGC |
| | | GGGAGACAGGT |
| 12 | Portion of SEQ ID NO: 1 | AGCCCGCCGGCCCCGGACTGCCCGGCCAGGAACCTGGC |
| | | GCGGGGAGGGACCGCGAGACCCAGAGCGGTTGCCCGGC |
| | | CGCGTGGGTCTCGGGGAACCGGGGGGCTGGACCAACACA |
| | | CGTCCTTGGGCCGGGGGGGGGGGGCCGCCTTCTGGAGC |
| | | GGGCGTTTCTG |
| 13 | SEQ ID NO: 4 fused to SEQ | ACAGAGGACAACGACCACAGCCATCCCTGAACCCCGCCCA |
| | ID NO: 12 | CGGCACAGCGCCGGAGCCGGGGTCTGGGGCGCCGCTTC |
| | | CTGGGGGGTCCCGACTCTCAGCCGCCCCCGCTTCACCCG |
| | | GGCCGCCAAGGGGCTGGGGGAGGCGGCGCTCGGGGTAA |
| | | CCGGGGGAGACTCAGGGCGCTGGGGGCACTTGGGGAACT |
| | | CATGGGGGCTCAAAGGAACTAGGAGATCGGGACCTCGAA |
| | | GGGGACTTGGGGGGTTCGGGGCTTTCGGGGGGGGTCGG |
| | | GGGTTCGCGGACCCGGGAAGCTCTGAGGACCCAGAGGCC |
| | | GGGCGCGCTCCGCCCGCGGCGCCGCCCCCTCCGTAACTT |
| | | TCCCAGTCTCCGAGGGAAGAGGGGGGGTGTGGGGTGCGG |
| | | TTAAAAGGCGCCACGGCGGGAGACAGGTGTTGCGGCCCC |
| | | GCAGCGCCCGCGCGCTCCTCTCCCCGACTCGGAGCCCCT |
| | | CGGCGGCGCCCGGCCCAGGACCCGCCTAGGAGCGCAGG |
| | | AGCCCCAGCGCAGAGACCCCAACGCCGAGACCCCCGCCC |
| | | CGGCCCCGCCGCGCTTCCTCCCGACGAGCCCGCCGGCCC |
| | | CGGACTGCCCGGCCAGGAACCTGGCGCGGGGAGGGACC |
| | | GCGAGACCCAGAGCGGTTGCCCGGCCGCGTGGGTCTCGG |
| | | GGAACCGGGGGGCTGGACCAACACACGTCCTTGGGCCGG |
| | | GGGGGGGGGCCGCCTTCTGGAGCGGGCGTTTCTG |
| 14 | SEQ ID NO: 5 fused to SEQ | ACAGAGGACAACGACCACAGCCATCCCTGAACCCCGCCCA |
| | ID NO: 12 | CGGCACAGCGCCGGAGCCGGGGTCTGGGGCGCCGCTTC |
| | | CTGGGGGGTCCCGACTCTCAGCCGCCCCCGCTTCACCCG |
| | | GGCCGCCAAGGGGCTGGGGGAGGCGGCGCTCGGGGTAA |
| | | CCGGGGGAGACTCAGGGCGCTGGGGGCACTTGGGGAACT |
| | | CATGGGGGCTCAAAGGAACTAGGAGATCGGGACCTCGAA |
| | | GGGGACTTGGGGGGTTCGGGGCTTTCGGGGGCGGTCGG |
| | | GGGTTCGCGGACCCGGGAAGCTCTGAGGACCCAGAGGCC |
| | | GGGCGCGCTCCGCCCGCGGCGCCGCCCCCTCCGTAACTT |
| | | TCCCAGTCTCCGAGGGAAGCCCGCCGGCCCCGGACTGCC |
| | | CGGCCAGGAACCTGGCGCGGGGAGGGACCGCGAGACCC |
| | | AGAGCGGTTGCCCGGCCGCGTGGGTCTCGGGGAACCGG |
| | | GGGGCTGGACCAACACACGTCCTTGGGCCGGGGGGCGG |
| | | GGGCCGCCTTCTGGAGCGGGCGTTTCTG |
| 15 | SEQ ID NO: 6 fused to SEQ | AGAGGCGGGGTGTGGGGTGCGGTTAAAAGGCGCCACGGC |
| | ID NO: 12 | GGGAGACAGGTGTTGCGGCCCCGCAGCGCCCGCGCGCTC |
| | | CTCTCCCCGACTCGGAGCCCCTAGCCCGCCGGCCCCGGA |
| | | CTGCCCGGCCAGGAACCTGGCGCGGGGAGGGACCGCGA |
| | | GACCCAGAGCGGTTGCCCGGCCGCGTGGGTCTCGGGGAA |
| | | CCGGGGGGCTGGACCAACACACGTCCTTGGGCCGGGGG |
| | | GCGGGGGCCGCCTTCTGGAGCGGGCGTTTCTG |
| 16 | SEQ ID NO: 7 fused to SEQ | GCTCAAAGGAACTAGGAGATCGGGACCTCGAAGGGGACTT |
| | ID NO: 12 | GGGGGGTTCGGGGCTTTCGGGGGGGGTCGGGGGTTCGC |
| | | GGACCCGGGAAGCTCTGAGGACCCAGAGGCCGGGCGCG |
| | | CTCCGCCCGCGGCGCCGCCCCCTCCGTAACTTTCCCAGTC |
| | | TCCGAGGGAAGAGGGGGGGTGTGGGGTGCGGTTAAAAGG |
| | | CGCCACGGCGGGAGACAGGTGTTGCGGCCCAGCCCGCC |
| | | GGCCCCGGACTGCCCGGCCAGGAACCTGGCGCGGGGAG |
| | | GGACCGCGAGACCCAGAGCGGTTGCCCGGCCGCGTGGGT |
| | | CTCGGGGAACCGGGGGGCTGGACCAACACACGTCCTTGG |
| | | GCCGGGGGGGGGGGGCCGCCTTCTGGAGCGGGCGTTTC |
| | | TG |
| 17 | SEQ ID NO: 9 fused to SEQ | ACAGAGGACAACGACCACAGCCATCCCTGAACCCCGCCCA |
| | ID NO: 12 | CGGCACAGCGCCGGAGCCGGGGTCTGGGGCGCCGCTTC |
| | | CTGGGGGGTCCCGACTCTCAGCCGCCCCCGCTTCACCCG |
| | | GGCCGCCAAGGGGCTGGGGGAGGCGGCGCTCGGGGTAA |
| | | CCGGGGGAGACTCAGGGCGCTGGGGGCACTTGGGGAACT |
| | | CATGGGGGCTCAAAGGAACTAGGAGATCGGGACCTCGAA |
| | | GGGGACTTGGGGGGTTCGGGGCTTTCGGGGGGGGTCGG |
| | | GGGTTCGCGGACCCGGGAAGCTCTGAGGACCCAGAGGCC |
| | | GGGCGCGCTCCGCCCGCGGCGCCGCCCCCTCCGTAACTT |
| | | TCCCAGTCTCCGAGGGAAGAGGGGGGGTGTGGGGTGCGG |
| | | TTAAAAGGCGCCACGGCGGGAGACAGGTGTTGCGGCCC |
| | | AGCCCGCCGGCCCCGGACTGCCCGGCCAGGAACCTGGC |
| | | GCGGGGAGGGACCGCGAGACCCAGAGCGGTTGCCCGGC |
| | | CGCGTGGGTCTCGGGGAACCGGGGGGCTGGACCAACACA |
| | | CGTCCTTGGGCCGGGGGGGGGGGGCCGCCTTCTGGAGC |
| | | GGGCGTTTCTG |
| 18 | SEQ ID NO: 10 fused to | CGGGAAGCTCTGAGGACCCAGAGGCCGGGCGCGCTCCGC |
| | SEQ ID NO: 12 | CCGCGGCGCCGCCCCCTCCGTAACTTTCCCAGTCTCCGAG |
| | | GGAAGAGGGGGGGTGTGGGGTGCGGTTAAAAGGCGCCAC |
| | | GGCGGGAGACAGCCCGCCGGCCCCGGACTGCCCGGCCA |
| | | GGAACCTGGCGCGGGGAGGGACCGCGAGACCCAGAGCG |
| | | GTTGCCCGGCCGCGTGGGTCTCGGGGAACCGGGGGGCT |
| | | GGACCAACACACGTCCTTGGGCCGGGGGGGGGGGGCCG |
| | | CCTTCTGGAGCGGGCGTTTCTG |
| 19 | SEQ ID NO: 11 fused to | AAGCTCTGAGGACCCAGAGGCCGGGCGCGCTCCGCCCGC |
| | SEQ ID NO: 12 | GGCGCCGCCCCCTCCGTAACTTTCCCAGTCTCCGAGGGAA |
| | | GAGGCGGGGTGTGGGGTGCGGTTAAAAGGCGCCACGGC |
| | | GGGAGACAGGTAGCCCGCCGGCCCCGGACTGCCCGGCC |
| | | AGGAACCTGGCGCGGGGAGGGACCGCGAGACCCAGAGC |
| | | GGTTGCCCGGCCGCGTGGGTCTCGGGGAACCGGGGGGC |
| | | TGGACCAACACACGTCCTTGGGCCGGGGGGGGGGGGCC |
| | | GCCTTCTGGAGCGGGCGTTTCTG |
| 20 | Portion of SEQ ID NO: 2 | TTTATGATGTGTTTAAAGATTGGGTGAATTACTCAGGTGAA |
| | | CAAGCTACTTTTTATCAGAGAACACCTAAAAACACGTTCAA |
| | | GAGGGTTTGGGAACTATACATTTAATCCTATGACAAACTAA |
| | | GTTGGTTCTGTCTTCACCTGTTTTGGTGAGGTTGTGTAAGA |
| | | GTTGGTGTTTGCTCAGGAAGAGATTTAAGCATGCTTGOTTA |
| | | CCCAGACTCAGAGAAGTCTCCCTGTTCTGTCCTAGCTAGT |
| | | GATTCCTGTGTTGTGTGCATTCGTCTTTTCCAGAGCAAACC |
| | | GCCCAGAGTAGAAG |
| 21 | Portion of SEQ ID NO: 2 | AAAACCACAGCCCACAATTATAATGCACTGTTGCAGCACTT |
| | | ATCAAAACAGATATGCTAACTGAGCCATCAGTGCCAGCCTG |
| | | ACAGTGAGGCCACCAAGCCATCCACAAAGCCTACACGAAA |
| | | GTCTGTGCTCACAGTGGCTTTTCTCCATGAAGAGGGCATTC |
| | | CTAACCTCTTCCTTTCACGTAGGAGGAAGCAAGGTCCTTTG |
| | | TAAAATTTTAACTCGGGGTGCCTCAAATGTAAACTTAACCA |
| | | CTGGTAACAACAGTTTCACTGCTACATGCCACGTCTGTGAA |
| | | AATTCATTCAAGACATTAAGGAAAGTGGCTCAGCAGAGAGA |
| | | CTAGACATCTTATCCTCACGGTTCTCCTGTACTTGGCCTCT |
| | | CAGCCTTTGAGCAAGGTTGGCCCAAGCTAGTATCGGCCCC |
| | | AGTGGTACAGCCAAAACTTGAGACTGCAAATGGATGCAGC |
| | | TGTTGAACGCTGAGTAACTTCTGCAGAGTCAGGAAGACCC |
| | | AAGGAAGCTCTGCAGAGGATGCAGGGGTACGGTCAGAAC |
| | | CCCTGAGTGCCTTTCAGCTAACGAGGACTTTATGACACTCC |
| | | CCAGCACAGCAAATTTTTATGATGTGTTTAAAGATTGGGTG |
| | | AATTACTCAGGTGAACAAGCTACTTTTTATCAGAGAACACC |
| | | TAAAAACACGTTCAAGAGGGTTTGGGAACTATACATTTAAT |
| | | CCTATGACAAACTAAGTTGGTTCTGTCTTCACCTGTTTTGG |
| | | TGAGGTTGTGTAAGAGTTGGTGTTTGCTCAGGAAGAGATTT |
| | | AAGCATGCTTGCTTACCCAGACTCAGAGAAGTCTCCCTGTT |
| | | CTGTCCTAGCTAGTGATTCCTGTGTTGTGTGCATTCGTCTT |
| | | TTCCAGAGCAAACCGCCCAGAGTAGAAG |
| 22 | Portion of SEQ ID NO: 2 | GCTTACCCAGACTCAGAGAAGTCTCCCTGTTCTGTCCTAGC |
| | | TAGTGATTCCTGTGTTGTGTGCATTCGTCTTTTCCAGAGCA |
| | | AACCGCCCAGAGTAGAAG |
| 23 | Portion of SEQ ID NO: 2 | TTCGTGGCCACCTCTCTCCTGTTAGCCTCTGACCAAGGTG |
| | | GCACTGGATGGTTTCTGCCTGACCTTGGTGCCCCGTGGCA |
| | | GCGACTGTGGGTCATGAAAGACATTCACTACGAGCCTGCT |
| | | TCTGGAGTCCATCAGAAAACGGGATGCAACTTGCCTAAAAT |
| | | GAGGAGAGGAGGATGCTTTTAAGAAAAAGAAGAAGGAGGA |
| | | TTCACTACCAGCTCTGAAGGGTGGAAAAGAGATGATTCATC |
| | | CGGATTGTGGAGAGGGTGGAATCTTGTTTAGGAGAGCGTT |
| | | GGTTGTGGCAGGCAGGGTGTAACTATGAATCAGTGAAGAC |
| | | AATTCACATCCTGGGATGAAAAGAAGGCCATGGGCTCACA |
| | | GGAGATTATCCACTGGCCTCTCCACATCCGCTTGCAGTAA |
| | | GGAGTGTGGGACTCTCCCAAGCTTCAGCGCTGAACTGCAA |
| | | TGCAGTGACGTCGCTTAGCTGGGCCAGTAACCGAGGGAGT |
| | | TGAATTTTCTGTCATTTTAAAATAATGTGTCTTTTAAGAAACA |
| | | CTTTGAAATT |
| 24 | Portion of SEQ ID NO: 2 | CATGAAAGACATTCACTACGAGCCTGCTTCTGGAGTCCATC |
| | | AGAAAACGGGATGCAACTTGCCTAAAATGAGGAGAGGAGG |
| | | ATGCTTTTAAGAAAAAGAAGAAGGAGGATTCACTACCAGCT |
| | | CTGAAGGGTGGAAAAGAGATGATTCATCCGGATTGTGGAG |
| | | AGGGTGGAATCTTGTTTAGGAGAGCGTTGGTTGTGGCAGG |
| | | CAGGGTGTAACTATGAATCAGTGAAGACAATTCACATCCTG |
| | | GGATGAAAAGAAGGCCATGGGCTCACAGGAGATTATCCAC |
| | | TGGCCTCTCCACATCC |
| 25 | SEQ ID NO: 23 fused to | TTCGTGGCCACCTCTCTCCTGTTAGCCTCTGACCAAGGTG |
| | SEQ ID NO: 20 | GCACTGGATGGTTTCTGCCTGACCTTGGTGCCCCGTGGCA |
| | | GCGACTGTGGGTCATGAAAGACATTCACTACGAGCCTGCT |
| | | TCTGGAGTCCATCAGAAAACGGGATGCAACTTGCCTAAAAT |
| | | GAGGAGAGGAGGATGCTTTTAAGAAAAAGAAGAAGGAGGA |
| | | TTCACTACCAGCTCTGAAGGGTGGAAAAGAGATGATTCATC |
| | | CGGATTGTGGAGAGGGTGGAATCTTGTTTAGGAGAGCGTT |
| | | GGTTGTGGCAGGCAGGGTGTAACTATGAATCAGTGAAGAC |
| | | AATTCACATCCTGGGATGAAAAGAAGGCCATGGGCTCACA |
| | | GGAGATTATCCACTGGCCTCTCCACATCCGCTTGCAGTAA |
| | | GGAGTGTGGGACTCTCCCAAGCTTCAGCGCTGAACTGCAA |
| | | TGCAGTGACGTCGCTTAGCTGGGCCAGTAACCGAGGGAGT |
| | | TGAATTTTCTGTCATTTTAAAATAATGTGTCTTTTAAGAAACA |
| | | CTTTGAAATTTTTATGATGTGTTTAAAGATTGGGTGAATTAC |
| | | TCAGGTGAACAAGCTACTTTTTATCAGAGAACACCTAAAAA |
| | | CACGTTCAAGAGGGTTTGGGAACTATACATTTAATCCTATG |
| | | ACAAACTAAGTTGGTTCTGTCTTCACCTGTTTTGGTGAGGT |
| | | TGTGTAAGAGTTGGTGTTTGCTCAGGAAGAGATTTAAGCAT |
| | | GCTTGCTTACCCAGACTCAGAGAAGTCTCCCTGTTCTGTCC |
| | | TAGCTAGTGATTCCTGTGTTGTGTGCATTCGTCTTTTCCAG |
| | | AGCAAACCGCCCAGAGTAGAAG |
| 26 | SEQ ID NO: 24 fused to | CATGAAAGACATTCACTACGAGCCTGCTTCTGGAGTCCATC |
| | SEQ ID NO: 20 | AGAAAACGGGATGCAACTTGCCTAAAATGAGGAGAGGAGG |
| | | ATGCTTTTAAGAAAAAGAAGAAGGAGGATTCACTACCAGCT |
| | | CTGAAGGGTGGAAAAGAGATGATTCATCCGGATTGTGGAG |
| | | AGGGTGGAATCTTGTTTAGGAGAGCGTTGGTTGTGGCAGG |
| | | CAGGGTGTAACTATGAATCAGTGAAGACAATTCACATCCTG |
| | | GGATGAAAAGAAGGCCATGGGCTCACAGGAGATTATCCAC |
| | | TGGCCTCTCCACATCCTTTATGATGTGTTTAAAGATTGGGT |
| | | GAATTACTCAGGTGAACAAGCTACTTTTTATCAGAGAACAC |
| | | CTAAAAACACGTTCAAGAGGGTTTGGGAACTATACATTTAA |
| | | TCCTATGACAAACTAAGTTGGTTCTGTCTTCACCTGTTTTG |
| | | GTGAGGTTGTGTAAGAGTTGGTGTTTGCTCAGGAAGAGAT |
| | | TTAAGCATGCTTGCTTACCCAGACTCAGAGAAGTCTCCCTG |
| | | TTCTGTCCTAGCTAGTGATTCCTGTGTTGTGTGCATTCGTC |
| | | TTTTCCAGAGCAAACCGCCCAGAGTAGAAG |
| 27 | SEQ ID NO: 23 fused to | TTCGTGGCCACCTCTCTCCTGTTAGCCTCTGACCAAGGTG |
| | SEQ ID NO: 22 | GCACTGGATGGTTTCTGCCTGACCTTGGTGCCCCGTGGCA |
| | | GCGACTGTGGGTCATGAAAGACATTCACTACGAGCCTGCT |
| | | TCTGGAGTCCATCAGAAAACGGGATGCAACTTGCCTAAAAT |
| | | GAGGAGAGGAGGATGCTTTTAAGAAAAAGAAGAAGGAGGA |
| | | TTCACTACCAGCTCTGAAGGGTGGAAAAGAGATGATTCATC |
| | | CGGATTGTGGAGAGGGTGGAATCTTGTTTAGGAGAGCGTT |
| | | GGTTGTGGCAGGCAGGGTGTAACTATGAATCAGTGAAGAC |
| | | AATTCACATCCTGGGATGAAAAGAAGGCCATGGGCTCACA |
| | | GGAGATTATCCACTGGCCTCTCCACATCCGCTTGCAGTAA |
| | | GGAGTGTGGGACTCTCCCAAGCTTCAGCGCTGAACTGCAA |
| | | TGCAGTGACGTCGCTTAGCTGGGCCAGTAACCGAGGGAGT |
| | | TGAATTTTCTGTCATTTTAAAATAATGTGTCTTTTAAGAAACA |
| | | CTTTGAAATTGOTTACCCAGACTCAGAGAAGTCTCCCTGTT |
| | | CTGTCCTAGCTAGTGATTCCTGTGTTGTGTGCATTCGTCTT |
| | | TTCCAGAGCAAACCGCCCAGAGTAGAAG |
| 28 | SEQ ID NO: 24 fused to | CATGAAAGACATTCACTACGAGCCTGCTTCTGGAGTCCATC |
| | SEQ ID NO: 22 | AGAAAACGGGATGCAACTTGCCTAAAATGAGGAGAGGAGG |
| | | ATGCTTTTAAGAAAAAGAAGAAGGAGGATTCACTACCAGCT |
| | | CTGAAGGGTGGAAAAGAGATGATTCATCCGGATTGTGGAG |
| | | AGGGTGGAATCTTGTTTAGGAGAGCGTTGGTTGTGGCAGG |
| | | CAGGGTGTAACTATGAATCAGTGAAGACAATTCACATCCTG |
| | | GGATGAAAAGAAGGCCATGGGCTCACAGGAGATTATCCAC |
| | | TGGCCTCTCCACATCCGCTTACCCAGACTCAGAGAAGTCT |
| | | CCCTGTTCTGTCCTAGCTAGTGATTCCTGTGTTGTGTGCAT |
| | | TCGTCTTTTCCAGAGCAAACCGCCCAGAGTAGAAG |
| 29 | SEQ ID NO: 1 fused to SEQ | CCGCAGAATCCTATCAGTTTCCCCCTTTCGTGCTGTGTGCA |
| | ID NO: 2 | TCGAGCAGGAAGGGGCTTGGCAGGTTTTACCTGCCCTCTT |
| | | TCCTTTCTGAAAAGTCTGGGCCTCCTCACCCCGAAAGGAG |
| | | TCACCTCCTTGCAGTTCCCCAGTTGCGAAAAGAGGAGGAA |
| | | GTTGGCTGGGCCGGGGGCCGGGGGGGGCACCCTCCGCA |
| | | GATGGCGGGACCCCCCTGCCGGCCATGGCAAAAACGAGG |
| | | CTTGTCTCTCCCACCGCCCCCAACCTTAGTCCTTGGCACAT |
| | | TGTTGAAAGTAATTGAATAAAATCGGAAATTCGAGAAGGCG |
| | | TTCGTTCGGATTGGTGAGATTTTGAGGGGAGAAAGAAGCG |
| | | GGGACTTCGCCGGCACCAGCGGCGCCCCCTCCTCGGCCA |
| | | CCGTTAACCCCCATTCCAGAGGGCACTGCCCCGCCACCCA |
| | | GCCTAGGTCCCCCTGCGAGAGCCTCGCGGGCCCGCGCAG |
| | | CCTCCGCGACTCGAACAGATCTTCAGTCCTTGGAGGAATG |
| | | CCTGTTTCTCTAACAATAAAAAATTAAAGAAGCGCTCATAAA |
| | | TGCCAAGTCCTCTCGCACTATGCGGAGTACAGAGGACAAC |
| | | GACCACAGCCATCCCTGAACCCCGCCCACGGCACAGCGC |
| | | CGGAGCCGGGGTCTGGGGCGCCGCTTCCTGGGGGGTCC |
| | | CGACTCTCAGCCGCCCCCGCTTCACCCGGGCCGCCAAGG |
| | | GGCTGGGGGAGGCGGCGCTCGGGGTAACCGGGGGAGAC |
| | | TCAGGGCGCTGGGGGCACTTGGGGAACTCATGGGGGCTC |
| | | AAAGGAACTAGGAGATCGGGACCTCGAAGGGGACTTGGG |
| | | GGGTTCGGGGCTTTCGGGGGGGGTCGGGGGTTCGCGGA |
| | | CCCGGGAAGCTCTGAGGACCCAGAGGCCGGGCGCGCTCC |
| | | GCCCGCGGCGCCGCCCCCTCCGTAACTTTCCCAGTCTCC |
| | | GAGGGAAGAGGGGGGGTGTGGGGTGCGGTTAAAAGGCGC |
| | | CACGGCGGGAGACAGGTGTTGCGGCCCCGCAGCGCCCG |
| | | CGCGCTCCTCTCCCCGACTCGGAGCCCCTCGGCGGCGCC |
| | | CGGCCCAGGACCCGCCTAGGAGCGCAGGAGCCCCAGCG |
| | | CAGAGACCCCAACGCCGAGACCCCCGCCCCGGCCCCGCC |
| | | GCGCTTCCTCCCGACGCAGGTGAGCCCGCCGGCCCCGGA |
| | | CTGCCCGGCCAGGAACCTGGCGCGGGGAGGGACCGCGA |
| | | GACCCAGAGCGGTTGCCCGGCCGCGTGGGTCTCGGGGAA |
| | | CCGGGGGGCTGGACCAACACACGTCCTTGGGCCGGGGG |
| | | GCGGGGGCCGCCTTCTGGAGCGGGCGTTTCTGCGGCCGA |
| | | GCTCCGGAGCTGGAATGGGGCGGCCGGGGAAGTGGACG |
| | | CGATGGCACCGCCCGGGGTGCGAGTGGGGCCGGGCGCG |
| | | CGCGGGAGGGGAAAAAGGCGCGGGCGAGCCGCCAGCGC |
| | | GAGGTTTGTGGTGTCGCCGATGTCCCTTCGGGGTACTCTA |
| | | GCGCAGCCGCCTGGCTACTTGACCCACTGCCACCAAACGT |
| | | TTTAAATTCACCGTTCGTGGCCACCTCTCTCCTGTTAGCCT |
| | | CTGACCAAGGTGGCACTGGATGGTTTCTGCCTGACCTTGG |
| | | TGCCCCGTGGCAGCGACTGTGGGTCATGAAAGACATTCAC |
| | | TACGAGCCTGCTTCTGGAGTCCATCAGAAAACGGGATGCA |
| | | ACTTGCCTAAAATGAGGAGAGGAGGATGCTTTTAAGAAAAA |
| | | GAAGAAGGAGGATTCACTACCAGCTCTGAAGGGTGGAAAA |
| | | GAGATGATTCATCCGGATTGTGGAGAGGGTGGAATCTTGT |
| | | TTAGGAGAGCGTTGGTTGTGGCAGGCAGGGTGTAACTATG |
| | | AATCAGTGAAGACAATTCACATCCTGGGATGAAAAGAAGG |
| | | CCATGGGCTCACAGGAGATTATCCACTGGCCTCTCCACAT |
| | | CCGCTTGCAGTAAGGAGTGTGGGACTCTCCCAAGCTTCAG |
| | | CGCTGAACTGCAATGCAGTGACGTCGCTTAGCTGGGCCAG |
| | | TAACCGAGGGAGTTGAATTTTCTGTCATTTTAAAATAATGTG |
| | | TCTTTTAAGAAACACTTTGAAATTAAAACCACAGCCCACAAT |
| | | TATAATGCACTGTTGCAGCACTTATCAAAACAGATATGCTA |
| | | ACTGAGCCATCAGTGCCAGCCTGACAGTGAGGCCACCAAG |
| | | CCATCCACAAAGCCTACACGAAAGTCTGTGCTCACAGTGG |
| | | CTTTTCTCCATGAAGAGGGCATTCCTAACCTCTTCCTTTCA |
| | | CGTAGGAGGAAGCAAGGTCCTTTGTAAAATTTTAACTCGGG |
| | | GTGCCTCAAATGTAAACTTAACCACTGGTAACAACAGTTTC |
| | | ACTGCTACATGCCACGTCTGTGAAAATTCATTCAAGACATT |
| | | AAGGAAAGTGGCTCAGCAGAGAGACTAGACATCTTATCCT |
| | | CACGGTTCTCCTGTACTTGGCCTCTCAGCCTTTGAGCAAG |
| | | GTTGGCCCAAGCTAGTATCGGCCCCAGTGGTACAGCCAAA |
| | | ACTTGAGACTGCAAATGGATGCAGCTGTTGAACGCTGAGT |
| | | AACTTCTGCAGAGTCAGGAAGACCCAAGGAAGCTCTGCAG |
| | | AGGATGCAGGGGTACGGTCAGAACCCCTGAGTGCCTTTCA |
| | | GCTAACGAGGACTTTATGACACTCCCCAGCACAGCAAATTT |
| | | TTATGATGTGTTTAAAGATTGGGTGAATTACTCAGGTGAAC |
| | | AAGCTACTTTTTATCAGAGAACACCTAAAAACACGTTCAAG |
| | | AGGGTTTGGGAACTATACATTTAATCCTATGACAAACTAAG |
| | | TTGGTTCTGTCTTCACCTGTTTTGGTGAGGTTGTGTAAGAG |
| | | TTGGTGTTTGCTCAGGAAGAGATTTAAGCATGCTTGCTTAC |
| | | CCAGACTCAGAGAAGTCTCCCTGTTCTGTCCTAGCTAGTGA |
| | | TTCCTGTGTTGTGTGCATTCGTCTTTTCCAGAGCAAACCGC |
| | | CCAGAGTAGAAG |
| 30 | SEQ ID NO: 3 fused to SEQ | ACAGAGGACAACGACCACAGCCATCCCTGAACCCCGCCCA |
| | ID NO: 20 | CGGCACAGCGCCGGAGCCGGGGTCTGGGGCGCCGCTTC |
| | | CTGGGGGGTCCCGACTCTCAGCCGCCCCCGCTTCACCCG |
| | | GGCCGCCAAGGGGCTGGGGGAGGCGGCGCTCGGGGTAA |
| | | CCGGGGGAGACTCAGGGCGCTGGGGGCACTTGGGGAACT |
| | | CATGGGGGCTCAAAGGAACTAGGAGATCGGGACCTCGAA |
| | | GGGGACTTGGGGGGTTCGGGGCTTTCGGGGGGGGTCGG |
| | | GGGTTCGCGGACCCGGGAAGCTCTGAGGACCCAGAGGCC |
| | | GGGCGCGCTCCGCCCGCGGCGCCGCCCCCTCCGTAACTT |
| | | TCCCAGTCTCCGAGGGAAGAGGGGGGGTGTGGGGTGCGG |
| | | TTAAAAGGCGCCACGGCGGGAGACAGGTGTTGCGGCCCC |
| | | GCAGCGCCCGCGCGCTCCTCTCCCCGACTCGGAGCCCCT |
| | | CGGCGGCGCCCGGCCCAGGACCCGCCTAGGAGCGCAGG |
| | | AGCCCCAGCGCAGAGACCCCAACGCCGAGACCCCCGCCC |
| | | CGGCCCCGCCGCGCTTCCTCCCGACGCAGGTGAGCCCGC |
| | | CGGCCCCGGACTGCCCGGCCAGGAACCTGGCGCGGGGA |
| | | GGGACCGCGAGACCCAGAGCGGTTGCCCGGCCGCGTGG |
| | | GTCTCGGGGAACCGGGGGGCTGGACCAACACACGTCCTT |
| | | GGGCCGGGGGGGGGGGGCCGCCTTCTGGAGCGGGCGTT |
| | | TCTGTTTATGATGTGTTTAAAGATTGGGTGAATTACTCAGGT |
| | | GAACAAGCTACTTTTTATCAGAGAACACCTAAAAACACGTT |
| | | CAAGAGGGTTTGGGAACTATACATTTAATCCTATGACAAAC |
| | | TAAGTTGGTTCTGTCTTCACCTGTTTTGGTGAGGTTGTGTA |
| | | AGAGTTGGTGTTTGCTCAGGAAGAGATTTAAGCATGCTTGC |
| | | TTACCCAGACTCAGAGAAGTCTCCCTGTTCTGTCCTAGCTA |
| | | GTGATTCCTGTGTTGTGTGCATTCGTCTTTTCCAGAGCAAA |
| | | CCGCCCAGAGTAGAAG |
| 31 | SEQ ID NO: 3 fused to SEQ | ACAGAGGACAACGACCACAGCCATCCCTGAACCCCGCCCA |
| | ID NO: 2 | CGGCACAGCGCCGGAGCCGGGGTCTGGGGCGCCGCTTC |
| | | CTGGGGGGTCCCGACTCTCAGCCGCCCCCGCTTCACCCG |
| | | GGCCGCCAAGGGGCTGGGGGAGGCGGCGCTCGGGGTAA |
| | | CCGGGGGAGACTCAGGGCGCTGGGGGCACTTGGGGAACT |
| | | CATGGGGGCTCAAAGGAACTAGGAGATCGGGACCTCGAA |
| | | GGGGACTTGGGGGGTTCGGGGCTTTCGGGGGCGGTCGG |
| | | GGGTTCGCGGACCCGGGAAGCTCTGAGGACCCAGAGGCC |
| | | GGGCGCGCTCCGCCCGCGGCGCCGCCCCCTCCGTAACTT |
| | | TCCCAGTCTCCGAGGGAAGAGGCGGGGTGTGGGGTGCGG |
| | | TTAAAAGGCGCCACGGCGGGAGACAGGTGTTGCGGCCCC |
| | | GCAGCGCCCGCGCGCTCCTCTCCCCGACTCGGAGCCCCT |
| | | CGGCGGCGCCCGGCCCAGGACCCGCCTAGGAGCGCAGG |
| | | AGCCCCAGCGCAGAGACCCCAACGCCGAGACCCCCGCCC |
| | | CGGCCCCGCCGCGCTTCCTCCCGACGCAGGTGAGCCCGC |
| | | CGGCCCCGGACTGCCCGGCCAGGAACCTGGCGCGGGGA |
| | | GGGACCGCGAGACCCAGAGCGGTTGCCCGGCCGCGTGG |
| | | GTCTCGGGGAACCGGGGGGCTGGACCAACACACGTCCTT |
| | | GGGCCGGGGGGGGGGGGCCGCCTTCTGGAGCGGGCGTT |
| | | TCTGTTCGTGGCCACCTCTCTCCTGTTAGCCTCTGACCAAG |
| | | GTGGCACTGGATGGTTTCTGCCTGACCTTGGTGCCCCGTG |
| | | GCAGCGACTGTGGGTCATGAAAGACATTCACTACGAGCCT |
| | | GCTTCTGGAGTCCATCAGAAAACGGGATGCAACTTGCCTA |
| | | AAATGAGGAGAGGAGGATGCTTTTAAGAAAAAGAAGAAGG |
| | | AGGATTCACTACCAGCTCTGAAGGGTGGAAAAGAGATGAT |
| | | TCATCCGGATTGTGGAGAGGGTGGAATCTTGTTTAGGAGA |
| | | GCGTTGGTTGTGGCAGGCAGGGTGTAACTATGAATCAGTG |
| | | AAGACAATTCACATCCTGGGATGAAAAGAAGGCCATGGGC |
| | | TCACAGGAGATTATCCACTGGCCTCTCCACATCCGCTTGCA |
| | | GTAAGGAGTGTGGGACTCTCCCAAGCTTCAGCGCTGAACT |
| | | GCAATGCAGTGACGTCGCTTAGCTGGGCCAGTAACCGAGG |
| | | GAGTTGAATTTTCTGTCATTTTAAAATAATGTGTCTTTTAAG |
| | | AAACACTTTGAAATTAAAACCACAGCCCACAATTATAATGCA |
| | | CTGTTGCAGCACTTATCAAAACAGATATGCTAACTGAGCCA |
| | | TCAGTGCCAGCCTGACAGTGAGGCCACCAAGCCATCCACA |
| | | AAGCCTACACGAAAGTCTGTGCTCACAGTGGCTTTTCTCCA |
| | | TGAAGAGGGCATTCCTAACCTCTTCCTTTCACGTAGGAGGA |
| | | AGCAAGGTCCTTTGTAAAATTTTAACTCGGGGTGCCTCAAA |
| | | TGTAAACTTAACCACTGGTAACAACAGTTTCACTGCTACAT |
| | | GCCACGTCTGTGAAAATTCATTCAAGACATTAAGGAAAGTG |
| | | GCTCAGCAGAGAGACTAGACATCTTATCCTCACGGTTCTCC |
| | | TGTACTTGGCCTCTCAGCCTTTGAGCAAGGTTGGCCCAAG |
| | | CTAGTATCGGCCCCAGTGGTACAGCCAAAACTTGAGACTG |
| | | CAAATGGATGCAGCTGTTGAACGCTGAGTAACTTCTGCAG |
| | | AGTCAGGAAGACCCAAGGAAGCTCTGCAGAGGATGCAGG |
| | | GGTACGGTCAGAACCCCTGAGTGCCTTTCAGCTAACGAGG |
| | | ACTTTATGACACTCCCCAGCACAGCAAATTTTTATGATGTG |
| | | TTTAAAGATTGGGTGAATTACTCAGGTGAACAAGCTACTTT |
| | | TTATCAGAGAACACCTAAAAACACGTTCAAGAGGGTTTGGG |
| | | AACTATACATTTAATCCTATGACAAACTAAGTTGGTTCTGTC |
| | | TTCACCTGTTTTGGTGAGGTTGTGTAAGAGTTGGTGTTTGC |
| | | TCAGGAAGAGATTTAAGCATGCTTGCTTACCCAGACTCAGA |
| | | GAAGTCTCCCTGTTCTGTCCTAGCTAGTGATTCCTGTGTTG |
| | | TGTGCATTCGTCTTTTCCAGAGCAAACCGCCCAGAGTAGAA |
| | | G |
| 32 | SEQ ID NO: 3 fused to SEQ | ACAGAGGACAACGACCACAGCCATCCCTGAACCCCGCCCA |
| | ID NO: 21 | CGGCACAGCGCCGGAGCCGGGGTCTGGGGCGCCGCTTC |
| | | CTGGGGGGTCCCGACTCTCAGCCGCCCCCGCTTCACCCG |
| | | GGCCGCCAAGGGGCTGGGGGAGGCGGCGCTCGGGGTAA |
| | | CCGGGGGAGACTCAGGGCGCTGGGGGCACTTGGGGAACT |
| | | CATGGGGGCTCAAAGGAACTAGGAGATCGGGACCTCGAA |
| | | GGGGACTTGGGGGGTTCGGGGCTTTCGGGGGGGGTCGG |
| | | GGGTTCGCGGACCCGGGAAGCTCTGAGGACCCAGAGGCC |
| | | GGGCGCGCTCCGCCCGCGGCGCCGCCCCCTCCGTAACTT |
| | | TCCCAGTCTCCGAGGGAAGAGGGGGGGTGTGGGGTGCGG |
| | | TTAAAAGGCGCCACGGCGGGAGACAGGTGTTGCGGCCCC |
| | | GCAGCGCCCGCGCGCTCCTCTCCCCGACTCGGAGCCCCT |
| | | CGGCGGCGCCCGGCCCAGGACCCGCCTAGGAGCGCAGG |
| | | AGCCCCAGCGCAGAGACCCCAACGCCGAGACCCCCGCCC |
| | | CGGCCCCGCCGCGCTTCCTCCCGACGCAGGTGAGCCCGC |
| | | CGGCCCCGGACTGCCCGGCCAGGAACCTGGCGCGGGGA |
| | | GGGACCGCGAGACCCAGAGCGGTTGCCCGGCCGCGTGG |
| | | GTCTCGGGGAACCGGGGGGCTGGACCAACACACGTCCTT |
| | | GGGCCGGGGGGGGGGGGCCGCCTTCTGGAGCGGGCGTT |
| | | TCTGAAAACCACAGCCCACAATTATAATGCACTGTTGCAGC |
| | | ACTTATCAAAACAGATATGCTAACTGAGCCATCAGTGCCAG |
| | | CCTGACAGTGAGGCCACCAAGCCATCCACAAAGCCTACAC |
| | | GAAAGTCTGTGCTCACAGTGGCTTTTCTCCATGAAGAGGG |
| | | CATTCCTAACCTCTTCCTTTCACGTAGGAGGAAGCAAGGTC |
| | | CTTTGTAAAATTTTAACTCGGGGTGCCTCAAATGTAAACTTA |
| | | ACCACTGGTAACAACAGTTTCACTGCTACATGCCACGTCTG |
| | | TGAAAATTCATTCAAGACATTAAGGAAAGTGGCTCAGCAGA |
| | | GAGACTAGACATCTTATCCTCACGGTTCTCCTGTACTTGGC |
| | | CTCTCAGCCTTTGAGCAAGGTTGGCCCAAGCTAGTATCGG |
| | | CCCCAGTGGTACAGCCAAAACTTGAGACTGCAAATGGATG |
| | | CAGCTGTTGAACGCTGAGTAACTTCTGCAGAGTCAGGAAG |
| | | ACCCAAGGAAGCTCTGCAGAGGATGCAGGGGTACGGTCA |
| | | GAACCCCTGAGTGCCTTTCAGCTAACGAGGACTTTATGACA |
| | | CTCCCCAGCACAGCAAATTTTTATGATGTGTTTAAAGATTG |
| | | GGTGAATTACTCAGGTGAACAAGCTACTTTTTATCAGAGAA |
| | | CACCTAAAAACACGTTCAAGAGGGTTTGGGAACTATACATT |
| | | TAATCCTATGACAAACTAAGTTGGTTCTGTCTTCACCTGTTT |
| | | TGGTGAGGTTGTGTAAGAGTTGGTGTTTGCTCAGGAAGAG |
| | | ATTTAAGCATGCTTGCTTACCCAGACTCAGAGAAGTCTCCC |
| | | TGTTCTGTCCTAGCTAGTGATTCCTGTGTTGTGTGCATTCG |
| | | TCTTTTCCAGAGCAAACCGCCCAGAGTAGAAG |
| 33 | SEQ ID NO: 3 fused to SEQ | ACAGAGGACAACGACCACAGCCATCCCTGAACCCCGCCCA |
| | ID NO: 25 | CGGCACAGCGCCGGAGCCGGGGTCTGGGGCGCCGCTTC |
| | | CTGGGGGGTCCCGACTCTCAGCCGCCCCCGCTTCACCCG |
| | | GGCCGCCAAGGGGCTGGGGGAGGCGGCGCTCGGGGTAA |
| | | CCGGGGGAGACTCAGGGCGCTGGGGGCACTTGGGGAACT |
| | | CATGGGGGCTCAAAGGAACTAGGAGATCGGGACCTCGAA |
| | | GGGGACTTGGGGGGTTCGGGGCTTTCGGGGGGGGTCGG |
| | | GGGTTCGCGGACCCGGGAAGCTCTGAGGACCCAGAGGCC |
| | | GGGCGCGCTCCGCCCGCGGCGCCGCCCCCTCCGTAACTT |
| | | TCCCAGTCTCCGAGGGAAGAGGCGGGGTGTGGGGTGCGG |
| | | TTAAAAGGCGCCACGGGGGGAGACAGGTGTTGCGGCCCC |
| | | GCAGCGCCCGCGCGCTCCTCTCCCCGACTCGGAGCCCCT |
| | | CGGCGGCGCCCGGCCCAGGACCCGCCTAGGAGCGCAGG |
| | | AGCCCCAGCGCAGAGACCCCAACGCCGAGACCCCCGCCC |
| | | CGGCCCCGCCGCGCTTCCTCCCGACGCAGGTGAGCCCGC |
| | | CGGCCCCGGACTGCCCGGCCAGGAACCTGGCGCGGGGA |
| | | GGGACCGCGAGACCCAGAGCGGTTGCCCGGCCGCGTGG |
| | | GTCTCGGGGAACCGGGGGGCTGGACCAACACACGTCCTT |
| | | GGGCCGGGGGGGGGGGGCCGCCTTCTGGAGCGGGCGTT |
| | | TCTGTTCGTGGCCACCTCTCTCCTGTTAGCCTCTGACCAAG |
| | | GTGGCACTGGATGGTTTCTGCCTGACCTTGGTGCCCCGTG |
| | | GCAGCGACTGTGGGTCATGAAAGACATTCACTACGAGCCT |
| | | GCTTCTGGAGTCCATCAGAAAACGGGATGCAACTTGCCTA |
| | | AAATGAGGAGAGGAGGATGCTTTTAAGAAAAAGAAGAAGG |
| | | AGGATTCACTACCAGCTCTGAAGGGTGGAAAAGAGATGAT |
| | | TCATCCGGATTGTGGAGAGGGTGGAATCTTGTTTAGGAGA |
| | | GCGTTGGTTGTGGCAGGCAGGGTGTAACTATGAATCAGTG |
| | | AAGACAATTCACATCCTGGGATGAAAAGAAGGCCATGGGC |
| | | TCACAGGAGATTATCCACTGGCCTCTCCACATCCGCTTGCA |
| | | GTAAGGAGTGTGGGACTCTCCCAAGCTTCAGCGCTGAACT |
| | | GCAATGCAGTGACGTCGCTTAGCTGGGCCAGTAACCGAGG |
| | | GAGTTGAATTTTCTGTCATTTTAAAATAATGTGTCTTTTAAG |
| | | AAACACTTTGAAATTTTTATGATGTGTTTAAAGATTGGGTGA |
| | | ATTACTCAGGTGAACAAGCTACTTTTTATCAGAGAACACCT |
| | | AAAAACACGTTCAAGAGGGTTTGGGAACTATACATTTAATC |
| | | CTATGACAAACTAAGTTGGTTCTGTCTTCACCTGTTTTGGT |
| | | GAGGTTGTGTAAGAGTTGGTGTTTGCTCAGGAAGAGATTTA |
| | | AGCATGCTTGCTTACCCAGACTCAGAGAAGTCTCCCTGTTC |
| | | TGTCCTAGCTAGTGATTCCTGTGTTGTGTGCATTCGTCTTT |
| | | TCCAGAGCAAACCGCCCAGAGTAGAAG |
| 34 | SEQ ID NO: 3 fused to SEQ | ACAGAGGACAACGACCACAGCCATCCCTGAACCCCGCCCA |
| | ID NO: 26 | CGGCACAGCGCCGGAGCCGGGGTCTGGGGCGCCGCTTC |
| | | CTGGGGGGTCCCGACTCTCAGCCGCCCCCGCTTCACCCG |
| | | GGCCGCCAAGGGGCTGGGGGAGGCGGCGCTCGGGGTAA |
| | | CCGGGGGAGACTCAGGGCGCTGGGGGCACTTGGGGAACT |
| | | CATGGGGGCTCAAAGGAACTAGGAGATCGGGACCTCGAA |
| | | GGGGACTTGGGGGGTTCGGGGCTTTCGGGGGGGGTCGG |
| | | GGGTTCGCGGACCCGGGAAGCTCTGAGGACCCAGAGGCC |
| | | GGGCGCGCTCCGCCCGCGGCGCCGCCCCCTCCGTAACTT |
| | | TCCCAGTCTCCGAGGGAAGAGGGGGGGTGTGGGGTGCGG |
| | | TTAAAAGGCGCCACGGGGGGAGACAGGTGTTGCGGCCCC |
| | | GCAGCGCCCGCGCGCTCCTCTCCCCGACTCGGAGCCCCT |
| | | CGGCGGCGCCCGGCCCAGGACCCGCCTAGGAGCGCAGG |
| | | AGCCCCAGCGCAGAGACCCCAACGCCGAGACCCCCGCCC |
| | | CGGCCCCGCCGCGCTTCCTCCCGACGCAGGTGAGCCCGC |
| | | CGGCCCCGGACTGCCCGGCCAGGAACCTGGCGCGGGGA |
| | | GGGACCGCGAGACCCAGAGCGGTTGCCCGGCCGCGTGG |
| | | GTCTCGGGGAACCGGGGGGCTGGACCAACACACGTCCTT |
| | | GGGCCGGGGGGGGGGGGCCGCCTTCTGGAGCGGGCGTT |
| | | TCTGCATGAAAGACATTCACTACGAGCCTGCTTCTGGAGTC |
| | | CATCAGAAAACGGGATGCAACTTGCCTAAAATGAGGAGAG |
| | | GAGGATGCTTTTAAGAAAAAGAAGAAGGAGGATTCACTACC |
| | | AGCTCTGAAGGGTGGAAAAGAGATGATTCATCCGGATTGT |
| | | GGAGAGGGTGGAATCTTGTTTAGGAGAGCGTTGGTTGTGG |
| | | CAGGCAGGGTGTAACTATGAATCAGTGAAGACAATTCACAT |
| | | CCTGGGATGAAAAGAAGGCCATGGGCTCACAGGAGATTAT |
| | | CCACTGGCCTCTCCACATCCTTTATGATGTGTTTAAAGATT |
| | | GGGTGAATTACTCAGGTGAACAAGCTACTTTTTATCAGAGA |
| | | ACACCTAAAAACACGTTCAAGAGGGTTTGGGAACTATACAT |
| | | TTAATCCTATGACAAACTAAGTTGGTTCTGTCTTCACCTGTT |
| | | TTGGTGAGGTTGTGTAAGAGTTGGTGTTTGCTCAGGAAGA |
| | | GATTTAAGCATGCTTGCTTACCCAGACTCAGAGAAGTCTCC |
| | | CTGTTCTGTCCTAGCTAGTGATTCCTGTGTTGTGTGCATTC |
| | | GTCTTTTCCAGAGCAAACCGCCCAGAGTAGAAG |
| 35 | SEQ ID NO: 1 fused to SEQ | CCGCAGAATCCTATCAGTTTCCCCCTTTCGTGCTGTGTGCA |
| | ID NO: 22 | TCGAGCAGGAAGGGGCTTGGCAGGTTTTACCTGCCCTCTT |
| | | TCCTTTCTGAAAAGTCTGGGCCTCCTCACCCCGAAAGGAG |
| | | TCACCTCCTTGCAGTTCCCCAGTTGCGAAAAGAGGAGGAA |
| | | GTTGGCTGGGCCGGGGGCCGGGGGGGGCACCCTCCGCA |
| | | GATGGCGGGACCCCCCTGCCGGCCATGGCAAAAACGAGG |
| | | CTTGTCTCTCCCACCGCCCCCAACCTTAGTCCTTGGCACAT |
| | | TGTTGAAAGTAATTGAATAAAATCGGAAATTCGAGAAGGCG |
| | | TTCGTTCGGATTGGTGAGATTTTGAGGGGAGAAAGAAGCG |
| | | GGGACTTCGCCGGCACCAGCGGCGCCCCCTCCTCGGCCA |
| | | CCGTTAACCCCCATTCCAGAGGGCACTGCCCCGCCACCCA |
| | | GCCTAGGTCCCCCTGCGAGAGCCTCGCGGGCCCGCGCAG |
| | | CCTCCGCGACTCGAACAGATCTTCAGTCCTTGGAGGAATG |
| | | CCTGTTTCTCTAACAATAAAAAATTAAAGAAGCGCTCATAAA |
| | | TGCCAAGTCCTCTCGCACTATGCGGAGTACAGAGGACAAC |
| | | GACCACAGCCATCCCTGAACCCCGCCCACGGCACAGCGC |
| | | CGGAGCCGGGGTCTGGGGCGCCGCTTCCTGGGGGGTCC |
| | | CGACTCTCAGCCGCCCCCGCTTCACCCGGGCCGCCAAGG |
| | | GGCTGGGGGAGGCGGCGCTCGGGGTAACCGGGGGAGAC |
| | | TCAGGGCGCTGGGGGCACTTGGGGAACTCATGGGGGCTC |
| | | AAAGGAACTAGGAGATCGGGACCTCGAAGGGGACTTGGG |
| | | GGGTTCGGGGCTTTCGGGGGGGGTCGGGGGTTCGCGGA |
| | | CCCGGGAAGCTCTGAGGACCCAGAGGCCGGGCGCGCTCC |
| | | GCCCGCGGCGCCGCCCCCTCCGTAACTTTCCCAGTCTCC |
| | | GAGGGAAGAGGGGGGTGTGGGGTGCGGTTAAAAGGCGC |
| | | CACGGCGGGAGACAGGTGTTGCGGCCCCGCAGCGCCCG |
| | | CGCGCTCCTCTCCCCGACTCGGAGCCCCTCGGCGGCGCC |
| | | CGGCCCAGGACCCGCCTAGGAGCGCAGGAGCCCCAGCG |
| | | CAGAGACCCCAACGCCGAGACCCCCGCCCCGGCCCCGCC |
| | | GCGCTTCCTCCCGACGCAGGTGAGCCCGCCGGCCCCGGA |
| | | CTGCCCGGCCAGGAACCTGGCGCGGGGAGGGACCGCGA |
| | | GACCCAGAGCGGTTGCCCGGCCGCGTGGGTCTCGGGGAA |
| | | CCGGGGGGCTGGACCAACACACGTCCTTGGGCCGGGGG |
| | | GCGGGGGCCGCCTTCTGGAGCGGGCGTTTCTGCGGCCGA |
| | | GCTCCGGAGCTGGAATGGGGCGGCCGGGGAAGTGGACG |
| | | CGATGGCACCGCCCGGGGTGCGAGTGGGGCCGGGCGCG |
| | | CGCGGGAGGGGAAAAAGGCGCGGGCGAGCCGCCAGCGC |
| | | GAGGTTTGTGGTGTCGCCGATGTCCCTTCGGGGTACTCTA |
| | | GCGCAGCCGCCTGGCTACTTGACCCACTGCCACCAAACGT |
| | | TTTAAATTCACCGGCTTACCCAGACTCAGAGAAGTCTCCCT |
| | | GTTCTGTCCTAGCTAGTGATTCCTGTGTTGTGTGCATTCGT |
| | | CTTTTCCAGAGCAAACCGCCCAGAGTAGAAG |
| 36 | SEQ ID NO: 3 joined to | ACAGAGGACAACGACCACAGCCATCCCTGAACCCCGCCCA |
| | SEQ ID NO: 2 by the | CGGCACAGCGCCGGAGCCGGGGTCTGGGGCGCCGCTTC |
| | endogenous intervening | CTGGGGGGTCCCGACTCTCAGCCGCCCCCGCTTCACCCG |
| | nucleic acid sequence | GGCCGCCAAGGGGCTGGGGGAGGCGGCGCTCGGGGTAA |
| | | CCGGGGGAGACTCAGGGCGCTGGGGGCACTTGGGGAACT |
| | | CATGGGGGCTCAAAGGAACTAGGAGATCGGGACCTCGAA |
| | | GGGGACTTGGGGGGTTCGGGGCTTTCGGGGGGGGTCGG |
| | | GGGTTCGCGGACCCGGGAAGCTCTGAGGACCCAGAGGCC |
| | | GGGCGCGCTCCGCCCGCGGCGCCGCCCCCTCCGTAACTT |
| | | TCCCAGTCTCCGAGGGAAGAGGGGGGGTGTGGGGTGCGG |
| | | TTAAAAGGCGCCACGGCGGGAGACAGGTGTTGCGGCCCC |
| | | GCAGCGCCCGCGCGCTCCTCTCCCCGACTCGGAGCCCCT |
| | | CGGCGGCGCCCGGCCCAGGACCCGCCTAGGAGCGCAGG |
| | | AGCCCCAGCGCAGAGACCCCAACGCCGAGACCCCCGCCC |
| | | CGGCCCCGCCGCGCTTCCTCCCGACGCAGGTGAGCCCGC |
| | | CGGCCCCGGACTGCCCGGCCAGGAACCTGGCGCGGGGA |
| | | GGGACCGCGAGACCCAGAGCGGTTGCCCGGCCGCGTGG |
| | | GTCTCGGGGAACCGGGGGGCTGGACCAACACACGTCCTT |
| | | GGGCCGGGGGGGGGGGGCCGCCTTCTGGAGCGGGCGTT |
| | | TCTGCGGCCGAGCTCCGGAGCTGGAATGGGGCGGCCGG |
| | | GGAAGTGGACGCGATGGCACCGCCCGGGGTGCGAGTGG |
| | | GGCCGGGCGCGCGCGGGAGGGGAAAAAGGCGCGGGCGA |
| | | GCCGCCAGCGCGAGGTTTGTGGTGTCGCCGATGTCCCTTC |
| | | GGGGTACTCTAGCGCAGCCGCCTGGCTACTTGACCCACTG |
| | | CCACCAAACGTTTTAAATTCACCGAAAGCTTAGCTTCGAAG |
| | | CAAAGCTCCGTTTCGCCGGTGAAGCAGGAAGCCTTCGCTG |
| | | CAGGAACTGACCTTTACCTCTTGGAGCGGCTTCTGCAGAA |
| | | AAATCCCCGGGCAGAGATTTGGGGGGAGTTTGCCTAGAAC |
| | | TAACGCGGAGCCAGCCGATCCCGGCCTACCCCGGGGCCA |
| | | AGATTTCAGTGGCTTCCCTTTTTCCTAAACACTTCACGAGG |
| | | GTCTGTTTCCGGGCTGTGCTCCCCGCCTAGAAGGAAAATT |
| | | TTTAGGACCCTTGTTCGCGAAGAGGTGGTGTGCGGCTGAG |
| | | ACCCGCGTCCTCAGGACGGTTCCATCAGTGCCTCGATCCT |
| | | GCCCCACTGGAGGAGGAAGGCAGCCCGAACAGCGCTCAC |
| | | CTAACTAACAGCTGCTGAGAGCTGGGTTCCGTGGCCATGC |
| | | ACCTGGGACTGCCTTGAGAAGCGTGGTACGGCCGTGTCC |
| | | CCATGTGACCTTAGAGTCCCTTTCGAAACTGCTGTGCACAG |
| | | TCGGTCACAATTTCAGACACTGGTGAGAAGGGTGGAGGAA |
| | | CCCTCTGGGGACAGCCAGGCAAGGTCGACCACCCATCAC |
| | | CTAAGGGTGGAGAAATTTAAGGGGTGAAGAGTCCCTTTTG |
| | | CCTTTTCTGGATCCTGGTGATTCACCTAGTGTCTTCCCTAA |
| | | GGAACTGAACCAACTCCTCCGCTGGCCTCTGGCAGCCCTC |
| | | CAGGCGGTGCAGGATGGCGTGGGCCCGGTAGGAAGCTGC |
| | | ATGTAACCGCCCAGGGTCGGGAGGCCAGGAGGGCAGCTC |
| | | CTCCTCTGACTTGAATATTGAAAACAACTTCGTCCTGCTTCT |
| | | GAGCCCCTCTTAACCCATGACCCCCTAGCCCATTGGGGAG |
| | | TAAATCTTAATTTACTCCTCTTCCTGAAAAAGGATOTTTAAA |
| | | ACAGGTAGCTTCAACTCAAGCTTTATAAAATAACAATATAG |
| | | GGTTTCTCGGAACTGTATTTTTCTCAGCTGATGGTAACTGG |
| | | ACAGGTCTGTAGAAGGGTGTATGACCTGGGTTTGGCAGGT |
| | | GGAAGAGGGCAAAGGATAAACCCCTCCTCCTGCAGCCCCA |
| | | TATTCTTGGCCAGGTGTATTGTTGTAAACCAGGAGAGAGTT |
| | | TACTTCGGGGAGTATCCTGTTTTCCACTCAGTGAGGGCCA |
| | | ATGAAGAATGTCTAATTCCATAAGATGCTTTTGTTAAAATCG |
| | | GAATGTTGCTGTCCTCGGTGGTTCTGCTGTTGGGACGGGA |
| | | CTGGCCTGAGCTGTGGGTGCTGTAGCAGGACAACCAGCTC |
| | | ACCTAAGGGCCTCCCAGTCTGGATTATCAATGGGTCAGTG |
| | | CTGAACCTGGGCTAAAATATTGTTTTTTCCAATGATGTTGTC |
| | | TTTCCCAAGCTCAGTGAAGCTAAATGTTTCACAGGCCTATG |
| | | TCAATCTGATGTAACTTTCGTGGCCACCTCTCTCCTGTTAG |
| | | CCTCTGACCAAGGTGGCACTGGATGGTTTCTGCCTGACCT |
| | | TGGTGCCCCGTGGCAGCGACTGTGGGTCATGAAAGACATT |
| | | CACTACGAGCCTGCTTCTGGAGTCCATCAGAAAACGGGAT |
| | | GCAACTTGCCTAAAATGAGGAGAGGAGGATGCTTTTAAGA |
| | | AAAAGAAGAAGGAGGATTCACTACCAGCTCTGAAGGGTGG |
| | | AAAAGAGATGATTCATCCGGATTGTGGAGAGGGTGGAATC |
| | | TTGTTTAGGAGAGCGTTGGTTGTGGCAGGCAGGGTGTAAC |
| | | TATGAATCAGTGAAGACAATTCACATCCTGGGATGAAAAGA |
| | | AGGCCATGGGCTCACAGGAGATTATCCACTGGCCTCTCCA |
| | | CATCCGCTTGCAGTAAGGAGTGTGGGACTCTCCCAAGCTT |
| | | CAGCGCTGAACTGCAATGCAGTGACGTCGCTTAGCTGGGC |
| | | CAGTAACCGAGGGAGTTGAATTTTCTGTCATTTTAAAATAAT |
| | | GTGTCTTTTAAGAAACACTTTGAAATTAAAACCACAGCCCA |
| | | CAATTATAATGCACTGTTGCAGCACTTATCAAAACAGATAT |
| | | GCTAACTGAGCCATCAGTGCCAGCCTGACAGTGAGGCCAC |
| | | CAAGCCATCCACAAAGCCTACACGAAAGTCTGTGCTCACA |
| | | GTGGCTTTTCTCCATGAAGAGGGCATTCCTAACCTCTTCCT |
| | | TTCACGTAGGAGGAAGCAAGGTCCTTTGTAAAATTTTAACT |
| | | CGGGGTGCCTCAAATGTAAACTTAACCACTGGTAACAACA |
| | | GTTTCACTGCTACATGCCACGTCTGTGAAAATTCATTCAAG |
| | | ACATTAAGGAAAGTGGCTCAGCAGAGAGACTAGACATCTTA |
| | | TCCTCACGGTTCTCCTGTACTTGGCCTCTCAGCCTTTGAGC |
| | | AAGGTTGGCCCAAGCTAGTATCGGCCCCAGTGGTACAGCC |
| | | AAAACTTGAGACTGCAAATGGATGCAGCTGTTGAACGCTG |
| | | AGTAACTTCTGCAGAGTCAGGAAGACCCAAGGAAGCTCTG |
| | | CAGAGGATGCAGGGGTACGGTCAGAACCCCTGAGTGCCTT |
| | | TCAGCTAACGAGGACTTTATGACACTCCCCAGCACAGCAA |
| | | ATTTTTATGATGTGTTTAAAGATTGGGTGAATTACTCAGGTG |
| | | AACAAGCTACTTTTTATCAGAGAACACCTAAAAACACGTTC |
| | | AAGAGGGTTTGGGAACTATACATTTAATCCTATGACAAACT |
| | | AAGTTGGTTCTGTCTTCACCTGTTTTGGTGAGGTTGTGTAA |
| | | GAGTTGGTGTTTGCTCAGGAAGAGATTTAAGCATGCTTGCT |
| | | TACCCAGACTCAGAGAAGTCTCCCTGTTCTGTCCTAGCTAG |
| | | TGATTCCTGTGTTGTGTGCATTCGTCTTTTCCAGAGCAAAC |
| | | CGCCCAGAGTAGAAG |
| 37 | SEQ ID NO: 36 with SEQ ID | ACAGAGGACAACGACCACAGCCATCCCTGAACCCCGCCCA |
| | NO: 23 removed | CGGCACAGCGCCGGAGCCGGGGTCTGGGGCGCCGCTTC |
| | | CTGGGGGGTCCCGACTCTCAGCCGCCCCCGCTTCACCCG |
| | | GGCCGCCAAGGGGCTGGGGGAGGCGGCGCTCGGGGTAA |
| | | CCGGGGGAGACTCAGGGCGCTGGGGGCACTTGGGGAACT |
| | | CATGGGGGCTCAAAGGAACTAGGAGATCGGGACCTCGAA |
| | | GGGGACTTGGGGGGTTCGGGGCTTTCGGGGGCGGTCGG |
| | | GGGTTCGCGGACCCGGGAAGCTCTGAGGACCCAGAGGCC |
| | | GGGCGCGCTCCGCCCGCGGCGCCGCCCCCTCCGTAACTT |
| | | TCCCAGTCTCCGAGGGAAGAGGGGGGTGTGGGGTGCGG |
| | | TTAAAAGGCGCCACGGCGGGAGACAGGTGTTGCGGCCCC |
| | | GCAGCGCCCGCGCGCTCCTCTCCCCGACTCGGAGCCCCT |
| | | CGGCGGCGCCCGGCCCAGGACCCGCCTAGGAGCGCAGG |
| | | AGCCCCAGCGCAGAGACCCCAACGCCGAGACCCCCGCCC |
| | | CGGCCCCGCCGCGCTTCCTCCCGACGCAGGTGAGCCCGC |
| | | CGGCCCCGGACTGCCCGGCCAGGAACCTGGCGCGGGGA |
| | | GGGACCGCGAGACCCAGAGCGGTTGCCCGGCCGCGTGG |
| | | GTCTCGGGGAACCGGGGGGCTGGACCAACACACGTCCTT |
| | | GGGCCGGGGGGGGGGGGCCGCCTTCTGGAGCGGGCGTT |
| | | TCTGCGGCCGAGCTCCGGAGCTGGAATGGGGCGGCCGG |
| | | GGAAGTGGACGCGATGGCACCGCCCGGGGTGCGAGTGG |
| | | GGCCGGGCGCGCGCGGGAGGGGAAAAAGGCGCGGGCGA |
| | | GCCGCCAGCGCGAGGTTTGTGGTGTCGCCGATGTCCCTTC |
| | | GGGGTACTCTAGCGCAGCCGCCTGGCTACTTGACCCACTG |
| | | CCACCAAACGTTTTAAATTCACCGAAAGCTTAGCTTCGAAG |
| | | CAAAGCTCCGTTTCGCCGGTGAAGCAGGAAGCCTTCGCTG |
| | | CAGGAACTGACCTTTACCTCTTGGAGCGGCTTCTGCAGAA |
| | | AAATCCCCGGGCAGAGATTTGGGCGGAGTTTGCCTAGAAC |
| | | TAACGCGGAGCCAGCCGATCCCGGCCTACCCCGGGGCCA |
| | | AGATTTCAGTGGCTTCCCTTTTTCCTAAACACTTCACGAGG |
| | | GTCTGTTTCCGGGCTGTGCTCCCCGCCTAGAAGGAAAATT |
| | | TTTAGGACCCTTGTTCGCGAAGAGGTGGTGTGCGGCTGAG |
| | | ACCCGCGTCCTCAGGACGGTTCCATCAGTGCCTCGATCCT |
| | | GCCCCACTGGAGGAGGAAGGCAGCCCGAACAGCGCTCAC |
| | | CTAACTAACAGCTGCTGAGAGCTGGGTTCCGTGGCCATGC |
| | | ACCTGGGACTGCCTTGAGAAGCGTGGTACGGCCGTGTCC |
| | | CCATGTGACCTTAGAGTCCCTTTCGAAACTGCTGTGCACAG |
| | | TCGGTCACAATTTCAGACACTGGTGAGAAGGGTGGAGGAA |
| | | CCCTCTGGGGACAGCCAGGCAAGGTCGACCACCCATCAC |
| | | CTAAGGGTGGAGAAATTTAAGGGGTGAAGAGTCCCTTTTG |
| | | CCTTTTCTGGATCCTGGTGATTCACCTAGTGTCTTCCCTAA |
| | | GGAACTGAACCAACTCCTCCGCTGGCCTCTGGCAGCCCTC |
| | | CAGGCGGTGCAGGATGGCGTGGGCCCGGTAGGAAGCTGC |
| | | ATGTAACCGCCCAGGGTCGGGAGGCCAGGAGGGCAGCTC |
| | | CTCCTCTGACTTGAATATTGAAAACAACTTCGTCCTGCTTCT |
| | | GAGCCCCTCTTAACCCATGACCCCCTAGCCCATTGGGGAG |
| | | TAAATCTTAATTTACTCCTCTTCCTGAAAAAGGATCTTTAAA |
| | | ACAGGTAGCTTCAACTCAAGCTTTATAAAATAACAATATAG |
| | | GGTTTCTCGGAACTGTATTTTTCTCAGCTGATGGTAACTGG |
| | | ACAGGTCTGTAGAAGGGTGTATGACCTGGGTTTGGCAGGT |
| | | GGAAGAGGGCAAAGGATAAACCCCTCCTCCTGCAGCCCCA |
| | | TATTCTTGGCCAGGTGTATTGTTGTAAACCAGGAGAGAGTT |
| | | TACTTCGGGGAGTATCCTGTTTTCCACTCAGTGAGGGCCA |
| | | ATGAAGAATGTCTAATTCCATAAGATGCTTTTGTTAAAATCG |
| | | GAATGTTGCTGTCCTCGGTGGTTCTGCTGTTGGGACGGGA |
| | | CTGGCCTGAGCTGTGGGTGCTGTAGCAGGACAACCAGCTC |
| | | ACCTAAGGGCCTCCCAGTCTGGATTATCAATGGGTCAGTG |
| | | CTGAACCTGGGCTAAAATATTGTTTTTTCCAATGATGTTGTC |
| | | TTTCCCAAGCTCAGTGAAGCTAAATGTTTCACAGGCCTATG |
| | | TCAATCTGATGTAAAAAACCACAGCCCACAATTATAATGCA |
| | | CTGTTGCAGCACTTATCAAAACAGATATGCTAACTGAGCCA |
| | | TCAGTGCCAGCCTGACAGTGAGGCCACCAAGCCATOCACA |
| | | AAGCCTACACGAAAGTCTGTGCTCACAGTGGCTTTTCTCCA |
| | | TGAAGAGGGCATTCCTAACCTCTTCCTTTCACGTAGGAGGA |
| | | AGCAAGGTCCTTTGTAAAATTTTAACTCGGGGTGCCTCAAA |
| | | TGTAAACTTAACCACTGGTAACAACAGTTTCACTGCTACAT |
| | | GCCACGTCTGTGAAAATTCATTCAAGACATTAAGGAAAGTG |
| | | GCTCAGCAGAGAGACTAGACATCTTATCCTCACGGTTCTCC |
| | | TGTACTTGGCCTCTCAGCCTTTGAGCAAGGTTGGCCCAAG |
| | | CTAGTATCGGCCCCAGTGGTACAGCCAAAACTTGAGACTG |
| | | CAAATGGATGCAGCTGTTGAACGCTGAGTAACTTCTGCAG |
| | | AGTCAGGAAGACCCAAGGAAGCTCTGCAGAGGATGCAGG |
| | | GGTACGGTCAGAACCCCTGAGTGCCTTTCAGCTAACGAGG |
| | | ACTTTATGACACTCCCCAGCACAGCAAATTTTTATGATGTG |
| | | TTTAAAGATTGGGTGAATTACTCAGGTGAACAAGCTACTTT |
| | | TTATCAGAGAACACCTAAAAACACGTTCAAGAGGGTTTGGG |
| | | AACTATACATTTAATCCTATGACAAACTAAGTTGGTTCTGTC |
| | | TTCACCTGTTTTGGTGAGGTTGTGTAAGAGTTGGTGTTTGC |
| | | TCAGGAAGAGATTTAAGCATGCTTGCTTACCCAGACTCAGA |
| | | GAAGTCTCCCTGTTCTGTCCTAGCTAGTGATTCCTGTGTTG |
| | | TGTGCATTCGTCTTTTCCAGAGCAAACCGCCCAGAGTAGAA |
| | | G |

Additional polynucleotides useful in conjunction with the compositions and methods described herein include nucleic acid molecules that have at least 85% sequence identity (e.g., 85%, 86%, 87%, 88%, 89%, 90%, 91%, 92%, 93%, 94%, 95%, 96%, 97%, 98%, 99%, or more, sequence identity) to the nucleic acid sequences set forth in Table 2 as well as functional portions or derivatives of the nucleic acid sequences set forth in Table 2.

The foregoing promoter sequences can be included in a nucleic acid vector and operably linked to a polynucleotide encoding an expression product (e.g., a polynucleotide encoding a protein of interest or an inhibitory RNA) to express the expression product specifically in GJB2-expressing cells (e.g., in GJB2-expressing inner ear cells, such as cochlear supporting cells). In some embodiments, the polynucleotide operably linked to a GJB2 promoter described herein (e.g., a polynucleotide that contains a first region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1 or a functional portion or derivative thereof and/or a second region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or a functional portion or derivative thereof, optionally containing a linker joining the first region and the second region) is a transgene that encodes a wild-type form of the GJB2 gene. In some embodiments, a polynucleotide encoding wild-type Gjb2, or a variant thereof, such as a polynucleotide sequence that encodes a protein having at least 85% sequence identity (e.g., 85%, 86%, 87%, 88%, 89%, 90%, 91%, 92%, 93%, 94%, 95%, 96%, 97%, 98%, 99%, or more, sequence identity) to the amino acid sequence of wild-type mammalian (e.g., human or mouse) Gjb2 (e.g., SEQ ID NO: 38 or SEQ ID NO: 45) is operably linked to a GJB2 promoter described herein. In some embodiments, the polynucleotide sequence encoding a Gjb2 protein encodes an amino acid sequence that contains one or more conservative amino acid substitutions relative to SEQ ID NO: 38 (e.g., 1, 2, 3, 4, 5, 6, 7, 8, 9, 10, 11, 12, 13, 14, 15, 16, 17, 18, 19, 20 or more conservative amino acid substitutions), provided that the Gjb2 analog encoded retains the therapeutic function of wild-type Gjb2 (e.g., the ability to form functional connexin hemichannels). No more than 10% of the amino acids in the Gjb2 protein may be replaced with conservative amino acid substitutions. In some embodiments, the polynucleotide sequence that encodes Gjb2 is any polynucleotide sequence that, by redundancy of the genetic code, encodes SEQ ID NO: 38. The polynucleotide sequence that encodes Gjb2 can be partially or fully codon-optimized for expression (e.g., in human cochlear supporting cells). Exemplary codon-optimized polynucleotide sequences that encode Gjb2 are SEQ ID NOs: 41-44. The Gjb2 protein may also be encoded by a polynucleotide having single nucleotide polymorphisms (SNPs) that have been found to be non-pathogenic in human subjects (e.g., SNPs that do not result in hearing loss). Human Gjb2 may be encoded by a polynucleotide having the sequence of any one of SEQ ID NOs: 39-44. Murine Gjb2 may be encoded by a polynucleotide having the sequence of SEQ ID NO: 46. The Gjb2 protein may be a human Gjb2 protein or may be a homolog of the human Gjb2 protein from another mammalian species (e.g., mouse, rat, cow, horse, goat, sheep, donkey, cat, dog, rabbit, guinea pig, or other mammal). Exemplary Gjb2 amino acid and polynucleotide sequences are listed in Table 3, below. A nucleic acid vector (e.g., an AAV vector) containing a GJB2 promoter described herein operably linked to a polynucleotide encoding Gjb2 can be administered to a subject to treat, reduce, or prevent GJB2-related hearing loss, such as hearing loss in a subject having DFNB1, DFNA3, Bart-Pumphrey syndrome, hystrix-like ichthyosis with deafness, keratitis-ichthyosis-deafness syndrome, palmoplantar keratoderma with deafness, or Vohwinkel syndrome.

TABLE 3
Gjb2 sequences

| SEQ ID | Description of | |
|---|---|---|
| NO: | sequence | Sequence |
| 38 | Human Gjb2 amino | MDWGTLQTILGGVNKHSTSIGKIWLTVLFIFRIMILVVAAKEVWGDE |
| | acid sequence | QADFVCNTLQPGCKNVCYDHYFPISHIRLWALQLIFVSTPALLVAMH |
| | (UniProt P29033) | VAYRRHEKKRKFIKGEIKSEFKDIEEIKTQKVRIEGSLWWTYTSSIFF |
| | | RVIFEAAFMYVFYVMYDGFSMQRLVKCNAWPCPNTVDCFVSRPTE |
| | | KTVFTVFMIAVSGICILLNVTELCYLLIRYCSGKSKKPV |
| 39 | Nucleic acid | ATGGATTGGGGCACGCTGCAGACGATCCTGGGGGGTGTGAACA |
| | sequence encoding | AACACTCCACCAGCATTGGAAAGATCTGGCTCACCGTCCTCTTC |
| | human Gjb2 | ATTTTTCGCATTATGATCCTCGTTGTGGCTGCAAAGGAGGTGTG |
| | | GGGAGATGAGCAGGCCGACTTTGTCTGCAACACCCTGCAGCCA |
| | | GGCTGCAAGAACGTGTGCTACGATCACTACTTCCCCATCTCCCA |
| | | CATCCGGCTATGGGCCCTGCAGCTGATCTTCGTGTCCACGCCA |
| | | GCGCTCCTAGTGGCCATGCACGTGGCCTACCGGAGACATGAGA |
| | | AGAAGAGGAAGTTCATCAAGGGGGAGATAAAGAGTGAATTTAAG |
| | | GACATCGAGGAGATCAAAACCCAGAAGGTCCGCATCGAAGGCT |
| | | CCCTGTGGTGGACCTACACAAGCAGCATCTTCTTCCGGGTCATC |
| | | TTCGAAGCCGCCTTCATGTACGTCTTCTATGTCATGTACGACGG |
| | | CTTCTCCATGCAGCGGCTGGTGAAGTGCAACGCCTGGCCTTGT |
| | | CCCAACACTGTGGACTGCTTTGTGTCCCGGCCCACGGAGAAGA |
| | | CTGTCTTCACAGTGTTCATGATTGCAGTGTCTGGAATTTGCATCC |
| | | TGCTGAATGTCACTGAATTGTGTTATTTGCTAATTAGATATTGTTC |
| | | TGGGAAGTCAAAAA AGCCAGTT |
| 40 | Nucleic acid | ATGGATTGGGGCACGCTGCAGACGATCCTGGGGGGTGTGAACA |
| | sequence encoding | AACACTCCACCAGCATTGGAAAGATCTGGCTCACCGTCCTCTTC |
| | human Gjb2 | ATTTTTCGCATTATGATCCTCGTTGTGGCTGCAAAGGAGGTGTG |
| | including stop | GGGAGATGAGCAGGCCGACTTTGTCTGCAACACCCTGCAGCCA |
| | codon | GGCTGCAAGAACGTGTGCTACGATCACTACTTCCCCATCTCCCA |
| | | CATCCGGCTATGGGCCCTGCAGCTGATCTTCGTGTCCACGCCA |
| | | GCGCTCCTAGTGGCCATGCACGTGGCCTACCGGAGACATGAGA |
| | | AGAAGAGGAAGTTCATCAAGGGGGAGATAAAGAGTGAATTTAAG |
| | | GACATCGAGGAGATCAAAACCCAGAAGGTCCGCATCGAAGGCT |
| | | CCCTGTGGTGGACCTACACAAGCAGCATCTTCTTCCGGGTCATC |
| | | TTCGAAGCCGCCTTCATGTACGTCTTCTATGTCATGTACGACGG |
| | | CTTCTCCATGCAGCGGCTGGTGAAGTGCAACGCCTGGCCTTGT |
| | | CCCAACACTGTGGACTGCTTTGTGTCCCGGCCCACGGAGAAGA |
| | | CTGTCTTCACAGTGTTCATGATTGCAGTGTCTGGAATTTGCATCC |
| | | TGCTGAATGTCACTGAATTGTGTTATTTGCTAATTAGATATTGTTC |
| | | TGGGAAGTCAAAAAAGCCAGTTTAA |
| 41 | Codon optimized | ATGGACTGGGGCACCCTGCAGACTATCCTGGGGGGCGTCAATA |
| | nucleic acid | AGCATTCAACTAGCATCGGAAAGATTTGGCTGACTGTCCTGTTTA |
| | sequence encoding | TCTTTCGGATCATGATCCTGGTGGTGGCAGCAAAGGAAGTGTGG |
| | human Gjb2 | GGCGACGAGCAGGCCGATTTCGTGTGCAACACACTGCAGCCAG |
| | | GCTGCAAGAACGTGTGCTACGACCACTATTTTCCCATCTCTCAC |
| | | ATCAGGCTGTGGGCCCTGCAGCTGATCTTCGTGAGCACCCCTG |
| | | CCCTGCTGGTGGCAATGCACGTGGCCTATCGGAGACACGAGAA |
| | | GAAGCGCAAGTTTATCAAGGGCGAGATCAAGAGCGAGTTCAAG |
| | | GATATCGAGGAGATCAAGACACAGAAGGTGAGGATCGAGGGCT |
| | | CCCTGTGGTGGACCTACACAAGCTCCATCTTCTTTCGCGTGATC |
| | | TTCGAGGCCGCCTTTATGTACGTGTTCTATGTGATGTACGACGG |
| | | CTTTTCTATGCAGCGGCTGGTGAAGTGCAACGCCTGGCCCTGTC |
| | | CTAATACAGTGGATTGTTTCGTGTCCAGACCCACCGAGAAGACA |
| | | GTGTTCACCGTGTTTATGATCGCCGTGTCTGGCATCTGCATCCT |
| | | GCTGAACGTGACCGAGCTGTGCTATCTGCTGATCCGGTACTGTA |
| | | GTGGAAAGAGCAAAA AACCCGTG |
| 42 | Codon optimized | ATGGACTGGGGAACATTGCAAACTATTTTGGGAGGAGTCAACAA |
| | nucleic acid | GCATTCAACTAGCATCGGGAAGATCTGGCTGACCGTGCTGTTCA |
| | sequence encoding | TCTTTCGCATCATGATTCTCGTGGTGGCCGCTAAGGAAGTCTGG |
| | human Gjb2 | GGCGATGAACAGGCCGACTTCGTGTGTAACACGCTGCAGCCCG |
| | | GTTGCAAAAACGTCTGCTACGATCACTACTTCCCCATCTCACACA |
| | | TTAGACTGTGGGCGCTGCAGCTGATTTTCGTGTCCACCCCGGCA |
| | | CTTCTTGTGGCGATGCACGTGGCCTACCGGCGGCACGAGAAGA |
| | | AAAGGAAGTTCATTAAGGGCGAAATCAAGTCCGAGTTCAAGGAC |
| | | ATCGAAGAAATCAAGACCCAGAAGGTCCGCATTGAGGGCTCCCT |
| | | CTGGTGGACCTACACCTCGTCCATCTTCTTCCGGGTCATATTCG |
| | | AGGCCGCCTTTATGTACGTGTTTTACGTGATGTACGACGGTTTC |
| | | AGCATGCAAAGACTCGTCAAGTGCAACGCTTGGCCTTGCCCCAA |
| | | TACCGTGGATTGCTTCGTGTCCCGCCCGACCGAGAAAACTGTGT |
| | | TCACTGTGTTCATGATCGCCGTGTCCGGCATCTGCATCCTGCTG |
| | | AACGTGACCGAGCTGTGCTATCTCCTGATCCGGTACTGTAGCGG |
| | | AAAGTCGAAGAAGCCTGTG |
| 43 | Codon optimized | ATGGATTGGGGGACGCTCCAGACTATACTTGGCGGGGTAAACA |
| | nucleic acid | AACATTCCACCTCAATTGGCAAAATCTGGCTCACAGTCCTCTTCA |
| | sequence encoding | TCTTCAGAATAATGATACTCGTGGTTGCCGCTAAAGAAGTTTGG |
| | human Gjb2 | GGTGACGAGCAAGCCGATTTCGTCTGTAACACCCTCCAACCAG |
| | | GTTGCAAAAATGTCTGTTACGATCACTACTTTCCTATTAGCCATA |
| | | TTAGACTCTGGGCCCTGCAACTTATCTTCGTTTCCACTCCTGCTC |
| | | TGCTCGTCGCTATGCACGTTGCCTATCGCCGCCATGAAAAAAAA |
| | | CGGAAATTCATTAAGGGAGAGATTAAGAGTGAATTCAAGGATATT |
| | | GAAGAGATTAAAACGCAAAAAGTTAGAATTGAGGGATCACTGTG |
| | | GTGGACTTATACCAGTAGCATCTTTTTTAGGGTCATTTTCGAAGC |
| | | TGCTTTCATGTATGTTTTCTATGTAATGTACGACGGTTTCTCCAT |
| | | GCAACGCTTGGTTAAATGTAACGCCTGGCCATGCCCTAATACGG |
| | | TTGATTGCTTTGTCTCCCGCCCTACTGAAAAGACAGTGTTTACCG |
| | | TTTTCATGATCGCCGTAAGTGGAATTTGTATCCTTCTTAACGTGA |
| | | CCGAGTTGTGCTATCTCCTTATTCGCTACTGTTCAGGAAAAAGTA |
| | | AAAAACCAGTA |
| 44 | Codon optimized | ATGGACTGGGGCACGCTGCAGACTATCCTGGGGGGTGTCAACA |
| | nucleic acid | AGCATTCAACTAGCATCGGAAAGATCTGGCTGACCGTCCTGTTC |
| | sequence encoding | ATCTTTCGCATCATGATCCTCGTGGTGGCCGCTAAGGAAGTGTG |
| | human Gjb2 | GGGCGACGAGCAGGCCGATTTCGTGTGTAACACCCTGCAGCCA |
| | | GGTTGCAAAAACGTCTGCTACGATCACTACTTTCCCATCTCCCA |
| | | CATTAGACTGTGGGCCCTGCAGCTGATCTTCGTGTCCACCCCTG |
| | | CGCTGCTAGTGGCCATGCACGTGGCCTATCGGCGACACGAGAA |
| | | GAAACGGAAGTTCATTAAGGGCGAGATCAAGAGCGAGTTCAAG |
| | | GATATCGAAGAGATCAAGACCCAGAAGGTCCGCATTGAGGGCT |
| | | CCCTGTGGTGGACCTACACCAGCTCCATCTTCTTTCGGGTCATC |
| | | TTCGAGGCCGCCTTTATGTACGTGTTCTATGTGATGTACGACGG |
| | | TTTCTCCATGCAACGGCTGGTGAAGTGCAACGCCTGGCCTTGCC |
| | | CTAATACTGTGGATTGCTTCGTGTCCCGCCCCACCGAGAAGACA |
| | | GTGTTCACCGTGTTCATGATCGCCGTGTCTGGCATCTGCATCCT |
| | | GCTGAACGTGACCGAGCTGTGCTATCTCCTGATCCGGTACTGTA |
| | | GTGGAAAGTCAAAAAAACCAGTGTAA |
| 45 | Murine Gjb2 amino | MDWGTLQSILGGVNKHSTSIGKIWLTVLFIFRIMILVVAAKEVWGDE |
| | acid sequence | QADFVCNTLQPGCKNVCYDHHFPISHIRLWALQLIMVSTPALLVAM |
| | (UniProt Q00977) | HVAYRRHEKKRKFMKGEIKNEFKDIEEIKTQKVRIEGSLWWTYTTSI |
| | | FFRVIFEAVFMYVFYIMYNGFFMQRLVKCNAWPCPNTVDCFISRPT |
| | | EKTVFTVFMISVSGICILLNITELCYLFVRYCSGKSKRPV |
| 46 | Nucleic acid | ATGGATTGGGGCACACTCCAGAGCATCCTCGGGGGTGTCAACA |
| | sequence encoding | AACACTCCACCAGCATTGGAAAGATCTGGCTCACGGTCCTCTTC |
| | murine Gjb2 | ATCTTCCGCATCATGATCCTCGTGGTGGCTGCAAAGGAGGTGTG |
| | | GGGAGATGAGCAAGCCGATTTTGTCTGCAACACGCTCCAGCCT |
| | | GGCTGCAAGAATGTATGCTACGACCACCACTTCCCCATCTCTCA |
| | | CATCCGGCTCTGGGCTCTGCAGCTGATCATGGTGTCCACGCCA |
| | | GCCCTCCTGGTAGCTATGCATGTGGCCTACCGGAGACATGAAA |
| | | AGAAACGGAAGTTCATGAAGGGAGAGATAAAGAACGAGTTTAAG |
| | | GACATCGAAGAGATCAAAACCCAGAAGGTCCGTATCGAAGGGT |
| | | CCCTGTGGTGGACCTACACCACCAGCATCTTCTTCCGGGTCATC |
| | | TTTGAAGCCGTCTTCATGTACGTCTTTTACATCATGTACAATGGC |
| | | TTCTTCATGCAACGTCTGGTGAAATGCAACGCTTGGCCCTGCCC |
| | | CAATACAGTGGACTGCTTCATTTCCAGGCCCACAGAAAAGACTG |
| | | TCTTCACCGTGTTTATGATTTCTGTGTCTGGAATTTGCATTCTGC |
| | | TAAATATCACAGAGCTGTGCTATTTGTTCGTTAGGTATTGCTCAG |
| | | GAAAGTCCAAAAGACCAGTC |

In some embodiments, a polynucleotide encoding wild-type Gjb6 (also known as Connexin 30), or a variant thereof, such as a polynucleotide sequence that encodes a protein having at least 85% sequence identity (e.g., 85%, 86%, 87%, 88%, 89%, 90%, 91%, 92%, 93%, 94%, 95%, 96%, 97%, 98%, 99%, or more, sequence identity) to the amino acid sequence of wild-type mammalian (e.g., human or mouse) Gjb6 (e.g., SEQ ID NO: 47 or SEQ ID NO: 50) is operably linked to a GJB2 promoter described herein (e.g., a polynucleotide that contains a first region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1 or a functional portion or derivative thereof and/or a second region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or a functional portion or derivative thereof, optionally containing a linker joining the first region and the second region). In some embodiments, the polynucleotide sequence encoding a Gjb6 protein encodes an amino acid sequence that contains one or more conservative amino acid substitutions relative to SEQ ID NO: 47 (e.g., 1, 2, 3, 4, 5, 6, 7, 8, 9, 10, 11, 12, 13, 14, 15, 16, 17, 18, 19, 20 or more conservative amino acid substitutions), provided that the Gjb6 analog encoded retains the therapeutic function of wild-type Gjb6 (e.g., the ability to form functional connexin hemichannels). No more than 10% of the amino acids in the Gjb6 protein may be replaced with conservative amino acid substitutions. In some embodiments, the polynucleotide sequence that encodes Gjb6 is any polynucleotide sequence that, by redundancy of the genetic code, encodes SEQ ID NO: 47. The polynucleotide sequence that encodes Gjb6 can be partially or fully codon-optimized for expression (e.g., in human cochlear supporting cells). The Gjb6 protein may also be encoded by a polynucleotide having single nucleotide polymorphisms (SNPs) that have been found to be non-pathogenic in human subjects (e.g., SNPs that do not result in hearing loss). Human Gjb6 may be encoded by a polynucleotide having the sequence of SEQ ID NO: 48 or SEQ ID NO: 49. Murine Gjb6 may be encoded by a polynucleotide having the sequence of SEQ ID NO: 51. The Gjb6 protein may be a human Gjb6 protein or may be a homolog of the human Gjb6 protein from another mammalian species (e.g., mouse, rat, cow, horse, goat, sheep, donkey, cat, dog, rabbit, guinea pig, or other mammal). Exemplary Gjb6 amino acid and polynucleotide sequences are listed in Table 4, below. Mutations in GJB6 are also associated with DFNB1 and DFNA3, accordingly, a nucleic acid vector (e.g., an AAV vector) containing a GJB2 promoter described herein operably linked to a polynucleotide encoding Gjb6 can be administered to a subject to treat, reduce, or prevent GJB6-related hearing loss, such as hearing loss in a subject having DFNB1 or DFNA3.

TABLE 4
Gjb6 sequences

| SEQ ID | Description of | |
|---|---|---|
| NO: | sequence | Sequence |
| 47 | Human Gjb6 amino | MDWGTLHTFIGGVNKHSTSIGKVWITVIFIFRVMILVVAAQEVWGDE |
| | acid sequence | QEDFVCNTLQPGCKNVCYDHFFPVSHIRLWALQLIFVSTPALLVAM |
| | (UniProt O95452) | HVAYYRHETTRKFRRGEKRNDFKDIEDIKKQKVRIEGSLWWTYTSS |
| | | IFFRIIFEAAFMYVFYFLYNGYHLPWVLKCGIDPCPNLVDCFISRPTE |
| | | KTVFTIFMISASVICMLLNVAELCYLLLKVCFRRSKRAQTQKNHPNH |
| | | ALKESKQNEMNELISDSGQNAITGFPS |
| 48 | Nucleic acid | ATGGATTGGGGGACGCTGCACACTTTCATCGGGGGTGTCAACA |
| | sequence encoding | AACACTCCACCAGCATCGGGAAGGTGTGGATCACAGTCATCTTT |
| | human Gjb6 | ATTTTCCGAGTCATGATCCTCGTGGTGGCTGCCCAGGAAGTGTG |
| | | GGGTGACGAGCAAGAGGACTTCGTCTGCAACACACTGCAACCG |
| | | GGATGCAAAAATGTGTGCTATGACCACTTTTTCCCGGTGTCCCA |
| | | CATCCGGCTGTGGGCCCTCCAGCTGATCTTCGTCTCCACCCCA |
| | | GCGCTGCTGGTGGCCATGCATGTGGCCTACTACAGGCACGAAA |
| | | CCACTCGCAAGTTCAGGCGAGGAGAGAAGAGGAATGATTTCAAA |
| | | GACATAGAGGACATTAAAAAGCAGAAGGTTCGGATAGAGGGGT |
| | | CGCTGTGGTGGACGTACACCAGCAGCATCTTTTTCCGAATCATC |
| | | TTTGAAGCAGCCTTTATGTATGTGTTTTACTTCCTTTACAATGGG |
| | | TACCACCTGCCCTGGGTGTTGAAATGTGGGATTGACCCCTGCCC |
| | | CAACCTTGTTGACTGCTTTATTTCTAGGCCAACAGAGAAGACCG |
| | | TGTTTACCATTTTTATGATTTCTGCGTCTGTGATTTGCATGCTGCT |
| | | TAACGTGGCAGAGTTGTGCTACCTGCTGCTGAAAGTGTGTTTTA |
| | | GGAGATCAAAGAGAGCACAGACGCAAAAAAATCACCCCAATC |
| | | ATGCCCTAAAGGAGAGTAAGCAGAATGAAATGAATGAGCTGATT |
| | | TCAGATAGTGGTCAAAATGCAATCACAGGTTTCCCAAGC |
| 49 | Nucleic acid | ATGGATTGGGGGACGCTGCACACTTTCATCGGGGGTGTCAACA |
| | sequence encoding | AACACTCCACCAGCATCGGGAAGGTGTGGATCACAGTCATCTTT |
| | human Gjb6 | ATTTTCCGAGTCATGATCCTCGTGGTGGCTGCCCAGGAAGTGTG |
| | including stop | GGGTGACGAGCAAGAGGACTTCGTCTGCAACACACTGCAACCG |
| | codon | GGATGCAAAAATGTGTGCTATGACCACTTTTTCCCGGTGTCCCA |
| | | CATCCGGCTGTGGGCCCTCCAGCTGATCTTCGTCTCCACCCCA |
| | | GCGCTGCTGGTGGCCATGCATGTGGCCTACTACAGGCACGAAA |
| | | CCACTCGCAAGTTCAGGCGAGGAGAGAAGAGGAATGATTTCAAA |
| | | GACATAGAGGACATTAAAAAGCAGAAGGTTCGGATAGAGGGGT |
| | | CGCTGTGGTGGACGTACACCAGCAGCATCTTTTTCCGAATCATC |
| | | TTTGAAGCAGCCTTTATGTATGTGTTTTACTTCCTTTACAATGGG |
| | | TACCACCTGCCCTGGGTGTTGAAATGTGGGATTGACCCCTGCCC |
| | | CAACCTTGTTGACTGCTTTATTTCTAGGCCAACAGAGAAGACCG |
| | | TGTTTACCATTTTTATGATTTCTGCGTCTGTGATTTGCATGCTGCT |
| | | TAACGTGGCAGAGTTGTGCTACCTGCTGCTGAAAGTGTGTTTTA |
| | | GGAGATCAAAGAGAGCACAGACGCAAAAAAATCACCCCAATCAT |
| | | GCCCTAAAGGAGAGTAAGCAGAATGAAATGAATGAGCTGATTTC |
| | | AGATAGTGGTCAAAATGCAATCACAGGTTTCCCAAGCTAA |
| 50 | Murine Gjb6 amino | MDWGTLHTVIGGVNKHSTSIGKVWITVIFIFRVMILVVAAQEVWGDE |
| | acid sequence | QEDFVCNTLQPGCKNVCYDHFFPVSHIRLWALQLIFVSTPALLVAM |
| | (UniProt P70689) | HVAYYRHETARKFIRGEKRNEFKDLEDIKRQKVRIEGSLWWTYTSSI |
| | | FFRIIFEAAFMYVFYFLYNGYHLPWVLKCGIDPCPNLVDCFISRPTEK |
| | | TVFTVFMISASVICMLLNVAELCYLLLKLCFRRSKRTQAQRNHPNHA |
| | | LKESKQNEMNELISDSGQNAITSFPS |
| 51 | Nucleic acid | ATGGACTGGGGGACCCTGCACACCGTCATCGGTGGCGTGAACA |
| | sequence encoding | AGCACTCTACCAGCATAGGGAAGGTGTGGATCACGGTCATCTTT |
| | murine Gjb6 | ATTTTCCGAGTCATGATCCTAGTGGTGGCTGCCCAGGAAGTGTG |
| | | GGGTGATGAGCAGGAGGACTTTGTCTGCAACACTCTGCAGCCA |
| | | GGGTGCAAGAACGTCTGCTATGACCATTTCTTCCCGGTGTCTCA |
| | | CATCCGGCTCTGGGCCCTGCAGCTGATCTTTGTGTCTACCCCAG |
| | | CCCTGTTGGTGGCCATGCACGTGGCCTACTACAGACATGAAACT |
| | | GCCCGAAAGTTTATACGTGGGGAGAAGAGAAACGAGTTTAAAGA |
| | | CCTGGAGGACATCAAACGGCAGAAGGTGCGCATTGAGGGCTCC |
| | | CTGTGGTGGACGTACACCAGCAGCATTTTCTTCCGCATCATCTT |
| | | CGAAGCCGCCTTCATGTATGTGTTCTACTTCCTCTACAATGGGTA |
| | | CCACCTACCCTGGGTACTGAAATGTGGCATTGACCCCTGCCCCA |
| | | ATCTCGTGGACTGCTTCATTTCGAGGCCAACTGAGAAAACGGTG |
| | | TTCACTGTTTTTATGATTTCCGCATCCGTGATTTGCATGCTGCTC |
| | | AATGTGGCCGAGTTGTGTTACCTGCTGCTTAAATTGTGCTTTAG |
| | | GAGATCCAAAAGAACACAGGCGCAGAGAAACCACCCCAACCAT |
| | | GCCCTGAAAGAGAGCAAGCAGAATGAAATGAATGAGCTGATCTC |
| | | AGATAGTGGCCAGAATGCAATCACAAGTTTCCCAAGT |

### Expression of Exogenous Polynucleotides in Mammalian Cells

Mutations in a variety of genes, such as GJB2, GJB6, SLC26A4, and GAS2, have been linked to sensorineural hearing loss. The compositions and methods described herein can be used to induce or increase the expression of exogenous polynucleotides (e.g., a gene that is endogenously expressed in GJB2-expressing cells, the wild-type form of a gene that is endogenously expressed in GJB2-expressing inner ear cells that is mutated in a subject with hearing loss, a polynucleotide encoding a protein that regulates the differentiation of cochlear supporting cells into cochlear hair cells, or an inhibitory RNA designed to downregulate a gene that inhibits the differentiation of cochlear supporting cells into cochlear hair cells) specifically in GJB2-expressing cells (e.g., GJB2-expressing inner ear cells, such as cochlear supporting cells) by administering a nucleic acid vector that contains a GJB2 promoter described herein (e.g., a polynucleotide that contains a first region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1 or a functional portion or derivative thereof and/or a second region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or a functional portion or derivative thereof, optionally containing a linker joining the first region and the second region) operably linked to a polynucleotide sequence that encodes an expression product (e.g., a protein of interest or an inhibitory RNA). A wide array of methods has been established for the delivery of proteins to mammalian cells and for the stable expression of polynucleotides encoding proteins in mammalian cells.

The nucleic acid vectors (e.g., AAV vectors) described herein (e.g., a nucleic acid vector that contains a GJB2 promoter described herein (e.g., a polynucleotide that contains a first region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1 or a functional portion or derivative thereof and/or a second region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or a functional portion or derivative thereof, optionally containing a linker joining the first region and the second region)) can be used to express a polynucleotide in one or more GJB2-expressing cells (e.g., GJB2-expressing inner ear cells). Exemplary polynucleotides that can be expressed using a nucleic acid vector described herein include polynucleotides encoding proteins that are expressed in healthy GJB2-expressing cells, polynucleotides encoding proteins that promote differentiation of cochlear supporting cells into cochlear hair cells, polynucleotides that correspond to a wild-type form of a gene that is endogenously expressed in a GJB2-expressing inner ear cell and is mutated in a subject with hearing loss, deafness, or tinnitus, and other polynucleotides that can be expressed in GJB2-expressing inner ear cells to treat hearing loss, deafness, or tinnitus. The nucleic acid vectors described herein can also be used to express a short hairpin RNA (shRNA), an antisense oligonucleotide (ASO), a component of a gene editing system (e.g., a nuclease, such as a CRISPR Associated Protein 9 (Cas9), Transcription Activator-Like Effector Nuclease (TALEN), or Zinc Finger Nuclease (ZFN), or a guide RNA (gRNA)), or a microRNA (e.g., miR-183, miR-96, or miR-182) in GJB2-expressing cells (e.g., GJB2-expressing inner ear cells, such as cochlear supporting cells).

In some embodiments, a GJB2 promoter described herein (e.g., a polynucleotide that contains a first region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1 or a functional portion or derivative thereof and/or a second region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or a functional portion or derivative thereof, optionally containing a linker joining the first region and the second region) is operably linked to a polynucleotide encoding brain-derived neurotrophic factor (BDNF) or neurotrophin-3 (NTF3). A nucleic acid vector (e.g., an AAV vector) containing a GJB2 promoter described herein operably linked to a polynucleotide encoding BDNF or NTF3 can be administered to a subject having or at risk of developing hearing loss (e.g., sensorineural hearing loss, such as age-related hearing loss, noise-induced hearing loss, ototoxic drug-induced hearing loss, head trauma-related hearing loss, or disease or infection-related hearing loss) to treat the subject's hearing loss (e.g., improve hearing), reduce the progression of hearing loss, or delay or prevent the development of hearing loss (e.g., in a subject at risk of developing hearing loss due to age, infection, or exposure to ototoxic drugs, loud noise, or head trauma).

In some embodiments, a GJB2 promoter described herein (e.g., a polynucleotide that contains a first region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1 or a functional portion or derivative thereof and/or a second region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or a functional portion or derivative thereof, optionally containing a linker joining the first region and the second region) is operably linked to a polynucleotide encoding an expression product that is implicated in cochlear hair cell regeneration. In some embodiments, the expression product is a protein that can induce or increase the differentiation of cochlear supporting cells into cochlear hair cells, such as Atoh1 (e.g., wild-type Atoh1 or an Atoh1 variant having one or more amino acid substitutions selected from the group consisting of S328A, S331A, S334A, S328A/S331A, S328A/S334A, S331A/S334A, and S328A/S331A/S334 as described in U.S. Publication No. US20190203210A1, which is incorporated herein by reference), Pou4F3, Gfi1, or Ikzf2, or a protein that can promote cochlear supporting cell proliferation, such as Lgr5, Yap1, or Tead2. In some embodiments, the expression product is an inhibitory RNA directed to a gene that suppresses cochlear supporting cell differentiation or proliferation, such as LATS1 and/or LATS2. Additional polynucleotides that can be expressed to promote cochlear hair cell regeneration (e.g., polynucleotides that can induce cochlear supporting cell proliferation or differentiation) are provided in Table 5 below along with accession numbers for their reference sequence transcripts. A GJB2 enhancer having the sequence of SEQ ID NO: 59 or a GJB2 enhancer having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 59 (e.g., one or more copies of a GJB2 enhancer having at least 85% sequence identity to SEQ ID NO: 59) can be operably linked to a GJB2 promoter described herein to regulate expression of an expression product involved in cochlear hair cell regeneration. A nucleic acid vector (e.g., an AAV vector) containing a GJB2 enhancer having at least 85% sequence identity to SEQ ID NO: 59 operably linked to a GJB2 promoter described herein that is operably linked to a polynucleotide encoding an expression product that is implicated in cochlear hair cell regeneration can be administered to a subject having or at risk of developing hearing loss associated with loss of cochlear hair cells (e.g., age-related hearing loss, noise-induced hearing loss, ototoxic drug-induced hearing loss, head trauma-related hearing loss, or disease or infection-related hearing loss).

TABLE 5
Polynucleotides that can be expressed to promote cochlear hair cell regeneration

| | NCBI |
|---|---|
| | Accession |
| Gene name | number |
| Atoh1, Atonal BHLH transcription factor 1 | NM_005172 |
| Pou4f3, POU class 4 homeobox 3 | NM_002700 |
| Gfi1, Growth factor independent 1 transcriptional repressor (variant 1) | NM_005263 |
| Gfi1, Growth factor independent 1 transcriptional repressor (variant 2) | NM_001127215 |
| Gfi1, Growth factor independent 1 transcriptional repressor (variant 3) | NM_001127216 |
| Isl1, ISL LIM homeobox 1 | NM_00220 |
| Tead2, TEA domain transcription factor 2 (variant 1) | NM_001256658 |
| Tead2, TEA domain transcription factor 2 (variant 2) | NM_001256659 |
| Tead2, TEA domain transcription factor 2 (variant 3) | NM_001256660 |
| Tead2, TEA domain transcription factor 2 (variant 4) | NM_003598 |
| Tead2, TEA domain transcription factor 2 (variant 5) | NM_001256661 |
| Tead2, TEA domain transcription factor 2 (variant 6) | NM_001256662 |
| Yap1, Yes1 associated transcriptional regulator (variant 1) | NM_001130145 |
| Yap1, Yes1 associated transcriptional regulator (variant 2) | NM_006106 |
| Yap1, Yes1 associated transcriptional regulator (variant 3) | NM_001195044 |
| Yap1, Yes1 associated transcriptional regulator (variant 4) | NM_001195045 |
| Yap1, Yes1 associated transcriptional regulator (variant 5) | NM_001282098 |
| Yap1, Yes1 associated transcriptional regulator (variant 6) | NM_001282097 |
| Yap1, Yes1 associated transcriptional regulator (variant 7) | NM_001282099 |
| Yap1, Yes1 associated transcriptional regulator (variant 8) | NM_001282100 |
| Yap1, Yes1 associated transcriptional regulator (variant 9) | NM_001282101 |
| Sox9, SRY-box 9 | NM_000346 |
| Sox10, SRY-box 10 | NM_006941 |
| Sall2, Spalt like transcription factor 2 (variant 1) | NM_005407 |
| Sall2, Spalt like transcription factor 2 (variant 2) | NM_001291446 |
| Sall2, Spalt like transcription factor 2 (variant 3) | NM_001291447 |
| Sall2, Spalt like transcription factor 2 (variant 6) | NM_001364564 |
| Camta1, Calmodulin binding transcription activator 1 (variant 1) | NM_015215 |
| Camta1, Calmodulin binding transcription activator 1 (variant 2) | NM_00119556 |
| Camta1, Calmodulin binding transcription activator 1 (variant 3) | NM_001242701 |
| Camta1, Calmodulin binding transcription activator 1 (variant 5) | NM_001349608 |
| Camta1, Calmodulin binding transcription activator 1 (variant 6) | NM_001349609 |
| Camta1, Calmodulin binding transcription activator 1 (variant 7) | NM_001349610 |
| Camta1, Calmodulin binding transcription activator 1 (variant 8) | NM_001349612 |
| Camta1, Calmodulin binding transcription activator 1 (variant 9) | NM_001349613 |
| Camta1, Calmodulin binding transcription activator 1 (variant 10) | NM_001349614 |
| Camta1, Calmodulin binding transcription activator 1 (variant 11) | NM_001349615 |
| Camta1, Calmodulin binding transcription activator 1 (variant 12) | NM_001349616 |
| Camta1, Calmodulin binding transcription activator 1 (variant 13) | NM_001349617 |
| Camta1, Calmodulin binding transcription activator 1 (variant 14) | NM_001349618 |
| Camta1, Calmodulin binding transcription activator 1 (variant 15) | NM_00134961 |
| Camta1, Calmodulin binding transcription activator 1 (variant 16) | NM_001349620 |
| Camta1, Calmodulin binding transcription activator 1 (variant 17) | NM_001349621 |
| Camta1, Calmodulin binding transcription activator 1 (variant 18) | NM_001349622 |
| Camta1, Calmodulin binding transcription activator 1 (variant 19) | NM_001349623 |
| Camta1, Calmodulin binding transcription activator 1 (variant 20) | NM_001349624 |
| Camta1, Calmodulin binding transcription activator 1 (variant 21) | NM_001349625 |
| Camta1, Calmodulin binding transcription activator 1 (variant 22) | NM_001349626 |
| Camta1, Calmodulin binding transcription activator 1 (variant 23) | NM_001349627 |
| Hey1, Hes related family bHLH transcription factor with YRPW | NM_012258 |
| motif 1 (variant 1) | |
| Hey1, Hes related family bHLH transcription factor with YRPW | NM_001040708 |
| motif 1 (variant 2) | |
| Hey1, Hes related family bHLH transcription factor with YRPW | NM_001282851 |
| motif 1 (variant 3) | |
| Hey2, Hes related family bHLH transcription factor with YRPW | NM_012259 |
| motif 2 | |
| Gata2, GATA binding protein 2 (variant 1) | NM_001145661 |
| Gata2, GATA binding protein 2 (variant 2) | NM_032638 |
| Gata2, GATA binding protein 2 (variant 3) | NM_001145662 |
| Gata3, GATA binding protein 3 (variant 1) | NM_001002295 |
| Gata3, GATA binding protein 3 (variant 2) | NM_002051 |
| Lass2, Ceramide synthase 2 (variant 1) | NM_181746 |
| Lass2, Ceramide synthase 2 (variant 2) | NM_022075 |
| Cux1, Cut like homeobox 1 (variant 1) | NM_181552 |
| Cux1, Cut like homeobox 1 (variant 2) | NM_001913 |
| Cux1, Cut like homeobox 1 (variant 3) | NM_181500 |
| Cux1, Cut like homeobox 1 (variant 4) | NM_001202543 |
| Cux1, Cut like homeobox 1 (variant 5) | NM_001202544 |
| Cux1, Cut like homeobox 1 (variant 6) | NM_001202545 |
| Cux1, Cut like homeobox 1 (variant 7) | NM_001202546 |
| Nr2f1, Nuclear receptor subfamily 2 group F member 1 | NM_005654 |
| Hes1, Hes family bHLH transcription factor 1 | NM_005524 |
| Rorb, RAR related orphan receptor B (variant 1) | NM_006914 |
| Rorb, RAR related orphan receptor B (variant 2) | NM_001365023 |
| Jun, Jun proto-oncogene AP-1 transcription factor subunit | NM_002228 |
| Zfp667 (human Znf667), Zinc finger protein 667 (variant 1) | NM_022103 |
| Zfp667 (human Znf667), Zinc finger protein 667 (variant 2) | NM_00132135 |
| Zfp667 (human Znf667), Zinc finger protein 667 (variant 3) | NM_001321355 |
| Lhx3, Lim homeobox 3 (variant 1) | NM_178138 |
| Lhx3, Lim homeobox 3 (variant 2) | NM_014564 |
| Lhx3, Lim homeobox 3 (variant 3) | NM_001363746 |
| Nhlh1, Nescient helix-loop-helix 1 | NM_005598 |
| Zmiz1, Zinc finger MIZ-type containing 1 | NM_020338 |
| Myt1, Myelin transcription factor 1 | NM_004535 |
| Stat3, Signal transducer and activator of transcription 3 (variant 1) | NM_139276 |
| Stat3, Signal transducer and activator of transcription 3 (variant 2) | NM_003150 |
| Stat3, Signal transducer and activator of transcription 3 (variant 3) | NM_213662 |
| Barhl1, BarH like homeobox 1 | NM_020064 |
| Tox, Thymocyte selection associated high mobility group box | NM_014729 |
| Prox1, Prospero homeobox 1 (variant 1) | NM_001270616 |
| Prox1, Prospero homeobox 1 (variant 2) | NM_002763 |
| Nfia, Nuclear factor I A (variant 1) | NM_00113467 |
| Nfia, Nuclear factor I A (variant 2) | NM_005595 |
| Nfia, Nuclear factor I A (variant 3) | NM_001145511 |
| Nfia, Nuclear factor I A (variant 4) | NM_001145512 |
| Thrb, Thyroid hormone receptor beta (variant 1) | NM_000461 |
| Thrb, Thyroid hormone receptor beta (variant 2) | NM_001128176 |
| Thrb, Thyroid hormone receptor beta (variant 3) | NM_001128177 |
| Thrb, Thyroid hormone receptor beta (variant 4) | NM_001252634 |
| Thrb, Thyroid hormone receptor beta (variant 5) | NM_001354708 |
| Thrb, Thyroid hormone receptor beta (variant 6) | NM_001354709 |
| Thrb, Thyroid hormone receptor beta (variant 7) | NM_001354710 |
| Thrb, Thyroid hormone receptor beta (variant 8) | NM_001354711 |
| Thrb, Thyroid hormone receptor beta (variant 9) | NM_001354712 |
| Thrb, Thyroid hormone receptor beta (variant 10) | NM_001354713 |
| Thrb, Thyroid hormone receptor beta (variant 11) | NM_001354714 |
| Thrb, Thyroid hormone receptor beta (variant 12) | NM_001354715 |
| Mycl1, MYCL proto-oncogene BHLH transcription factor (variant 1) | NM_001033081 |
| Mycl1, MYCL proto-oncogene BHLH transcription factor (variant 2) | NM_001033082 |
| Mycl1, MYCL proto-oncogene BHLH transcription factor (variant 3) | NM_005376 |
| Kdm5a, Lysine demethylase 5A | NM_001042603 |
| Creb3l4, cAMP responsive element binding protein 3 like 4 (variant 1) | NM_130898 |
| Creb3l4, cAMP responsive element binding protein 3 like 4 (variant 2) | NM_001255978 |
| Creb3l4, cAMP responsive element binding protein 3 like 4 (variant 3) | NM_001255979 |
| Creb3l4, cAMP responsive element binding protein 3 like 4 (variant 4) | NM_001255980 |
| Creb3l4, cAMP responsive element binding protein 3 like 4 (variant 5) | NM_001255981 |
| Creb3l4, cAMP responsive element binding protein 3 like 4 (variant 6) | NR_045658 |
| Etv1, ETS variant 1 (variant 1) | NM_004956 |
| Etv1, ETS variant 1 (variant 2) | NM_001163147 |
| Etv1, ETS variant 1 (variant 3) | NM_001163148 |
| Etv1, ETS variant 1 (variant 4) | NM_001163149 |
| Etv1, ETS variant 1 (variant 5) | NM_001163150 |
| Etv1, ETS variant 1 (variant 6) | NM_001163151 |
| Etv1, ETS variant 1 (variant 7) | NM_001163152 |
| Peg3, Paternally expressed 3 (variant 1) | NM_006210 |
| Peg3, Paternally expressed 3 (variant 2) | NM_001146184 |
| Peg3, Paternally expressed 3 (variant 3) | NM_001146185 |
| Peg3, Paternally expressed 3 (variant 4) | NM_001146186 |
| Peg3, Paternally expressed 3 (variant 5) | NM_001146187 |
| Bach2, BTB domain and CNC homolog 2 (variant 1) | NM_021813 |
| Bach2, BTB domain and CNC homolog 2 (variant 2) | NM_001170794 |
| Zbtb38, Zinc finger and BTB domain containing 38 (variant 1) | NM_001080412 |
| Zbtb38, Zinc finger and BTB domain containing 38 (variant 2) | NM_001350099 |
| Zbtb38, Zinc finger and BTB domain containing 38 (variant 3) | NM_001350100 |
| Lbh, Limb bud and heart development | NM_030915 |
| Tub, Tubby bipartite transcription factor (variant 1) | NM_003320 |
| Tub, Tubby bipartite transcription factor (variant 2) | NM_177972 |
| Hmg20, High mobility group20A (variant 1) | NM_018200 |
| Hmg20, High mobility group20A (variant 2) | NM_001304504 |
| Hmg20, High mobility group20A (variant 3) | NM_001304505 |
| Rest, RE1 silencing transcription factor (variant 1) | NM_005612 |
| Rest, RE1 silencing transcription factor (variant 2) | NM_001193508 |
| Rest, RE1 silencing transcription factor (variant 3) | NM_001363453 |
| Zfp827 (human Znf827;), Zinc finger protein 827 (variant 1) | NM_001306215 |
| Zfp827 (human Znf827;), Zinc finger protein 827 (variant 2) | NM_178835 |
| Aff3, AFR/FMR2 family member 3 (variant 1) | NM_002285 |
| Aff3, AFR/FMR2 family member 3 (variant 2) | NM_001025108 |
| Pknox2, PBX/knotted homeobox 2 | NM_022062 |
| Arid3b, AT-rich interaction domain 3B (variant 1) | NM_001307939 |
| Arid3b, AT-rich interaction domain 3B (variant 2) | NM_006465 |
| Mixip, MLX interacting protein | NM_014938 |
| Zfp532 (human Znf532), Zinc finger protein 532 (variant 1) | NM_018181 |
| Zfp532 (human Znf532), Zinc finger protein 532 (variant 2) | NM_001318726 |
| Zfp532 (human Znf532), Zinc finger protein 532 (variant 3) | NM_001318727 |
| Zfp532 (human Znf532), Zinc finger protein 532 (variant 4) | NM_001318728 |
| Zfp532 (human Znf532), Zinc finger protein 532 (variant 5) | NM_001353525 |
| Zfp532 (human Znf532), Zinc finger protein 532 (variant 6) | NM_001353526 |
| Zfp532 (human Znf532), Zinc finger protein 532 (variant 7) | NM_001353527 |
| Zfp532 (human Znf532), Zinc finger protein 532 (variant 8) | NM_001353528 |
| Zfp532 (human Znf532), Zinc finger protein 532 (variant 9) | NM_001353529 |
| Zfp532 (human Znf532), Zinc finger protein 532 (variant 10) | NM_001353530 |
| Zfp532 (human Znf532), Zinc finger protein 532 (variant 11) | NM_001353531 |
| Zfp532 (human Znf532), Zinc finger protein 532 (variant 12) | NM_001353532 |
| Zfp532 (human Znf532), Zinc finger protein 532 (variant 13) | NM_001353533 |
| Zfp532 (human Znf532), Zinc finger protein 532 (variant 14) | NM_001353534 |
| Zfp532 (human Znf532), Zinc finger protein 532 (variant 15) | NM_001353535 |
| Zfp532 (human Znf532), Zinc finger protein 532 (variant 16) | NM_001353536 |
| Zfp532 (human Znf532), Zinc finger protein 532 (variant 17) | NM_001353537 |
| Zfp532 (human Znf532), Zinc finger protein 532 (variant 18) | NM_001353538 |
| Ikzf2, IKAROS family zinc finger 2 (variant 1) | NM_016260 |
| Ikzf2, IKAROS family zinc finger 2 (variant 2) | NM_001079526 |
| Sall1, Spalt like transcription factor 1 (variant 1) | NM_00296 |
| Sall1, Spalt like transcription factor 1 (variant 2) | NM_001127892 |
| Six2, SIX homeobox 2 | NM_016932 |
| Sall3, Spalt like transcription factor 3 | NM_171999 |
| Lin28b, Lin-28 homolog B | NM_001004317 |
| Rfx7, Regulatory factor X7 | NM_022841 |
| Sox4, SRY-box 4 | NM_003107 |
| Sox11, SRY-box 11 | NM_003108 |
| Mxd4, Max dimerization protein 4 | NM_006454 |

In some embodiments, a GJB2 promoter described herein (e.g., a polynucleotide that contains a first region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1 or a functional portion or derivative thereof and/or a second region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or a functional portion or derivative thereof, optionally containing a linker joining the first region and the second region) is operably linked to a transgene corresponding to a wild-type version of a gene that is expressed in cochlear supporting cells and known to be mutated in a disease associated with hearing loss (e.g., a monogenic form of hearing loss). For example, the transgene can correspond to a wild-type form of SLC26A4, PAX3, NDP, or COCH, mutations in which are associated with Pendred Syndrome, Waardenburg Syndrome, Norrie disease, and DFNA9, respectively. Table 6 below provides a list of diseases that feature hearing loss and are associated with mutations in genes expressed in cochlear supporting cells along with the accession numbers for reference sequence transcripts for the genes. A GJB2 enhancer having the sequence of SEQ ID NO: 59 or a GJB2 enhancer having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 59 (e.g., one or more copies of a GJB2 enhancer having at least 85% sequence identity to SEQ ID NO: 59) can be operably linked to a GJB2 promoter described herein to regulate expression of a transgene corresponding to the wild-type form of a cochlear supporting cell gene that is known to be mutated in a subject with hearing loss (e.g., a gene listed in Table 6, below). A nucleic acid vector (e.g., an AAV vector) containing a GJB2 enhancer having at least 85% sequence identity to SEQ ID NO: 59 operably linked to a GJB2 promoter described herein that is operably linked to a transgene corresponding to the wild-type form of any of the genes listed in the table below or the wild-type form of any cochlear supporting cell gene (e.g., a gene expressed in cochlear supporting cells) that is known to be mutated in a subject with hearing loss can be administered to a subject carrying said mutation to treat the associated disease (e.g., improve or restore hearing).

TABLE 6
Genes that can be expressed to treat genetic forms of hearing loss

| Disease | Gene mutated in disease | NCBI Accession number |
|---|---|---|
| Pendred Syndrome | SLC26A4, Solute carrier family 26 | NM_000441 |
| | member 4 | |
| Waardenburg Syndrome | PAX3, Paired box 3 | NM_181457 (variant PAX3), |
| | | NM_000438 (variant PAX3A), |
| | | NM_013942 (variant PAX3B), |
| | | NM_181458 (variant PAX3D), |
| | | NM_181459 (variant PAX3E), |
| | | NM_181461 (variant PAX3G), |
| | | NM_181460 (variant PAX3H), |
| | | NM_001127366 (variant PAX3I) |
| Stickler Syndrome | COL1A2, Collagen type I alpha 2 | NM_000089 |
| | chain | |
| | COL11A1, Collagen type XI | NM_001854 (variant A), |
| | alpha 1 chain | NM_080629 (variant B), |
| | | NM_080630 (variant C), |
| | | NM_001190709 (variant E) |
| | COL11A2, Collagen type XI | NM_080680 (variant 1), |
| | alpha 2 chain | NM_080681 (variant 2), |
| | | NM_080679 (variant 3), |
| | | NM_001163771 (variant 4) |
| Jervell and Lange-Nielsen | KCNQ1, Potassium voltage-gated | NM_000218 (variant 1), |
| Syndrome | channel subfamily Q member 1 | NM_181798 (variant2) |
| | KCNE1, Potassium voltage-gated | NM_001127670 (variant 1), |
| | channel subfamily E regulatory | NM_000219 (variant 2), |
| | subunit 1 | NM_001127668 (variant 3), |
| | | NM_001127669 (variant 4), |
| | | NM_001270402 (variant 5), |
| | | NM_001270403 (variant 6), |
| | | NM_001270404 (variant 7), |
| | | NM_001270405 (variant 8) |
| Norrie disease | NDP, Norrin cystine knot growth | NM_000266 |
| | factor NDP | |
| DFNA9 | COCH, Cochlin | NM_001135058 (variant 1), |
| | | NM_004086 (variant 2), |
| | | NM_001347720 (variant 3) |
| DFNB8, DFNB10 | TMPRSS3, Transmembrane serine | NM_024022 (variant A), |
| | protease 3 | NM_032404 (variant C), |
| | | NM_032405 (variant D), |
| | | NM_001256317 (variant F) |
| DFNB29 | CLDN14, Claudin 14 | NM_144492 (variant 1), |
| | | NM_001146077 (variant 3), |
| | | NM_001146079 (variant 5), |
| | | NM_012130 (variant epsilon), |
| | | NM_001146078 (variant |
| | | gamma) |
| DFNA12 | TECTA, Tectorin alpha | NM_005422 |
| DFNB22 | OTOA, Otoancorin | NM_144672 (variant 1), |
| | | NM_170664 (variant 2), |
| | | NM_001161683 (variant 3) |
| DFNB18B | OTOG, Otogelin | NM_001277269 (variant 1), |
| | | NM_001292063 (variant 2) |
| DFNB84B | OTOGL, Otogelin like | NM_173591 (variant 1), |
| | | NM_001368062 (variant 2), |
| | | NM_001378609 (variant 3), |
| | | NM_001378610 (variant 4) |
| DFNA4B | CEACAM16, Carcinoembryonic | NM_001039213 |
| | antigen related cell adhesion | |
| | molecule 16 | |
| DFNA41 | P2RX2, Purinergic receptor P2X 2 | NM_170682 (variant 1), |
| | | NM_174873 (variant 2), |
| | | NM_016318 (variant 3), |
| | | NM_170683 (variant 4), |
| | | NM_174872 (variant 5), |
| | | NM_012226 (variant 6), |
| | | NM_001282164 (variant 7), |
| | | NM_001282165 (variant 8) |
| DFNA5 | GSDME, Gasdermin E | NM_004403 (variant 1), |
| | | NM_001127453 (variant 2), |
| | | NM_001127454 (variant 3) |

### Polynucleotides Encoding Proteins of Interest

One platform that can be used to achieve therapeutically effective intracellular concentrations of proteins of interest in mammalian cells is via the stable expression of the gene encoding the protein of interest (e.g., by integration into the nuclear or mitochondrial genome of a mammalian cell, or by episomal concatemer formation in the nucleus of a mammalian cell). The gene is a polynucleotide that encodes the primary amino acid sequence of the corresponding protein. In order to introduce exogenous genes into a mammalian cell, genes can be incorporated into a vector. Vectors can be introduced into a cell by a variety of methods, including transformation, transfection, transduction, direct uptake, projectile bombardment, and by encapsulation of the vector in a liposome. Examples of suitable methods of transfecting or transforming cells include calcium phosphate precipitation, electroporation, microinjection, infection, lipofection and direct uptake. Such methods are described in more detail, for example, in Green, et al., Molecular Cloning: A Laboratory Manual, Fourth Edition (Cold Spring Harbor University Press, New York 2014); and Ausubel, et al., Current Protocols in Molecular Biology (John Wiley & Sons, New York 2015), the disclosures of each of which are incorporated herein by reference.

Proteins of interest can also be introduced into a mammalian cell by targeting a vector containing a gene encoding a protein of interest to cell membrane phospholipids. For example, vectors can be targeted to the phospholipids on the extracellular surface of the cell membrane by linking the vector molecule to a VSV-G protein, a viral protein with affinity for all cell membrane phospholipids. Such a construct can be produced using methods well known to those of skill in the field.

Recognition and binding of the polynucleotide encoding a protein of interest by mammalian RNA polymerase is important for gene expression. As such, one may include sequence elements within the polynucleotide that exhibit a high affinity for transcription factors that recruit RNA polymerase and promote the assembly of the transcription complex at the transcription initiation site. Such sequence elements include, e.g., a mammalian promoter, the sequence of which can be recognized and bound by specific transcription initiation factors and ultimately RNA polymerase. Examples of mammalian promoters have been described in Smith, et al., Mol. Sys. Biol., 3:73, online publication, the disclosure of which is incorporated herein by reference. In some embodiments, the promoter used in the methods and compositions described herein is a GJB2 promoter described herein (e.g., a polynucleotide that contains a first region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1 or a functional portion or derivative thereof and/or a second region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or a functional portion or derivative thereof, optionally containing a linker joining the first region and the second region).

Once a polynucleotide encoding a protein of interest has been incorporated into a mammalian cell, the transcription of this polynucleotide can be induced by methods known in the art. For example, expression can be induced by exposing the mammalian cell to an external chemical reagent, such as an agent that modulates the binding of a transcription factor and/or RNA polymerase to the mammalian promoter and thus regulates gene expression. The chemical reagent can serve to facilitate the binding of RNA polymerase and/or transcription factors to the mammalian promoter, e.g., by removing a repressor protein that has bound the promoter. Alternatively, the chemical reagent can serve to enhance the affinity of the mammalian promoter for RNA polymerase and/or transcription factors such that the rate of transcription of the gene located downstream of the promoter is increased in the presence of the chemical reagent. Examples of chemical reagents that potentiate polynucleotide transcription by the above mechanisms include tetracycline and doxycycline. These reagents are commercially available (Life Technologies, Carlsbad, CA) and can be administered to a mammalian cell in order to promote gene expression according to established protocols.

Other DNA sequence elements that may be included in polynucleotides for use in the compositions and methods described herein include enhancer sequences. Enhancers represent another class of regulatory elements that induce a conformational change in the polynucleotide containing the gene of interest such that the DNA adopts a three-dimensional orientation that is favorable for binding of transcription factors and RNA polymerase at the transcription initiation site. Thus, polynucleotides for use in the compositions and methods described herein include those that encode a protein of interest and additionally include a mammalian enhancer sequence. Many enhancer sequences are now known from mammalian genes, and examples include enhancers from the genes that encode mammalian globin, elastase, albumin, α-fetoprotein, and insulin. Enhancers for use in the compositions and methods described herein also include those that are derived from the genetic material of a virus capable of infecting a eukaryotic cell. Examples include the SV40 enhancer on the late side of the replication origin (bp 100-270), the cytomegalovirus early promoter enhancer, the polyoma enhancer on the late side of the replication origin, and adenovirus enhancers. Additional enhancer sequences that induce activation of eukaryotic gene transcription include the CMV enhancer and RSV enhancer. An enhancer may be spliced into a vector containing a polynucleotide encoding a protein of interest, for example, at a position 5′ or 3′ to this gene. In a preferred orientation, the enhancer is positioned at the 5′ side of the promoter, which in turn is located 5′ relative to the polynucleotide encoding a protein of interest.

The present inventors have identified GJB2 enhancers that can be operably linked to a GJB2 promoter described herein (e.g., a polynucleotide that contains a first region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1 or a functional portion or derivative thereof and/or a second region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or a functional portion or derivative thereof, optionally containing a linker joining the first region and the second region) that is operably linked to a polynucleotide encoding an expression product (e.g., a polynucleotide encoding a protein or a polynucleotide that can be transcribed to produce an inhibitory RNA molecule) to increase the number of GJB2-expressing cells that express the expression product and/or the expression level of the expression product in these cells. Operably linking a GJB2 promoter described herein to a GJB2 enhancer described herein can also reduce or eliminate off-target expression in non-GJB2-expressing cells (e.g., cochlear hair cells). Accordingly, operably linking one or more of the GJB2 enhancers to a GJB2 promoter described herein for use in gene therapy may improve therapeutic efficacy. In some embodiments, the compositions and methods described herein include one or more GJB2 enhancers listed in Table 7 (e.g., one or more of SEQ ID NOs: 52-63), such as polynucleotide sequences that have at least 85% sequence identity (e.g., 85%, 86%, 87%, 88%, 89%, 90%, 91%, 92%, 93%, 94%, 95%, 96%, 97%, 98%, 99%, or more, sequence identity) to any one of SEQ ID NOs: 52-63. In some embodiments, the GJB2 enhancer has the sequence of any one of SEQ ID NOs:52-63. In some embodiments, the compositions described herein contain two or more (e.g., 2, 3, 4, 5, 6, 7, 8, 9, 10 or more) GJB2 enhancers, which can have the same sequence (e.g., multiple copies of the same GJB2 enhancer) or different sequences (e.g., one or more copies of at least two different GJB2 enhancers). For example, the compositions and methods described herein may include four GJB2 enhancers having different sequences, such as SEQ ID NOs: 53, 54, 55, and 56 or SEQ ID NOs: 52, 57, 58, and 59. In some embodiments, the compositions and methods described herein contain two or more copies of the same GJB2 enhancer (e.g., 2, 3, 4, 5, 6, 7, 8, 9, 10 or more copies of the same GJB2 enhancer) and two or more copies of different GJB2 enhancers (e.g., 2, 3, 4, 5, 6, 7, or 8 different GJB2 enhancers). In embodiments in which a composition contains two or more enhancers (e.g., SEQ ID NOs: 52, 57, 58, and 59) the enhancers can be included in any order and may be positioned directly next to one another (e.g., joined without any intervening sequence between the enhancer sequences, e.g., the 3′ end of a first enhancer is positioned directly before the 5′ end of a second enhancer) or may be joined by a nucleic acid linker (e.g., a nucleic acid linker may be positioned between each enhancer sequence included in the composition or between at least two of the enhancer sequences in the composition). The one or more GJB2 enhancers can be positioned 5′ of the promoter or 3′ of the promoter (e.g., 5′ of the promoter or 3′ of the coding sequence).

Exemplary GJB2 enhancer sequences are listed in Table 7.

TABLE 7
GJB2 enhancer sequences

| SEQ | | |
|---|---|---|
| ID NO: | Enhancer | Enhancer sequence |
| 52 | Enhancer 1 | AAAAACTCTTACATAATTGTAACAGATTTGAGTTTCCTCTGGTTCAG |
| | | GTTTTCTTGCCTCTTTGATAATCAAATGATCTGAAGAAAGGCATAG |
| | | AATTTCAAGGGAGAATCTGCATGACAGGATTACAATAAGGCTATTC |
| | | ATGGAGACTCTTTTATTAGCTTACACAGGATCTGCATCATTCTTCG |
| | | TCTCTGGCTTTGCTACAAGGCTCCATTTAAACTTAACCCAACTTGC |
| | | AGGCTTAACTGATCCAGGAATGACTCAATACAATGAGCCAGCTAC |
| | | TGTAGCTCTTTTTCCCCTTTTGATAAGGGGAGTTAACACAATGGGC |
| | | TTTACAGTTCTTAAATGA |
| 53 | Enhancer 2 | TTTCCGACAAGTCTCAGTGCCATCTACTGTGCGCTCTGGGTATTG |
| | | CAATTGCtttt |
| 54 | Enhancer 3 | ATATAAGGGAACGCTGCTGTTCTGTTGTAGAGATTACAGAGAGCA |
| | | ATTTAGGAGAGCCAGGCACTGGGGCCAAGAGGGAAATGAAACAA |
| | | AAACCGAAGGGATTTGTTTAGGAAGAAAAATGAAAACAGATAAAA |
| | | GGTGTTCATTTCAAAGCTTCCCTCTTTCCCAGCATTTTTCTGAAGT |
| | | AGAGTTTGAAAAGAAAGCAAAATAACTGCAAACCAATACAGCGGC |
| | | ACGGAGTTCACTGACGcagagctagaaatgacgttcagagatccgccagcccagcc |
| | | tcctGTTCTGGGTCACCTGGCTCCTTGACAACCCTGAAAGCTGCCTG |
| | | TGCAAATCCCCAGGCATGTTATACCCATGAGCAGGGACGCGTGG |
| | | CACTGACAAAGGGACTCGTACACCTTTGAA |
| 55 | Enhancer 4 | ACACAGCAGTCAAGTTCTCAAACAATCTTCAGGAGCCTTCCAAAAC |
| | | TGGACATaaaaaaaagtaccttcattggggaaaagaaaagaaaaaaaaaaGAATCA |
| | | CAATTGCCACCAAGGCTCTACATTTTTGCAAAAGTCCAGCATTTAA |
| | | AAGAAACTTCCCTGGATGGCCTACATCTGCTGATTGGTAATTTGTC |
| | | ATTCAGGTTAAAAACAAAACAGAAGTGGGCATTGTTGTGATATCAT |
| | | CCTTGATAACATCCCAAGAAAACTCTAGAGCTGGCAGGAGAGGAA |
| | | AGCAGATAATGGTCAAAGCTGTCATCTGAGTTTTAAAAACACTGTG |
| | | ATTTTTCTTTTAAAGGAACATCTTCAGTTTCCAAGGCCATACACACA |
| | | GTTCCTAACTGCAGCTTCAAATTTTCCACCGGGCTCCCTTCTGAGA |
| | | ACAAACGCTATTCAGTGGCGAGCCCCGGACACCACTGCGCTTTCA |
| | | AAGGCAGCTGCCAGAGGACACTCAGGACTTCACAGCCGCCGGTG |
| | | AGCCAGACTGGGGTCAGTCACTCCCCCATCAGAATTATTTTGTTTC |
| | | TCC |
| 56 | Enhancer 5 | CCAGTGGTTCCCTACTGTGCCTAAGTCATAGGAGTACTGGCTAAA |
| | | AATGCAGACTCCAGACATCTCCCCCAGAATTACAAGCATCCCAAG |
| | | GAATTCCGCTAAAGGTGATATGCAAACCACATTCTGATAAGCAGAT |
| | | AACTGGGGACCAGGGGCTGCACAAGTCCCACACCCACTAGGAGT |
| | | GGCATTTATTCCATCTCTTCACCTGACAGGTACAAAGTCTTTCATC |
| | | TTTGGAAAGAATGGTTTTTCCTTGGGGCATCTCAAGCACTACTTTC |
| | | ATGGAAGAGGAGGTAGTATGGGACTGTTATTGCTTTCTATGGGaaa |
| | | aaaaaaTTGAAAACTAATATTGTTTTGCTTTGGGGTGAAATGAAGAG |
| | | TAGTAATTGGACCTTTTGTTTTCTTTGTCTTGATTGTCCATTTTTGT |
| | | AAGAAAGTGAATGCCAAGCAAAATACAAATGGATATAGAAAATTCT |
| | | GCCTAACTCAGCAATGTCTTTAAATGTAAATACTGCACTAGAAACA |
| | | GCTAATCTCCAGATCACACACCAT |
| 57 | Enhancer 6 | CCGTTCCTGCAGGTATTGGAAAACCTGCATCGTATGCCTGCTGCT |
| | | TTAGGAGACAACAAACTCAGCAGCCCCTCCCCCGAAGCTGAAGTG |
| | | CCAAACTTAAGGCTCCAGGTAAACTGCCCTTCCGATGAGAGCTTT |
| | | ACATTTAGGCAGTAGCCACAGGCCTAAACAAAGTCAAATGGATGA |
| | | AACTTGTGATCTCTCCTTGGAAATATGGCCAAGAGTGAAACAACTA |
| | | CTTAGTTATTATTTTAAACGCAGGCTGCTAAATGGCTTGTGTTGGT |
| | | ATGTAATGGCATCCTAGATAATGGGGGCTGCCAAGAAAATATTCA |
| | | CACTGGCAAGTCAATTAAAATACCGTTCTCTTCCTAAGAGGCCTG |
| | | GCTGGATTCCAGAGCTTCCTTTAGGGTAACGCTCAAACCTGTCAT |
| | | AGTTTATTTTGTTTCTGTTTTCAGGCAACAGATTTGGTTATTGCTTT |
| | | TATTGTATGAGTGTTGAAATGGTGTCCTAACAGCCATCTGTACTGC |
| | | CATCCCCTGG |
| 58 | Enhancer 7 | TCTCTCTGCCTTTCCAGAAAGGAGATGAAGTTGCTGGAGCAGGCA |
| | | GGTTCTCTGAAAGGCTCCCTGAGTGTGGAAGAGCAGCTGTCACTC |
| | | ATCAGTGGTTGTCCCAATATCCAAGAAGCAGTAGAGGGTGCCATG |
| | | CACATTCAGGTGAGTGAAGGACACTGGCGCCGCATCTGTGGGGC |
| | | CTGAGACATTGTCTCCCAGCCTCTCCGGAGGAACAGGGCAGGAT |
| | | ACAGAGCAGTGGCAGGTGTGGGCATTTTGCATCTTC |
| 59 | Enhancer 8 | TGTAATCTCCAAACAAAGTCTGCGCTAGGCCCTTTCAGGCTATAAA |
| | | GGACAGGGTACTCTGATTTCTGCTCTTGATGAGGGTTGATTGTGA |
| | | TACCAGGCGAGGGAGTAAGAGATACAGGACACCATCTCTGCGGC |
| | | TGCATCTTTGTCGCAGAAACAGACATTGTTCAGGGGACACCTGAG |
| | | CCTCTGATAGTCAGCCAGTGGCTCTGGCAGCTCACAGCAGTTATA |
| | | GGGTGGCTGTGTCCTTTTCTAAAGTCCTGACTTTTTATGGCCTGTG |
| | | GTCATGGTGACTCCACTACGGGCTGTGTTTCTGTTG |
| 60 | Enhancer GH | TGAACTTAAGTCATTTGCCCAAGGTTATACAGCCAGTAGGCTGGA |
| | | AAGCCAGGGTAAGGAGCCGGCCGGCTGGCTCCACAGGCCAGAA |
| | | CCACCATTCTGAGGGCCCGTCTTTCAGGGGTGTTGTTTGTAAAAG |
| | | GAGCCAAAAGGCAATGGCACGCTGGCCTCCTGTTTCTCAGAAACT |
| | | ATTTCATTAGATTAAAAATAAATCCTAAAATATTTGCACTAGTTCTA |
| | | GATAAATGGTAGCTTTTCAGGCTAATAACTACCCTTAACCTGAGAT |
| | | GCTGGCTTTATGAAAACAGTATCAGAAAAGAAGAAAAATTCTCCCA |
| | | ACTCCAATCTTGATATATTCTGGGTGACACAGGGAAGAATGATAAA |
| | | CATTTCTATGGGTTCCCAGCCAGATCTTATAACTCTCCAACTCTGG |
| | | TCTTTGGAATGCTCTGAAGTTGAAAACTACATCCGTCCACCATTAT |
| | | CATATGAAGCCAATTAGGTTTTCTTATCGTATCTGCTTTGTTTGAAA |
| | | AATTGTTATGGGCCTCCTTCCTGTCTGCCACTCTATTTGATTCTTG |
| | | AGTTTCTGGTTTCATTGGCTTCTGTTTCTATTTGTTTGAAAGTCAAA |
| | | GCTGTTCTCAGTTCTGTCCCTATCTATTTCTTATGATTCCTCAATTA |
| | | TCTTATTTTTCTCAAGAAAAAAAAGGGAGAAAGAAAAACACAGTGG |
| | | TTATCAGGCAGCGTCTGTATACATTTCAAATGTATAAATTTCTATAC |
| | | ATTTCAAATGCTTAAGTATATATACAAATCTATATACATTTCAAATG |
| | | CTTAAATATGCTCACAAGTTTTGAGGTTCTGGTTGAAAACACCATT |
| | | TGAAATGATCTAAGACTTTGTGTTAATAATACAAGAAAACTCTTGTT |
| | | TCACTGCATGAGGCCATCTTTCTCTACATTATOTTCTTCTATTCAGT |
| | | GGTTATCTTCCCATAAAACTAACACACACGTGCTGGAGCAGATTC |
| | | CCCTCTTCCCTTCACTCCTCTTAGCTCTTCTTAGCTTGATTCTGATA |
| | | AGGACTGAAGCAGAGAACACACGGCCCATCCACTTCAGAAATCAT |
| | | CTGTGCACCTGCTGTATGCAGGAACATGTCTAGTAAAGCTTCCTT |
| | | GAATTATTCATTGGACAGCATATTTCATTAACTACTGTGAAAATTGA |
| | | CTGAACTATTACCAAAATGTAATACTGAAATTATAGAACTGAAGTC |
| | | AAACAGACTGAGAGAGGTCACCAACTCTCAACCTCCCCTCTCGCA |
| | | GAGGGAGTTTCCTCTCAACATCTAAACCTCCCACAGGCAGGCAAC |
| | | CACATCCCCTTTTCAGCTTACCCACTTAAAGCATGGGCCTTTCCTG |
| | | GACATCAGCTCCTGTTACTAGGAAGTTCTTCCTGCCATGTACTGA |
| | | GCCCAAACCTGTCTGTCTGCTTGCCATTTCCAGTCTTTCACTCCAC |
| | | TTCCGCCCCCACAAAACATACAGACCGAGAGACATACGAGAGACT |
| | | CTGGTCAC |
| 61 | Enhancer GHA | AAGGAGCCAAAAGGCAATGGCACGCTGGCCTCCTGTTTCTCAGAA |
| | | ACTATTTCATTAGATTAAAAATAAATCCTAAAATATTTGCACTAGTT |
| | | CTAGATAAATGGTAGCTTTTCAGGCTAATAACTACCCTTAACCTGA |
| | | GATGCTGGCTTTATGAAAACAGTATCAGAAAAGAAGAAAAATTCTC |
| | | CCAACTCCAATCTTGATATATTCTGGGTGACACAGGGAAGAATGAT |
| | | AAACATTTCTATGGGTTCCCAGCCAGATCTTATAACTCTCCAACTC |
| | | TGGTCTTTGGAATGCTCTGAAGTTGAAAACTACATCCGTCCACCAT |
| | | TATCATATGAAGCCAATTAGGTTTTCTTATCGTATCTGCTTTGTTTG |
| | | AAAAATTGTTATGGGCCTCCTTCCTGTCTGCCACTCTATTTGATTC |
| | | TTGAGTTTCTGGTTTCATTGGCTTCTGTTTCTATTTGTTTGAAAGTC |
| | | AAAGCTGTTCTCAGTTCTGTCCCTATCTATTTCTTATGATTCCTCAA |
| | | TTATCTTATTTTTCTCAAGAAAAAAAAGGGAGAAAGAAAAACACAG |
| | | TGGTTATCAGGCAGCGTCTGTATACATTTCAAATGTATAAATTTCTA |
| | | TACATTTCAAATGCTTAAGTATATATACAAATCTATATACATTTCAAA |
| | | TGCTTAAATATGCTCACAAGTTTTGAGGTTCTGGTTGAAAACACCA |
| | | TTTGAAATGATCTAAGACTTTGTGTTAATAATACAAGAAAACTCTTG |
| | | TTTCACTGCATGAGGCCA |
| 62 | Enhancer 9 | CTGCTTCGGCCTCCAAATAGCTGGGATTACAGGCATGCGTCACCA |
| | | CGCCCAGCTAATTTTGTATTTTTAGTAGAGACACAGTTTCTCCATG |
| | | TTGGTCAGGCTGGTCTCAAACTCCTGACCTCAAGTGATCCACCCG |
| | | CCTCAGCCTCCCAAAGTGCTGGGATTACAGGTGTGAGCCACCACA |
| | | TCTGGATAATTTTGTATTTTTAGTAGCGATGGGGTTTCTCCATGTT |
| | | GGTCAGACTGGTCTCAAACTCCTGACCTCAAGTGATCCACCCAGA |
| | | GTGCTGAGATTACAGGTGTGAGCCACCACATCCGGCCTAGATTAA |
| | | GTTTATATTGAGAACTACCACAATGATTCATGAGTCTCTCATGAGA |
| | | AACTAGTTGGAATTATGAGTTTGTTCCCTCCACTTATACATACTGTA |
| | | CCCACATGTTTTCGTTTTCTATACTCTTCACCACATGTTGCTTCCC |
| | | CCACATGCAACAGGAAACTGGCTCCACGGCAAGGGTGAAAAAGA |
| | | TCTGACCCAGTGGTTCCCACCCACGCCACCTTCCAGGGAGAAGG |
| | | TGAGGCCAAGATGAGACTTTAATAAGTCGAAAATAAAAGACTGCTA |
| | | AGTGCTGCCCCACAATTACTCACAGGCTTGTGGTCAGGTTTGTGA |
| | | GCTGCTTGGCCTGTTGCCGGCACTTACAAGCTTTCTTGGAAGCAA |
| | | GACACAGACATTCCTGCGGAATGACTTGAGCAAATTTTGCTAACG |
| | | CTACAAGGGTTGATTGAACACCACCACATTGAACTGATGCAAGAG |
| | | GACACTGAATCACACCCATAAAACACCCGTCCTGTCAAGAGTCTT |
| | | CCAAGAGGACGCGCAGGAGTCAGCGCTCCGGGGAAAGAAGATG |
| | | AGAAACACCAGGTGGACCACCCTGGCCAGGCTGCTGGGAACAAG |
| | | CCTACGTGCACAGCTGCCGGAGGAGAGGGGGGTCTCCCCTCTGG |
| | | GAGGCATCTCAGCTACATCTATCAAGTTATAAAAATGTTACTCTCC |
| | | CGCAGGCTGAGCCCACTCCCAGAATTATTCCAGAAAAGACTCCAG |
| | | AAGAAG |
| 63 | Enhancer 9C | CACCACATCCGGCCTAGATTAAGTTTATATTGAGAACTACCACAAT |
| | | GATTCATGAGTCTCTCATGAGAAACTAGTTGGAATTATGAGTTTGT |
| | | TCCCTCCACTTATACATACTGTACCCACATGTTTTCGTTTTCTATAC |
| | | TCTTCACCACATGTTGCTTCCCCCACATGCAACAGGAAACTGGCT |
| | | CCACGGCAAGGGTGAAAAAGATCTGACCCAGTGGTTCCCACCCA |
| | | CGCCACCTTCCAGGGAGAAGGTGAGGCCAAGATGAGACTTTAATA |
| | | AGTCGAAAATAAAAGACTGCTAAGTGCTGCCCCACAATTACTCACA |
| | | GGCTTGTGGTCAGGTTTGTGAGCTGCTTGGCCTGTTGCCGGCACT |
| | | TACAAGCTTTCTTGGAAGCAAGACACAGACATTCCTGCGGAATGA |
| | | CTTGAGCAAATTTTGCTAACGCTACAAGGGTTGATTGAACACCACC |
| | | ACATTGAACTGATGCAAGAGGACACTGAATCACACCCATAAAACA |
| | | CCCGTCCTGTCAAGAGTCTTCCAAGAGGACGCGCAGGAGTCAGC |
| | | GCTCCGGGGAAAGAAGATGAGAAACACCAGGTGGACCACCCTGG |
| 64 | Enhancer GHB | TTGACTGAACTATTACCAAAATGTAATACTGAAATTATAGAACTGAA |
| | | GTCAAACAGACTGAGAGAGGTCACCAACTCTCAACCTCCCCTCTC |
| | | GCAGAGGGAGTTTCCTCTCAACATCTAAACCTCCCACAGGCAGGC |
| | | AACCACATCCCCTTTTCAGCTTACCCACTTAAAGCATGGGCCTTTC |
| | | CTGGACATCAGCTCCTGTTACTAGGAAGTTCTTCCTGCCATGTACT |
| | | GAGCCCAAACCTGTCTGTCTGCTTGCCATTTCCAGTCTTTCACTCC |
| | | ACTTCCGCCCCCACAAAACATACAGACCGAGAGACATACGAGAGA |
| | | CTCTGGTCAC |
| 65 | Enhancer GHC | AGTATATATACAAATCTATATACATTTCAAATGCTTAAATATGCTCA |
| | | CAAGTTTTGAGGTTCTGGTTGAAAACACCATTTGAAATGATCTAAG |
| | | ACTTTGTGTTAATAATACAAGAAAACTCTTGTTTCACTGCATGAGG |
| | | CCATCTTTCTCTACATTATCTTCTTCTATTCAGTGGTTATCTTCCCA |
| | | TAAAACTAACACACACGTGCTGGAGCAGATTCCCCTCTTCCCTTCA |
| | | CTCCTCTTAGCTCTTCTTAGCTTGATTCTGATAAGGACTGAAGCAG |
| | | AGAACACACGGCCCATCCACTTCAGAAATCATCTGTGCACCTGCT |
| | | GTATGCAGGAACATGTCTAGTAAAGCTTCCTTGAATTATTCATTGG |
| | | ACAGCATATTTCATTAACTACTGTGAAAATTGACTGAACTATTACCA |
| | | AAATGTAATACTGAAATTATAGAACTGAAGTCAAACAGACTGAGAG |
| | | AGGTCACCAACTCTCAACCTCCCCTCTCGCAGAGGGAGTTTCCTC |
| | | TCAACATCTAAACCTCCCACAGGCAGGCAACCACATCCCCTTTTC |
| | | AGCTTACCCACTTAAAGCATGGGCCTTTCCTGGACATCAGCTCCT |
| | | GTTACTAGGAAGTTCTTCCTGCCATGTACTGAGCCCAAACCTGTCT |
| | | GTCTGCTTGCCATTTCCAGTCTTTCACTCCACTTCCGCCCCCACAA |
| | | AACATACAGACCGAGAGACATACGAGAGACTCTGGTCAC |

The foregoing enhancer sequences (e.g., a GJB2 enhancer having at least 85% sequence identity (e.g., 85%, 86%, 87%, 88%, 89%, 90%, 91%, 92%, 93%, 94%, 95%, 96%, 97%, 98%, 99%, or more, sequence identity) to any one of SEQ ID NOs: 52-63, such as a GJB2 enhancer having at least 85% sequence identity to any one of SEQ ID NOs: 52-59) can be included in a nucleic acid vector and operably linked to a promoter, which can itself be operably linked to a polynucleotide encoding an expression product (e.g., a polynucleotide encoding a protein of interest or an inhibitory RNA) to express the expression product specifically in GJ1B2-expressing cells (e.g., in GJ1B2-expressing inner ear cells, such as cochlear supporting cells). In some embodiments, the polynucleotide encoding an expression product is a transgene that encodes a wild-type form of Gjb2 or a variant thereof (e.g., a polynucleotide sequence that encodes a protein having at least 85% sequence identity (e.g., 85%, 86%, 87%, 88%, 89%, 90%, 91%, 92%, 93%, 94%, 95%, 96%, 97%, 98%, 99%, or more, sequence identity) to the amino acid sequence of wild-type mammalian (e.g., human or mouse) Gjb2 (e.g., SEQ ID NO: 38 or SEQ ID NO: 45), such as a transgene having the sequence of any one of SEQ ID NOs: 39-44 and 46). In some embodiments, the polynucleotide encoding an expression product is a transgene that encodes a wild-type form of Gjb6 or a variant thereof (e.g., a polynucleotide sequence that encodes a protein having at least 85% sequence identity (e.g., 85%, 86%, 87%, 88%, 89%, 90%, 91%, 92%, 93%, 94%, 95%, 96%, 97%, 98%, 99%, or more, sequence identity) to the amino acid sequence of wild-type mammalian (e.g., human or mouse) Gjb6 (e.g., SEQ ID NO: 47 or SEQ ID NO: 50), such as a transgene having the sequence of SEQ ID NO: 48, SEQ ID NO: 49, or SEQ ID NO: 51). In some embodiments, the polynucleotide encoding an expression product is a polynucleotide encoding a protein that is endogenously expressed in GJB2-expressing cells, a polynucleotide encoding a protein that promotes differentiation of cochlear supporting cells into cochlear hair cells, a polynucleotide that corresponds to a wild-type form of a gene that is endogenously expressed in a GJB2-expressing inner ear cell and is mutated in a subject with hearing loss, deafness, or tinnitus, or another polynucleotide that can be expressed in GJB2-expressing inner ear cells to treat hearing loss, deafness, or tinnitus. In some embodiments, the polynucleotide encoding an expression product is a transgene that encodes BDNF or NTF3. In some embodiments, the polynucleotide encoding an expression product is a polynucleotide encoding a shRNA, an ASO, a component of a gene editing system (e.g., a nuclease, such as a Cas9, TALEN, or ZFN, or a gRNA), or a microRNA (e.g., miR-183, miR-96, or miR-182).

In some embodiments, a GJB2 enhancer described herein (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 59) is operably linked to a promoter that is operably linked to a polynucleotide encoding an expression product that can promote cochlear hair cell regeneration and/or that can induce or increase the differentiation of cochlear supporting cells into cochlear hair cells, such as Atoh1 (e.g., wild-type Atoh1 or an Atoh1 variant having one or more amino acid substitutions selected from the group consisting of S328A, S331A, S334A, S328A/S331A, S328A/S334A, S331A/S334A, and S328A/S331A/S334 as described in U.S. Publication No. US20190203210A1, which is incorporated herein by reference), Pou4F3, Gfi1, or Ikzf2, or an expression product that can promote cochlear supporting cell proliferation, such as Lgr5, Yap1, or Tead2. In some embodiments, the expression product is an inhibitory RNA directed to a gene that suppresses cochlear supporting cell differentiation or proliferation, such as LATS1 and/or LATS2. Additional polynucleotides that can be expressed to promote cochlear hair cell regeneration (e.g., polynucleotides that can induce cochlear supporting cell proliferation or differentiation) are provided in Table 5. A promoter that can be operably linked to a GJB2 enhancer having at least 85% sequence identity to SEQ ID NO: 59 for expression of such a polynucleotide is a GJB2 promoter (e.g., a GJB2 promoter described herein above, a GJB2 promoter having at least 85% sequence identity to any one of SEQ ID NOs: 1 and 8-11, or a GJB2 promoter having at least 85% sequence identity to a promoter listed in Table 8) or a supporting cell promoter (e.g., a cochlear supporting cell promoter listed in Table 9). A nucleic acid vector (e.g., an AAV vector) containing a GJB2 enhancer having at least 85% sequence identity to SEQ ID NO: 59 operably linked to a promoter that is operably linked to a polynucleotide encoding an expression product that is implicated in cochlear hair cell regeneration can be administered to a subject having or at risk of developing hearing loss associated with loss of cochlear hair cells (e.g., age-related hearing loss, noise-induced hearing loss, ototoxic drug-induced hearing loss, head trauma-related hearing loss, or disease or infection-related hearing loss).

In some embodiments, a GJB2 enhancer described herein (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 59) is operably linked to a promoter that is operably linked to a transgene corresponding to a wild-type version of a gene that is expressed in cochlear supporting cells and known to be mutated in a disease associated with hearing loss (e.g., a monogenic form of hearing loss, such as a disease listed in Table 6, which can be treated by expressing a wild-type form of the gene in the same row of Table 6). For example, the transgene can correspond to a wild-type form of SLC26A4, PAX3, NDP, or COCH, mutations in which are associated with Pendred Syndrome, Waardenburg Syndrome, Norrie disease, and DFNA9, respectively. A promoter that can be operably linked to a GJB2 enhancer having at least 85% sequence identity to SEQ ID NO: 59 for expression of such a transgene is a GJB2 promoter (e.g., a GJB2 promoter described herein above, a GJB2 promoter having at least 85% sequence identity to any one of SEQ ID NOs: 1 and 8-11, or a GJB2 promoter having at least 85% sequence identity to a promoter listed in Table 8) or a supporting cell promoter (e.g., a cochlear supporting cell promoter listed in Table 9). A nucleic acid vector (e.g., an AAV vector) containing a GJB2 enhancer having at least 85% sequence identity to SEQ ID NO: 59 operably linked to a promoter that is operably linked to a transgene corresponding to the wild-type form of any of the genes listed in Table 6 or the wild-type form of any cochlear supporting cell gene (e.g., a gene expressed in cochlear supporting cells) that is known to be mutated in a subject with hearing loss can be administered to a subject carrying said mutation to treat the associated disease (e.g., improve or restore hearing).

In some embodiments, the promoter that is operably linked to a GJB2 enhancer described herein is a GJB2 promoter. For example, a nucleic acid vector can contain a GJB2 enhancer described herein operably linked to a GJB2 promoter that is operably linked to a polynucleotide encoding Gjb2 (e.g., for the treatment of GJB2-related hearing loss), Gjb6 (e.g., for the treatment of DFNB1 or DFNA3), BDNF (e.g., for the treatment of sensorineural hearing loss), or NTF3 (e.g., for the treatment of sensorineural hearing loss). In another example, a nucleic acid vector can contain a GJB2 enhancer described herein operably linked to a GJB2 promoter that is operably linked to a polynucleotide listed in Table 5 (e.g., to promote cochlear hair cell regeneration, e.g., for treating hearing loss associated with loss of cochlear hair cells), a transgene corresponding to the wild-type form of a gene listed in Table 6 (e.g., to treat a corresponding disease listed in Table 6), or a polynucleotide encoding a protein or RNA molecule that is endogenously expressed in a GJB2-expressing cell. In some embodiments, the GJB2 promoter is a GJB2 promoter described herein (e.g., a polynucleotide that contains a first region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1 or a functional portion or derivative thereof and/or a second region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or a functional portion or derivative thereof, optionally containing a linker joining the first region and the second region, such as a GJB2 promoter having at least 85% sequence identity to any one of SEQ ID NOs: 29-35). In some embodiments, the GJB2 promoter that is operably linked to a GJB2 enhancer described herein (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to any one of SEQ ID NOs: 52-63, such as a polynucleotide having at least 85% sequence identity to any one of SEQ ID NOs: 52-59) is a portion of a GJB2 promoter described herein, such as a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to any one of SEQ ID NOs: 1 and 8-11. Additional GJB2 promoters that can be operably linked to a GJB2 enhancer described herein (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to any one of SEQ ID NOs: 52-63, such as a polynucleotide having at least 85% sequence identity to any one of SEQ ID NOs: 52-59) are provided in Table 8 below. In some embodiments, a GJB2 enhancer described herein is operably linked to a GJB2 promoter having least 85% sequence identity (e.g., 85%, 86%, 87%, 88%, 89%, 90%, 91%, 92%, 93%, 94%, 95%, 96%, 97%, 98%, 99%, or 100% sequence identity) to any one of SEQ ID NOs: 66-68.

TABLE 8
Additional GJB2 promoters

| SEQ ID | Description | |
|---|---|---|
| NO: | of Promoter | Promoter Sequence |
| 66 | 1000 bp | CTCATAAATGCCAAGTCCTCTCGCACTATGCGGAGTACAGAGGACAAC |
| | promoter | GACCACAGCCATCCCTGAACCCCGCCCACGGCACAGCGCCGGAGCC |
| | | GGGGTCTGGGGCGCCGCTTCCTGGGGGGTCCCGACTCTCAGCCGCC |
| | | CCCGCTTCACCCGGGCCGCCAAGGGGCTGGGGGAGGCGGCGCTCG |
| | | GGGTAACCGGGGGAGACTCAGGGCGCTGGGGGCACTTGGGGAACTC |
| | | ATGGGGGCTCAAAGGAACTAGGAGATCGGGACCTCGAAGGGGACTT |
| | | GGGGGGTTCGGGGCTTTCGGGGGCGGTCGGGGGTTCGCGGACCCG |
| | | GGAAGCTCTGAGGACCCAGAGGCCGGGCGCGCTCCGCCCGCGGCG |
| | | CCGCCCCCTCCGTAACTTTCCCAGTCTCCGAGGGAAGAGGGGGGGT |
| | | GTGGGGTGCGGTTAAAAGGCGCCACGGCGGGAGACAGGTGTTGCGG |
| | | CCCCGCAGCGCCCGCGCGCTCCTCTCCCCGACTCGGAGCCCCTCGG |
| | | CGGCGCCCGGCCCAGGACCCGCCTAGGAGCGCAGGAGCCCCAGCG |
| | | CAGAGACCCCAACGCCGAGACCCCCGCCCCGGCCCCGCCGCGCTTC |
| | | CTCCCGACGCAGGTGAGCCCGCCGGCCCCGGACTGCCCGGCCAGGA |
| | | ACCTGGCGCGGGGAGGGACCGCGAGACCCAGAGCGGTTGCCCGGC |
| | | CGCGTGGGTCTCGGGGAACCGGGGGGCTGGACCAACACACGTCCTT |
| | | GGGCCGGGGGGGGGGGGCCGCCTTCTGGAGCGGGCGTTTCTGCGG |
| | | CCGAGCTCCGGAGCTGGAATGGGGCGGCCGGGGAAGTGGACGCGAT |
| | | GGCACCGCCCGGGGTGCGAGTGGGGCCGGGCGCGCGCGGGAGGG |
| | | GAAAAAGGCGCGGGCGAGCCGCCAGCGCGAGGTTTGTGGTGTCGCC |
| | | GATGTCCCTTCGGGGTACTCTAGCGCAGCCGCCTGGCTACTTGACCC |
| | | ACTGCCACCAAACGTTTTAAATTCACCGAAAGCTTAGCT |
| 67 | 1553 bp | AAGCTTCGGTGAATTTAAAACGTTTGGTGGCAGTGGGTCAAGTAGCCA |
| | promoter | GGCGGCTGCGCTAGAGTACCCCGAAGGGACATCGGCGACACCACAA |
| | | ACCTCGCGCTGGCGGCTCGCCCGCGCCTTTTTCCCCTCCCGCGCGC |
| | | GCCCGGCCCCACTCGCACCCCGGGGGGTGCCATCGCGTCCACTTCC |
| | | CCGGCCGCCCCATTCCAGCTCCGGAGCTCGGCCGCAGAAACGCCCG |
| | | CTCCAGAAGGCGGCCCCCGCCCCCCGGCCCAAGGACGTGTGTTGGT |
| | | CCAGCCCCCCGGTTCCCCGAGACCCACGCGGCCGGGCAACCGCTCT |
| | | GGGTCTCGCGGTCCCTCCCCGCGCCAGGTTCCTGGCCGGGCAGTCC |
| | | GGGGCCGGGGGGCTCACCTGCGTCGGGAGGAAGCGCGGGGGGGCC |
| | | GGGGGGGGGGTCTCGGCGTTGGGGTCTCTGCGCTGGGGCTCCTGCG |
| | | CTCCTAGGCGGGTCCTGGGCCGGGCGCCGCCGAGGGGCTCCGAGT |
| | | CGGGGAGAGGAGCGCGCGGGCGCTGCGGGGCCGCAACACCTGTCT |
| | | CCCGCCGTGGCGCCTTTTAACCGCACCCCACACCCCGCCTCTTCCCT |
| | | CGGAGACTGGGAAAGTTACGGAGGGGGGGGCGCCGCGGGCGGAGC |
| | | GCGCCCGGCCTCTGGGTCCTCAGAGCTTCCCGGGTCCGCGAACCCC |
| | | CGACCGCCCCCGAAAGCCCCGAACCCCCCAAGTCCCCTTCGAGGTC |
| | | CCGATCTCCTAGTTCCTTTGAGCCCCCATGAGTTCCCCAAGTGCCCCC |
| | | AGCGCCCTGAGTCTCCCCCGGTTACCCCGAGCGCCGCCTCCCCCAG |
| | | CCCCTTGGCGGCCCGGGTGAAGCGGGGGGGGCTGAGAGTCGGGAC |
| | | CCCCCAGGAAGCGGCGCCCCAGACCCCGGCTCCGGCGCTGTGCCGT |
| | | GGGGGGGGTTCAGGGATGGCTGTGGTCGTTGTCCTCTGTACTCCGCA |
| | | TAGTGCGAGAGGACTTGGCATTTATGAGCGCTTCTTTAATTTTTTATTG |
| | | TTAGAGAAACAGGCATTCCTCCAAGGACTGAAGATCTGTTCGAGTCGC |
| | | GGAGGCTGCGCGGGCCCGCGAGGCTCTCGCAGGGGGACCTAGGCT |
| | | GGGTGGGGGGCAGTGCCCTCTGGAATGGGGGTTAACGGTGGCCGA |
| | | GGAGGGGGCGCCGCTGGTGCCGGCGAAGTCCCCGCTTCTTTCTCCC |
| | | CTCAAAATCTCACCAATCCGAACGAACGCCTTCTCGAATTTCCGATTTT |
| | | ATTCAATTACTTTCAACAATGTGCCAAGGACTAAGGTTGGGGGGGGTG |
| | | GGAGAGACAAGCCTCGTTTTTGCCATGGCCGGCAGGGGGGTCCCGC |
| | | CATCTGCGGAGGGTGCCCCCCGCGGCCCCCGGCCCAGCCAACTTCC |
| | | TCCTCTTTTCGCAACTGGGGAACTGCAAGGAGGTGACTCCTTTCGGG |
| | | GTGAGGAGGCCCAGACTTTTCAGAAAGGAAAGAGGGCAGGTAAAACC |
| | | TGCCAAGCCCCTTCCTGCTCGATGCACACAGCACGAAAGGGGGAAAC |
| | | TGATAGGATTCTGCGGAAGCTT |
| 68 | 1010 bp | AAGCTTCCGCAGAATCCTATCAGTTTCCCCCTTTCGTGCTGTGTGCAT |
| | promoter | CGAGCAGGAAGGGGCTTGGCAGGTTTTACCTGCCCTCTTTCCTTTCTG |
| | | AAAAGTCTGGGCCTCCTCACCCCGAAAGGAGTCACCTCCTTGCAGTT |
| | | CCCCAGTTGCGAAAAGAGGAGGAAGTTGGCTGGGCCGGGGGCCGCG |
| | | GGGGGCACCCTCCGCAGATGGCGGGACCCCCCTGCCGGCCATGGCA |
| | | AAAACGAGGCTTGTCTCTCCCACCGCCCCCAACCTTAGTCCTTGGCAC |
| | | ATTGTTGAAAGTAATTGAATAAAATCGGAAATTCGAGAAGGCGTTCGTT |
| | | CGGATTGGTGAGATTTTGAGGGGAGAAAGAAGCGGGGACTTCGCCG |
| | | GCACCAGCGGCGCCCCCTCCTCGGCCACCGTTAACCCCCATTCCAGA |
| | | GGGCACTGCCCCGCCACCCAGCCTAGGTCCCCCTGCGAGAGCCTCG |
| | | CGGGCCCGCGCAGCCTCCGCGACTCGAACAGATCTTCAGTCCTTGGA |
| | | GGAATGCCTGTTTCTCTAACAATAAAAAATTAAAGAAGCGCTCATAAAT |
| | | GCCAAGTCCTCTCGCACTATGCGGAGTACAGAGGACAACGACCACAG |
| | | CCATCCCTGAACCCCGCCCACGGCACAGCGCCGGAGCCGGGGTCTG |
| | | GGGCGCCGCTTCCTGGGGGGTCCCGACTCTCAGCCGCCCCCGCTTC |
| | | ACCCGGGCCGCCAAGGGGCTGGGGGAGGCGGCGCTCGGGGTAACC |
| | | GGGGGAGACTCAGGGCGCTGGGGGCACTTGGGGAACTCATGGGGG |
| | | CTCAAAGGAACTAGGAGATCGGGACCTCGAAGGGGACTTGGGGGGTT |
| | | CGGGGCTTTCGGGGGCGGTCGGGGGTTCGCGGACCCGGGAAGCTCT |
| | | GAGGACCCAGAGGCCGGGCGCGCTCCGCCCGCGGCGCCGCCCCCT |
| | | CCGTAACTTTCCCAGTCTCCGAGGGAAGAGGCGGGGTGTGGGGTGC |
| | | GGTTAAAAGGCGCCACGGCGGGAGACAGGT |

In some embodiments, the promoter that is operably linked to a GJB2 enhancer described herein is an inner ear cell type-specific promoter. For example, a nucleic acid vector can contain a GJB2 enhancer described herein operably linked to an inner ear cell-type specific promoter that is operably linked to a polynucleotide encoding Gjb2 (e.g., for the treatment of GJB2-related hearing loss), Gjb6 (e.g., for the treatment of DeNB1 or DFNA3), BDNF (e.g., for the treatment of sensorineural hearing loss), or NTF3 (e.g., for the treatment of sensorineural hearing loss). In another example, a nucleic acid vector can contain a GJB2 enhancer described herein operably linked to an inner ear cell-type specific promoter that is operably linked to a polynucleotide listed in Table 5 (e.g., to promote cochlear hair cell regeneration, e.g., for treating hearing loss associated with loss of cochlear hair cells), a transgene corresponding to the wild-type form of a gene listed in Table 6 (e.g., to treat a corresponding disease listed in Table 6), or a polynucleotide encoding a protein or RNA molecule that is endogenously expressed in a GJ2-expressing cell. Cell type-specific promoters that can be operably linked to one or more GJB2 enhancers (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to any one of SEQ ID NOs: 52-59, such as a polynucleotide having at least 85% sequence identity to any one of SEQ ID NOs: 25-29) to express a polynucleotide encoding an expression product (e.g., GJB6, GJB2, BDNF, NTF3, a polynucleotide listed in Table 5, or a gene listed in Table 6) in one or more GJB2-expressing inner ear cells are provided in Table 9, below.

TABLE 9
Promoters for GJB2-expressing inner ear cells

| Cell Type | Promoter |
|---|---|
| Supporting cells | SRY-Box 2 (SOX2), Glial Acidic Fibrillary Protein |
| | (GFAP) |
| Supporting cell progenitors | Leucine Rich Repeat Containing G Protein- |
| | Coupled Receptor 5 (LGR5) |
| Border cells (cochlear supporting cell | GLAST |
| subtype) | |
| Inner phalangeal cells (cochlear supporting | GLAST |
| cell subtype) | |
| Pillar cells (cochlear supporting cell | CD44 Molecule (CD44) |
| subtype) | |
| Deiters' cells (cochlear supporting cell | Fibroblast Growth Factor Receptor 3 (FGFR3) |
| subtype) | |
| Hensen's cells (cochlear supporting cell | Frizzled Related Protein (FRZB) |
| subtype) | |
| Claudius cells (cochlear supporting cell | FRZB |
| subtype) | |
| Spiral prominence cells | SLC26A4 |
| Root cells | SLC26A4 |
| Interdental cells | CEACAM16 |
| Basal cells of the SV | Claudin 11 (CLDN11) |
| Intermediate cells of the SV | Tyrosinase (TYR), Potassium Voltage-Gated |
| | Channel Subfamily J Member 10 (KCNJ10) |
| Vestibular dark cells | KCNE1 |
| Fibrocytes/mesenchyme | POU3F4 |

The nucleic acid vectors containing a GJB2 promoter and/or a GJB2 enhancer described herein may include a Woodchuck Posttranscriptional Regulatory Element (WPRE). The WPRE acts at the mRNA level, by promoting nuclear export of transcripts and/or by increasing the efficiency of polyadenylation of the nascent transcript, thus increasing the total amount of mRNA in the cell. The addition of the WPRE to a vector can result in a substantial improvement in the level of transgene expression from several different promoters, both in vitro and in vivo.

In some embodiments, the nucleic acid vectors containing a GJB2 promoter and/or a GJB2 enhancer described herein include a reporter sequence, which can be useful in verifying the expression of a gene operably linked to a GJB2 promoter, for example, in cells and tissues (e.g., in GJB2-expressing cells, such as cochlear supporting cells). Reporter sequences that may be provided in a transgene include DNA sequences encoding β-lactamase, β-galactosidase (LacZ), alkaline phosphatase, thymidine kinase, green fluorescent protein (GFP), chloramphenicol acetyltransferase (CAT), luciferase, and others well known in the art. When associated with regulatory elements that drive their expression, such as a GJB2 promoter, the reporter sequences provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA), and immunohistochemistry. For example, where the marker sequence is the LacZ gene, the presence of the vector carrying the signal is detected by assays for β-galactosidase activity. Where the transgene is green fluorescent protein or luciferase, the vector carrying the signal may be measured visually by color or light production in a luminometer.

### Methods for the Delivery of Exogenous Polynucleotides to Target Cells

Techniques that can be used to introduce a polynucleotide, such as a polynucleotide operably linked to a GJB2 promoter described herein (e.g., a polynucleotide that contains a first region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1 or a functional portion or derivative thereof and/or a second region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or a functional portion or derivative thereof, optionally containing a linker joining the first region and the second region) or a polynucleotide that is operably linked to a promoter that is operably linked to a GJB2 enhancer described herein, into a target cell (e.g., a mammalian cell) are well known in the art. For instance, electroporation can be used to permeabilize mammalian cells (e.g., human target cells) by the application of an electrostatic potential to the cell of interest. Mammalian cells, such as human cells, subjected to an external electric field in this manner are subsequently predisposed to the uptake of exogenous polynucleotides. Electroporation of mammalian cells is described in detail, e.g., in Chu et al., Nucleic Acids Research 15:1311 (1987), the disclosure of which is incorporated herein by reference. A similar technique, Nucleofection™, utilizes an applied electric field in order to stimulate the uptake of exogenous polynucleotides into the nucleus of a eukaryotic cell. Nucleofection™ and protocols useful for performing this technique are described in detail, e.g., in Distler et al., Experimental Dermatology 14:315 (2005), as well as in US 2010/0317114, the disclosures of each of which are incorporated herein by reference.

Additional techniques useful for the transfection of target cells include the squeeze-poration methodology. This technique induces the rapid mechanical deformation of cells in order to stimulate the uptake of exogenous DNA through membranous pores that form in response to the applied stress. This technology is advantageous in that a vector is not required for delivery of polynucleotides into a cell, such as a human target cell. Squeeze-poration is described in detail, e.g., in Sharei et al., Journal of Visualized Experiments 81:e50980 (2013), the disclosure of which is incorporated herein by reference.

Lipofection represents another technique useful for transfection of target cells. This method involves the loading of polynucleotides into a liposome, which often presents cationic functional groups, such as quaternary or protonated amines, towards the liposome exterior. This promotes electrostatic interactions between the liposome and a cell due to the anionic nature of the cell membrane, which ultimately leads to uptake of the exogenous polynucleotides, for instance, by direct fusion of the liposome with the cell membrane or by endocytosis of the complex. Lipofection is described in detail, for instance, in U.S. Pat. No. 7,442,386, the disclosure of which is incorporated herein by reference. Similar techniques that exploit ionic interactions with the cell membrane to provoke the uptake of foreign polynucleotides include contacting a cell with a cationic polymer-polynucleotide complex. Exemplary cationic molecules that associate with polynucleotides so as to impart a positive charge favorable for interaction with the cell membrane include activated dendrimers (described, e.g., in Dennig, Topics in Current Chemistry 228:227 (2003), the disclosure of which is incorporated herein by reference) polyethylenimine, and diethylaminoethyl (DEAE)-dextran, the use of which as a transfection agent is described in detail, for instance, in Gulick et al., Current Protocols in Molecular Biology 40:1:9.2:9.2.1 (1997), the disclosure of which is incorporated herein by reference. Magnetic beads are another tool that can be used to transfect target cells in a mild and efficient manner, as this methodology utilizes an applied magnetic field in order to direct the uptake of polynucleotides. This technology is described in detail, for instance, in US 2010/0227406, the disclosure of which is incorporated herein by reference.

Another useful tool for inducing the uptake of exogenous polynucleotides by target cells is laserfection, also called optical transfection, a technique that involves exposing a cell to electromagnetic radiation of a particular wavelength in order to gently permeabilize the cells and allow polynucleotides to penetrate the cell membrane. The bioactivity of this technique is similar to, and in some cases found superior to, electroporation.

Impalefection is another technique that can be used to deliver genetic material to target cells. It relies on the use of nanomaterials, such as carbon nanofibers, carbon nanotubes, and nanowires. Needle-like nanostructures are synthesized perpendicular to the surface of a substrate. DNA containing the gene, intended for intracellular delivery, is attached to the nanostructure surface. A chip with arrays of these needles is then pressed against cells or tissue. Cells that are impaled by nanostructures can express the delivered gene(s). An example of this technique is described in Shalek et al., PNAS 107: 1870 (2010), the disclosure of which is incorporated herein by reference.

Magnetofection can also be used to deliver polynucleotides to target cells. The magnetofection principle is to associate polynucleotides with cationic magnetic nanoparticles. The magnetic nanoparticles are made of iron oxide, which is fully biodegradable, and coated with specific cationic proprietary molecules varying upon the applications. Their association with the gene vectors (DNA, siRNA, viral vector, etc.) is achieved by salt-induced colloidal aggregation and electrostatic interaction. The magnetic particles are then concentrated on the target cells by the influence of an external magnetic field generated by magnets. This technique is described in detail in Scherer et al., Gene Therapy 9:102 (2002), the disclosure of which is incorporated herein by reference.

Another useful tool for inducing the uptake of exogenous polynucleotides by target cells is sonoporation, a technique that involves the use of sound (typically ultrasonic frequencies) for modifying the permeability of the cell plasma membrane to permeabilize the cells and allow polynucleotides to penetrate the cell membrane. This technique is described in detail, e.g., in Rhodes et al., Methods in Cell Biology 82:309 (2007), the disclosure of which is incorporated herein by reference.

Microvesicles represent another potential vehicle that can be used to modify the genome of a target cell according to the methods described herein. For instance, microvesicles that have been induced by the co-overexpression of the glycoprotein VSV-G with, e.g., a genome-modifying protein, such as a nuclease, can be used to efficiently deliver proteins into a cell that subsequently catalyze the site-specific cleavage of an endogenous polynucleotide sequence so as to prepare the genome of the cell for the covalent incorporation of a polynucleotide of interest, such as a gene or regulatory sequence. The use of such vesicles, also referred to as Gesicles, for the genetic modification of eukaryotic cells is described in detail, e.g., in Quinn et al., Genetic Modification of Target Cells by Direct Delivery of Active Protein [abstract]. In: Methylation changes in early embryonic genes in cancer [abstract], in: Proceedings of the 18th Annual Meeting of the American Society of Gene and Cell Therapy; 2015 May 13, Abstract No. 122.

### Vectors for Delivery of Exogenous Polynucleotides to Target Cells

In addition to achieving high rates of transcription and translation, stable expression of an exogenous polynucleotide in a mammalian cell can be achieved by integration of the polynucleotide into the nuclear genome of the mammalian cell. A variety of vectors for the delivery and integration of polynucleotides encoding exogenous proteins into the nuclear DNA of a mammalian cell have been developed. Examples of expression vectors are described in, e.g., Gellissen, Production of Recombinant Proteins: Novel Microbial and Eukaryotic Expression Systems (John Wiley & Sons, Marblehead, MA, 2006). Expression vectors for use in the compositions and methods described herein contain a GJB2 promoter described herein (e.g., a polynucleotide that contains a first region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1 or a functional portion or derivative thereof and/or a second region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or a functional portion or derivative thereof, optionally containing a linker joining the first region and the second region) operably linked to a polynucleotide encoding an expression product (e.g., a polynucleotide that encodes a protein of interest or that can be transcribed to produce an inhibitory RNA) or a GJB2 enhancer described herein (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to any one of SEQ ID NOs: 52-59) operably linked to a promoter that is operably linked polynucleotide encoding an expression product (e.g., a polynucleotide that encodes a protein of interest or that can be transcribed to produce an inhibitory RNA), as well as, e.g., additional sequence elements used for the expression of these agents and/or the integration of these polynucleotide sequences into the genome of a mammalian cell. Vectors that can contain a GJB2 promoter and/or a GJB2 enhancer operably linked to polynucleotide encoding an expression product (e.g., a transgene encoding a protein of interest) include plasmids (e.g., circular DNA molecules that can autonomously replicate inside a cell), cosmids (e.g., pWE or sCos vectors), artificial chromosomes (e.g., a human artificial chromosome (HAC), a yeast artificial chromosome (YAC), a bacterial artificial chromosome (BAC), or a P1-derived artificial chromosome (PAC)), and viral vectors. Certain vectors that can be used for the expression of an expression product (e.g., a protein of interest) include plasmids that contain regulatory sequences, such as enhancer regions (e.g., a GJB2 enhancer described herein), which direct gene transcription. Other useful vectors for expression of an expression product (e.g., a protein of interest) contain polynucleotide sequences that enhance the rate of translation of these genes or improve the stability or nuclear export of the mRNA that results from gene transcription. These sequence elements include, e.g., 5′ and 3′ untranslated regions, an internal ribosomal entry site (IRES), and polyadenylation signal site in order to direct efficient transcription of the gene carried on the expression vector. The expression vectors suitable for use with the compositions and methods described herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

### Viral Vectors for Polynucleotide Delivery

Viral genomes provide a rich source of vectors that can be used for the efficient delivery of a gene of interest into the genome of a target cell (e.g., a mammalian cell, such as a human cell). Viral genomes are particularly useful vectors for gene delivery because the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Examples of viral vectors include a retrovirus (e.g., Retroviridae family viral vector), adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses include avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology, Third Edition (Lippincott-Raven, Philadelphia, 1996)). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, U.S. Pat. No. 5,801,030, the disclosure of which is incorporated herein by reference as it pertains to viral vectors for use in gene therapy.

### AAV Vectors for Polynucleotide Delivery

In some embodiments, polynucleotides of the compositions and methods described herein are incorporated into rAAV vectors and/or virions in order to facilitate their introduction into a cell. In some embodiments, rAAV vectors useful in the compositions and methods described herein are recombinant polynucleotide constructs that include (1) a GJB2 promoter described herein (e.g., a polynucleotide that contains a first region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1 or a functional portion or derivative thereof and/or a second region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or a functional portion or derivative thereof, optionally containing a linker joining the first region and the second region), (2) a sequence to be expressed (e.g., a polynucleotide encoding a protein or a polynucleotide that can be transcribed to produce an inhibitory RNA), and (3) viral sequences that facilitate integration and expression of the sequence to be expressed. In some embodiments, rAAV vectors useful in the compositions and methods described herein are recombinant polynucleotide constructs that include (1) at least one GJB2 enhancer described herein (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to any one of SEQ ID NOs: 52-59), (2) a promoter, (3) a sequence to be expressed, and (4) viral sequences that facilitate integration and expression of the sequence to be expressed. The viral sequences may include those sequences of AAV that are required in cis for replication and packaging (e.g., functional ITRs) of the DNA into a virion. In typical applications, the sequence to be expressed encodes a protein that can promote cochlear hair cell regeneration (e.g., differentiation of cochlear supporting cells into cochlear hair cells), cochlear supporting cell survival, cochlear supporting cell proliferation, or a wild-type form of a cochlear supporting cell protein that is mutated in subjects with forms of hereditary hearing loss that may be useful for improving hearing in subjects carrying mutations that have been associated with hearing loss, deafness, tinnitus, or auditory neuropathy. Such rAAV vectors may also contain marker or reporter genes. Useful rAAV vectors have one or more of the AAV WT genes deleted in whole or in part but retain functional flanking ITR sequences. The AAV ITRs may be of any serotype suitable for a particular application. For use in the methods and compositions described herein, the ITRs can be AAV2 ITRs. Methods for using rAAV vectors are described, for example, in Tal et al., J. Biomed. Sci. 7:279 (2000), and Monahan and Samulski, Gene Delivery 7:24 (2000), the disclosures of each of which are incorporated herein by reference as they pertain to AAV vectors for gene delivery.

The polynucleotides and vectors described herein (e.g., a GJB2 promoter operably linked to a polynucleotide encoding an expression product or a GJB2 enhancer operably linked to a promoter that is operably linked to a polynucleotide encoding an expression product) can be incorporated into a rAAV virion in order to facilitate introduction of the polynucleotide or vector into a cell. The capsid proteins of AAV compose the exterior, non-nucleic acid portion of the virion and are encoded by the AAV cap gene. The cap gene encodes three viral coat proteins, VP1, VP2 and VP3, which are required for virion assembly. The construction of rAAV virions has been described, for instance, in U.S. Pat. Nos. 5,173,414; 5,139,941; 5,863,541; 5,869,305; 6,057,152; and 6,376,237; as well as in Rabinowitz et al., J. Virol. 76:791 (2002) and Bowles et al., J. Virol. 77:423 (2003), the disclosures of each of which are incorporated herein by reference as they pertain to AAV vectors for gene delivery.

rAAV virions useful in conjunction with the compositions and methods described herein include those derived from a variety of AAV serotypes including AAV 1, 2, 3, 4, 5, 6, 7, 8, 9, 10, 11, rh10, rh39, rh43, rh74, Anc80, Anc80L65, DJ, DJ/8, DJ/9, 7m8, PHP.B, PHP.eb, and PHP.S. For targeting GJB2-expressing cells, AAV1, AAV2, AAV2quad(Y-F), AAV6, AAV8, AAV9, Anc80, Anc80L65, AAV-DJ, AAV-DJ/9, 7m8, and PHP.B may be particularly useful. Serotypes evolved for transduction of the retina may also be used in the methods and compositions described herein. Construction and use of AAV vectors and AAV proteins of different serotypes are described, for instance, in Chao et al., Mol. Ther. 2:619 (2000); Davidson et al., Proc. Natl. Acad. Sci. USA 97:3428 (2000); Xiao et al., J. Virol. 72:2224 (1998); Halbert et al., J. Virol. 74:1524 (2000); Halbert et al., J. Virol. 75:6615 (2001); and Auricchio et al., Hum. Molec. Genet. 10:3075 (2001), the disclosures of each of which are incorporated herein by reference as they pertain to AAV vectors for gene delivery.

Also useful in conjunction with the compositions and methods described herein are pseudotyped rAAV vectors. Pseudotyped vectors include AAV vectors of a given serotype (e.g., AAV9) pseudotyped with a capsid gene derived from a serotype other than the given serotype (e.g., AAV1, AAV2, AAV2quad(Y-F), AAV3, AAV4, AAV5, AAV6, AAV7, AAV8, etc.). Techniques involving the construction and use of pseudotyped rAAV virions are known in the art and are described, for instance, in Duan et al., J. Virol. 75:7662 (2001); Halbert et al., J. Virol. 74:1524 (2000); Zolotukhin et al., Methods, 28:158 (2002); and Auricchio et al., Hum. Molec. Genet. 10:3075 (2001).

AAV virions that have mutations within the virion capsid may be used to infect particular cell types more effectively than non-mutated capsid virions. For example, suitable AAV mutants may have ligand insertion mutations for the facilitation of targeting AAV to specific cell types. The construction and characterization of AAV capsid mutants including insertion mutants, alanine screening mutants, and epitope tag mutants is described in Wu et al., J. Virol. 74:8635 (2000). Other rAAV virions that can be used in methods described herein include those capsid hybrids that are generated by molecular breeding of viruses as well as by exon shuffling. See, e.g., Soong et al., Nat. Genet., 25:436 (2000) and Kolman and Stemmer, Nat. Biotechnol. 19:423 (2001).

### Pharmaceutical Compositions

The GJB2 promoters described herein (e.g., a polynucleotide that contains a first region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1 or a functional portion or derivative thereof and/or a second region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or a functional portion or derivative thereof, optionally containing a linker joining the first region and the second region) and/or the GJB2 enhancers described herein (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to any one of SEQ ID NOs: 52-63) may be operably linked to a polynucleotide encoding an expression product (e.g., a transgene encoding a protein of interest) and incorporated into a vehicle for administration into a patient, such as a human patient suffering from sensorineural hearing loss. Pharmaceutical compositions containing vectors, such as viral vectors, that contain a GJB2 promoter and/or a GJB2 enhancer described herein operably linked to a polynucleotide encoding an expression product can be prepared using methods known in the art. For example, such compositions can be prepared using, e.g., physiologically acceptable carriers, excipients, or stabilizers (Remington: The Science and Practice of Pharmacology 22nd edition, Allen, L. Ed. (2013); incorporated herein by reference), and in a desired form, e.g., in the form of lyophilized formulations or aqueous solutions.

Mixtures of nucleic acid vectors (e.g., viral vectors) containing a GJB2 promoter described herein (e.g., a polynucleotide that contains a first region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1 or a functional portion or derivative thereof and/or a second region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or a functional portion or derivative thereof, optionally containing a linker joining the first region and the second region) and/or a GJB2 enhancer described herein (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to any one of SEQ ID NOs: 52-63) operably linked to a polynucleotide encoding an expression product may be prepared in water suitably mixed with one or more excipients, carriers, or diluents. Dispersions may also be prepared in glycerol, liquid polyethylene glycols, and mixtures thereof and in oils. Under ordinary conditions of storage and use, these preparations may contain a preservative to prevent the growth of microorganisms. The pharmaceutical forms suitable for injectable use include sterile aqueous solutions or dispersions and sterile powders for the extemporaneous preparation of sterile injectable solutions or dispersions (described in U.S. Pat. No. 5,466,468, the disclosure of which is incorporated herein by reference). In any case the formulation may be sterile and may be fluid to the extent that easy syringability exists. Formulations may be stable under the conditions of manufacture and storage and may be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For example, a solution containing a pharmaceutical composition described herein may be suitably buffered, if necessary, and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion. Some variation in dosage will necessarily occur depending on the condition of the subject being treated. For local administration to the inner ear, the composition may be formulated to contain a synthetic perilymph solution. An exemplary synthetic perilymph solution includes 20-200 mM NaCl, 1-5 mM KCl, 0.1-10 mM CaCl₂), 1-10 mM glucose, and 2-50 mM HEPEs, with a pH between about 6 and 9 and an osmolality of about 300 mOsm/kg. The person responsible for administration will, in any event, determine the appropriate dose for the individual subject. Moreover, for human administration, preparations may meet sterility, pyrogenicity, general safety, and purity standards as required by FDA Office of Biologics standards.

### Methods of Treatment

The compositions described herein may be administered to a subject having or at risk of developing sensorineural hearing loss by a variety of routes, such as local administration to the middle or inner ear (e.g., administration into the perilymph or endolymph, such as to or through the oval window, round window, or semicircular canal (e.g., the horizontal canal), or by transtympanic or intratympanic injection, e.g., administration to a GJB2-expressing inner ear cell), intravenous, parenteral, intradermal, transdermal, intramuscular, intranasal, subcutaneous, percutaneous, intratracheal, intraperitoneal, intraarterial, intravascular, inhalation, perfusion, lavage, and oral administration. The most suitable route for administration in any given case will depend on the particular composition administered, the patient, pharmaceutical formulation methods, administration methods (e.g., administration time and administration route), the patient's age, body weight, sex, severity of the disease being treated, the patient's diet, and the patient's excretion rate. Compositions may be administered once, or more than once (e.g., once annually, twice annually, three times annually, bi-monthly, monthly, or bi-weekly).

Subjects that may be treated as described herein are subjects having or at risk of developing sensorineural hearing loss. In some embodiments, the compositions described herein are used to treat GJB2-related hearing loss (e.g., DFNB1 or DFNA3, or hearing loss associated with Bart-Pumphrey syndrome, hystrix-like ichthyosis with deafness, keratitis-ichthyosis-deafness syndrome, palmoplantar keratoderma with deafness, or Vohwinkel syndrome). DFNB1 and DFNA3 can be treated by administration of a nucleic acid vector containing a GJB2 promoter described herein (e.g., a polynucleotide that contains a first region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1 or a functional portion or derivative thereof and/or a second region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or a functional portion or derivative thereof, optionally containing a linker joining the first region and the second region) and/or a GJB2 enhancer described herein (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to any one of SEQ ID NOs: 52-63) (e.g., a GJB2 enhancer operably linked to a promoter, such as a GJB2 promoter or an inner ear cell-type specific promoter) operably linked to a polynucleotide encoding Gjb2 or Gjb6, while the hearing loss associated with Bart-Pumphrey syndrome, hystrix-like ichthyosis with deafness, keratitis-ichthyosis-deafness syndrome, palmoplantar keratoderma with deafness, and Vohwinkel syndrome can be treated by administration of a nucleic acid vector containing a GJB2 promoter described herein and/or a GJB2 enhancer described herein (e.g., a GJB2 enhancer operably linked to a promoter, such as a GJB2 promoter or an inner ear cell-type specific promoter) operably linked to a polynucleotide encoding Gjb2. The subject may have or be identified as having a mutation in GJB2 and/or GJB6 and may have severe, moderate, or mild hearing loss when treatment is initiated or may be treated prior to symptom onset (e.g., preventative treatment).

In some embodiments, the compositions described herein are used to treat hearing loss associated with a mutation in a gene expressed in GJB2-expressing inner ear cells (e.g., cochlear supporting cells). Hearing loss associated with a mutation in a gene expressed in GJB2-expressing inner ear cells can be treated by administration of a nucleic acid vector containing a GJB2 enhancer having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 59 operably linked to a GJB2 promoter described herein (e.g., a polynucleotide that contains a first region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1 or a functional portion or derivative thereof and/or a second region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or a functional portion or derivative thereof, optionally containing a linker joining the first region and the second region, such as a GJB2 promoter having at least 85% sequence identity to any one of SEQ ID NOs: 29-35), a GJB2 promoter having at least 85% sequence identity (e.g., 85%, 86%, 87%, 88%, 89%, 90%, 91%, 92%, 93%, 94%, 95%, 96%, 97%, 98%, 99%, or more, sequence identity) to any one of SEQ ID NOs: 1, 8-11, and 66-68, or an inner ear cell-type specific promoter (e.g., a promoter listed in Table 9) that is operably linked to a transgene corresponding to a wild-type form of the mutated gene (e.g., a disease listed in Table 6 can be treated by administering a nucleic acid vector having at least 85% sequence identity to SEQ ID NO: 59 operably linked to a GJB2 promoter described herein, a GJB2 promoter having at least 85% sequence identity to any one of SEQ ID NOs: 1, 8-11, and 66-68, or an inner ear cell-type specific promoter (e.g., a promoter listed in Table 9) that is operably linked to a wild-type form of the corresponding gene listed in Table 6). In embodiments in which a composition described herein is used to deliver a polynucleotide listed in Table 6, the composition or method may improve or restore the function and/or structure of the GJB2-expressing inner ear cell (e.g., a cochlear supporting cell) or improve its health or survival.

In some embodiments, the compositions described herein are used to treat subjects having or at risk of developing hearing loss that is associated with damage to or loss of cochlear hair cells (e.g., damage to or loss of cochlear hair cells related to acoustic trauma, disease or infection, head trauma, ototoxic drugs, or aging). Accordingly, the compositions can be used to treat subjects who have been treated with ototoxic drugs or who are currently undergoing or soon to begin treatment with ototoxic drugs. Ototoxic drugs are toxic to the cells of the inner ear, and can cause sensorineural hearing loss, tinnitus, or a combination of these symptoms. Drugs that have been found to be ototoxic include aminoglycoside antibiotics (e.g., gentamycin, neomycin, streptomycin, tobramycin, kanamycin, vancomycin, and amikacin), viomycin, antineoplastic drugs (e.g., platinum-containing chemotherapeutic agents, such as cisplatin, carboplatin, and oxaliplatin), loop diuretics (e.g., ethacrynic acid and furosemide), salicylates (e.g., aspirin, particularly at high doses), and quinine. The disease associated with damage to or loss of cochlear hair cells may be an autoimmune disease or condition in which an autoimmune response contributes to cochlear hair cell damage or death. Autoimmune diseases linked to sensorineural hearing loss include autoimmune inner ear disease (AIED), polyarteritis nodosa (PAN), Cogan's syndrome, relapsing polychondritis, systemic lupus erythematosus (SLE), Wegener's granulomatosis, Sj6gren's syndrome, and Behget's disease. Some infectious conditions, such as Lyme disease and syphilis can also cause hearing loss (e.g., by triggering autoantibody production). Viral infections, such as rubella, cytomegalovirus (CMV), lymphocytic choriomeningitis virus (LCMV), HSV types 1 &2, West Nile virus (WNV), human immunodeficiency virus (HIV) varicella zoster virus (VZV), measles, and mumps, can also cause hearing loss. Hearing loss associated with damage to or loss of cochlear hair cells can be treated by administration of a nucleic acid vector containing a GJB2 enhancer having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 59 operably linked to a GJB2 promoter described herein (e.g., a polynucleotide that contains a first region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1 or a functional portion or derivative thereof and/or a second region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or a functional portion or derivative thereof, optionally containing a linker joining the first region and the second region, such as a GJB2 promoter having at least 85% sequence identity to any one of SEQ ID NOs: 29-35), a GJB2 promoter having at least 85% sequence identity (e.g., 85%, 86%, 87%, 88%, 89%, 90%, 91%, 92%, 93%, 94%, 95%, 96%, 97%, 98%, 99%, or more, sequence identity) to any one of SEQ ID NOs: 1, 8-11, and 66-68, or an inner ear cell-type specific promoter (e.g., a promoter listed in Table 9) that is operably linked to a polynucleotide encoding an expression product that can promote cochlear hair cell regeneration (e.g., a polynucleotide encoding a protein that can promote the differentiation of cochlear supporting cells into cochlear hair cells or the proliferation of cochlear supporting cells, such as a polynucleotide listed in Table 5, or a polynucleotide that can be transcribed to produce an inhibitory RNA that targets a gene known to suppress or prevent cochlear hair cell regeneration, such as LATS1 and/or LATS2).

In some embodiments, the compositions described herein are used to treat sensorineural hearing loss (e.g., acquired sensorineural hearing loss, such as age-related hearing loss, noise-induced hearing loss, ototoxic-drug induced hearing loss, disease or infection-related hearing loss, or head trauma-related hearing loss, e.g., hearing loss associated with damage to or loss of cochlear hair cells). Sensorineural hearing loss can be treated by administration of a nucleic acid vector containing a GJB2 promoter described herein (e.g., a polynucleotide that contains a first region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1 or a functional portion or derivative thereof and/or a second region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or a functional portion or derivative thereof, optionally containing a linker joining the first region and the second region) and/or a GJB2 enhancer described herein (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to any one of SEQ ID NOs: 52-63) (e.g., a GJB2 enhancer operably linked to a promoter, such as a GJB2 promoter or an inner ear cell-type specific promoter) operably linked to a polynucleotide encoding BDNF or NTF3.

The methods described herein may include a step of screening a subject for one or more mutations in genes known to be associated with hearing loss prior to treatment with or administration of the compositions described herein. A subject can be screened for a genetic mutation using standard methods known to those of skill in the art (e.g., genetic testing). The methods described herein may also include a step of assessing hearing in a subject prior to treatment with or administration of the compositions described herein. Hearing can be assessed using standard tests, such as audiometry, auditory brainstem response (ABR), electrocochleography (ECOG), and otoacoustic emissions. These tests can also be used to assess hearing in a subject after treatment with or administration of the compositions described herein. The compositions and methods described herein may also be administered as a preventative treatment to patients at risk of developing hearing loss, e.g., patients who have a family history of hearing loss (e.g., inherited hearing loss), patients carrying a genetic mutation associated with hearing loss who do not yet exhibit hearing impairment, or patients exposed to risk factors for acquired hearing loss (e.g., acoustic trauma, disease or infection, head trauma, ototoxic drugs, or aging).

The compositions and methods described herein can be used to induce or increase cochlear hair cell regeneration in a subject by inducing cochlear supporting cells to differentiate into cochlear hair cells or by inducing the proliferation of cochlear supporting cells. Subjects that may benefit from compositions that induce or increase cochlear hair cell regeneration include subjects suffering from hearing loss as a result of loss of cochlear hair cells (e.g., loss of cochlear hair cells related to trauma (e.g., acoustic trauma or head trauma), disease or infection, ototoxic drugs, or aging), and subjects with abnormal cochlear hair cells (e.g., cochlear hair cells that do not function properly when compared to normal cochlear hair cells), damaged cochlear hair cells (e.g., cochlear hair cell damage related to trauma (e.g., acoustic trauma or head trauma), disease or infection, ototoxic drugs, or aging), or reduced cochlear hair cell numbers due to genetic mutations or congenital abnormalities. Cochlear hair cell regeneration can be induced or increased by contacting a cochlear supporting cell with a nucleic acid vector containing a GJB2 enhancer having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 59 operably linked to a GJB2 promoter described herein (e.g., a polynucleotide that contains a first region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1 or a functional portion or derivative thereof and/or a second region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or a functional portion or derivative thereof, optionally containing a linker joining the first region and the second region, such as a GJB2 promoter having at least 85% sequence identity to any one of SEQ ID NOs: 29-35), a GJB2 promoter having at least 85% sequence identity (e.g., 85%, 86%, 87%, 88%, 89%, 90%, 91%, 92%, 93%, 94%, 95%, 96%, 97%, 98%, 99%, or more, sequence identity) to any one of SEQ ID NOs: 1, 8-11, and 66-68, or an inner ear cell-type specific promoter (e.g., a promoter listed in Table 9) that is operably linked to a polynucleotide encoding a protein that can promote the differentiation of cochlear supporting cells into cochlear hair cells or the proliferation of cochlear supporting cells, such as a polynucleotide listed in Table 5, or a polynucleotide that can be transcribed to produce an inhibitory RNA that targets a gene known to suppress or prevent cochlear hair cell regeneration, such as LATS1 and/or LATS2. The contacting can occur in vivo.

The polynucleotide encoding an expression product this is operably linked to a GJB2 promoter and/or a GJB2 enhancer for treatment of a subject as described herein can be a polynucleotide that encodes a protein expressed in healthy cochlear supporting cells (e.g., a protein that plays a role in cochlear supporting cell development, cochlear supporting cell function, cochlear supporting cell structure, or cochlear supporting cell survival, or a protein encoded by the wild-type version of a cochlear supporting cell gene (e.g., a gene expressed in cochlear supporting cells) that is mutated in a subject with sensorineural hearing loss), a polynucleotide that encodes another protein of interest (e.g., a reporter protein, such as a fluorescent protein, lacZ, or luciferase), a polynucleotide that encodes an expression product that can induce differentiation of cochlear supporting cells into cochlear hair cells or promote cochlear supporting cell proliferation, or a polynucleotide that can be transcribed to produce an shRNA, an ASO, a component of a gene editing system (e.g., a nuclease, such as a CRISPR Associated Protein 9 (Cas9), Transcription Activator-Like Effector Nuclease (TALEN), or Zinc Finger Nuclease (ZFN), or a guide RNA (gRNA)), or a microRNA. The polynucleotide may be selected based on the cause of the subject's hearing loss (e.g., if the subject's hearing loss is associated with a particular genetic mutation, the polynucleotide can be a wild-type form of the gene that is mutated in the subject, or if the subject has hearing loss associated with loss of cochlear hair cells, the polynucleotide can encode a protein that promotes cochlear hair cell regeneration), the severity of the subject's hearing loss, the health of the subject's hair cells, the subject's age, the subject's family history of hearing loss, or other factors.

Treatment may include administration of a composition containing a nucleic acid vector (e.g., an AAV vector) containing a GJB2 promoter and/or a GJB2 enhancer described herein in various unit doses. Each unit dose will ordinarily contain a predetermined quantity of the therapeutic composition. The quantity to be administered, and the particular route of administration and formulation, are within the skill of those in the clinical arts. A unit dose need not be administered as a single injection but may comprise continuous infusion over a set period of time. Dosing may be performed using a syringe pump to control infusion rate in order to minimize damage to the inner ear (e.g., the cochlea and/or vestibular system). In cases in which the nucleic acid vectors are AAV vectors (e.g., AAV1, AAV2, AAV2quad(Y-F), AAV3, AAV4, AAV5, AAV6, AAV7, AAV8, AAV9, AAV10, rh10, rh39, rh43, rh74, Anc80, Anc80L65, DJ, DJ/8, DJ/9, 7m8, PHP.B, PHP.eb, or PHP.S vectors), the viral vectors may be administered to the patient at a dose of, for example, from about 1×10⁹vector genomes (VG)/mL to about 1×10¹⁶VG/mL (e.g., 1×10⁹ VG/mL, 2×10⁹ VG/mL, 3×10⁹ VG/mL, 4×10⁹ VG/mL, 5×10⁹ VG/mL, 6×10⁹ VG/mL, 7×10⁹ VG/mL, 8×10⁹ VG/mL, 9×10⁹ VG/mL, 1×10¹⁰ VG/mL, 2×10¹⁰ VG/mL, 3×10¹⁰ VG/mL, 4×10¹⁰ VG/mL, 5×10¹⁰ VG/mL, 6×10¹⁰ VG/mL, 7×10¹⁰ VG/mL, 8×10¹⁰ VG/mL, 9×10¹⁰ VG/mL, 1×10¹¹ VG/mL, 2×10¹¹ VG/mL, 3×10¹¹ VG/mL, 4×10¹¹ VG/mL, 5×10¹¹ VG/mL, 6×10¹¹ VG/mL, 7×10¹¹ VG/mL, 8×10¹¹ VG/mL, 9×10¹¹ VG/mL, 1×10¹² VG/mL, 2×10¹² VG/mL, 3×10¹² VG/mL, 4×10¹² VG/mL, 5×10¹² VG/mL, 6×10¹² VG/mL, 7×10¹² VG/mL, 8×10¹² VG/mL, 9×10¹² VG/mL, 1×10¹³ VG/mL, 2×10¹³ VG/mL, 3×10¹³ VG/mL, 4×10¹³ VG/mL, 5×10¹³ VG/mL, 6×10¹³ VG/mL, 7×10¹³ VG/mL, 8×10¹³ VG/mL, 9×10¹³ VG/mL, 1×10¹⁴ VG/mL, 2×10¹⁴ VG/mL, 3×10¹⁴ VG/mL, 4×10¹⁴ VG/mL, 5×10¹⁴ VG/mL, 6×10¹⁴ VG/mL, 7×10¹⁴ VG/mL, 8×10¹⁴ VG/mL, 9×10¹⁴ VG/mL, 1×10¹⁵ VG/mL, 2×10¹⁵ VG/mL, 3×10¹⁵ VG/mL, 4×10¹⁵ VG/mL, 5×10¹⁵ VG/mL, 6×10¹⁵ VG/mL, 7×10¹⁵ VG/mL, 8×10¹⁵ VG/mL, 9×10¹⁵ VG/mL, or 1×10¹⁶ VG/mL) in a volume of 1 μL to 200 μL (e.g., 1, 2, 3, 5, 6, 7, 8, 9, 10, 15, 20, 25, 30, 35, 40, 45, 50, 55, 60, 65, 70, 75, 80, 85, 90, 95, 100, 110,120, 130,140,150,160,170, 180, 190, or 200 μL). The AAV vectors may be administered to the subject at a dose of about 1×10⁷ VG/ear to about 2×10¹⁵ VG/ear (e.g., 1×10⁷ VG/ear, 2×10⁷ VG/ear, 3×10⁷ VG/ear, 4×10⁷ VG/ear, 5×10⁷ VG/ear, 6×10⁷ VG/ear, 7×10⁷ VG/ear, 8×10⁷ VG/ear, 9×10⁷ VG/ear, 1×10⁸ VG/ear, 2×10⁸ VG/ear, 3×10⁸ VG/ear, 4×10⁸ VG/ear, 5×10⁸ VG/ear, 6×10⁸ VG/ear, 7×10⁸ VG/ear, 8×10⁸ VG/ear, 9×10⁸ VG/ear, 1×10⁹ VG/ear, 2×10⁹ VG/ear, 3×10⁹ VG/ear, 4×10⁹ VG/ear, 5×10⁹ VG/ear, 6×10⁹ VG/ear, 7×10⁹ VG/ear, 8×10⁹ VG/ear, 9×10⁹ VG/ear, 1×10¹⁰ VG/ear, 2×10¹⁰ VG/ear, 3×10¹⁰ VG/ear, 4×10¹⁰ VG/ear, 5×10¹⁰ VG/ear, 6×10¹⁰ VG/ear, 7×10¹⁰ VG/ear, 8×10¹⁰ VG/ear, 9×10¹⁰ VG/ear, 1×10¹¹ VG/ear, 2×10¹¹ VG/ear, 3×10¹¹ VG/ear, 4×10¹¹ VG/ear, 5×10¹¹ VG/ear, 6×10¹¹ VG/ear, 7×10¹¹ VG/ear, 8×10¹¹ VG/ear, 9×10¹¹ VG/ear, 1×10¹² VG/ear, 2×10¹² VG/ear, 3×10¹² VG/ear, 4×10¹² VG/ear, 5×10¹² VG/ear, 6×10¹² VG/ear, 7×10¹² VG/ear, 8×10¹² VG/ear, 9×10¹² VG/ear, 1×10¹³ VG/ear, 2×10¹³ VG/ear, 3×10¹³ VG/ear, 4×10¹³ VG/ear, 5×10¹³ VG/ear, 6×10¹³ VG/ear, 7×10¹³ VG/ear, 8×10¹³ VG/ear, 9×10¹³ VG/ear, 1×10¹⁴ VG/ear, 2×10¹⁴ VG/ear, 3×10¹⁴ VG/ear, 4×10¹⁴ VG/ear, 5×10¹⁴ VG/ear, 6×10¹⁴ VG/ear, 7×10¹⁴ VG/ear, 8×10¹⁴ VG/ear, 9×10¹⁴ VG/ear, 1×10¹⁵ VG/ear, or 2×10¹⁵ VG/ear).

The compositions described herein are administered in an amount sufficient to improve hearing, reduce tinnitus, increase or induce expression of an expression product in GJB2-expressing cells (e.g., cochlear supporting cells), increase cochlear supporting cell proliferation, promote or increase cochlear supporting cell survival, induce or increase the differentiation of cochlear supporting cells into cochlear hair cells (i.e., cochlear hair cell regeneration), or improve cochlear supporting cell function. Hearing may be evaluated using standard hearing tests (e.g., audiometry, ABR, electrocochleography (ECOG), and otoacoustic emissions) and may be improved by 5% or more (e.g., 5%, 10%, 15%, 20%, 30%, 40%, 50%, 60%, 70%, 80%, 90%, 100%, 125%, 150%, 200% or more) compared to hearing measurements obtained prior to treatment. In some embodiments, the compositions are administered in an amount sufficient to improve the subject's ability to understand speech. The compositions described herein may also be administered in an amount sufficient to slow or prevent the development or progression of sensorineural hearing loss (e.g., in subjects who carry a genetic mutation associated with hearing loss, who have a family history of hearing loss (e.g., hereditary hearing loss), or who have been exposed to risk factors associated with hearing loss (e.g., ototoxic drugs, head trauma, disease or infection, or acoustic trauma) but do not exhibit hearing impairment, or in subjects exhibiting mild to moderate hearing loss). Expression of a protein encoded by a transgene operably linked to a GJB2 promoter and/or a GJB2 enhancer described herein in the nucleic acid vector administered to the subject may be evaluated using immunohistochemistry, Western blot analysis, quantitative real-time PCR, or other methods known in the art for detection protein or mRNA, and may be increased by 5% or more (e.g., 5%, 10%, 15%, 20%, 30%, 40%, 50%, 60%, 70%, 80%, 90%, 100%, 125%, 150%, 200% or more) compared to expression prior to administration of the compositions described herein. Cochlear supporting cell differentiation, cochlear supporting cell function, or function of a protein encoded by a transgene operably linked to a GJB2 promoter and/or a GJB2 enhancer described herein in the nucleic acid vector administered to the subject may be evaluated indirectly based on hearing tests, and may be increased by 5% or more (e.g., 5%, 10%, 15%, 20%, 30%, 40%, 50%, 60%, 70%, 80%, 90%, 100%, 125%, 150%, 200% or more) compared to cochlear supporting cell differentiation, cochlear supporting cell function, or function of the protein prior to administration of the compositions described herein. The compositions and methods described herein may also reduce the toxicity associated with administration of a nucleic acid vector compared to the toxicity observed after the administration of a nucleic acid vector that does not contain a GJB2 promoter and/or a GJB2 enhancer described herein (e.g., administration of a nucleic acid vector in which the same transgene is expressed using a ubiquitous promoter). These effects may occur, for example, within 1 week, 2 weeks, 3 weeks, 4 weeks, 5 weeks, 6 weeks, 7 weeks, 8 weeks, 9 weeks, 10 weeks, 15 weeks, 20 weeks, 25 weeks, or more, following administration of the compositions described herein. The patient may be evaluated 1 month, 2 months, 3 months, 4 months, 5 months, 6 months, or more following administration of the composition depending on the dose and route of administration used for treatment. Depending on the outcome of the evaluation, the patient may receive additional treatments.

### Kits

The compositions described herein can be provided in a kit for use in treating sensorineural hearing loss. Compositions may include a GJB2 promoter described herein (e.g., a polynucleotide that contains a first region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1 or a functional portion or derivative thereof and/or a second region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or a functional portion or derivative thereof, optionally containing a linker joining the first region and the second region), a nucleic acid vector containing such a polynucleotide, or a nucleic acid vector containing a GJB2 promoter described herein operably linked to a polynucleotide encoding an expression product (e.g., a transgene encoding a protein of interest, such as a protein that can be expressed in GJB2-expressing inner ear cells to treat hearing loss). The GJB2 promoter in any of the foregoing compositions may be operably linked to a GJB2 enhancer described herein. Compositions may also include one or more GJB2 enhancers described herein (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to any one of SEQ ID NOs: 52-63), a nucleic acid vector containing such a polynucleotide, or a nucleic acid vector containing a GJB2 enhancer described herein operably linked to promoter (e.g., a GJB2 promoter or an inner ear cell-type specific promoter) that is operably linked to a polynucleotide encoding an expression product (e.g., a transgene encoding a protein of interest, such as a protein that can be expressed in GJB2-expressing inner ear cells to treat hearing loss). The nucleic acid vector may be packaged in an AAV virus capsid (e.g., AAV1, AAV2, AAV2quad(Y-F), AAV6, AAV8, AAV9, Anc80, Anc80L65, AAV-DJ, DJ/9, 7m8, or PHP.B). The kit can further include a package insert that instructs a user of the kit, such as a physician, to perform the methods described herein. The kit may optionally include a syringe or other device for administering the composition.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regard as their invention.

### Example 1. GJB2 Expression Using a Ubiquitous Promoter LED to Elevated Hearing Thresholds and Inner Hair Cell Death in Wild-Type Mice

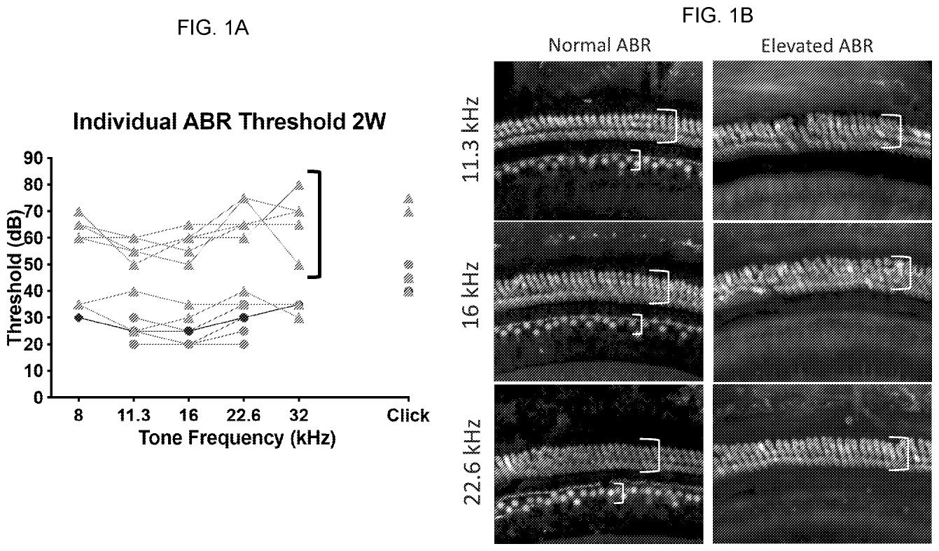

Six- to eight-week-old C57BL/6J mice were injected with an AAV vector including the murine Gjb2 coding sequence driven by a ubiquitous promoter (AAV-CMV-mGjb2) via fenestration in the posterior semicircular canal. After two weeks, animals were anesthetized with ketamine and xylazine and the auditory brainstem response (ABR) was measured. Following ABR measurements, the animals were sacrificed and fixed in 10% NBF via cardiac perfusion and their temporal bones were harvested and kept in 10% NBF for an additional 16 hours. After two days decalcification in 8% EDTA, ears were microdissected and utricles and organs of Corti prepared for immunohistochemistry. Whole mount tissue preparations of the organ of Corti were counterstained with Myosin7a antibody to visualize all hair cells and imaged under a confocal microscope (Leica SP8). Seven out of nine wild-type ears treated with AAV-CMV-Gjb2 showed elevated ABR thresholds (FIG. 1A, bracket). As shown in FIG. 1B, animals with elevated hearing thresholds lacked inner hair cells.

### Example 2. Evaluation of Luciferase Expression Driven by GJB2 Promoters in HeLa Cells

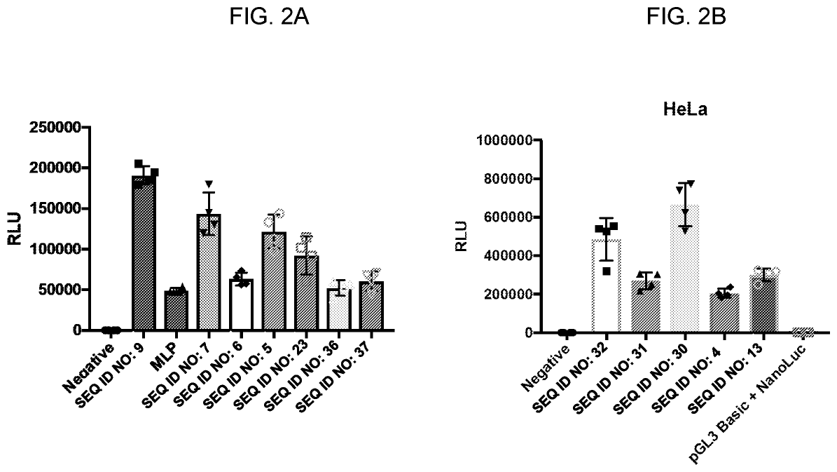

To test for the ability of various GJB2 promoters to drive expression, GJB2 promoters having the sequences of SEQ ID NOs: 4-7, 9, 13, 30-32, 36, and 37 were cloned upstream of a NanoLuc reporter. HeLa cells were transfected with plasmids containing the promoter and reporter. Twenty-four hours later, the Nano-Glo Luciferase Assay (Promega Catalog #N1110) was used to detect and quantify NanoLuc expression. Results are shown in FIGS. 2A-2B. MLP=control minimal promoter (unrelated to GJB2). pGL3 Basic+NanoLuc=control vector without a promoter.

### Example 3. Bashed GJB2 Promoters Alone and Paired with GJB2 Enhancers Drive Increased Expression and Cell Type Specificity in Murine Cochlear Explants

Cochlea from P0-P5 pups were dissected and cultured on collagen matrices in DMEM supplemented with 5% FBS and penicillin-G. Organ of Corti explant cultures were established and infected with AAVs expressing nuclear (H2B)-GFP under the control of various GJB2 promoter and enhancer combinations. 2e10 vg/culture was added to the culture media. Samples were incubated in the presence of virus for 72 hours and then fixed for fluorescence imaging. All samples were counterstained with an anti-Myo7a antibody (hair cells), anti-Tuj1 antibody (neurons), an anti-GJB2 antibody, and an anti-GFP antibody and imaged on a Zeiss 810 confocal microscope.

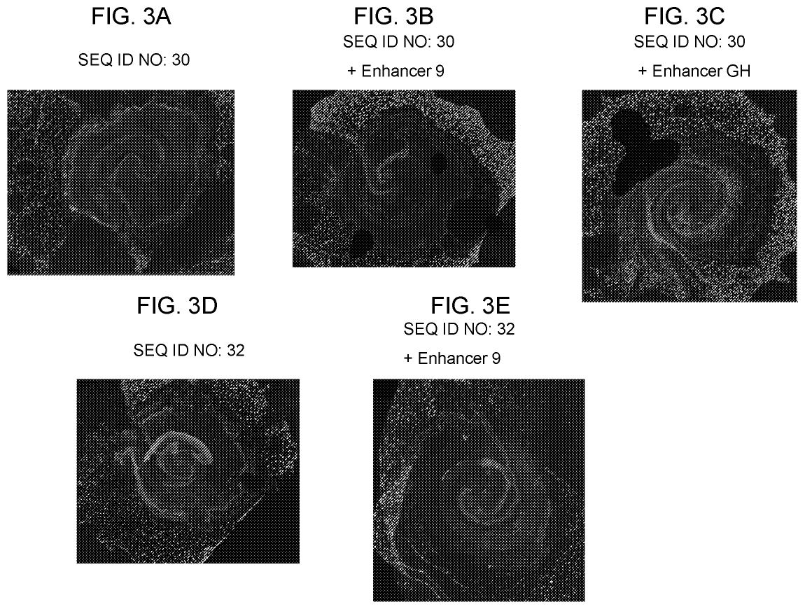

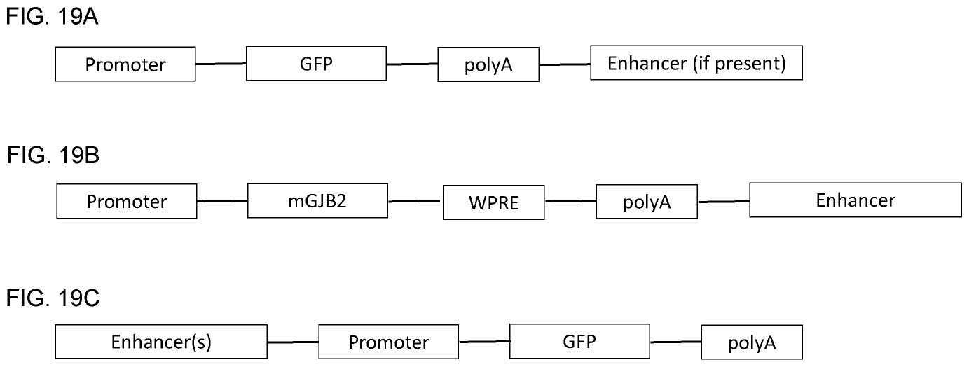

In a first experiment, samples infected with vectors containing a GJB2 promoter alone (AAV1-p.hGJB2(SEQ ID NO: 30)-H2B-GFP (FIG. 3A) and AAV1-p.hGJB2(SEQ ID NO: 32)-H2B-GFP (FIG. 3D)) showed lower expression of the reporter protein compared to samples infected with a vector containing a GJB2 promoter and an enhancer (AAV1-p.hGJB2(SEQ ID NO: 30)-H2B-GFP-Enhancer 9 (SEQ ID NO: 62) (FIG. 3B), AAV1-p.hGJB2(SEQ ID NO: 30)-H2B-GFP-Enhancer GH (SEQ ID NO: 60) (FIG. 3C), and AAV1-p.hGJB2(SEQ ID NO: 32)-H2B-GFP-Enhancer 9 (SEQ ID NO: 62) (FIG. 3E)). The enhancers were positioned 3′ of the coding sequence in the vectors. A schematic depicting the order of the elements in the vectors used in the experiments of Example 3 is provided in FIG. 19A.

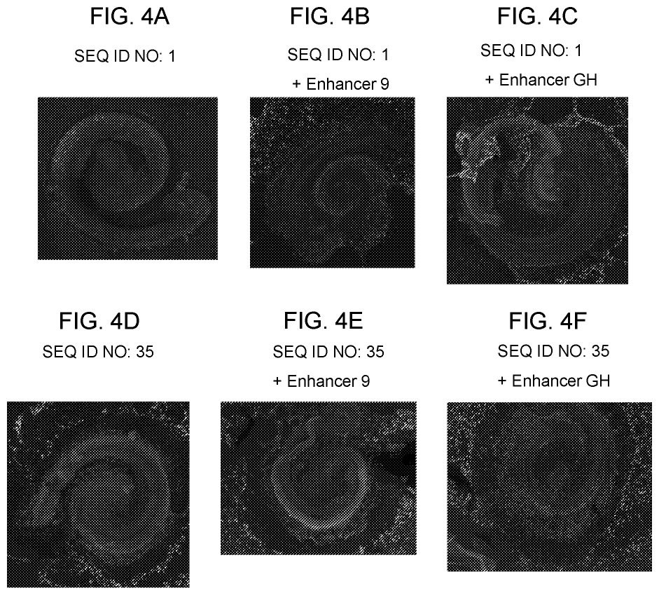

In a second experiment, samples infected with a vector containing a GJB2 promoter alone AAV1-p.hGJB2(SEQ ID NO: 1)-H2B-GFP showed the lowest expression of the reporter protein (FIG. 4A). Adding a Splice Acceptor (SEQ ID NO: 22) produced a promoter (SEQ ID NO: 35) that resulted in increased expression (AAV1-p.hGJB2(SEQ ID NO: 35)-H2B-GFP, FIG. 4D). For both the promoter and promoter+spice acceptor, the addition of enhancer elements showed the highest levels of reporter protein expression (AAV1-p.hGJB2(SEQ ID NO: 1)-H2B-GFP-Enhancer 9 (SEQ ID NO: 62) (FIG. 4B), AAV1-p.hGJB2(SEQ ID NO: 1)-H2B-GFP-Enhancer GH (SEQ ID NO: 60) (FIG. 4C), AAV1-p.hGJB2(SEQ ID NO: 35)-H2B-GFP-Enhancer 9 (SEQ ID NO: 62) (FIG. 4E), and AAV1-p.hGJB2(SEQ ID NO: 35)-H2B-GFP-Enhancer GH (SEQ ID NO: 60) (FIG. 4F)). The enhancers were positioned 3′ of the coding sequence in the vectors.

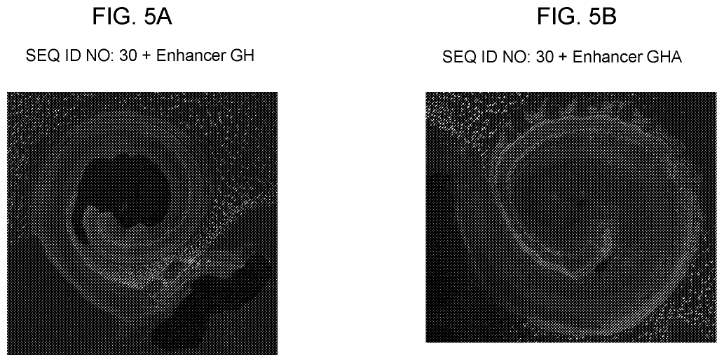

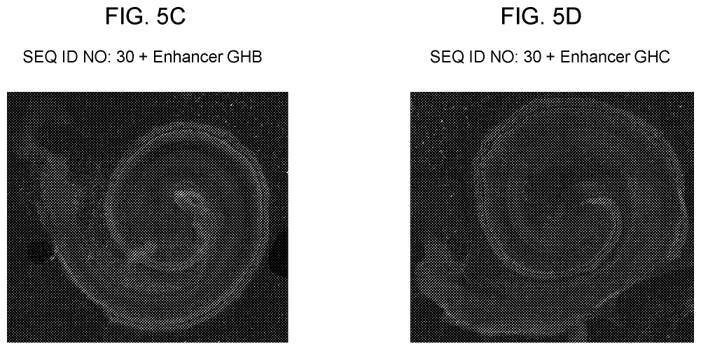

In a third experiment, truncations of Enhancer GH were tested. Explant cultures infected with AAV1-p.hGJB2(SEQ ID NO: 30)-H2B-GFP-Enhancer GH (SEQ ID NO: 60) (FIG. 5A) or AAV1-p.hGJB2(SEQ ID NO: 30)-H2B-GFP-Enhancer GHA (SEQ ID NO: 61) (FIG. 5B) retained strong reporter expression, whereas truncations of Enhancer GH (AAV1-p.hGJB2(SEQ ID NO: 30)-H2B-GFP-Enhancer GHB (SEQ ID NO: 64) (FIG. 5C), and AAV1-p.hGJB2(SEQ ID NO: 30)-H2B-GFP-Enhancer GHC (SEQ ID NO: 65) (FIG. 5D)) lost enhancer activity. The enhancers were positioned 3′ of the coding sequence in the vectors.

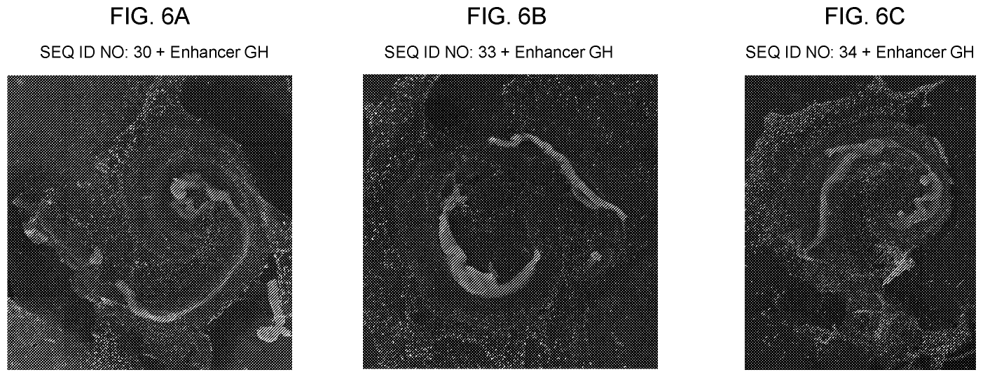

In a fourth experiment, the GJB2 promoter of SEQ ID NO: 30 was modified by the addition of DNA elements corresponding to a predicted histone mark found within intron 1 of the GJB2 locus. Similar levels of reporter protein were detected in all samples (AAV1-p.hGJB2(SEQ ID NO: 30)-H2B-GFP-Enhancer GH (SEQ ID NO: 60) (FIG. 6A), AAV1-p.hGJB2(SEQ ID NO: 33)-H2B-GFP-Enhancer GH (SEQ ID NO: 60) (FIG. 6B) AAV1-p.hGJB2(SEQ ID NO: 34)-H2B-GFP-Enhancer GH (SEQ ID NO: 60) (FIG. 6C)) indicating the methylated DNA (histone mark) does not influence expression. Enhancers were positioned 3′ of the coding sequence in the vectors.

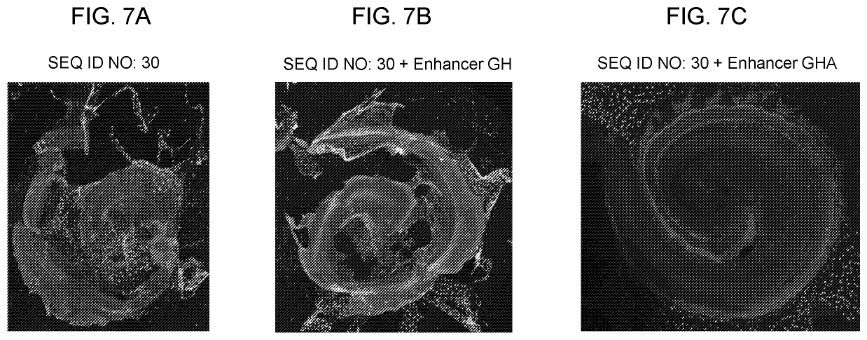

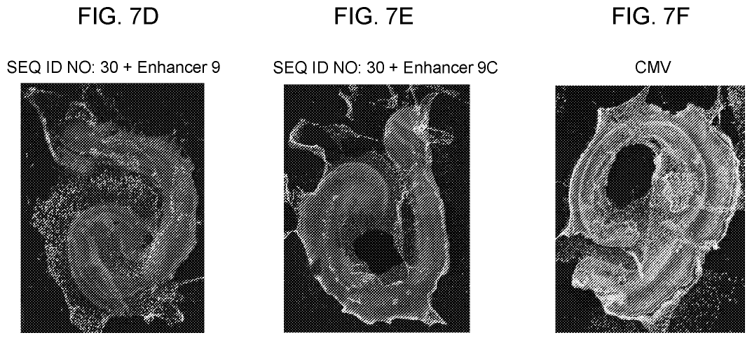

In a fifth experiment, the GJB2 promoter of SEQ ID NO: 30 was tested in combination with different enhancers (full-length and truncated enhancers). Samples infected with the combination of the GJB2 promoter and the full-length enhancers (Enhancer GH and Enhancer 9) showed higher expression of the reporter protein (AAV1-p.hGJB2(SEQ ID NO: 30)-H2B-GFP-Enhancer GH (SEQ ID NO: 60) (FIG. 7B) and AAV1-p.hGJB2(SEQ ID NO: 30)-H2B-GFP-Enhancer 9 (SEQ ID NO: 62) (FIG. 7D)) when compared to the combinations containing the truncated enhancers (AAV1-p.hGJB2(SEQ ID NO: 30)-H2B-GFP-Enhancer GHA (SEQ ID NO: 61) (FIG. 7C) and AAV1-p.hGJB2(SEQ ID NO: 30)-H2B-GFP-Enhancer 9C (SEQ ID NO: 63) (FIG. 7E)). Expression of the reporter protein was observed in all cells in the control sample (AAV1-CMV-H2B-GFP, FIG. 7F). Enhancers were positioned 3′ of the coding sequence in the vectors.

Promoter activity observed in these experiments is summarized in Table 10, below.

TABLE 10
GJB2 promoter activity

| | | Ex- | Ex- | | |
|---|---|---|---|---|---|
| | | pression | pression | Ex- | |
| | | in inner | in lateral | clusion | Level |
| | | support- | support- | from | of |
| | | ing | ing | hair | ex- |
| Promoter | Enhancer | cells | cells | cells | pression |
| SEQ ID NO: 30 | − | − | + | + | Low |
| SEQ ID NO: 30 | Enhancer 9 | − | + | + | Moderate |
| SEQ ID NO: 30 | Enhancer GH | + | + | + | High |
| SEQ ID NO: 32 | − | − | + | + | Low |
| SEQ ID NO: 32 | Enhancer 9 | − | + | + | Low |
| SEQ ID NO: 1 | − | − | − | + | Minimal |
| SEQ ID NO: 1 | Enhancer 9 | − | + | + | Very |
| | | | | | Low |
| SEQ ID NO: 1 | Enhancer GH | + | + | + | Low |
| SEQ ID NO: 35 | − | − | + | + | Minimal |
| SEQ ID NO: 35 | Enhancer 9 | − | + | + | Low |
| SEQ ID NO: 35 | Enhancer GH | + | + | + | Moderate |
| SEQ ID NO: 30 | Enhancer GHA | + | + | + | Moderate |
| SEQ ID NO: 30 | Enhancer GHB | − | + | + | Minimal |
| SEQ ID NO: 30 | Enhancer GHC | − | + | + | Minimal |
| SEQ ID NO: 30 | Enhancer 9C | − | + | + | Moderate |
| SEQ ID NO: 33 | Enhancer GH | + | + | + | Low |
| SEQ ID NO: 34 | Enhancer GH | + | + | + | Low |
| CMV | − | + | + | − | Highest |

For level of expression: minimal < very low < low < moderate < high < highest

### Example 4. GJB2 Promoter/Enhancer Driven GFP Expression is Enriched In Vivo in Murine GJB2-Expressing Cells

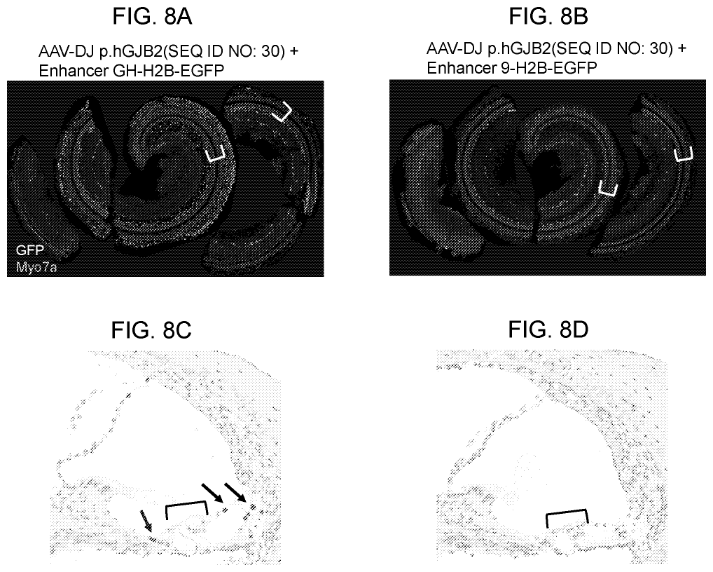

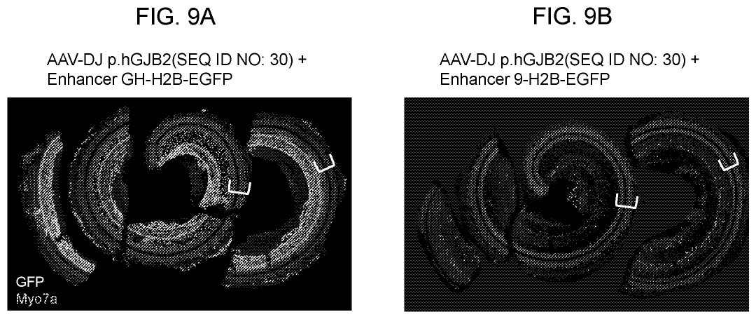

To test the specificity of the GJB2 promoter of SEQ ID NO: 30 in combination with different enhancers disclosed herein in vivo, wildtype C57BL/6J mice were injected with an AAV vector including eGFP operably linked to the GJB2 promoter of SEQ ID NO: 30 and a GJB2 enhancer described herein. In some instances, 1 μL of AAV vector was injected into early postnatal mice, two to three days after birth via fenestration in the posterior semicircular canal (see FIGS. 9A-91B, FIGS. 10A-10B, FIGS. 11A-11B, FIGS. 12A-12B, FIGS. 13A-13B). In other instances, 1 μL of AAV vector was injected into adult, 6- to 8-week-old animals via fenestration in the posterior semicircular canal (see FIGS. 8A-8D).

After four weeks in life, animals were sacrificed and fixed in 10% NBF via cardiac perfusion, their temporal bones were harvested and kept in 10% NBF for an additional 16 hours. After two days decalcification in 8% EDTA, ears were micro dissected and utricles and organs of Corti prepared for immunohistochemistry. Whole mount tissue preparations of the organ of Corti were counterstained with Myosin7a or Pou4f3 antibody to visualize all hair cells and imaged together with the virally mediated native GFP expression under a confocal microscope (Leica SP8, 20×/0.75 NA, 2 μm step size at 2 AU).

To visualize GFP expression in cross-sections of the murine cochleae, fixed and decalcified microdissected temporal bones were embedded in paraffin blocks, then mid-modiolar sections were obtained. Sections were baked overnight at 65° C., deparaffinization and tissue hydration was performed with Bound Dewax Solution for 30 seconds. In addition, antigen retrieval via 94° C. treatment for 25 min in pH 9.0 EDTA was leveraged. Primary antibody incubation against GFP (Abcam #183734) was performed for 15 min at room temperature, followed by a secondary antibody incubation of 30 min at room temperature, and 10 min+5 min room temperature incubation for Red Refine (secondary antibody and chromogenic reagent: BOUND Polymer Refine Red Detection). Finally, tissue was counterstained using hematoxylin for 7 min at room temperature.

To visualize GFP expression in the lateral wall and stria portions of the cochlea, the lateral wall of the cochlea was removed during cochlear dissection by slicing the tissue along the outer sulcus where the base of the spiral prominence meets the organ of Corti. Following detachment of the lateral wall, Reissner's membrane was removed using fine forceps. For immunohistochemistry, the segments of the lateral wall were incubated in 50 mM glycine/0.01 M phosphate buffered saline (PBS) for 30 minutes at room temperature. Next, tissue was washed in 0.1 M PBS 2×10 minutes. Tissue was then permeabilized with a blocking solution (5% bovine serum albumin, 5% normal donkey serum, 0.5% Tx-100, 0.01 M PBS) for 1 hr at RT. Next, tissue was incubated in DAPI solution (2 μg/mL) for 10 minutes at room temperature followed by washes in 0.1 M PBS 2×10 minutes each. Finally, tissue was mounted and coverslipped in SlowFade Diamond Antifade Media (S36967). Lateral wall segments were scanned using a Zeiss LSM800 confocal microscope. Z-stacks were acquired using a 40×/1.4 oil-immersion objective at a resolution of 0.3×0.3×1.0 μm per voxel.

In an AAV-DJ vector, Enhancer GH induced more GFP expression in adult mouse cochlea compared to Enhancer 9 (FIGS. 8A-8B). Cross-section of adult mouse cochlea confirmed that GFP expression was restricted to supporting cells (GFP+ nuclei, dark grey; black arrows) and excluded from hair cells in the sensory epithelium (bracket) (FIGS. 8C-8D). Enhancer GH also induced more GFP expression in neonatal mouse cochlea compared to Enhancer 9 in an AAV-DJ vector (FIGS. 9A-9B).

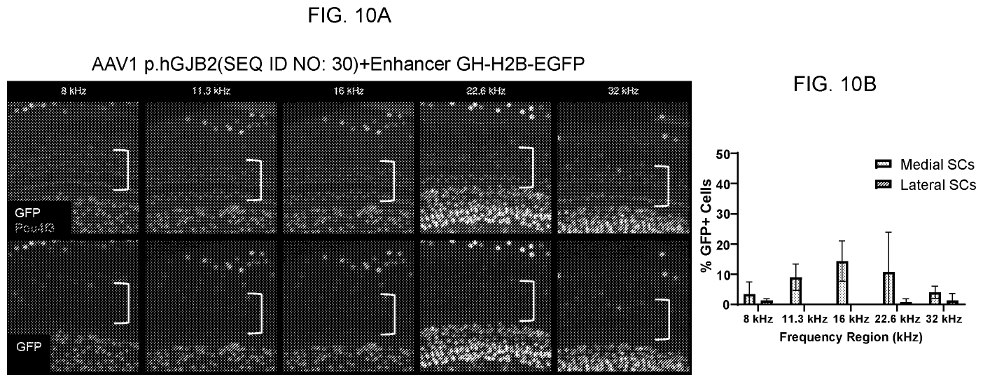

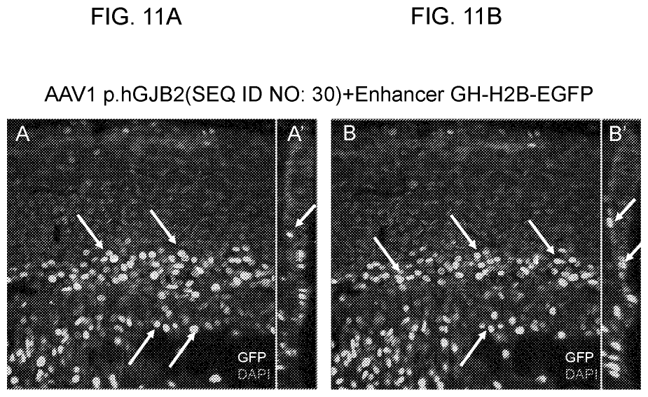

In an AAV1 vector, Enhancer GH induced GFP expression in supporting cells of the neonatal mouse ear (FIG. 10A, GFP, white; Pou4f3 hair cells, light grey). GFP expression was excluded from hair cells in the sensory epithelium (bracket). Quantification of GFP+ supporting cells shows more GFP expression in medial supporting cells compared to lateral supporting cells (FIG. 10B). Enhancer GH also induced GFP expression in the lateral wall and stria vascularis (FIGS. 11A-11B).

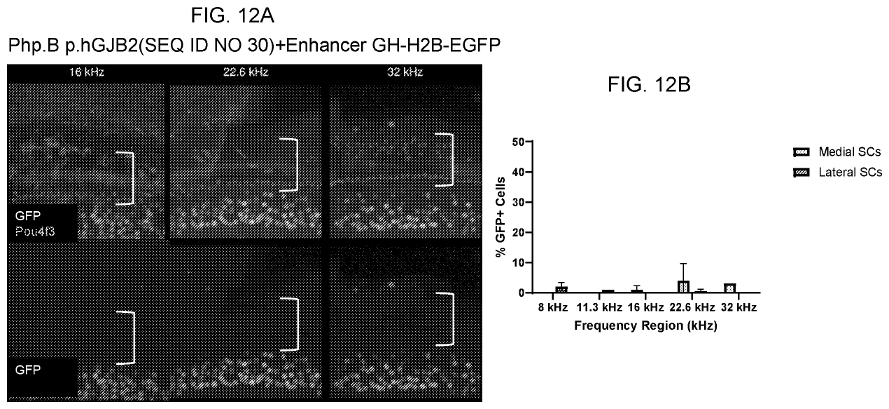

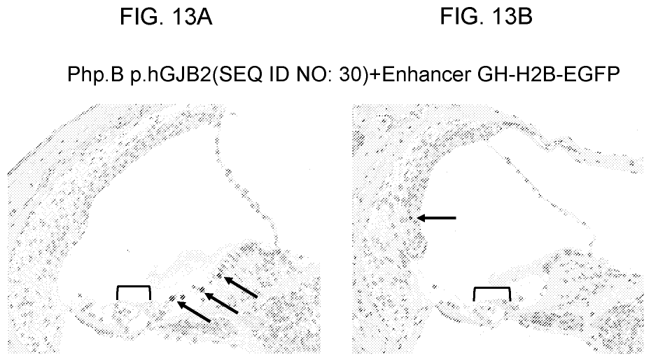

In a Php.B vector, Enhancer GH induced GFP expression in supporting cells of the neonatal mouse ear (FIG. 12A, GFP, white; Pou4f3 hair cells, light grey). GFP expression was excluded from hair cells in the sensory epithelium (bracket). FIG. 12B shows the quantification of GFP+ supporting cells. Cross-sections of mouse cochlea show GFP expression was restricted to supporting cells and the lateral wall (FIGS. 13A-13B; GFP+ nuclei, dark grey; black arrows) and excluded from hair cells in the sensory epithelium (bracket). A schematic depicting the order of the elements in the vectors used in the experiments of Example 4 is provided in FIG. 19A.

### Example 5. Expression of GJB2 Driven by a GJB2 Promoter/Enhancer Pair Leads to Hearing Recovery and Outer Hair Cell Retention in a Mouse Model of GJB2 Deficiency

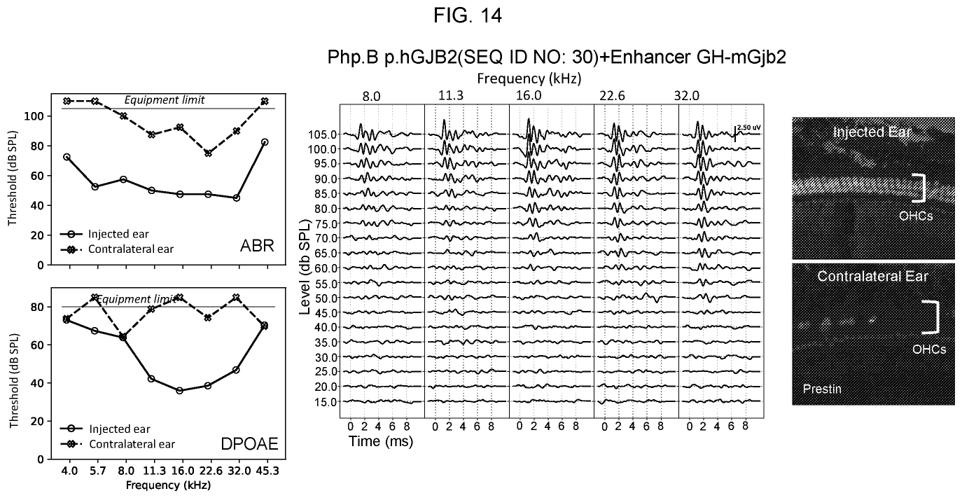

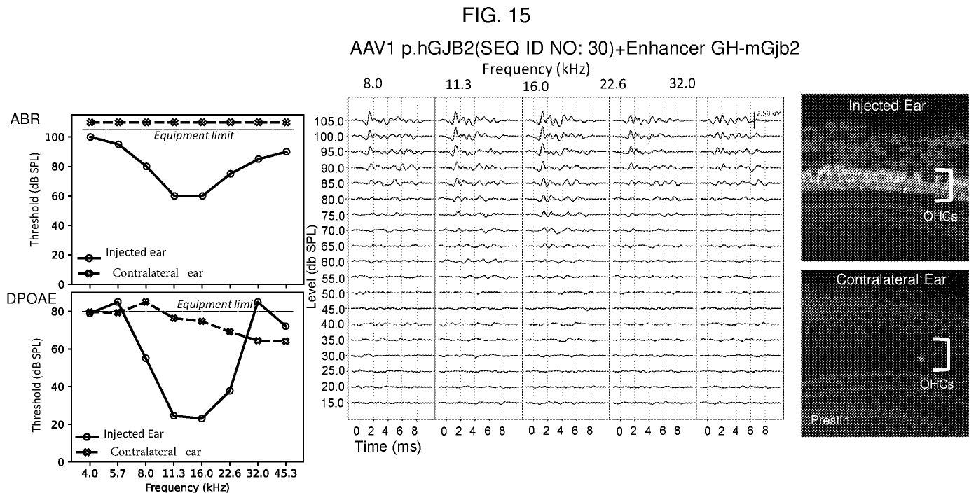

Neonatal GJB6-LacZ (GJB2-deficient mouse model) mice were injected unilaterally two to three days after birth via fenestration in the posterior semicircular canal with 1 μl of vector encoding murine Gjb2 driven by a GJB2 promoter/enhancer combination (AAV-p.hGJB2(SEQ ID NO: 30)+Enhancer GH-mGjb2). Four weeks later, animals were anesthetized with ketamine and xylazine and the auditory brainstem response (ABR) and distorted product otoacoustic emission (DPOAE) were measured in both ears (injected and uninjected) to test for hearing restoration. The animals were then sacrificed and fixed in 10% NBF via cardiac perfusion, and whole mount histology of the organ of Corti was performed as described in Example 4, omitting GFP, to evaluate the integrity of the hair cells. As shown in FIGS. 14 and 15, AAVPhp.B-p.hGJB2(SEQ ID NO: 30)+Enhancer GH-mGjb2 and AAV1-p.hGJB2(SEQ ID NO: 30)+Enhancer GH-mGjb2 restored hearing and prevented outer hair cell loss in a gene replacement gene therapy in a GJB2-deficient mouse model. Similar results were observed using an AAV-DJ serotype vector. A schematic depicting the order of the elements in the vectors used in the experiments of Example 5 is provided in FIG. 19B.

### Example 6. Characterization of Additional GJB2 Enhancers in Neonatal Mice

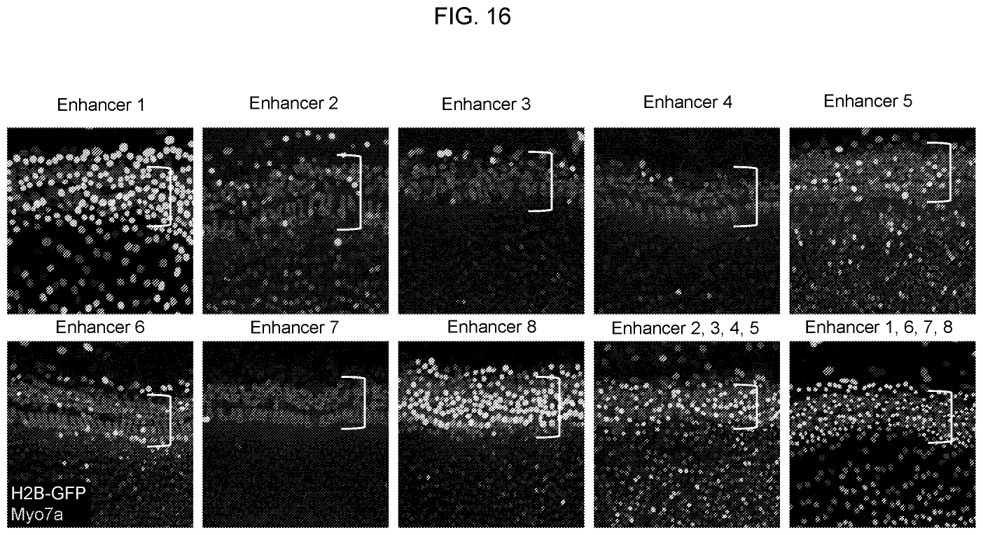

In a first experiment, the GJB2 promoter of SEQ ID NO: 30 was combined with one or more of Enhancers 1-8 (Enhancer 1: SEQ ID NO: 52; Enhancer 2: SEQ ID NO: 53; Enhancer 3: SEQ ID NO: 54; Enhancer 4: SEQ ID NO: 55; Enhancer 5: SEQ ID NO: 56; Enhancer 6: SEQ ID NO: 57; Enhancer 7: SEQ ID NO: 58; Enhancer 8: SEQ ID NO: 59) to evaluate GFP expression in neonatal cochlear explants. Enhancers driving histone-tagged GFP were screened in explants using AAV-DJ vectors. The enhancers were positioned 5′ of the GJB2 promoter in the vectors. A schematic depicting the order of the elements in the vectors used in the experiments of Example 6 is provided in FIG. 19C. Neonatal cochlear explants were established from P0-P2 wildtype C57BL/6 mice. Briefly, neonatal mice were euthanized, inner ears were removed from the head and the cochlea was carefully removed using microdissection. The modiolus was removed from the center of the coil and the roof of the cochlear duct was removed exposing the sensory epithelium. Cochlear explants were plated, lumenal side of the cochlear duct facing up, on prepared 3D rat tail collagen matrices. Cochlear explant culture media consisting of DMEM with 7% FBS and 1 U/μL penicillin G was added directly to the culture. Virus containing the GJB2 promoter of SEQ ID NO: 30 and one or more GJB2 enhancers (one or more of SEQ ID NOs: 52-59) driving GFP expression was added to the media at a concentration of 5e11 vgs/culture. Cultures were incubated for 48 hours with virus, followed by 72 hours in fresh media in standard culture conditions of 5% CO2 at 37° C. Cultures were then fixed in 4% formaldehyde, permeabilized with 0.01% TritonX100, and blocked with 10% normal donkey serum before staining with anti-Myo7a antibody (hair cells). Samples were imaged (Myo7a, native GFP) using confocal microscopy (Zeiss LSM 810) using uniform settings (laser intensity and gain) between samples. Expression pattern, GFP intensity, and exclusion from hair cells were assessed. High GFP expression was observed with Enhancer 1, Enhancer, 8, and the two enhancer combinations (Enhancers 1+6+7+8 and Enhancers 2+3+4+5). Complete exclusion from hair cells was observed with Enhancers 1, 4, 5, and 6 and with the two enhancer combinations. GFP was observed in the sensory epithelium region for all enhancers and enhancer combinations tested with the exception of Enhancer 7. The results are shown in FIG. 16. The sensory epithelium is marked with a bracket (GFP, white; Myo7a, gray).

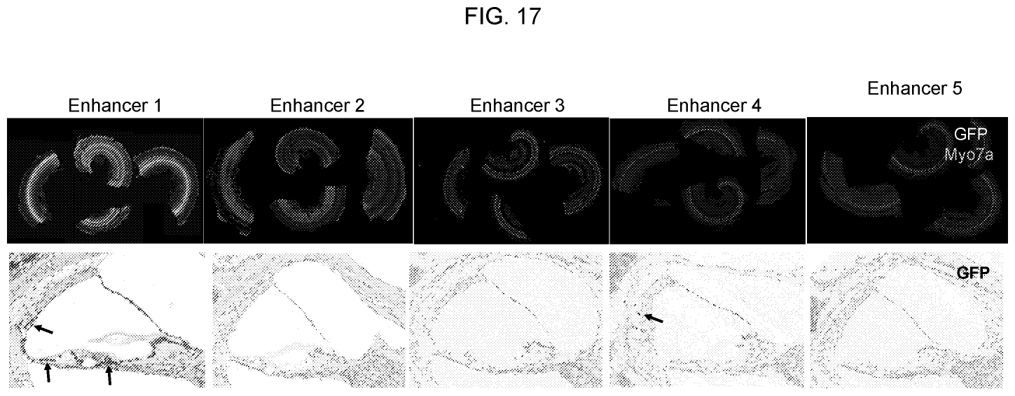

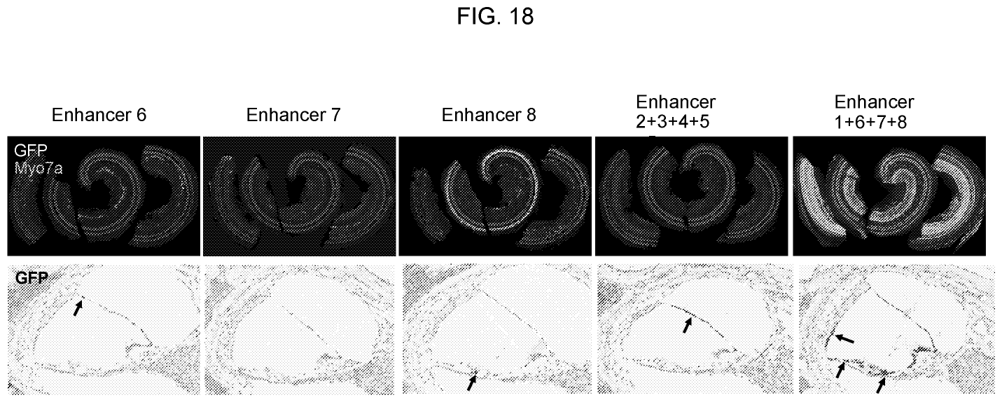

In a second experiment, the same vectors evaluated in the neonatal cochlear explants were injected into early postnatal C57BL/6J mice, two to three days after birth via fenestration in the posterior semicircular canal as described above in Example 4 to evaluate GFP expression in vivo. Whole mount tissue preparations of the organ of Corti and cross-sections of the murine cochleae were also prepared and imaged as described in Example 4. In Enhancer 1, GFP expression was present in all supporting cells of the sensory epithelium and many non-sensory cell types (top row, FIG. 17). GFP was excluded from hair cells for all enhancers tested (GFP, white; Myo7a hair cells, grey). GFP was detected in the lateral wall with Enhancer 4. Section histology confirms whole mount expression (bottom row; GFP, dark grey; black arrows indicate examples of GFP+ cells). In Enhancer 8, GFP expression was restricted to supporting cells of the sensory epithelium (top row, FIG. 18). GFP was excluded from hair cells for all enhancers tested. GFP expression was observed in Reissner's membrane for Enhancer 6 and the combination of Enhancers 2+3+4+5. In the combination of Enhancers 1+6+7+8, GFP expression was present in all supporting cells of the sensory epithelium, most non-sensory cells of the cochlear duct, cells of the lateral wall and stria vascularis (top row, FIG. 18; GFP, white; Myo7a hair cells, grey). Section histology confirms whole mount expression (bottom row; GFP, dark grey; black arrows indicate examples of GFP+ cells). The same promoter/enhancer combinations were tested using AAV1 vectors and similar results were observed.

### Example 7. GJB2 Expression Using a GJB2 Promoter/Enhancer Pair Preserved Hearing Thresholds and Hair Cells in Wild-Type Mice, as Compared to GJB2 Expression Using a Ubiquitous Promoter, which LED to Elevated Hearing Thresholds and Inner Hair Cell Death

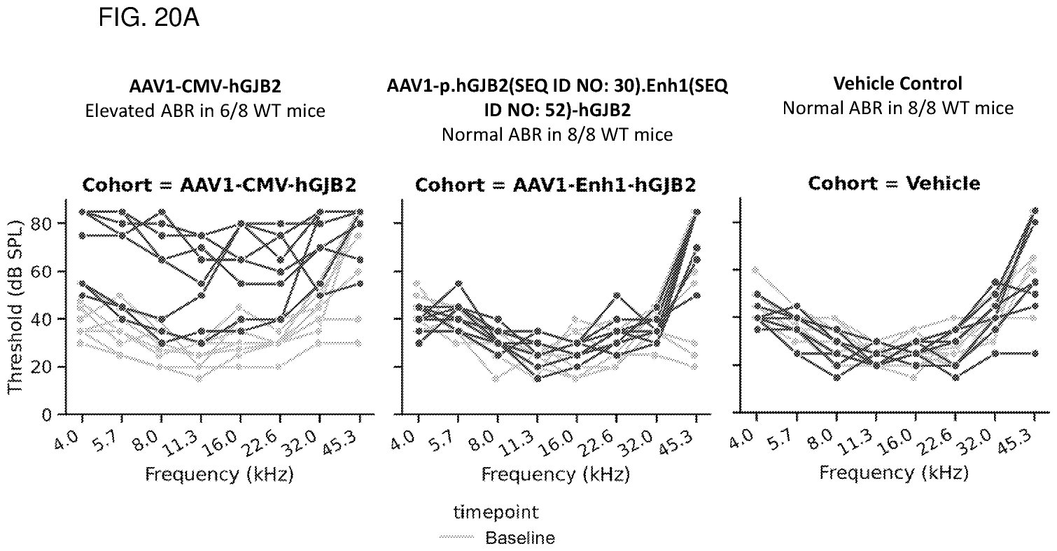

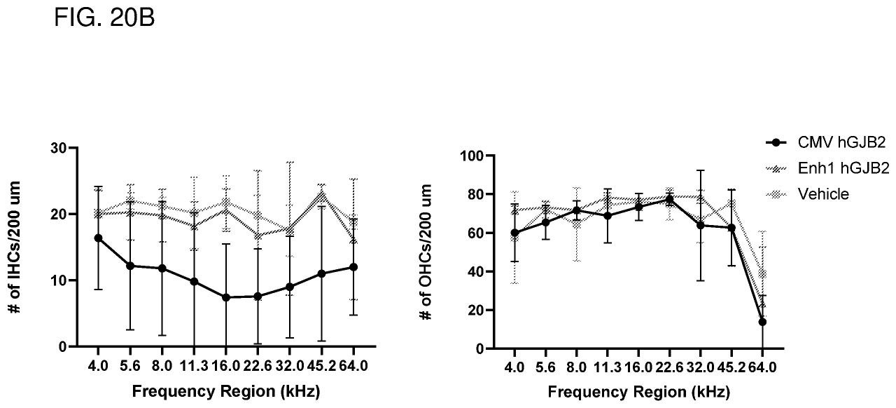

Six- to eight-week-old C57BL/6J mice were injected with a vehicle control, or an AAV1 vector including the human GJB2 coding sequence driven by a ubiquitous promoter (AAV-CMV-hGJB2) or driven by a GJB2 enhancer/promoter pair (AAV-p.hGJB2(SEQ ID NO: 30)+Enhancer 1(SEQ ID NO: 52)), via fenestration in the posterior semicircular canal. Enhancer 1 was positioned 5′ of the promoter in the vector. Hearing measurements, takedowns, and histological processing of the tissue was performed after two weeks as described in Example 1. Six out of eight wild-type ears treated with AAV-CMV-GJB2 showed elevated ABR thresholds, while hearing thresholds of animals treated with the vehicle control or the human GJB2 coding sequence driven by the GJB2 enhancer/promoter pair were comparable to baseline measurements (FIG. 20A). As shown in FIG. 20B, animals treated with AAV-CMV-GJB2 had inner hair cell loss, while hair cells were preserved in the other groups. This demonstrates that GJB2 overexpression using GJB2 regulatory elements that exclude expression from hair cells is safe.

### Example 8. Expression of GJB2 Driven by a GJB2 Promoter/Enhancer Combination LED to Hearing Recovery and Outer Hair Cell Retention in a Mouse Model of GJB2 Deficiency

Hearing recovery was assessed in a GJB2-deficient mouse model using methods similar to those described in Example 5 with the following modifications:

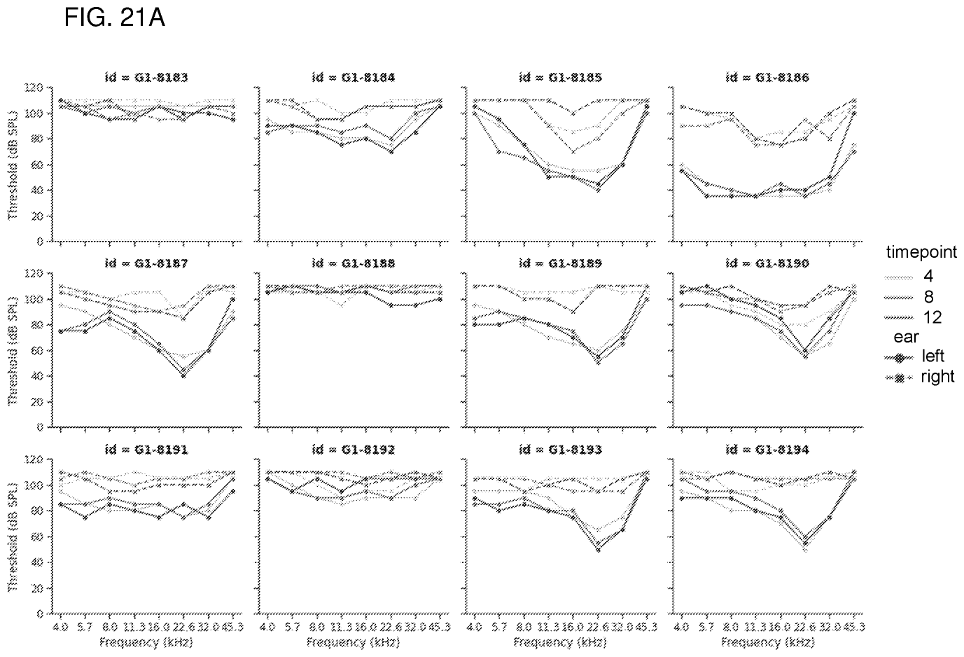

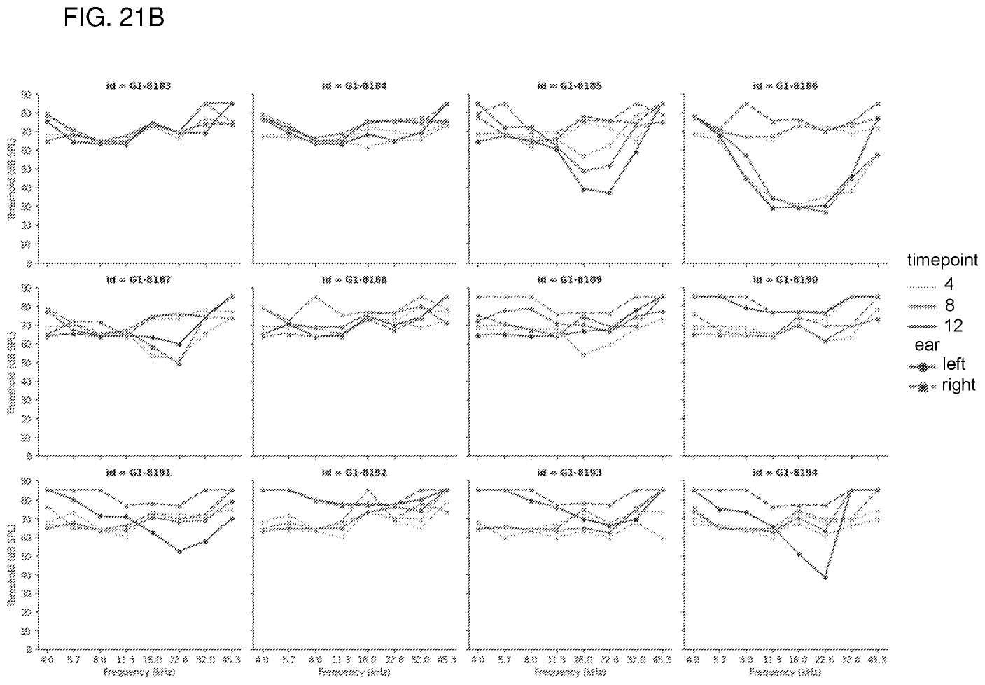

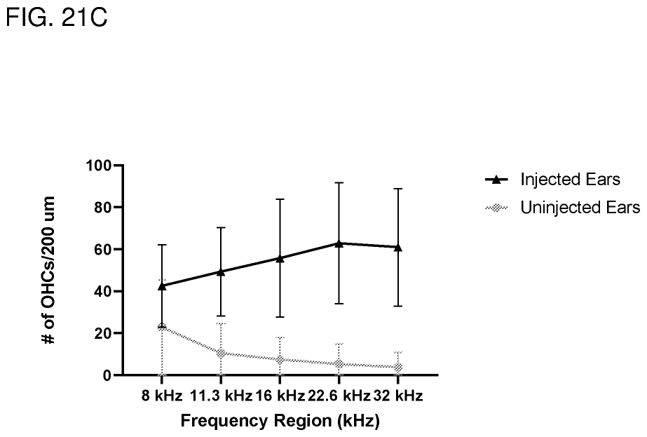

In a first experiment, animals were injected with AAV1 encoding human GJB2 driven by p.hGJB2(SEQ ID NO: 30)+Enhancer 1(SEQ ID NO: 52). Enhancer 1 was positioned 5′ of the promoter in the vector. Hearing measurements were performed at 4, 8, and 12 weeks post-injection, after which animals were taken down. The results of this experiment are shown in FIGS. 21A-21C.

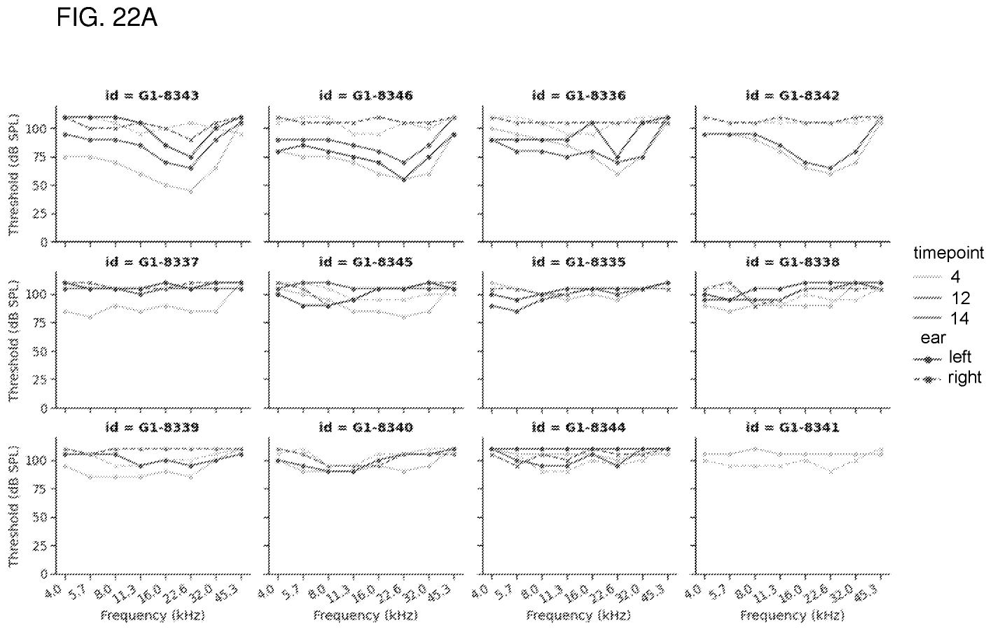

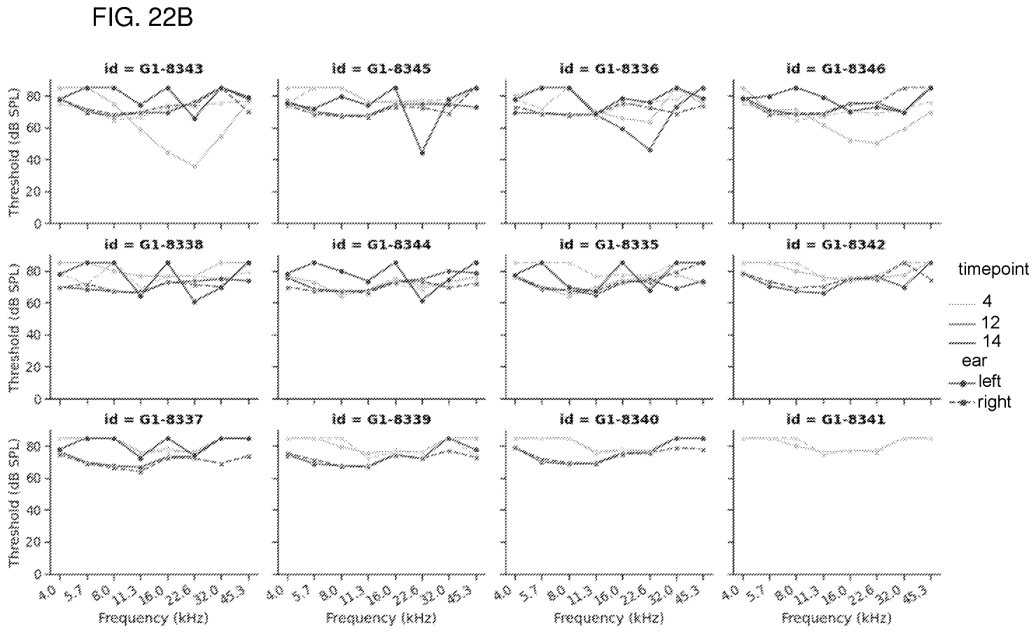

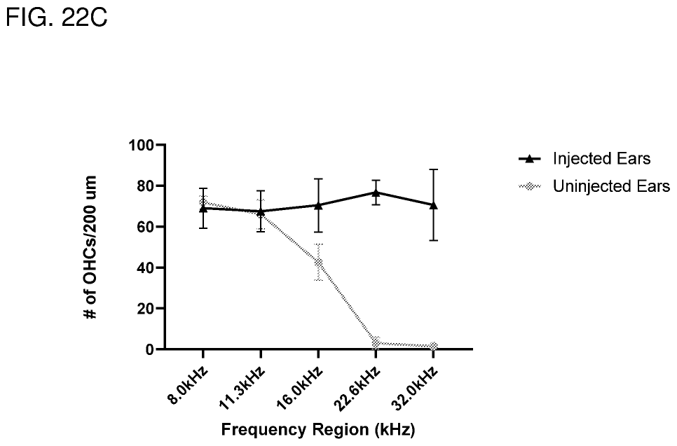

In a second experiment, Animals were injected with AAV1 encoding human GJB2 driven by AAV1-p.hGJB2(SEQ ID NO: 30)+Enhancers 1+6+7+8(SEQ ID NOs: 52, 57, 58, 59). The enhancers were positioned 5′ of the promoter in the vector. Hearing measurements were performed at 4, 12, and 14 weeks post-injection, after which animals were taken down. The results of this experiment are shown in FIGS. 22A-22C.

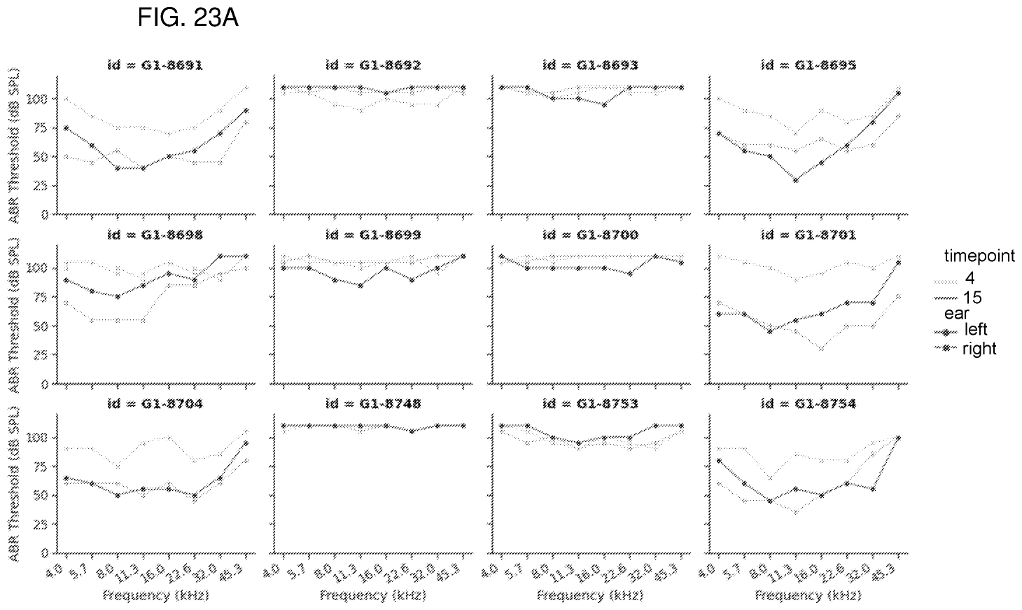

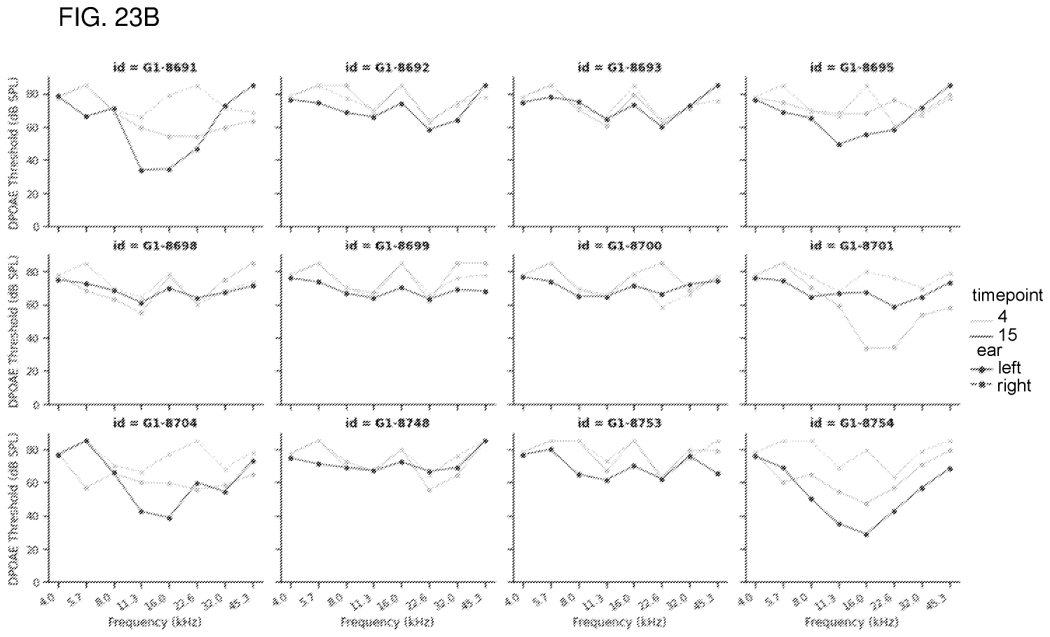

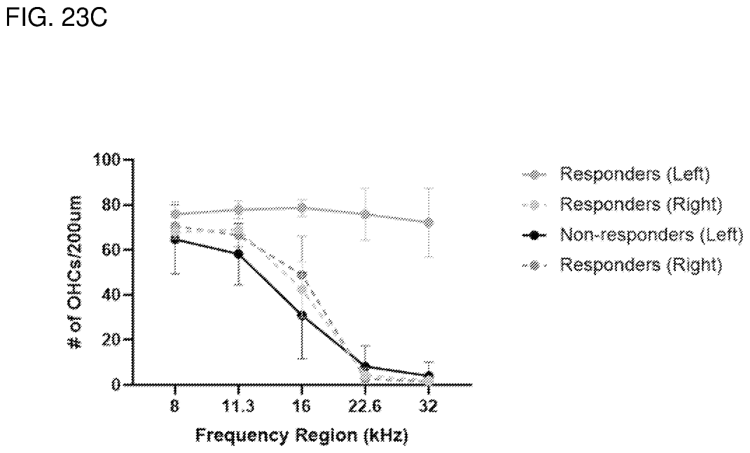

In a third experiment, animals were injected with AAV1 encoding human GJB2 driven by AAV1-p.hGJB2(SEQ ID NO: 30)+Enhancer 8(SEQ ID NO: 59). Enhancer 8 was positioned 5′ of the promoter in the vector. Hearing measurements were performed at 4 and 15 weeks post-injection, after which animals were taken down. The results of this experiment are shown in FIGS. 23A-23C.

As shown in FIGS. 21A-21C, 22A-22C, and 23A-23C, GJB2 gene replacement therapy with various GJB2 enhancer/promoter combinations in AAV1 was able to restore hearing and prevent outer hair cell loss in a GJB2-deficient mouse model. This shows that hearing restoration can be successful when leveraging regulatory element combinations that appropriately restrict GJB2 expression to GJB2-expressing cells.

### Example 9. GJB2 Enhancer/Promoter Combinations can be Used to Drive Expression of GFP in GJB2-Expressing Cells in Non-Human Primates

Adult non-human primates received a local injection to the round window of the inner ear at a flow rate of 6 μL/min with 60 μL of an AAV1-p.hGJB2(SEQ ID NO: 30)+GJB2enh-GFP. GJB2enh was made up of one of: Enhancer 1 (SEQ ID NO: 52), Enhancer GH (SEQ ID NO: 60), or Enhancers 1+6+7+8(SEQ ID NOs: 52, 57, 58, 59). Enhancer 1 and the combination of Enhancers 1, 6, 7, and 8 were positioned 5′ of the promoter in the vector. Enhancer GH was positioned 3′ of the transgene in the vector. The animals' ears were vented at the lateral semicircular canal to allow for outflow of perilymph during delivery. Four weeks post-injection, animals were sacrificed by cardiac perfusion of 10% neutral buffered formalin (NBF) and their temporal bones were harvested.

### Whole Mount Histology & Quantification

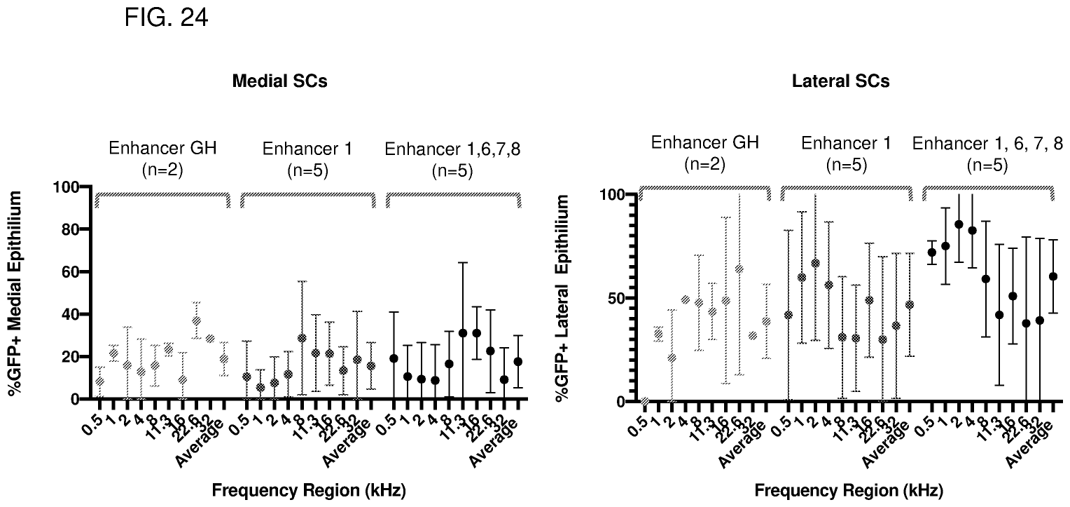

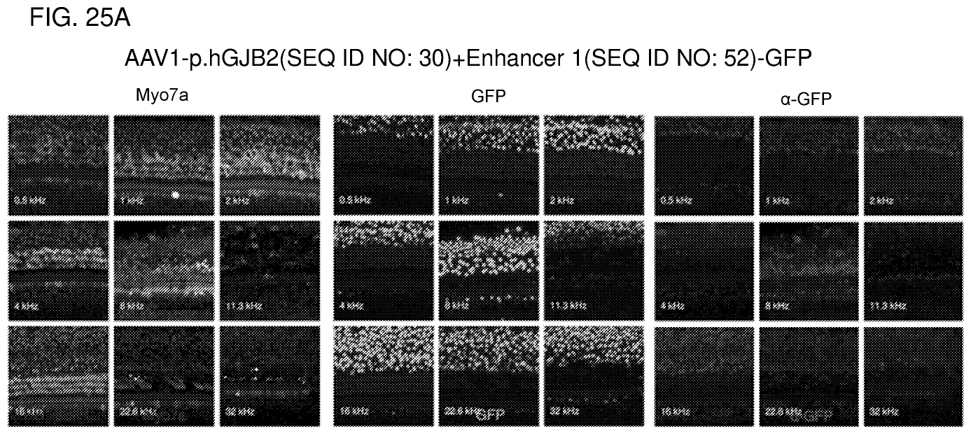

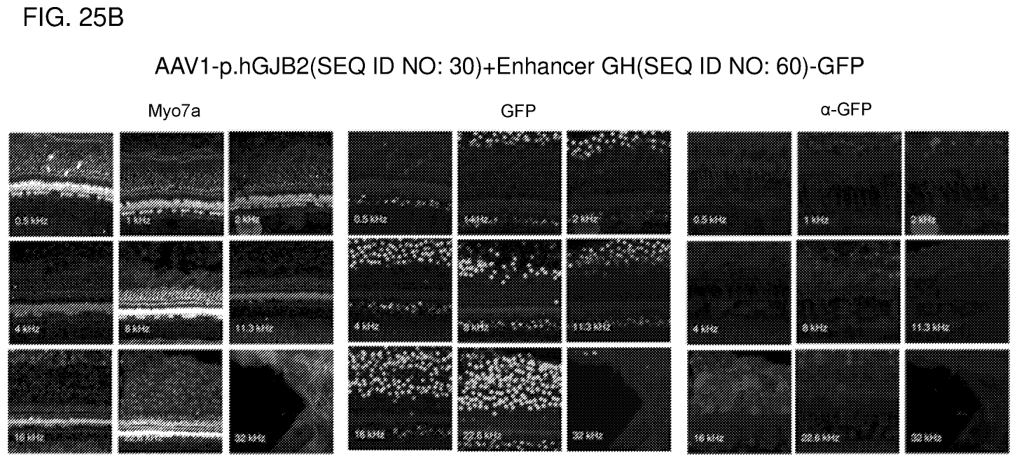

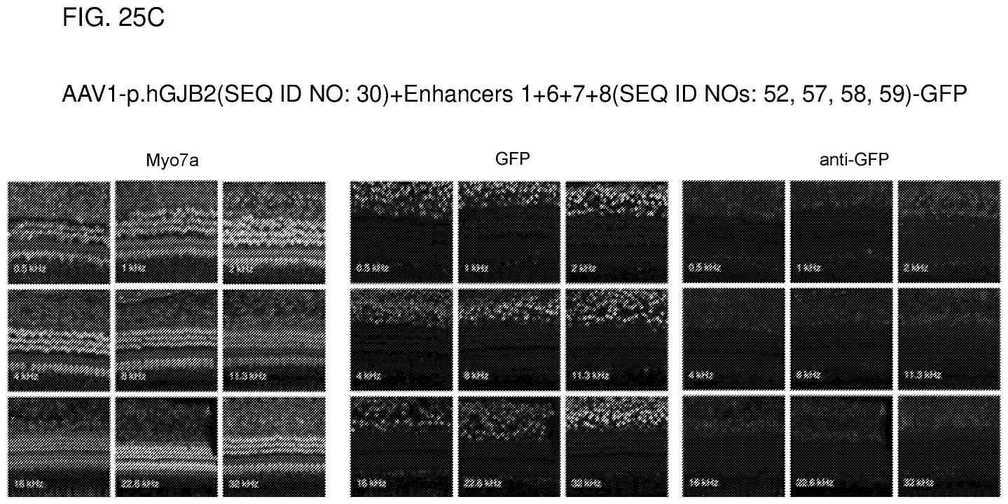

After decalcification in 0.5M EDTA, ears were micro dissected and organs of Corti prepared for immunohistochemistry. Whole mount tissue preparations of the organ of Corti were counterstained with DAPI, Myosin7a antibody, and anti-GFP antibody to visualize cell nuclei, hair cells and low GFP signal. These were imaged together with the virally mediated native GFP expression under a confocal microscope (Zeiss LSM 880, 40×/0.95 NA, 1 μm step size at 2 AU). In order to quantify the percentage of GFP-positive medial and lateral supporting cells, regions of interest at frequencies spanning the length of the cochlea were selected, DAPI was used to count the total number of medial and lateral supporting cells, and GFP-positive supporting cells were counted. Results are shown in FIG. 24. GFP expression was seen in ˜20% of medial supporting cells, and ˜50% of lateral supporting cells, and was relatively similar across groups. Representative images are shown in FIGS. 25A-25C. Minimal expression was detected in hair cells in all groups.

### Section Histology & Quantification

After decalcification in 0.5M EDTA, the temporal bones were embedded in paraffin, then mid-modiolar sections were obtained. Sections were baked overnight at 65° C., deparaffinization and tissue hydration was performed with Bound Dewax Solution for 30 seconds. In addition, antigen retrieval via 70° C. treatment for 2 hours in pH 9.0 EDTA was leveraged. Primary antibody incubation against GFP (Abcam #183734) was performed for 15 min at room temperature, followed by a secondary antibody incubation of 30 min at room temperature, and 10 min+5 min room temperature incubation for FastRed chromagen (secondary antibody and chromogenic reagent: BOND Polymer Refine Red Detection). Finally, tissue was counterstained using hematoxylin for 7 min at room temperature. Slides were imaged with a 20× objective using a Lecia Aperio Slide Scanner.

For RNAscope on NHP sections, epitope retrieval was performed using BOND ER Solution (pH 9) for 10 min at 90° C. After subsequent washes in BOND Wash (1×), slides were incubated in RNAscope 2.5 LSx Protease for 10 min at 40° C. followed by additional washes. Sections were incubated in hydrogen peroxide to block endogenous peroxidase activity. Following additional washes in BOND Wash (1×), probe hybridization was performed for 2 hours at 42° C. followed by 6 amplification steps. Sections were then incubated with the chromogenic reagent (BOND Polymer Refine Red) for 1 min+15 min at room temperature. Finally, tissue was counterstained using hematoxylin for 10 min at room temperature. Slides were imaged with a 40× objective using a Leica Aperio Slide Scanner.

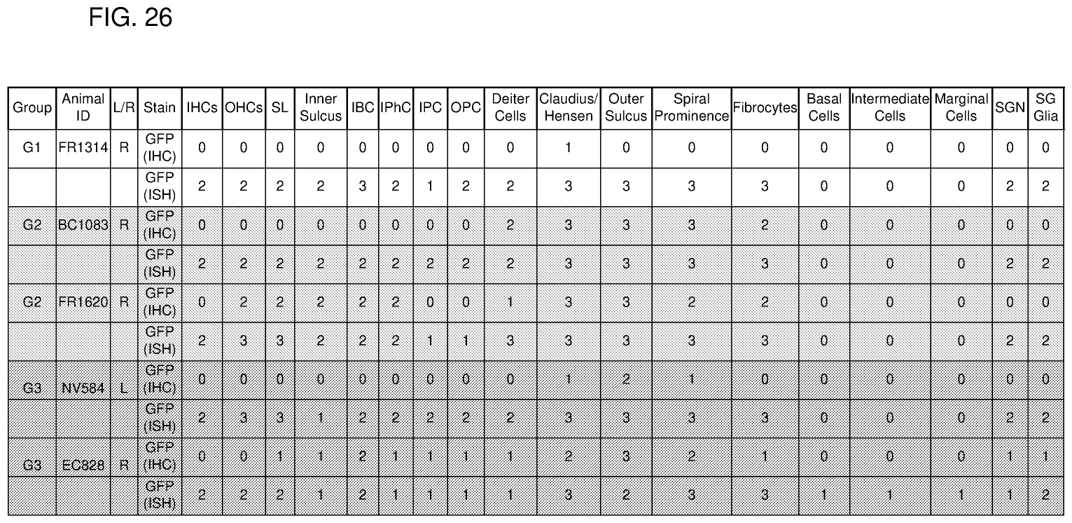

Cross-sections were used to score the magnitude of GFP from both IHC and ISH readouts across various cochlear cell types. Scores were from 0-3, for lowest to highest expression, and summarized in FIG. 26. Animals that showed a complete absence of labeling for both readouts were assumed to be the result of failed viral delivery and excluded from the analysis. Consistent with the fact that the GFP ISH probe labeled both viral genome and GFP mRNA, GFP ISH signal was detected broadly across cochlear cell types, including in the hair cells. However, quantification of the GFP IHC signal appeared to be more restricted to GJB2-expressing cell types. In particular, protein expression was largely absent from hair cells, but detected in various groups of supporting cells, including Deiter cells, as well as inner and outer sulcus, spiral prominence, and fibrocytes.

### Example 10. A GJB2 Enhancer/Promoter Pair Drives Expression of a FLAG-Tagged Human GJB2 Transgene in GJB2-Expressing Cells in the Non-Human Primate

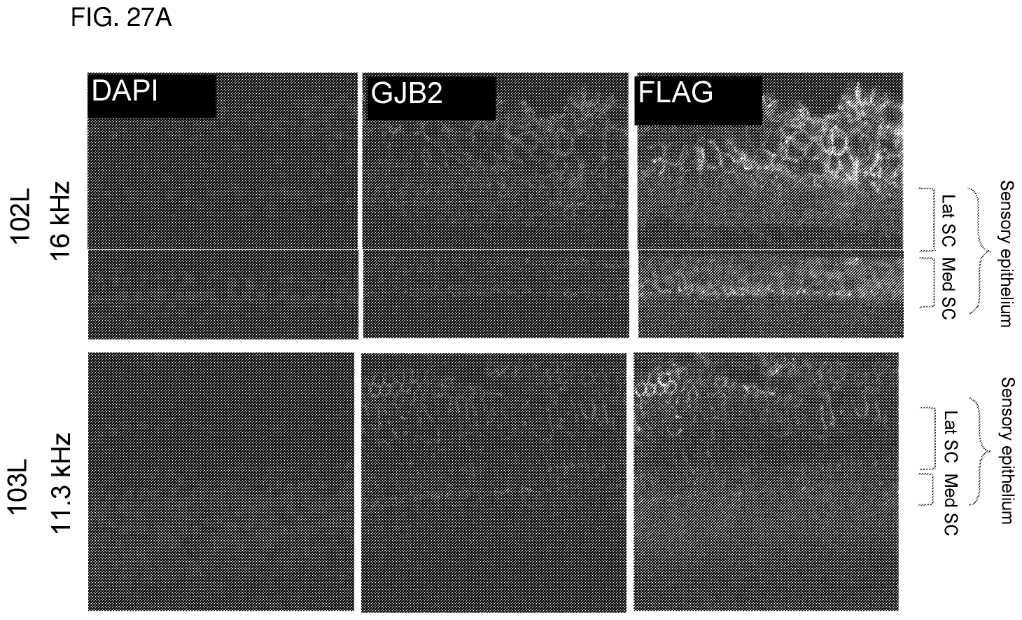

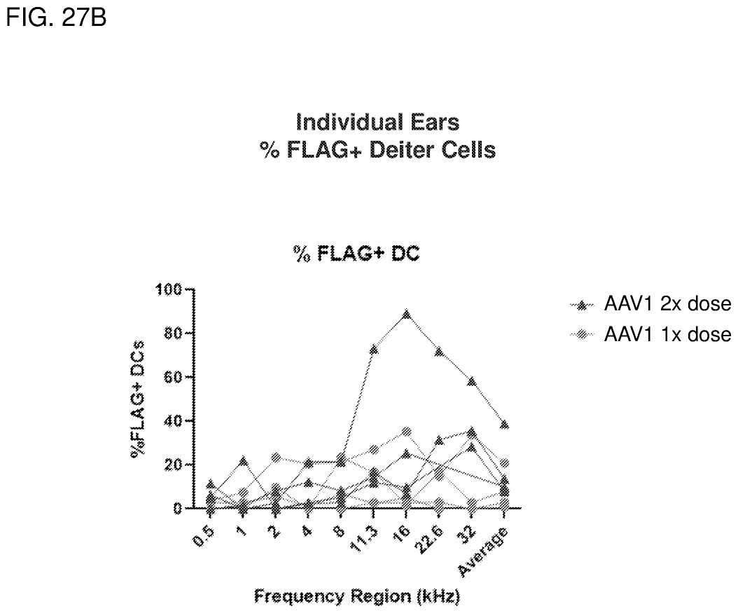

Adult non-human primates received a local injection to the round window of the inner ear at a flow rate of 6 μL/min with 60 μL of an AAV1-p.hGJB2(SEQ ID NO: 30)+Enhancer 1(SEQ ID NO: 52)-hGJB2-FLAG at a dose of 1× or 2×. Enhancer 1 was positioned 5′ of the promoter in the vector. The animals' ears were vented at the lateral semicircular canal to allow for outflow of perilymph during delivery. Processing of the tissue was performed similarly to Example 9, with the following modifications: whole mounts were counterstained with DAPI, GJB2 antibody, FLAG antibody, and Sox2 antibody, to label cell nuclei, endogenous GJB2 protein, tagged GJB2 transgene, and supporting cells, respectively. FLAG staining was leveraged to quantify the percentage of transgene-positive Deiter cells (FIG. 27B). Transgene expression was detected in Deiter cells, with a bias towards the higher frequencies. Representative images centered around the sensory epithelium are shown in FIG. 27A. Transgene (FLAG staining) was detected in both medial and lateral supporting cell regions, and the transgene staining pattern mirrored that of endogenous GJB2, thus showing that the transgene was properly expressed and incorporated into cell membrane plaques. Minimal transgene expression was seen in hair cells.

### Example 11. Enhancer 8 Retains Specificity when Paired with the GJB2 Promoter of SEQ ID NO: 1 in Murine Cochlear Explants

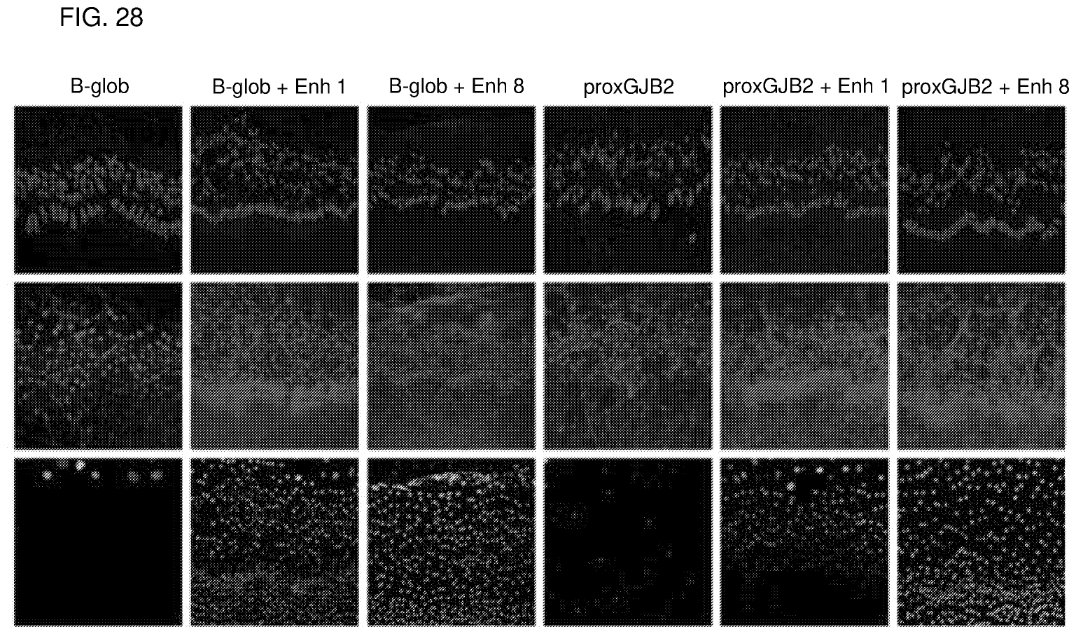

Murine cochlear explant cultures were established as previously described in Example 6, with the exception of AAV dose, which in this case was 5e11 vg/culture. To evaluate the contribution of the proximal promoter region to the specificity of the enhancer/promoter pair, Enhancer 1 (SEQ ID NO: 52) and Enhancer 8 (SEQ ID NO: 59) were paired with either a proximal GJB2 promoter sequence (proxGJB2-SEQ ID NO: 1), or a minimal beta-globin promoter (B-glob), driving nuclear GFP expression. Both promoters were also tested alone as controls. Expression pattern, GFP intensity, and exclusion from hair cells were assessed and compared to results from the same enhancers paired with the GJB2 promoter of SEQ ID NO: 30 (FIG. 16). Results are shown in FIG. 28. The proxGJB2 and B-glob promoters alone drove minimal GFP expression overall. Use of the B-glob promoter eliminated specificity from both Enhancers 1 and 8, with expression seen broadly in hair cells, supporting cells, and beyond the sensory epithelium. Enhancer 1 paired with proxGJB2 led to positive expression in the outer hair cells, compared to the GJB2 promoter of SEQ ID NO: 30, which fully excluded GFP expression from hair cells. Enhancer 8 paired with proxGJB2 excluded expression from hair cells, however supporting cell expression was not restricted to the sensory epithelium as compared to with the GJB2 promoter of SEQ ID NO: 30. This demonstrates that Enhancer 8 is able to exclude GFP expression from hair cells when paired with an alternative GJB2 promoter.

### Example 12. Administration of a Composition Containing a Nucleic Acid Vector Containing a GJB2 Promoter to a Subject with Sensorineural Hearing Loss

According to the methods disclosed herein, a physician of skill in the art can treat a patient, such as a human patient, with hearing loss (e.g., sensorineural hearing loss, such as GJB2-related hearing loss) so as to improve or restore hearing. To this end, a physician of skill in the art can administer to the human patient a composition containing an AAV vector (e.g., an AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ, DJ/8, DJ/9, 7m8, PHP.B, PHP.eB, or PHP.S vector) containing a GJB2 promoter described herein (e.g., a polynucleotide that contains a first region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1 or a functional portion or derivative thereof and/or a second region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or a functional portion or derivative thereof, optionally containing a linker joining the first region and the second region, such as a promoter of any one of SEQ ID NOs: 29-35) operably linked to a polynucleotide encoding an expression product, such as a polynucleotide encoding wild-type form of Gjb2 (e.g., a polynucleotide encoding the amino acid sequence of SEQ ID NO: 38). The composition containing the AAV vector may be administered to the patient, for example, by local administration to the inner ear (e.g., injection into the perilymph or through the round window membrane) to treat sensorineural hearing loss.

Following administration of the composition to a patient, a practitioner of skill in the art can monitor the patient's improvement in response to the therapy by a variety of methods. For example, a physician can monitor the patient's hearing by performing standard tests, such as audiometry, ABR, electrocochleography (ECOG), and otoacoustic emissions following administration of the composition. A finding that the patient exhibits improved hearing in one or more of the tests following administration of the composition compared to hearing test results prior to administration of the composition indicates that the patient is responding favorably to the treatment. Subsequent doses can be determined and administered as needed.

### Example 13. Administration of a Composition Containing a Nucleic Acid Vector Containing a GJB2 Enhancer to a Subject with Sensorineural Hearing Loss

According to the methods disclosed herein, a physician of skill in the art can treat a patient, such as a human patient, with hearing loss (e.g., sensorineural hearing loss) so as to improve or restore hearing. To this end, a physician of skill in the art can administer to the human patient a composition containing an AAV vector (e.g., an AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ, DJ/8, DJ/9, 7m8, PHP.B, PHP.eB, or PHP.S vector) containing a GJB2 enhancer described herein (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to any one of SEQ ID NOs: 52-63) operably linked to a promoter (e.g., a GJB2 promoter, such as a GJB2 promoter described herein or a GJB2 promoter having at least 85% sequence identity to any one of SEQ ID NOs: 1, 8-11, and 66-68, or an inner ear cell-type specific promoter, such as a promoter listed in Table 9) that is operably linked to a polynucleotide encoding an expression product (e.g., a wild-type version of a cochlear supporting cell gene associated with hearing loss that is mutated in the subject, such as GJB2, GJB6, GAS2, or a gene listed in Table 6, or a polynucleotide that encodes an expression product that can induce the differentiation of cochlear supporting cells into cochlear hair cells, such as a polynucleotide listed in Table 5). The composition containing the AAV vector may be administered to the patient, for example, by local administration to the inner ear (e.g., injection into the perilymph or through the round window membrane) to treat sensorineural hearing loss.

Following administration of the composition to a patient, a practitioner of skill in the art can monitor the patient's improvement in response to the therapy by a variety of methods. For example, a physician can monitor the patient's hearing by performing standard tests, such as audiometry, ABR, electrocochleography (ECOG), and otoacoustic emissions following administration of the composition. A finding that the patient exhibits improved hearing in one or more of the tests following administration of the composition compared to hearing test results prior to administration of the composition indicates that the patient is responding favorably to the treatment. Subsequent doses can be determined and administered as needed.

Exemplary embodiments of the invention are described in the enumerated paragraphs below.
E1. A polynucleotide comprising a GJB2 promoter comprising a first region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1 or a functional portion or derivative thereof comprising at least one of (e.g., one or more of) SEQ ID NOs: 3-12 operably linked to a second region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or a functional portion or derivative thereof comprising at least one of (e.g., one of more of) SEQ ID NOs: 20-24, wherein the distance between the first region and the second region in the polynucleotide (e.g., the distance between the 3′ end of the first region and the 5′ end of the second region) is no more than 1 kb, optionally comprising a linker comprising one to one hundred nucleotides between the first region and the second region.
E2. The polynucleotide of E1, wherein the distance between the first region and the second region in the polynucleotide is no more than 0.5 kb.
E3. The polynucleotide of E2, wherein the distance between the first region and the second region in the polynucleotide is no more than 0.25 kb.
E4. A polynucleotide comprising a GJB2 promoter comprising a first region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1 or a functional portion or derivative thereof comprising at least one of (e.g., one or more of) SEQ ID NOs: 3-12 operably linked to a second region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or a functional portion or derivative thereof comprising at least one of (e.g., one of more of) SEQ ID NOs: 20-24, wherein when the first region and the second region are not joined directly, the first region is operably linked to the second region by a nucleic acid sequence that differs from the intervening genomic sequence.
E5. A polynucleotide comprising a GJB2 promoter operably linked to a heterologous polynucleotide, the GJB2 promoter comprising a first region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1 or a functional portion or derivative thereof comprising at least one of (e.g., one or more of) SEQ ID NOs: 3-12 operably linked to a second region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or a functional portion or derivative thereof comprising at least one of (e.g., one of more of) SEQ ID NOs: 20-24, optionally comprising a linker comprising one to one hundred nucleotides between the first region and the second region.
E6. The polynucleotide of E5, wherein the heterologous polynucleotide is a codon-optimized nucleic acid sequence encoding wild-type GJB2 (e.g., a nucleic acid sequence set forth in SEQ ID NOs: 41-44).
E7. The polynucleotide of E5, wherein the heterologous polynucleotide encodes a Gjb2 protein comprising one or more conservative amino acid substitutions relative to SEQ ID NO: 38 that retains the therapeutic function of wild-type Gjb2.
E8. The polynucleotide of E5, wherein the heterologous polynucleotide encodes Gjb6 (e.g., encodes SEQ ID NO: 47).
E9. The polynucleotide of E5, wherein the heterologous polynucleotide encodes BDNF or NTF3.
E10. The polynucleotide of E5, wherein the heterologous polynucleotide is a polynucleotide encoding a protein or inhibitory RNA that can induce differentiation of a cochlear supporting cell into a cochlear hair cell or induce or increase cochlear supporting cell proliferation (e.g., a polynucleotide listed in Table 5), or a transgene corresponding to a wild-type form of a gene that is expressed in cochlear supporting cells and mutated in a subject with hearing loss (e.g., a transgene corresponding to a wild-type form of a gene listed in Table 6).
E11. The polynucleotide of E5, the heterologous polynucleotide encodes (e.g., can be transcribed to produce) a short hairpin RNA (shRNA), an antisense oligonucleotide (ASO), a component of a gene editing system (e.g., a nuclease, such as a CRISPR Associated Protein 9 (Cas9), Transcription Activator-Like Effector Nuclease (TALEN), or Zinc Finger Nuclease (ZFN), or a guide RNA (gRNA)), or a microRNA.
E12. The polynucleotide of any one of E1-E11, wherein the first region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1.
E13. The polynucleotide of E12, wherein the first region has the sequence of SEQ ID NO: 1.
E14. The polynucleotide of any one of E1-E11, wherein the first region has at least 85% sequence identity (e.g., 85%, 86%, 87%, 88%, 89%, 90%, 91%, 92%, 93%, 94%, 95%, 96%, 97%, 98%, 99%, or more, sequence identity) to any one of SEQ ID NOs: 3-12.
E15. The polynucleotide of E14, the first region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 3.
E16. The polynucleotide of E15, wherein the first region has the sequence of SEQ ID NO: 3.
E17. The polynucleotide of E14, the first region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 4.
E18. The polynucleotide of E17, wherein the first region has the sequence of SEQ ID NO: 4.
E19. The polynucleotide of E14, the first region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 5.
E20. The polynucleotide of E19, wherein the first region has the sequence of SEQ ID NO: 5.
E21. The polynucleotide of E14, the first region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 6.
E22. The polynucleotide of E21, wherein the first region has the sequence of SEQ ID NO: 6.
E23. The polynucleotide of E14, the first region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 7.
E24. The polynucleotide of E23, wherein the first region has the sequence of SEQ ID NO: 7.
E25. The polynucleotide of E14, the first region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 8.
E26. The polynucleotide of E25, wherein the first region has the sequence of SEQ ID NO: 8.
E27. The polynucleotide of E14, the first region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 9.
E28. The polynucleotide of E27, wherein the first region has the sequence of SEQ ID NO: 9.
E29. The polynucleotide of E14, the first region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 10.
E30. The polynucleotide of E29, wherein the first region has the sequence of SEQ ID NO: 10.
E31. The polynucleotide of E14, the first region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 11.
E32. The polynucleotide of E31, wherein the first region has the sequence of SEQ ID NO: 11.
E33. The polynucleotide of E14, the first region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 12.
E34. The polynucleotide of E33, wherein the first region has the sequence of SEQ ID NO: 12.
E35. The polynucleotide of any one of E1-E11, wherein the functional portion of SEQ ID NO: 1 comprises the sequence of SEQ ID NO: 4 and the sequence of SEQ ID NO: 12.
E36. The polynucleotide of E35, wherein the first region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 13.
E37. The polynucleotide of E36, wherein the first region has the sequence of SEQ ID NO: 13.
E38. The polynucleotide of any one of E1-E11, wherein the functional portion of SEQ ID NO: 1 comprises the sequence of SEQ ID NO: 5 and the sequence of SEQ ID NO: 12.
E39. The polynucleotide of E38, wherein the first region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 14.
E40. The polynucleotide of E39, wherein the first region has the sequence of SEQ ID NO: 14.
E41. The polynucleotide of any one of E1-E11, wherein the functional portion of SEQ ID NO: 1 comprises the sequence of SEQ ID NO: 6 and the sequence of SEQ ID NO: 12.
E42. The polynucleotide of E41, wherein the first region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 15.
E43. The polynucleotide of E42, wherein the first region has the sequence of SEQ ID NO: 15.
E44. The polynucleotide of any one of E1-E11, wherein the functional portion of SEQ ID NO: 1 comprises the sequence of SEQ ID NO: 7 and the sequence of SEQ ID NO: 12.
E45. The polynucleotide of E44, wherein the first region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 16.
E46. The polynucleotide of E45, wherein the first region has the sequence of SEQ ID NO: 16.
E47. The polynucleotide of any one of E1-E11, wherein the functional portion of SEQ ID NO: 1 comprises the sequence of SEQ ID NO: 9 and the sequence of SEQ ID NO: 12.
E48. The polynucleotide of E47, wherein the first region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 17.
E49. The polynucleotide of E48, wherein the first region has the sequence of SEQ ID NO: 17.
E50. The polynucleotide of any one of E1-E11, wherein the functional portion of SEQ ID NO: 1 comprises the sequence of SEQ ID NO: 10 and the sequence of SEQ ID NO: 12.
E51. The polynucleotide of E50, wherein the first region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 18.
E52. The polynucleotide of E51, wherein the first region has the sequence of SEQ ID NO: 18.
E53. The polynucleotide of any one of E1-E11, wherein the functional portion of SEQ ID NO: 1 comprises the sequence of SEQ ID NO: 11 and the sequence of SEQ ID NO: 12.
E54. The polynucleotide of E53, wherein the first region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 19.
E55. The polynucleotide of E54, wherein the first region has the sequence of SEQ ID NO: 19.
E56. The polynucleotide of any one of E35, E38, E41, E44, E47, E50, and E53, wherein the sequence of SEQ ID NO: 12 precedes the sequence of SEQ ID NO: 4, SEQ ID NO: 5, SEQ ID NO: 6, SEQ ID NO: 7, SEQ ID NO: 9, SEQ ID NO: 10, or SEQ ID NO: 11.
E57. The polynucleotide of any one of E1-E56, wherein the second region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2.
E58. The polynucleotide of E57, wherein the second region has the sequence of SEQ ID NO: 2.
E59. The polynucleotide of any one of E1-E56, wherein the second region has at least 85% sequence identity (e.g., 85%, 86%, 87%, 88%, 89%, 90%, 91%, 92%, 93%, 94%, 95%, 96%, 97%, 98%, 99%, or more, sequence identity) to any one of SEQ ID NOs: 20-24.
E60. The polynucleotide of E59, wherein the second region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 20.
E61. The polynucleotide of E60, wherein the second region has the sequence of SEQ ID NO: 20.
E62. The polynucleotide of E59, wherein the second region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 21.
E63. The polynucleotide of E62, wherein the second region has the sequence of SEQ ID NO: 21.
E64. The polynucleotide of E59, wherein the second region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 22.
E65. The polynucleotide of E64, wherein the second region has the sequence of SEQ ID NO: 22.
E66. The polynucleotide of E59, wherein the second region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 23.
E67. The polynucleotide of E66, wherein the second region has the sequence of SEQ ID NO: 23.
E68. The polynucleotide of E59, wherein the second region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 24.
E69. The polynucleotide of E68, wherein the second region has the sequence of SEQ ID NO: 24.
E70. The polynucleotide of any one of E1-E56, wherein the functional portion of SEQ ID NO: 2 comprises the sequence of SEQ ID NO: 23 and the sequence of SEQ ID NO: 20.
E71. The polynucleotide of E70, wherein the second region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 25.
E72. The polynucleotide of E71, wherein the second region has the sequence of SEQ ID NO: 25.
E73. The polynucleotide of any one of E1-E56, wherein the functional portion of SEQ ID NO: 2 comprises the sequence of SEQ ID NO: 24 and the sequence of SEQ ID NO: 20.
E74. The polynucleotide of E73, wherein the second region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 26.
E75. The polynucleotide of E74, wherein the second region has the sequence of SEQ ID NO: 26.
E76. The polynucleotide of any one of E1-E56, wherein the functional portion of SEQ ID NO: 2 comprises the sequence of SEQ ID NO: 23 and the sequence of SEQ ID NO: 22.
E77. The polynucleotide of E76, wherein the second region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 27.
E78. The polynucleotide of E77, wherein the second region has the sequence of SEQ ID NO: 27.
E79. The polynucleotide of any one of E1-E56, wherein the functional portion of SEQ ID NO: 2 comprises the sequence of SEQ ID NO: 24 and the sequence of SEQ ID NO: 22.
E80. The polynucleotide of E79, wherein the second region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 28.
E81. The polynucleotide of E80, wherein the second region has the sequence of SEQ ID NO: 28.
E82. The polynucleotide of any one of E70, E73, E76, and E79, wherein the sequence of SEQ ID NO: 20 precedes the sequence of SEQ ID NO: 23 or SEQ ID NO: 24; or wherein the sequence of SEQ ID NO: 22 precedes the sequence of SEQ ID NO: 23 or SEQ ID NO: 24.
E83. The polynucleotide of any one of E1-E82, wherein the first region is joined directly (i.e., fused) to the second region without a linker (e.g., the 3′ end of the first region directly precedes the 5′ end of the second region).
E84. The polynucleotide of E83, wherein the GJB2 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 29.
E85. The polynucleotide of E84, wherein the GJB2 promoter has the sequence of SEQ ID NO: 29.
E86. The polynucleotide of E83, wherein the GJB2 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 30.
E87. The polynucleotide of E86, wherein the GJB2 promoter has the sequence of SEQ ID NO: 30.
E88. The polynucleotide of E83, wherein the GJB2 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 31.
E89. The polynucleotide of E88, wherein the GJB2 promoter has the sequence of SEQ ID NO: 31.
E90. The polynucleotide of E83, wherein the GJB2 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 32.
E91. The polynucleotide of E90, wherein the GJB2 promoter has the sequence of SEQ ID NO: 32.
E92. The polynucleotide of E83, wherein the GJB2 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 33.
E93. The polynucleotide of E92, wherein the GJB2 promoter has the sequence of SEQ ID NO: 33.
E94. The polynucleotide of E83, wherein the GJB2 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 34.
E95. The polynucleotide of E94, wherein the GJB2 promoter has the sequence of SEQ ID NO: 34.
E96. The polynucleotide of E83, wherein the GJB2 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 35.
E97. The polynucleotide of E96, wherein the GJB2 promoter has the sequence of SEQ ID NO: 35.
E98. A polynucleotide comprising a GJB2 promoter having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 13.
E99. The polynucleotide of E98, wherein the GJB2 promoter has the sequence of SEQ ID NO: 13.
E100. A polynucleotide comprising a GJB2 promoter having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 14.
E101. The polynucleotide of E100, wherein the GJB2 promoter has the sequence of SEQ ID NO: 14.
E102. A polynucleotide comprising a GJB2 promoter having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 15.
E103. The polynucleotide of E102, wherein the GJB2 promoter has the sequence of SEQ ID NO: 15.
E104. A polynucleotide comprising a GJB2 promoter having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 16.
E105. The polynucleotide of E104, wherein the GJB2 promoter has the sequence of SEQ ID NO: 16.
E106. A polynucleotide comprising a GJB2 promoter having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 17.
E107. The polynucleotide of E106, wherein the GJB2 promoter has the sequence of SEQ ID NO: 17.
E108. A polynucleotide comprising a GJB2 promoter having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 18.
E109. The polynucleotide of E108, wherein the GJB2 promoter has the sequence of SEQ ID NO: 18.
E110. A polynucleotide comprising a GJB2 promoter having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 19.
E111. The polynucleotide of E110, wherein the GJB2 promoter has the sequence of SEQ ID NO: 19.
E112. A polynucleotide comprising a GJB2 promoter having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 25.
E113. The polynucleotide of E112, wherein the GJB2 promoter has the sequence of SEQ ID NO: 25.
E114. A polynucleotide comprising a GJB2 promoter having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 26.
E115. The polynucleotide of E114, wherein the GJB2 promoter has the sequence of SEQ ID NO: 26.
E116. A polynucleotide comprising a GJB2 promoter having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 27.
E117. The polynucleotide of E116, wherein the GJB2 promoter has the sequence of SEQ ID NO: 27.
E118. A polynucleotide comprising a GJB2 promoter having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 28.
E119. The polynucleotide of E118, wherein the GJB2 promoter has the sequence of SEQ ID NO: 28.
E120. A polynucleotide comprising a GJB2 promoter having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 37.
E121. The polynucleotide of E120, wherein the GJB2 promoter has the sequence of SEQ ID NO: 37.
E122. A polynucleotide comprising a GJB2 promoter comprising a first region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or a functional portion or derivative thereof comprising at least one of (e.g., one of more of) SEQ ID Nos: 20-24 operably linked to a second region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1 or a functional portion or derivative thereof comprising at least one of (e.g., one or more of) SEQ ID NOs: 3-12, optionally comprising a linker comprising one to one hundred nucleotides between the first region and the second region.
E123. The polynucleotide of any one of E1-E4 and E12-E122, wherein the GJB2 promoter is operably linked to a polynucleotide encoding an expression product.
E124. The polynucleotide of E123, wherein the expression product is a heterologous expression product.
E125. The polynucleotide of E124, wherein the heterologous expression product is BDNF or NTF3.
E126. The polynucleotide of E123, wherein the expression product is a protein, a short hairpin RNA (shRNA), an antisense oligonucleotide (ASO), a component of a gene editing system (e.g., a nuclease, such as a CRISPR Associated Protein 9 (Cas9), Transcription Activator-Like Effector Nuclease (TALEN), or Zinc Finger Nuclease (ZFN), or a guide RNA (gRNA)), or a microRNA.
E127. The polynucleotide of E123, wherein the expression product is an expression product that is endogenously expressed in a GJB2-expressing cell.
E128. The polynucleotide of E127, wherein the expression product is an expression product that is endogenously expressed in a GJB2-expressing inner ear cell.
E129. The polynucleotide of E127 or E128, wherein the expression product is Gjb2 or Gjb6.
E130. The polynucleotide of E123, wherein the polynucleotide encoding an expression product is a polynucleotide encoding a protein or inhibitory RNA that can induce differentiation of a cochlear supporting cell into a cochlear hair cell, a polynucleotide encoding a protein or inhibitory RNA that can induce or increase cochlear supporting cell proliferation, or a transgene corresponding to a wild-type form of a gene that is expressed in cochlear supporting cells and mutated in a subject with hearing loss.
E131. The polynucleotide of E130, wherein the polynucleotide encoding an expression product is a polynucleotide listed in Table 5 or a transgene corresponding to a wild-type form of a gene listed in Table 6.
E132. The polynucleotide of any one of E1-E131, wherein the polynucleotide further comprises a GJB2 enhancer operably linked to the GJB2 promoter.
E133. The polynucleotide of E132, wherein the GJB2 enhancer has at least 85% sequence identity (e.g., 85%, 86%, 87%, 88%, 89%, 90%, 91%, 92%, 93%, 94%, 95%, 96%, 97%, 98%, 99%, or more, sequence identity) to any one of SEQ ID NOs: 52-63.
E134. The polynucleotide of E131 or E132, wherein the GJB2 enhancer has the sequence of any one of SEQ ID NOs: 52-63.
E135. The polynucleotide of E134, wherein the GJB2 enhancer has the sequence of SEQ ID NO: 52.
E136. The polynucleotide of E134, wherein the GJB2 enhancer has the sequence of SEQ ID NO: 53.
E137. The polynucleotide of E134, wherein the GJB2 enhancer has the sequence of SEQ ID NO: 54.
E138. The polynucleotide of E134, wherein the GJB2 enhancer has the sequence of SEQ ID NO: 55.
E139. The polynucleotide of E134, wherein the GJB2 enhancer has the sequence of SEQ ID NO: 56.
E140. The polynucleotide of E134, wherein the GJB2 enhancer has the sequence of SEQ ID NO: 57.
E141. The polynucleotide of E134, wherein the GJB2 enhancer has the sequence of SEQ ID NO: 58.
E142. The polynucleotide of E134, wherein the GJB2 enhancer has the sequence of SEQ ID NO: 59.
E143. The polynucleotide of E134, wherein the GJB2 enhancer has the sequence of SEQ ID NO: 60.
E144. The polynucleotide of E134, wherein the GJB2 enhancer has the sequence of SEQ ID NO: 61.
E145. The polynucleotide of E134, wherein the GJB2 enhancer has the sequence of SEQ ID NO: 62.
E146. The polynucleotide of E134, wherein the GJB2 enhancer has the sequence of SEQ ID NO: 63.
E147. The polynucleotide of any one of claims E132-E146, wherein the GJB2 enhancer is located 5′ of the promoter.
E146. The polynucleotide of any one of claims E132-E146, wherein the GJB2 enhancer is located 3′ of the promoter.
E149. The polynucleotide of any one of E132-E146, wherein the polynucleotide comprises two or more different GJB2 enhancers, wherein each enhancer is independently selected from an enhancer having at least 85% sequence identity (e.g., 85%, 86%, 87%, 88%, 89%, 90%, 91%, 92%, 93%, 94%, 95%, 96%, 97%, 98%, 99%, or more, sequence identity) to any one of SEQ ID NOs: 52-63.
E150. The polynucleotide of E149, wherein each different GJB2 enhancer is independently selected from an enhancer having the sequence of one of SEQ ID NOs: 52-63.
E151. The polynucleotide of E149 or E150, wherein the polynucleotide comprises four different GJB2 enhancers.
E152. The polynucleotide of E151, wherein the four enhancers are SEQ ID NO: 52, SEQ ID NO: 57, SEQ ID NO: 58, and SEQ ID NO: 59.
E153. The polynucleotide of E151, wherein the four enhancers are SEQ ID NO: 53, SEQ ID NO: 54, SEQ ID NO: 55, and SEQ ID NO: 56.
E154. The polynucleotide of any one of E132-E153, wherein the polynucleotide comprises two or more copies of a GJB2 enhancer having at least 85% sequence identity (e.g., 85%, 86%, 87%, 88%, 89%, 90%, 91%, 92%, 93%, 94%, 95%, 96%, 97%, 98%, 99%, or more, sequence identity) to any one of SEQ ID NOs: 52-63.
E155. The polynucleotide of E154, wherein each copy of the two or more copies of the enhancer has the sequence of one of SEQ ID NOs: 52-63.
E156. A nucleic acid vector comprising the polynucleotide of any one of E1-E155.
E157. A nucleic acid vector comprising a polynucleotide comprising a GJB2 promoter having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 36.
E158. The nucleic acid vector of E157, wherein the GJB2 promoter has the sequence of SEQ ID NO: 36.
E159. A nucleic acid vector comprising a polynucleotide comprising a GJB2 promoter having at least 85% sequence identity (e.g., 85%, 86%, 87%, 88%, 89%, 90%, 91%, 92%, 93%, 94%, 95%, 96%, 97%, 98%, 99%, or more, sequence identity) to any one of SEQ ID NOs: 3-7.
E160. The nucleic acid vector of E159, wherein the GJB2 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 3.
E161. The nucleic acid vector of E160, wherein the GJB2 promoter has the sequence of SEQ ID NO: 3.
E162. The nucleic acid vector of E159, wherein the GJB2 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 4.
E163. The nucleic acid vector of E162, wherein the GJB2 promoter has the sequence of SEQ ID NO: 4.
E164. The nucleic acid vector of E159, wherein the GJB2 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 5.
E165. The nucleic acid vector of E164, wherein the GJB2 promoter has the sequence of SEQ ID NO: 5.
E166. The nucleic acid vector of E159, wherein the GJB2 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 6.
E167. The nucleic acid vector of E166, wherein the GJB2 promoter has the sequence of SEQ ID NO: 6.
E168. The nucleic acid vector of E159, wherein the GJB2 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 7.
E169. The nucleic acid vector of E168, wherein the GJB2 promoter has the sequence of SEQ ID NO: 7.
E170. A nucleic acid vector comprising a polynucleotide comprising a GJB2 promoter having at least 85% sequence identity (e.g., 85%, 86%, 87%, 88%, 89%, 90%, 91%, 92%, 93%, 94%, 95%, 96%, 97%, 98%, 99%, or more, sequence identity) to any one of SEQ ID NOs: 2 and 20-24.
E171. The nucleic acid vector of E170, wherein the GJB2 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2.
E172. The nucleic acid vector of E171, wherein the GJB2 promoter has the sequence of SEQ ID NO: 2.
E173. The nucleic acid vector of E170, wherein the GJB2 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 20.
E174. The nucleic acid vector of E173, wherein the GJB2 promoter has the sequence of SEQ ID NO: 20.
E175. The nucleic acid vector of E170, wherein the GJB2 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 21.
E176. The nucleic acid vector of E175, wherein the GJB2 promoter has the sequence of SEQ ID NO: 21.
E177. The nucleic acid vector of E170, wherein the GJB2 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 22.
E178. The nucleic acid vector of E177, wherein the GJB2 promoter has the sequence of SEQ ID NO: 22.
E179. The nucleic acid vector of E170, wherein the GJB2 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 23.
E180. The nucleic acid vector of E179, wherein the GJB2 promoter has the sequence of SEQ ID NO: 23.
E181. The nucleic acid vector of E170, wherein the GJB2 promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 24.
E182. The nucleic acid vector of E181, wherein the GJB2 promoter has the sequence of SEQ ID NO: 24.
E183. A nucleic acid vector comprising a polynucleotide comprising a GJB2 promoter comprising a first region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1 or a functional portion or derivative thereof comprising at least one of (e.g., one or more of) SEQ ID NOs: 3-12 operably linked to a second region having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or a functional portion or derivative thereof comprising at least one of (e.g., one of more of) SEQ ID NOs: 20-24, optionally comprising a linker comprising one to one hundred nucleotides between the first region and the second region.
E184. The nucleic acid vector of E183, wherein the first region has at least 85% sequence identity (e.g., 85%, 86%, 87%, 88%, 89%, 90%, 91%, 92%, 93%, 94%, 95%, 96%, 97%, 98%, 99%, or more, sequence identity) to any one of SEQ ID NOs: 3-19.
E185. The nucleic acid vector of E183 or E184, wherein the second region has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2.
E186. The nucleic acid vector of E183 or E184, wherein the second region has at least 85% sequence identity (e.g., 85%, 86%, 87%, 88%, 89%, 90%, 91%, 92%, 93%, 94%, 95%, 96%, 97%, 98%, 99%, or more, sequence identity) to any one of SEQ ID NOs: 20-28.
E187. The nucleic acid vector of any one of E183-E186, wherein the first region is joined directly to the second region without a linker (e.g., the 3′ end of the first region directly precedes the 5′ end of the second region).
E188. The nucleic acid vector of any one of E157-E187, wherein the GJB2 promoter is operably linked to a polynucleotide encoding an expression product.
E189. The nucleic acid vector of E188, wherein the expression product is a heterologous expression product.
E190. The nucleic acid vector of 189, wherein the heterologous expression product is BDNF or NTF3.
E191. The nucleic acid vector of E188, wherein the expression product is a protein, a short hairpin RNA (shRNA), an antisense oligonucleotide (ASO), a component of a gene editing system, or a microRNA.
E192. The nucleic acid vector of E188, wherein the expression product is an expression product that is endogenously expressed in a GJB2-expressing cell.
E193. The nucleic acid vector of E188, wherein the expression product is an expression product that is endogenously expressed in a GJB2-expressing inner ear cell.
E194. The nucleic acid vector e of E192 or E193, wherein the expression product is Gjb2 or Gjb6.
E195. The nucleic acid vector of E188, wherein the polynucleotide encoding an expression product is a polynucleotide encoding a protein or inhibitory RNA that can induce differentiation of a cochlear supporting cell into a cochlear hair cell, a polynucleotide encoding a protein or inhibitory RNA that can induce or increase cochlear supporting cell proliferation, or a transgene corresponding to a wild-type form of a gene that is expressed in cochlear supporting cells and mutated in a subject with hearing loss.
E196. The nucleic acid vector of E195, wherein the polynucleotide encoding an expression product is a polynucleotide listed in Table 5 or a transgene corresponding to a wild-type form of a gene listed in Table 6.
E197. The nucleic acid vector of any one of E157-E17, wherein the polynucleotide further comprises a GJB2 enhancer operably linked to the GJB2 promoter.
E198. The nucleic acid vector of E197, wherein the GJB2 enhancer has at least 85% sequence identity (e.g., 85%, 86%, 87%, 88%, 89%, 90%, 91%, 92%, 93%, 94%, 95%, 96%, 97%, 98%, 99%, or more, sequence identity) to any one of SEQ ID NOs: 52-63.
E199. The nucleic acid vector of E197 or E198, wherein the GJB2 enhancer has the sequence of any one of SEQ ID NOs: 52-63.
E200. The nucleic acid vector of E199, wherein the GJB2 enhancer has the sequence of SEQ ID NO: 52.
E201. The nucleic acid vector of E199, wherein the GJB2 enhancer has the sequence of SEQ ID NO: 53.
E202. The nucleic acid vector of E199, wherein the GJB2 enhancer has the sequence of SEQ ID NO: 54.
E203. The nucleic acid vector of E199, wherein the GJB2 enhancer has the sequence of SEQ ID NO: 55.
E204. The nucleic acid vector of E199, wherein the GJB2 enhancer has the sequence of SEQ ID NO: 56.
E205. The nucleic acid vector of E199, wherein the GJB2 enhancer has the sequence of SEQ ID NO: 57.
E206. The nucleic acid vector of E199, wherein the GJB2 enhancer has the sequence of SEQ ID NO: 58.
E207. The nucleic acid vector of E199, wherein the GJB2 enhancer has the sequence of SEQ ID NO: 59.
E208. The nucleic acid vector of E199, wherein the GJB2 enhancer has the sequence of SEQ ID NO: 60.
E209. The nucleic acid vector of E199, wherein the GJB2 enhancer has the sequence of SEQ ID NO: 61.
E210. The nucleic acid vector of E199, wherein the GJB2 enhancer has the sequence of SEQ ID NO: 62.
E211. The nucleic acid vector of E199, wherein the GJB2 enhancer has the sequence of SEQ ID NO: 63.
E212. The nucleic acid vector of any one of claims E197-E211, wherein the GJB2 enhancer is located 5′ of the promoter.
E213. The nucleic acid vector of any one of claims E197-E211, wherein the GJB2 enhancer is located 3′ of the promoter.
E214. The nucleic acid vector of any one of E197-E213, wherein the polynucleotide comprises two or more different GJB2 enhancers, wherein each enhancer is independently selected from an enhancer having at least 85% sequence identity (e.g., 85%, 86%, 87%, 88%, 89%, 90%, 91%, 92%, 93%, 94%, 95%, 96%, 97%, 98%, 99%, or more, sequence identity) to any one of SEQ ID NOs: 52-63.
E215. The nucleic acid vector of E214, wherein each different GJB2 enhancer is independently selected from an enhancer having the sequence of one of SEQ ID NOs: 52-63.
E216. The nucleic acid vector of E214 or E215, wherein the polynucleotide comprises four different GJB2 enhancers.
E217. The nucleic acid vector of E216, wherein the four enhancers are SEQ ID NO: 52, SEQ ID NO: 57, SEQ ID NO: 58, and SEQ ID NO: 59.
E218. The nucleic acid vector of E216, wherein the four enhancers are SEQ ID NO: 53, SEQ ID NO: 54, SEQ ID NO: 55, and SEQ ID NO: 56.
E219. The nucleic acid vector of any one of E197-E218, wherein the polynucleotide comprises two or more copies of a GJB2 enhancer having at least 85% sequence identity (e.g., 85%, 86%, 87%, 88%, 89%, 90%, 91%, 92%, 93%, 94%, 95%, 96%, 97%, 98%, 99%, or more, sequence identity) to any one of SEQ ID NOs: 52-63.
E220. The nucleic acid vector of E219, wherein each copy of the two or more copies of the enhancer has the sequence of one of SEQ ID NOs: 52-63.
E221. A polynucleotide comprising an enhancer having at least 85% sequence identity (e.g., 85%, 86%, 87%, 88%, 89%, 90%, 91%, 92%, 93%, 94%, 95%, 96%, 97%, 98%, 99%, or more, sequence identity) to any one of SEQ ID NOs: 52-63 operably linked to a promoter, wherein when the enhancer is positioned 5′ of the promoter the distance between the enhancer and the promoter in the polynucleotide is less than 3 kilobases (3 kb).
E222. The polynucleotide of E221, wherein when the enhancer is positioned 5′ of the promoter the distance between the enhancer and the promoter in the polynucleotide is less than 2 kb.
E223. The polynucleotide of E222, wherein when the enhancer is positioned 5′ of the promoter the distance between the enhancer and the promoter in the polynucleotide is less than 1 kb.
E224. The polynucleotide of E223, wherein when the enhancer is positioned 5′ of the promoter the distance between the enhancer and the promoter in the polynucleotide is less than 0.5 kb.
E225. The polynucleotide of any one of E122-E224, wherein the enhancer is positioned 5′ of the promoter in the polynucleotide.
E226. The polynucleotide of any one of E221-E224, wherein the enhancer is positioned 3′ of the promoter in the polynucleotide.
E227. The polynucleotide of any one of E221-E226, wherein the enhancer has the sequence of any one of SEQ ID NOs: 52-63.
E228. The polynucleotide of any one of E221-E227, wherein the enhancer has the sequence of SEQ ID NO: 52.
E229. The polynucleotide of any one of E221-E227, wherein the enhancer has the sequence of SEQ ID NO: 53.
E230. The polynucleotide of any one of E221-E227, wherein the enhancer has the sequence of SEQ ID NO: 54.
E231. The polynucleotide of any one of E221-E227, wherein the enhancer has the sequence of SEQ ID NO: 55.
E232. The polynucleotide of any one of E221-E227, wherein the enhancer has the sequence of SEQ ID NO: 56.
E233. The polynucleotide of any one of E221-E227, wherein the enhancer has the sequence of SEQ ID NO: 57.
E234. The polynucleotide of any one of E221-E227, wherein the enhancer has the sequence of SEQ ID NO: 58.
E235. The polynucleotide of any one of E221-E227, wherein the enhancer has the sequence of SEQ ID NO: 59.
E236. The polynucleotide of any one of E221-E227, wherein the enhancer has the sequence of SEQ ID NO: 60.
E237. The polynucleotide of any one of E221-E227, wherein the enhancer has the sequence of SEQ ID NO: 61.
E238. The polynucleotide of any one of E221-E227, wherein the enhancer has the sequence of SEQ ID NO: 62.
E239. The polynucleotide of any one of E221-E227, wherein the enhancer has the sequence of SEQ ID NO: 63.
E240. The polynucleotide of any one of E221-E239, wherein the promoter is a GJB2 promoter.
E241. The polynucleotide of E240, wherein the GJB2 promoter has at least 85% sequence (e.g., 85%, 86%, 87%, 88%, 89%, 90%, 91%, 92%, 93%, 94%, 95%, 96%, 97%, 98%, 99%, or more, sequence identity) identity to any one of SEQ ID NOs: 1 and 8-11.
E242. The polynucleotide of E241, wherein the GJB2 promoter has the sequence of any one of SEQ ID NOs: 1 and 8-11.
E243. The polynucleotide of E242, wherein the GJB2 promoter has the sequence of SEQ ID NO: 1.
E244. The polynucleotide of E242, wherein the GJB2 promoter has the sequence of SEQ ID NO: 8.
E245. The polynucleotide of E242, wherein the GJB2 promoter has the sequence of SEQ ID NO: 9.
E246. The polynucleotide of E242, wherein the GJB2 promoter has the sequence of SEQ ID NO: 10.
E247. The polynucleotide of E242, wherein the GJB2 promoter has the sequence of SEQ ID NO: 11.
E248. The polynucleotide of E240, wherein the GJB2 promoter has at least 85% sequence (e.g., 85%, 86%, 87%, 88%, 89%, 90%, 91%, 92%, 93%, 94%, 95%, 96%, 97%, 98%, 99%, or more, sequence identity) identity to a GJB2 promoter listed in Table 8 (any one of SEQ ID NOs: 66-68).
E249. The polynucleotide of E248, wherein the GJB2 promoter has the sequence of a GJB2 promoter listed in Table 8 (any one of SEQ ID NOs: 66-68).
E250. The polynucleotide of E249, wherein the GJB2 promoter has the sequence of SEQ ID NO: 66.
E251. The polynucleotide of E249, wherein the GJB2 promoter has the sequence of SEQ ID NO: 67.
E252. The polynucleotide of E249, wherein the GJB2 promoter has the sequence of SEQ ID NO: 68.
E253. The polynucleotide of any one of E221-E239, wherein the promoter is an inner ear cell type-specific promoter (e.g., a cochlear supporting cell-specific promoter).
E254. The polynucleotide of E253, wherein the inner ear cell type-specific promoter is a promoter listed in Table 9.
E255. The polynucleotide of E254, wherein the inner ear cell-type specific promoter is a GFAP promoter.
E256. The polynucleotide of E254, wherein the inner ear cell-type specific promoter is a GLAST promoter.
E257. The polynucleotide of E254, wherein the inner ear cell-type specific promoter is a FGFR3 promoter.
E258. The polynucleotide of E254, wherein the inner ear cell-type specific promoter is a LGR5 promoter.
E259. The polynucleotide of E254, wherein the inner ear cell-type specific promoter is a SOX2 promoter.
E260. The polynucleotide of E254, wherein the inner ear cell-type specific promoter is a CD44 promoter.
E261. The polynucleotide of E254, wherein the inner ear cell-type specific promoter is a FRZB promoter.
E262. The polynucleotide of E254, wherein the inner ear cell-type specific promoter is a SLC26A4 promoter.
E263. The polynucleotide of E254, wherein the inner ear cell-type specific promoter is a CEACAM16 promoter.
E264. The polynucleotide of E254, wherein the inner ear cell-type specific promoter is a TYR promoter.
E265. The polynucleotide of E254, wherein the inner ear cell-type specific promoter is a KCNJ10 promoter.
E266. The polynucleotide of E254, wherein the inner ear cell-type specific promoter is a CLDN11 promoter.
E267. The polynucleotide of E254, wherein the inner ear cell-type specific promoter is a KCNE1 promoter.
E268. The polynucleotide of E254, wherein the inner ear cell-type specific promoter is a POU3F4 promoter.
E269. The polynucleotide of any one of E221-E268, wherein the promoter is operably linked to a polynucleotide that can be transcribed to produce an expression product.
E270. The polynucleotide of E269, wherein the expression product is a heterologous expression product.
E271. The polynucleotide of E269, wherein the expression product is an expression product that is endogenously expressed in a GJB2-expressing cell.
E272. The polynucleotide of E271, wherein the expression product is an expression product that is endogenously expressed in a GJB2-expressing inner ear cell.
E273. The polynucleotide of E271 or E272, wherein the expression product is Gjb2 or Gjb6.
E274. The polynucleotide of E269, wherein the polynucleotide encoding an expression product is a polynucleotide encoding a protein or inhibitory RNA that can induce differentiation of a cochlear supporting cell into a cochlear hair cell, a polynucleotide encoding a protein or inhibitory RNA that can induce or increase cochlear supporting cell proliferation, or a transgene corresponding to a wild-type form of a gene that is expressed in cochlear supporting cells and mutated in a subject with hearing loss.
E275. The polynucleotide of E274, wherein the polynucleotide encoding an expression product is a polynucleotide listed in Table 5 or a transgene corresponding to a wild-type form of a gene listed in Table 6.
E276. The polynucleotide of E269 or E270, wherein the expression product is BDNF or NTF3.
E277. The polynucleotide of E269 or E270, wherein the expression product is a protein, a short hairpin RNA (shRNA), an antisense oligonucleotide (ASO), a component of a gene editing system (e.g., a nuclease, such as a CRISPR Associated Protein 9 (Cas9), Transcription Activator-Like Effector Nuclease (TALEN), or Zinc Finger Nuclease (ZFN), or a guide RNA (gRNA)), or a microRNA.
E278. The polynucleotide of any one of E221-E277, wherein the polynucleotide comprises two or more different enhancers, wherein each enhancer is independently selected from an enhancer having at least 85% sequence identity (e.g., 85%, 86%, 87%, 88%, 89%, 90%, 91%, 92%, 93%, 94%, 95%, 96%, 97%, 98%, 99%, or more, sequence identity) to any one of SEQ ID NOs: 52-63.
E279. The polynucleotide of E278, wherein each different enhancer is independently selected from an enhancer having the sequence of one of SEQ ID NOs: 52-63.
E280. The polynucleotide of E278 or E279, wherein the polynucleotide comprises four different enhancers.
E281. The polynucleotide of E280, wherein the four enhancers are SEQ ID NO: 52, SEQ ID NO: 57, SEQ ID NO: 58, and SEQ ID NO: 59.
E282. The polynucleotide of E280, wherein the four enhancers are SEQ ID NO: 53, SEQ ID NO: 54, SEQ ID NO: 55, and SEQ ID NO: 56.
E283. The polynucleotide of any one of E221-E282, wherein the polynucleotide comprises two or more copies of an enhancer having at least 85% sequence identity (e.g., 85%, 86%, 87%, 88%, 89%, 90%, 91%, 92%, 93%, 94%, 95%, 96%, 97%, 98%, 99%, or more, sequence identity) to any one of SEQ ID NOs: 52-63.
E284. The polynucleotide of E283, wherein each copy of the two or more copies of the enhancer has the sequence of one of SEQ ID NOs: 52-63.
E285. A nucleic acid vector comprising the polynucleotide of any one of E221-E284.
E286. A nucleic acid vector comprising a polynucleotide comprising an enhancer having at least 85% sequence identity (e.g., 85%, 86%, 87%, 88%, 89%, 90%, 91%, 92%, 93%, 94%, 95%, 96%, 97%, 98%, 99%, or more, sequence identity) to any one of SEQ ID NOs: 52-63.
E287. The nucleic acid vector of E286, wherein the enhancer has the sequence of any one of SEQ ID NOs: 52-63.
E288. The nucleic acid vector of E286 or E287, wherein the enhancer has the sequence of SEQ ID NO: 52.
E289. The nucleic acid vector of E286 or E287, wherein the enhancer has the sequence of SEQ ID NO: 53.
E290. The nucleic acid vector of E286 or E287, wherein the enhancer has the sequence of SEQ ID NO: 54.
E291. The nucleic acid vector of E286 or E287, wherein the enhancer has the sequence of SEQ ID NO: 55.
E292. The nucleic acid vector of E286 or E287, wherein the enhancer has the sequence of SEQ ID NO: 56.
E293. The nucleic acid vector of E286 or E287, wherein the enhancer has the sequence of SEQ ID NO: 57.
E294. The nucleic acid vector of E286 or E287, wherein the enhancer has the sequence of SEQ ID NO: 58.
E295. The nucleic acid vector of E286 or E287, wherein the enhancer has the sequence of SEQ ID NO: 59.
E296. The nucleic acid vector of E286 or E287, wherein the enhancer has the sequence of SEQ ID NO: 60.
E297. The nucleic acid vector of E286 or E287, wherein the enhancer has the sequence of SEQ ID NO: 61.
E298. The nucleic acid vector of E286 or E287, wherein the enhancer has the sequence of SEQ ID NO: 62.
E299. The nucleic acid vector of E286 or E287, wherein the enhancer has the sequence of SEQ ID NO: 63.
E300. The nucleic acid vector of any one of E286-E299, wherein the enhancer is operably linked to a promoter.
E301. The nucleic acid vector of E300, wherein the enhancer is positioned 5′ of the promoter.
E302. The nucleic acid vector of E300, wherein the enhancer is positioned 3′ of the promoter.
E303. The nucleic acid vector of any one of E300-E302, wherein the promoter is a GJB2 promoter.
E304. The nucleic acid vector of E303, wherein the GJB2 promoter has at least 85% sequence (e.g., 85%, 86%, 87%, 88%, 89%, 90%, 91%, 92%, 93%, 94%, 95%, 96%, 97%, 98%, 99%, or more, sequence identity) identity to any one of SEQ ID NOs: 1 and 8-11.
E305. The nucleic acid vector of E304, wherein the GJB2 promoter has the sequence of any one of SEQ ID NOs: 1 and 8-11.
E306. The nucleic acid vector of E305, wherein the GJB2 promoter has the sequence of SEQ ID NO: 1.
E307. The nucleic acid vector of E305, wherein the GJB2 promoter has the sequence of SEQ ID NO: 8.
E308. The nucleic acid vector of E305, wherein the GJB2 promoter has the sequence of SEQ ID NO: 9.
E309. The nucleic acid vector of E305, wherein the GJB2 promoter has the sequence of SEQ ID NO: 10.
E310. The nucleic acid vector of E305, wherein the GJB2 promoter has the sequence of SEQ ID NO: 11.
E311. The nucleic acid vector of E303, wherein the GJB2 promoter has at least 85% sequence (e.g., 85%, 86%, 87%, 88%, 89%, 90%, 91%, 92%, 93%, 94%, 95%, 96%, 97%, 98%, 99%, or more, sequence identity) identity to a GJB2 promoter listed in Table 8 (any one of SEQ ID NOs: 66-68).
E312. The nucleic acid vector of E311, wherein the GJB2 promoter has the sequence of a GJB2 promoter listed in Table 8 (any one of SEQ ID NOs: 66-68).
E313. The nucleic acid vector of E312, wherein the GJB2 promoter has the sequence of SEQ ID NO: 66.
E314. The nucleic acid vector of E312, wherein the GJB2 promoter has the sequence of SEQ ID NO: 67.
E315. The nucleic acid vector of E312, wherein the GJB2 promoter has the sequence of SEQ ID NO: 68.
E316. The nucleic acid vector of any one of E300-E302, wherein the promoter is an inner ear cell type-specific promoter (e.g., a cochlear supporting cell-specific promoter).
E317. The nucleic acid vector of E316, wherein the inner ear cell type-specific promoter is a promoter listed in Table 9.
E318. The nucleic acid vector of E317, wherein the inner ear cell-type specific promoter is a GFAP promoter.
E319. The nucleic acid vector of E317, wherein the inner ear cell-type specific promoter is a GLAST promoter.
E320. The nucleic acid vector of E317, wherein the inner ear cell-type specific promoter is a FGFR3 promoter.
E321. The nucleic acid vector of E317, wherein the inner ear cell-type specific promoter is a LGR5 promoter.
E322. The nucleic acid vector of E317, wherein the inner ear cell-type specific promoter is a SOX2 promoter.
E323. The nucleic acid vector of E317, wherein the inner ear cell-type specific promoter is a CD44 promoter.
E324. The nucleic acid vector of E317, wherein the inner ear cell-type specific promoter is a FRZB promoter.
E325. The nucleic acid vector of E317, wherein the inner ear cell-type specific promoter is a SLC26A4 promoter.
E326. The nucleic acid vector of E317, wherein the inner ear cell-type specific promoter is a CEACAM16 promoter.
E327. The nucleic acid vector of E317, wherein the inner ear cell-type specific promoter is a TYR promoter.
E328. The nucleic acid vector of E317, wherein the inner ear cell-type specific promoter is a KCNJ10 promoter.
E329. The nucleic acid vector of E317, wherein the inner ear cell-type specific promoter is a CLDN11 promoter.
E330. The nucleic acid vector of E317, wherein the inner ear cell-type specific promoter is a KCNE1 promoter.
E331. The nucleic acid vector of E317, wherein the inner ear cell-type specific promoter is a POU3F4 promoter.
E332. The nucleic acid vector of any one of E300-E331, wherein the promoter is operably linked to a polynucleotide that can be transcribed to produce an expression product.
E333. The nucleic acid vector of E332, wherein the expression product is a heterologous expression product.
E334. The nucleic acid vector of E332, wherein the expression product is an expression product that is endogenously expressed in a GJB2-expressing cell.
E335. The nucleic acid vector of E334, wherein the expression product is an expression product that is endogenously expressed in a GJB2-expressing inner ear cell.
E336. The nucleic acid vector of E334 or E335, wherein the expression product is Gjb2 or Gjb6.
E337. The nucleic acid vector of E332, wherein the polynucleotide encoding an expression product is a polynucleotide encoding a protein or inhibitory RNA that can induce differentiation of a cochlear supporting cell into a cochlear hair cell, a polynucleotide encoding a protein or inhibitory RNA that can induce or increase cochlear supporting cell proliferation, or a transgene corresponding to a wild-type form of a gene that is expressed in cochlear supporting cells and mutated in a subject with hearing loss.
E338. The nucleic acid vector of E337, wherein the polynucleotide encoding an expression product is a polynucleotide listed in Table 5 or a transgene corresponding to a wild-type form of a gene listed in Table 6.
E339. The nucleic acid vector of E332 or E333, wherein the expression product is BDNF or NTF3.
E340. The nucleic acid vector of E332 or E333, wherein the expression product is a protein, a short hairpin RNA (shRNA), an antisense oligonucleotide (ASO), a component of a gene editing system (e.g., a nuclease, such as a CRISPR Associated Protein 9 (Cas9), Transcription Activator-Like Effector Nuclease (TALEN), or Zinc Finger Nuclease (ZFN), or a guide RNA (gRNA)), or a microRNA.
E341. The nucleic acid vector of any one of E286-E340, wherein the polynucleotide comprises two or more different enhancers, wherein each enhancer is independently selected from an enhancer having at least 85% sequence identity (e.g., 85%, 86%, 87%, 88%, 89%, 90%, 91%, 92%, 93%, 94%, 95%, 96%, 97%, 98%, 99%, or more, sequence identity) to any one of SEQ ID NOs: 52-63.
E342. The nucleic acid vector of E341, wherein each different enhancer is independently selected from an enhancer having the sequence of one of SEQ ID NOs: 52-63.
E343. The nucleic acid vector of E341 or E342, wherein the polynucleotide comprises four different enhancers.
E344. The nucleic acid vector of E343, wherein the four enhancers are SEQ ID NO: 52, SEQ ID NO: 57, SEQ ID NO: 58, and SEQ ID NO: 59.
E345. The nucleic acid vector of E343, wherein the four enhancers are SEQ ID NO: 53, SEQ ID NO: 54, SEQ ID NO: 55, and SEQ ID NO: 56.
E346. The nucleic acid vector of any one of E286-E345, wherein the polynucleotide comprises two or more copies of an enhancer having at least 85% sequence identity (e.g., 85%, 86%, 87%, 88%, 89%, 90%, 91%, 92%, 93%, 94%, 95%, 96%, 97%, 98%, 99%, or more, sequence identity) to any one of SEQ ID NOs: 52-63.
E347. The nucleic acid vector of E346, wherein each copy of the two or more copies of the enhancer has the sequence of one of SEQ ID NOs: 52-63.
E348. The nucleic acid vector of any one of E156-E220 and E285-E347, wherein the nucleic acid vector is a viral vector, plasmid, cosmid, or artificial chromosome.
E349. The nucleic acid vector of any one of E156-E220 and E285-E3482, wherein the nucleic acid vector is a viral vector.
E350. The nucleic acid vector of E349, wherein the viral vector is selected from the group consisting of an adeno-associated virus (AAV), an adenovirus, and a lentivirus.
E351. The nucleic acid vector of E350, wherein the viral vector is an AAV vector.
E352. The nucleic acid vector of E351, wherein the AAV vector has an AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ, DJ/8, DJ/9, 7m8, PHP.B, PHP.eB, or PHP.S capsid.
E353. A composition comprising the nucleic acid vector of any one of E156-E220 and E285-E352 and a pharmaceutically acceptable carrier, diluent, or excipient.
E354. A cell comprising the polynucleotide of any one of E1-E155 and E221-E284 or the nucleic acid vector of any one of E156-E220 and E285-E352.
E355. The cell of E354, wherein the cell is a GJB2-expressing cell.
E356. The cell of E355, wherein the GJB2-expressing cell is a GJB2-expressing inner ear cell.
E357. The cell of any one of E354-E356, wherein the cell is a mammalian cell.
E358. The cell of E357, wherein the mammalian cell is a human cell.
E359. The cell of any one of E354-E358, wherein the cell is a cochlear supporting cell.
E360. A method of expressing an expression product in a GJB2-expressing cell, comprising contacting the GJB2-expressing cell with the nucleic acid vector of any one of E156-E220 and E285-E352 or the composition of E353.
E361. The method of E360, wherein the GJB2-expressing cell is a GJB2-expressing inner ear cell.
E362. The method of E361, wherein the GJB2-expressing inner ear cell is a cochlear supporting cell.
E363. The method of any one of E360-E362, wherein the contacting is in a subject (e.g., in vivo).
E364. A method of treating a subject having or at risk of developing GJB2-related hearing loss, comprising administering to an inner ear of the subject a therapeutically effective amount of the nucleic acid vector of any one of E156-E220 and E285-E352 or the composition of E353, wherein the expression product is Gjb2 or Gjb6.
E365. The method of E364, wherein the GJB2-related hearing loss is DFNB1, DFNA3, or hearing loss associated with Bart-Pumphrey syndrome, hystrix-like ichthyosis with deafness, keratitis-ichthyosis-deafness syndrome, palmoplantar keratoderma with deafness, or Vohwinkel syndrome.
E366. The method of E364, wherein the hearing loss is DFNB1 or DFNA3.
E367. The method of E366, wherein the subject has a mutation in GJB2, a mutation in GJB6, or a mutation in both GJB2 and GJB6.
E368. A method of treating a subject having or at risk of developing hearing loss (e.g., sensorineural hearing loss or deafness), comprising administering to an inner ear of the subject an effective amount of the nucleic acid vector of any one of E156-E220 and E285-E352 or the composition of E353.
E369. A method of treating a subject having or at risk of developing tinnitus, comprising administering to an inner ear of the subject an effective amount of the nucleic acid vector of any one of E156-E220 and E285-E352 or the composition of E353.
E370. A method of inducing or increasing cochlear hair cell regeneration in a subject in need thereof, comprising administering to an inner ear of the subject a therapeutically effective amount of the nucleic acid vector of any one of E156-E220 and E285-E352 or the composition of E353, wherein the enhancer has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 59 and the expression product is an expression product that can promote or increase cochlear supporting cell proliferation or differentiation of cochlear supporting cells into cochlear hair cells.
E371. A method of treating a subject having or at risk of developing hearing loss associated with damage to or loss of cochlear hair cells, comprising administering to an inner ear of the subject a therapeutically effective amount of the nucleic acid vector of any one of E156-E220 and E285-E352 or the composition of E353, wherein the enhancer has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 59 and the expression product is an expression product that can promote or increase cochlear supporting cell proliferation or differentiation of cochlear supporting cells into cochlear hair cells.
E372. The method of E370 or E371, wherein the polynucleotide encoding the expression product is a polynucleotide listed in Table 5.
E373. The method of E370 or E371, wherein the expression product is an inhibitory RNA directed to LATS1 and/or LATS2.
E374. A method of inducing or increasing differentiation of a cochlear supporting cell into a cochlear hair cell, comprising contacting the cochlear supporting cell with the nucleic acid vector of any one of E156-E220 and E285-E352 or the composition of E353, wherein the enhancer has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 59 and the expression product is Atoh1 or an Atoh1 variant, Pou4F3, Gfi1, or Ikzf2.
E375. A method of inducing or increasing cochlear supporting cell proliferation, comprising contacting the cochlear supporting cell with the nucleic acid vector of any one of E156-E220 and E285-E352 or the composition of E353, wherein the enhancer has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 59 and the expression product is Lgr5, Yap1, Tead2, or an inhibitory RNA directed to LATS1 and/or LATS2.
E376. The method of E374 or E375, wherein the contacting is in vivo (e.g., in a subject).
E377. A method of treating a subject having or at risk of developing genetic hearing loss associated with a mutation in a gene that is endogenously expressed in cochlear supporting cells, comprising administering to an inner ear of the subject a therapeutically effective amount of the nucleic acid vector of any one of E156-E220 and E285-E352 or the composition of E353, wherein the enhancer has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 59 and the polynucleotide encoding the expression product is a wild-type form of the gene that is mutated in cochlear supporting cells.
E378. The method of E377, wherein the genetic hearing loss is associated with a disease listed in Table 6 and the polynucleotide encoding the expression product is a transgene corresponding to a wild-type form of a gene that is mutated in said disease (e.g., a gene listed in the same row as the disease in Table 6).
E379. A method of improving cochlear supporting cell function or cochlear supporting cell survival, comprising contacting the cochlear supporting cell with the nucleic acid vector of any one of E156-E220 and E285-E352 or the composition of E353.
E380. The method of E379, wherein the contacting is in vivo (e.g., in a subject).
E381. A method of improving cochlear supporting cell function or cochlear supporting cell survival in a subject in need thereof, comprising administering to an inner ear of the subject a therapeutically effective amount of the nucleic acid vector of any one of E156-E220 and E285-E352 or the composition of E353.
E382. The method of any one of E363, E370, E376, E380, and E381, wherein the subject has or is at risk of developing hearing loss (e.g., sensorineural hearing loss or deafness).
E383. The method of any one of E368, E371-E373, and E382, wherein the hearing loss is acquired hearing loss.
E384. The method of E383, wherein the acquired hearing loss is noise-induced hearing loss, age-related hearing loss, disease or infection-related hearing loss, head trauma-related hearing loss, or ototoxic drug-induced hearing loss.
E385. The method of E368, E371-E373, and E382, wherein the hearing loss is genetic hearing loss.
E386. The method of any one of E377, E378, and E385, wherein the genetic hearing loss is autosomal dominant hearing loss, autosomal recessive hearing loss, or X-linked hearing loss.
E387. The method of any one of E362 and E370-E381, wherein the cochlear supporting cell is a mammalian cochlear supporting cell.
E388. The method of E387, wherein the mammalian cochlear supporting cell is a human cochlear supporting cell.
E389. The method E384, wherein the ototoxic drug is an aminoglycoside, an antineoplastic drug, ethacrynic acid, furosemide, a salicylate, or quinine.
E390. The method of any one of E363-E373, E376-E378, and E380-E389, wherein the method further comprises evaluating the hearing of the subject prior to administering the nucleic acid vector or composition.
E391. The method of any one of E363-E373, E376-E378, and E380-E390, wherein the method further comprises evaluating the hearing of the subject after administering the nucleic acid vector or composition.
E392. The method of any one of E363-E391, wherein the nucleic acid vector or composition is locally administered.
E393. The method of E392, wherein the nucleic acid vector or composition is administered to the inner ear.
E394. The method of E392, wherein the nucleic acid vector or composition is administered to the middle ear.
E395. The method of E392, wherein the nucleic acid vector or composition is administered transtympanically or intratympanically.
E396. The method of E392, wherein the nucleic acid vector or composition is administered into the perilymph.
E397. The method of E392, wherein the nucleic acid vector or composition is administered into the endolymph.
E398. The method of E392, wherein the nucleic acid vector or composition is administered to or through the oval window.
E399. The method of E392, wherein the nucleic acid vector or composition is administered to or through the round window.
E400. The method of any one of E363-E399, wherein the nucleic acid vector or composition is administered in an amount sufficient to prevent or reduce hearing loss, prevent or reduce tinnitus, delay the development of hearing loss, slow the progression of hearing loss, improve hearing, increase or induce expression of an expression product in GJB2-expressing cells, increase cochlear hair cell numbers, increase cochlear hair cell regeneration, increase cochlear supporting cell proliferation, promote or increase cochlear supporting cell survival, induce or increase the differentiation of cochlear supporting cells into cochlear hair cells, or improve cochlear supporting cell function.
E401. The method of any one of E363-E373, E376-E378, and E380-E400, wherein the subject is a human subject.
E402. A kit comprising the polynucleotide of any one of E1-E155 and E221-E284, the nucleic acid vector of any one of E156-E220 and E285-E352, or the composition of E353.

### Other Embodiments

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention. Other embodiments are in the claims.

1. A polynucleotide comprising a GJB2 promoter comprising a first region having at least 85% sequence identity to SEQ ID NO: 1 or a functional portion or derivative thereof comprising at least one of SEQ ID NOs: 3-12 operably linked to a second region having at least 85% sequence identity to SEQ ID NO: 2 or a functional portion or derivative thereof comprising at least one of SEQ ID NOs: 20-24, wherein the distance between the first region and the second region in the polynucleotide is no more than 1 kilobase (kb).

2. The polynucleotide of claim 1, wherein the first region has at least 85% sequence identity to SEQ ID NO: 1.

3. The polynucleotide of claim 2, wherein the first region has the sequence of SEQ ID NO: 1

4. The polynucleotide of claim 1, the first region has at least 85% sequence identity to any one of SEQ ID NOs: 3-19.

5. The polynucleotide of claim 4, the first region has the sequence of any one of SEQ ID NOs: 3-19.

6. The polynucleotide of claim 5, wherein the first region has the sequence of SEQ ID NO: 3.

7. The polynucleotide of any one of claims 1-6, wherein the second region has at least 85% sequence identity to SEQ ID NO: 2.

8. The polynucleotide of claim 7, wherein the second region has the sequence of SEQ ID NO: 2.

9. The polynucleotide of any one of claims 1-6, wherein the second region has at least 85% sequence identity to any one of SEQ ID NOs: 20-28.

10. The polynucleotide of claim 9, wherein the second region has the sequence of any one of SEQ ID NOs: 20-28.

11. The polynucleotide of claim 10, wherein the second region has the sequence of SEQ ID NO: 20.

12. The polynucleotide of claim 10, wherein the second region has the sequence of SEQ ID NO: 21.

13. The polynucleotide of claim 10, wherein the second region has the sequence of SEQ ID NO: 22.

14. The polynucleotide of claim 10, wherein the second region has the sequence of SEQ ID NO: 25.

15. The polynucleotide of claim 10, wherein the second region has the sequence of SEQ ID NO: 26.

16. The polynucleotide of any one of claims 1-15, wherein the first region is fused directly to the second region without a linker.

17. The polynucleotide of claim 16, wherein the GJB2 promoter has at least 85% sequence identity to SEQ ID NO: 30.

18. The polynucleotide of claim 17, wherein the GJB2 promoter has the sequence of SEQ ID NO: 30.

19. The polynucleotide of claim 16, wherein the GJB2 promoter has at least 85% sequence identity to SEQ ID NO: 31.

20. The polynucleotide of claim 19, wherein the GJB2 promoter has the sequence of SEQ ID NO: 31.

21. The polynucleotide of claim 16, wherein the GJB2 promoter has at least 85% sequence identity to SEQ ID NO: 32.

22. The polynucleotide of claim 21, wherein the GJB2 promoter has the sequence of SEQ ID NO: 32.

23. The polynucleotide of claim 16, wherein the GJB2 promoter has at least 85% sequence identity to SEQ ID NO: 33.

24. The polynucleotide of claim 23, wherein the GJB2 promoter has the sequence of SEQ ID NO: 33.

25. The polynucleotide of claim 16, wherein the GJB2 promoter has at least 85% sequence identity to SEQ ID NO: 34.

26. The polynucleotide of claim 25, wherein the GJB2 promoter has the sequence of SEQ ID NO: 34.

27. The polynucleotide of claim 16, wherein the GJB2 promoter has at least 85% sequence identity to SEQ ID NO: 35.

28. The polynucleotide of claim 27, wherein the GJB2 promoter has the sequence of SEQ ID NO: 35.

29. A polynucleotide comprising a GJB2 promoter having at least 85% sequence identity to SEQ ID NO: 13.

30. A polynucleotide comprising a GJB2 promoter having at least 85% sequence identity to SEQ ID NO: 37.

31. The polynucleotide of any one of claims 1-30, wherein the GJB2 promoter is operably linked to a polynucleotide encoding an expression product.

32. The polynucleotide of claim 31, wherein the expression product is a protein, a short hairpin RNA (shRNA), an antisense oligonucleotide (ASO), a component of a gene editing system, or a microRNA.

33. The polynucleotide of claim 31, wherein the expression product is an expression product that is endogenously expressed in a GJB2-expressing cell.

34. The polynucleotide of claim 31, wherein the expression product is an expression product that is endogenously expressed in a GJB2-expressing inner ear cell.

35. The polynucleotide of claim 33 or 34, wherein the expression product is Gjb2 or Gjb6.

36. The polynucleotide of claim 31, wherein expression product is a heterologous expression product.

37. The polynucleotide of any one of claims 1-36, wherein the polynucleotide further comprises a GJB2 enhancer operably linked to the GJB2 promoter.

38. The polynucleotide of claim 37, wherein the GJB2 enhancer has at least 85% sequence identity to a nucleotide sequence of any one of SEQ ID NOs: 52-63.

39. The polynucleotide of claim 37 or 38, wherein the GJB2 enhancer has the sequence of any one of SEQ ID NOs: 52-63.

40. The polynucleotide of any one of claims 37-39, wherein the GJB2 enhancer is located 5′ of the promoter.

41. The polynucleotide of any one of claims 37-39, wherein the GJB2 enhancer is located 3′ of the promoter.

42. A nucleic acid vector comprising the polynucleotide of any one of claims 1-41.

43. The nucleic acid vector of claim 42, wherein the nucleic acid vector is a viral vector.

44. The nucleic acid vector of claim 43, wherein the viral vector is an adeno-associated virus vector.

45. A composition comprising the nucleic acid vector of any one of claims 42-44 and a pharmaceutically acceptable carrier, diluent, or excipient.

46. A method of expressing an expression product in a GJB2-expressing cell, comprising contacting the GJB2-expressing cell with the nucleic acid vector of any one of claims 42-44 or the composition of claim 45.

47. The method of claim 46, wherein the GJB2-expressing cell is a GJB2-expressing inner ear cell.

48. The method of claim 46 or 47, wherein the contacting is in a subject.

49. A method of treating a subject having or at risk of developing GJB2-related hearing loss, comprising administering to an inner ear of the subject a therapeutically effective amount of the nucleic acid vector of any one of claims 42-44 or the composition of claim 45, wherein the expression product is Gjb2 or Gjb6.

50. The method of claim 49, wherein the GJB2-related hearing loss is DFNB1 or DFNA3.

51. The method of claim 50, wherein the subject has a mutation in GJB2, a mutation in GJB6, or a mutation in both GJB2 and GJB6.

52. A method of inducing or increasing cochlear hair cell regeneration in a subject in need thereof, comprising administering to an inner ear of the subject a therapeutically effective amount of the nucleic acid vector of any one of claims 42-44 or the composition of claim 45, wherein the nucleic acid vector comprises a GJB2 enhancer having at least 85% sequence identity to SEQ ID NO:59 and the expression product is an expression product that can promote or increase cochlear supporting cell proliferation or differentiation of cochlear supporting cells into cochlear hair cells.

53. A method of treating a subject having or at risk of developing hearing loss associated with damage to or loss of cochlear hair cells, comprising administering to an inner ear of the subject a therapeutically effective amount of the nucleic acid vector of any one of claims 42-44 or the composition of claim 45, wherein the nucleic acid vector comprises a GJB2 enhancer having at least 85% sequence identity to SEQ ID NO: 59 and the expression product is an expression product that can promote or increase cochlear supporting cell proliferation or differentiation of cochlear supporting cells into cochlear hair cells.

54. The method of claim 52 or 53, wherein the polynucleotide encoding the expression product is a polynucleotide listed in Table 5.

55. A method of treating a subject having or at risk of developing genetic hearing loss associated with a mutation in a gene that is endogenously expressed in cochlear supporting cells, comprising administering to an inner ear of the subject a therapeutically effective amount of the nucleic acid vector of any one of claims 42-44 or the composition of claim 45, wherein the enhancer has at least 85% sequence identity to SEQ ID NO: 59 and the polynucleotide encoding the expression product is a wild-type form of the gene that is mutated in cochlear supporting cells.

56. The method of claim 55, wherein the genetic hearing loss is associated with a disease listed in Table 6 and the polynucleotide encoding the expression product is a wild-type form of a gene that is mutated in said disease.
